(19) United States
(12) Patent Application Publication
(10) Pub. No.: US 2013/0195884 A1
(43) Pub. Date: Aug. 1, 2013

(54) NOVEL MODULATORS OF TRAIL SIGNALLING

(71) Applicants: Michael Boutros, Heidelberg (DE); Henning Walczak, London (GB); Sandra Steinbrink, Mainz (DE); Christina Falschlehner, Heidelberg (DE)

(72) Inventors: Michael Boutros, Heidelberg (DE); Henning Walczak, London (GB); Sandra Steinbrink, Mainz (DE); Christina Falschlehner, Heidelberg (DE)

(73) Assignee: Deutsches Krebsforschungszentrum, Heidelberg (DE)

(21) Appl. No.: 13/519,955

(22) PCT Filed: Dec. 30, 2010
(86) PCT No.: PCT/EP2010/070919

§ 371 Date: Nov. 14, 2012

Related U.S. Application Data
(60) Provisional application No. 61/291,490, filed on Dec. 31, 2009.

(51) Int. Cl. A61K 38/17; A61K 38/48; A61K 38/53; G01N 33/68; A61K 45/06; A61K 39/395; C12Q 1/68; A61K 38/46; A61K 38/50

(52) U.S. Cl. CPC A61K 38/1709; A61K 38/177; A61K 38/465; A61K 38/4813; A61K 38/53; A61K 38/50; A61K 45/06; A61K 39/3955; C12Q 1/6876; G01N 33/6863
USPC 424/158.1; 514/44 R; 514/18.9; 530/350; 536/23.5; 424/94.6; 435/196; 536/23.2; 424/94.62; 435/212; 424/94.5; 435/183; 435/227; 514/16.6; 514/17.9; 514/3.7; 514/17.7; 514/13.5; 514/7.3; 514/3.8; 424/172.1; 530/389.1; 435/6.12; 506/10; 435/7.21; 506/9; 604/501

(57) ABSTRACT

The present invention relates to an agent for use as a modulator of apoptosis-factor-associated cell death, apoptosis, cell survival, migration and/or proliferation, a method of diagnosing or monitoring apoptosis-factor-associated conditions or disorders as well as a method of identifying a modulator of apoptosis-factor-associated cell death, apoptosis, cell survival, migration and/or proliferation. Preferably, the invention relates to TRAIL-induced cell death and/or TRAIL-induced apoptosis. Preferably the agents are used to stimulate and/or enable TRAIL-induced cell death or to inhibit TRAIL-induced cell death. The preferable use of the diagnostic tools is to diagnose sensitivity or resistance to TRAIL-induced cell death or induction of sensitivity or resistance to TRAIL-induced cell death by an agent.

The present invention relates to an agent for use as a modulator of apoptosis-factor-associated cell death, apoptosis, cell survival, migration and/or proliferation, a method of diagnosing or monitoring apoptosis-factor-associated conditions or disorders as well as a method of identifying a modulator of apoptosis-factor-associated cell death, apoptosis, cell survival, migration and/or proliferation. Preferably, the invention relates to TRAIL-induced cell death and/or TRAIL-induced apoptosis. Preferably the agents are used to stimulate and/or enable TRAIL-induced cell death or to inhibit TRAIL-induced cell death. The preferable use of the diagnostic tools is to diagnose sensitivity or resistance to TRAIL-induced cell death or induction of sensitivity or resistance to TRAIL-induced cell death by an agent.

Apoptosis is a form of programmed cell death that has evolved to allow for tissue remodelling and homeostasis and to remove unwanted and potentially dangerous cells from an organism (Los, Wesselborg et al. 1999; Vaux and Korsmeyer 1999). For example, during embryogenesis apoptosis is required for the shaping of limbs. Since more than a thousand billion cells are created in an adult human being every day, an equal number has to die at the same time. A disturbance in this balance between proliferating and dying cells can lead to several disorders. In AIDS, stroke or neurodegenerative diseases cells are lost as a result of extensive cell death, whereas in cancer or autoimmune disorders a reduction in apoptosis can often be observed (Mattson 2000; Fischer and Schulze-Osthoff 2005).

### Forms of Cell Death

Mammalian cells can die through different, biochemically and morphologically distinct pathways. There are three main forms of cell death, namely apoptosis, necrosis and autophagy. Apoptosis is a form of programmed cell death, in which the cell dies in a controlled manner by regulated activation of distinct proteins. Typical morphological features like cell shrinkage, chromatin condensation and cytoplasmic membrane blebbing can be observed during apoptosis (Nagata 2000; Kihlmark, lmreh et al. 2001). Apoptotic cells shrink in size and break into smaller pieces called apoptotic bodies that are recognised and phagocytosed by other cells. An important biochemical feature of apoptotic cell death is the fragmentation of nuclear DNA into multiples of ˜200 base-pair (bp) oligonucleotide fragments (Wyllie 1980). These fragments form the characteristic “DNA ladder” of apoptotic cells in an agarose gel (Cohen and Duke 1984). Another feature of apoptotic cells is the exposure of phosphatidylserine (PS) on the outer plasma membrane which serves as an important “eat me” signal. Subsequently, these cells are phagocytosed by neighbouring cells and macrophages (Fadok, Voelker et al. 1992). As the release of cytoplasmic content from apoptotic cells is prevented, no inflammatory response is initiated. Two well-characterised signalling pathways are described to induce apoptosis in mammalian cells: the intrinsic and extrinsic pathways. The intrinsic pathway is induced by several stress situations, e.g. DNA damage, and leads to the release of certain proteins from the mitochondria. This pathway is also referred to as “Bcl-2 controlled” or “mitochondrial pathway” because it is triggered and controlled by members of the Bcl-2 protein family at mitochondrial outer membrane (Youle and Strasser 2008).

The extrinsic pathway is activated when cell death receptors are oligomerised by their cognate ligands. After binding of the ligand, the death-inducing signalling complex (DISC) is formed which is necessary for the subsequent signal transduction by intracellular proteins. The main players in this respect are cysteine-dependent, aspartate-specific proteases (caspases) which cleave a variety of cellular substrates, contributing to the destruction of the cell. All known death receptors belong to the tumor necrosis factor (TNF) receptor superfamily (Sprick and Walczak 2004; Hehlgans and Pfeffer 2005).

In contrast to apoptosis, necrosis is characterised by swelling of the cell, followed by plasma membrane disruption and the uncontrolled release of cytoplasmic content which provokes an inflammatory response. In addition, necrotic death is energy independent and no role for mitochondria or caspases has been described. Depending on their applied concentration, several drugs can lead to either apoptosis or necrosis (Kroemer, Petit et al. 1995). This observation suggests that apoptosis and necrosis are mechanistically linked. Furthermore, the blockage of an apoptotic signal by inhibition of caspases can result in necrotic cell death (Lemaire, Andreau et al. 1998; Vercammen, Beyaert et al. 1998).

A third process, autophagy, has also been proposed to be a form of programmed cell death. Autophagy is usually responsible for the degradation of long-lived proteins and is the only known pathway for the degradation of whole organelles (Klionsky and Emr 2000). It is important to bear in mind that under conditions of nutrient deprivation, autophagy is thought to act, at least initially, as a survival mechanism. During autophagy, long-lived proteins or organelles are sequestered into a double membrane vesicle called the autophagosome. Autophagy-related genes (atg) are essential for the formation of the autophagosome which then fuses with a lysosome and the contents are subjected to enzymatic digestion. The visualisation of autophagosomes in dying cells has led to the belief that autophagy is a non-apoptotic form of programmed cell death. However, in cells with an intact apoptotic machinery, it is unclear whether autophagy is indeed a direct death execution pathway. Autophagic cell death has mainly been observed in cells in which the apoptotic machinery was non-functional or blocked, e.g. when caspases were blocked by the use of the caspase-inhibitor zVAD-fmk. Under these conditions, autophagic cell death is characterised by the appearance of autophagic vacuoles and the early degradation of organelles.

In contrast to apoptosis, caspase activation and DNA fragmentation only occur, if at all, at a very late stage during autophagy. In many cases, morphologic features of autophagic and apoptotic cell death or of autophagic and necrotic cell death are observed in the same cell. Studies using RNA interference against two autophagy-related genes, atg7 and atg6 (also known as Beclin-1), and the caspase inhibitor zVAD-fmk, excluded the possibility that autophagy directly triggers apoptosis and thereby leads to cell death (Yu, Alva et al. 2004). Another study using embryonic fibroblasts from Bax/Bak double knockout mice, which are resistant to mitochondrial pathway of apoptosis induction, demonstrated that those cells underwent non-apoptotic death after stimulation by the chemotherapeutic agent etoposide or staurosporine. By means of electronic microscopy it was confirmed that this cell death was associated with autophagosomes (Shimizu, Kanaseki et al. 2004). These data support the theory that cells will preferentially die by apoptosis and will only use alternative mechanisms, like autophagy, if exposed to very strong death stimuli and a non-functional apoptosis machinery (Lockshin and Zakeri 2004). Interestingly, atg-6 (Beclin-1) was recently identified as a novel BH3-only protein which is capable of binding to Bcl-2 and Bcl-X_{L} (Oberstein, Jeffrey et al. 2007). Beclin-1 knockout mice (beclin-1_{-/-}) die early in embryogenesis and heterozygous beclin-1 knockout mice (beclin-1_{+/-}) have an increased incidence of spontaneous tumor formation which could be due to defective apoptosis mechanisms (Yue, Jin et al. 2003). Bcl-2 has been shown to directly inhibit autophagic cell death via interaction with Beclin-1 (Pattingre, Tassa et al. 2005). Furthermore, BH3 mimetics can induce autophagy by competitively disrupting the interaction between Beclin-1 and Bcl-2/Bcl-X_{L} (Maiuri, Criollo et al. 2007). These data suggest that there is functional crosstalk between apoptosis and autophagy involving members of the Bcl-2 family.

Besides apoptosis, necrosis and autophagy, other forms of cell death have been suggested including “necroptosis” and “caspase-independent cell death”. The term necroptosis was first observed by Degterev et al. (Degterev, Huang et al. 2005). Necroptosis is characterised by necrotic cell death morphology and activation of autophagy. It seems to contribute to delayed ischemic brain injury in mice through a mechanism distinct from apoptosis. The authors indentified a molecule called necrostatin-1, which inhibited necroptosis induced by Fas ligand (FasL/CD95L) and TNF-α in the absence of caspase activity. However, this type of cell death has not been observed under normal physiological conditions, i.e. in the presence of an intact apoptotic machinery.

The same is true for caspase-independent cell death. As the name implies, this type of cell death occurs independently of caspase activity. However, in experiments where caspase independent cell death was observed, the normal apoptotic machinery was blocked. Cathepsins have been implicated in playing an important role in caspase-independent cell death. Yet, caspase and cathepsin activation can occur in parallel and it is unclear whether cathepsins alone, without any caspase activation, can induce cell death (Vercammen, Beyaert et al. 1998; Foghsgaard, Wissing et al. 2001).

### The Bcl-2 Family

The B cell lymphoma 2 (Bcl-2) protein family consists of three subfamilies which share four characteristic domains called the Bcl-2 homology (BH) domains, BH1-BH4. The BH domains are known to be crucial for the function of Bcl-2 family members, as deletion of these domains affects survival/apoptosis rates. The anti-apoptotic Bcl-2 proteins, such as Bcl-2 and Bcl-X_{L}, conserve all four BH domains. The BH domains also serve to subdivide the pro-apoptotic Bcl-2 proteins into the ones with 11 several BH domains (e.g., Bax and Bak) and the ones which have only the BH3 domain (e.g., Bid, Bim, Bad), also called the “BH3-only” proteins.

The anti-apoptotic members like Bcl-2, BCI-X_{L} or Mcl-1 are associated with the mitochondrial outer membrane and stabilise mitochondrial integrity. Their function is counteracted by the multidomain pro-apoptotic members like Bak, Bax and Bok, which associate with the outer mitochondrial membrane during apoptosis, destabilising its integrity to release pro-apoptotic factors from the intermembrane space. The pro-apoptotic Bcl-2 family members are activated by interaction with the BH3-only proteins. Besides activating pro-apoptotic Bcl-2 proteins, the BH3-only proteins can, upon their own activation, interact with pro-survival Bcl-2 members and neutralise their anti-apoptotic function (Strasser 2005). Furthermore, it has been shown that the BH3-only proteins Bim and Puma bind promiscuously to anti-apoptotic Bcl-2 family members whereas other BH3-only proteins bind selectively to them (Willis, Fletcher et al. 2007; Huang and Sinicrope 2008).

### Caspases—the Executioner of Cell Death

Cysteine aspartate specific proteases (caspases) belong to the family of cystein proteases and play an essential role in apoptosis. Upon apoptosis induction a caspase cascade is initiated that leads to the cleavage of a variety of cellular substrates, contributing to the destruction of the cell and ultimately leading to cell death. Caspases are highly specific proteases that cleave their substrates after specific tetrapeptide motifs (P4-P3-P2-P1) where P1 is always an aspartate (Asp) residue, for example DEVD (Asp-Glu-Val-Asp), the tetrapeptide that is recognized by caspase-3. Furthermore, caspases are synthesized as inactive enzyme precursors (zymogens) to ensure rapid activation in response to appropriate stimuli. The caspase family can be divided into different groups according to their structure and function. Initiator caspases are synthesized as inactive procaspases which consist of a long prodomain, a large subunit and a small subunit. The prodomain either contains a caspase recruitment domain (CARD), as is the case for caspases 2 and 9, or a death effector domain (DED) as is the case for caspases 8 and 10. These prodomains enable the caspases to interact with other proteins that regulate their activation. Activation of initiator caspases occurs at multiprotein complexes including the DISC (Walczak and Haas 2008), the apoptosome (Riedl and Salvesen 2007), the inflammasome (Martinon and Tschopp 2007) and the piddosome (Tinel and Tschopp 2004).

### The TNF/TNF-Receptor Superfamily

In the middle of the 20th century, a substance capable of inducing tumor regression in tumor-bearing mice was isolated from cell-free extracts of gram-negative bacteria. This substance turned out to be a constituent of the bacterial cell wall, called lipopolysaccharide (LPS) or endotoxin. However, LPS itself did not lead to necrosis of the tumor but instead a factor found in the serum of LPS-treated mice. This was named tumor necrosis factor (TNF), due to its ability to cause necrosis of the tumor tissue (Carswell, Old et al. 1975). In the following years, several other factors were isolated that were able to induce killing of lymphocytes or tumor cells. Of note, all of them showed considerable similarity to TNF. After cloning of the respective receptors, it became clear that the TNF/TNF-R (TNF-receptor) superfamily is a complex network consisting of several receptors and interacting ligands.

The receptors of the TNF family exert their effects mainly via two intracellular signal transduction pathways. The first causes changes in gene expression, principally by activating the NF-κB (nuclear factor kappa-light-chain-enhancer of activated B cells) or JNK (c-Jun Nterminal kinase) signalling cascade, leading to differentiation or proliferation (Gaur and Aggarwal 2003). The second pathway leads to the induction of a caspase cascade, which utimately results in cell death (Denault and Salvesen 2008).

A subgroup of the TNF-R family, the death receptors, activates the extrinsic death pathway. Death receptors are characterised by the presence of an intracellular protein domain, referred to as the death domain (DD). This is necessary for the assembly of the DISC which acts as an activation platform for the initiator caspases 8 and 10.

Amongst the death receptors, the TRAIL system stands out due to its complex receptor system. TRAIL can bind two apoptosis-inducing receptors, TRAIL-R1 (DR4) and TRAIL-R2 (DR5), as well as two cell-bound receptors incapable of transmitting an apoptotic signal, TRAIL-R3 (LIT, DcR1) and TRAIL-R4 (TRUNDD, DcR2), sometimes also called decoy receptors, and lastly, a soluble receptor called osteoprotegerin (OPG).

TRAIL was first identified based on its sequence homology to other TNF superfamily members (Wiley, Schooley et al. 1995). Human TRAIL is expressed as a type II transmembrane protein consisting of 281 amino acids and shows the highest homology to CD95L. Similarly to CD95L and TNF, the membrane-bound form of TRAIL can be cleaved by metalloproteases to form a soluble trimer. A zinc ion, buried at the trimer interface, is coordinated by the single cysteine residue of each monomer to stabilize the soluble trimer (Cha, Kim et al. 1999; Cha, Shin et al. 1999; Hymowitz, O'Connell et al. 2000).

In contrast to the human TRAIL/TRAIL-R system, mice only possess one apoptosis-inducing receptor called murine TRAIL-R (MK—murine killer, mDR5) which is equally homologous to human TRAIL-R1 and TRAIL-R2 (Wu, Burns et al. 1999). The other murine receptors, mDcR1, mDcR2L and the splice variant mDcR2S share a clustered locus (Schneider, Olson et al. 2003), but have otherwise not been studied so far.

TRAIL has been shown to kill a variety of tumor cells while normal cells are resistant to TRAIL-induced apoptosis (Ashkenazi, Pai et al. 1999; Walczak, Miller et al. 1999). This property has made TRAIL and agonistic antibodies to TRAIL-R1 and TRAIL-R2 promising novel biotherapeutic agents for cancer therapy. Binding of TRAIL to TRAIL-R1 or TRAIL-R2 leads to receptor trimerisation and assembly of the DISC (Walczak and Haas, 2008). The adaptor molecule Fas Associated Death Domain (FADD) translocates to the DISC where it interacts with the DD of the receptors. Via its second functional domain, the death effector domain (DED), FADD recruits procaspase-8 and -10 to the DISC where these caspases are auto-catalytically activated. The initiator caspases 8 and 10 then activate the downstream effector caspase-3 which in turn activates further effector caspases leading to the cleavage of several proteins, finally leading to cell death.

The enzyme poly (ADP-ribose) polymerase (PARP) was one of the first proteins identified as a substrate of the effector caspase-3. PARP is usually involved in repair of DNA damage, but upon caspase-3 cleavage of PARP DNA repair is inhibited. Caspase cleavage also leads to the degradation of lamins that maintain the shape of the nucleus, resulting in chromatin condensation. Furthermore, the inhibitor of caspase activated DNase (ICAD) is cleaved, thereby releasing caspase activated DNase (CAD) which leads to the fragmentation of DNA, a classical hallmark of apoptotic cells (Wyllie 1980). Depending on the strength of DISC formation and the requirement of the mitochondrial pathway for cell death execution, cells are classified as type I or type II. In type I cells, the DISC-induced caspase cascade is sufficient for apoptotic cell death and so overexpression of Bcl-2 does not affect the apoptotic outcome. In contrast, type II cells show weaker DISC formation and to undergo apoptosis they depend on activation of the mitochondrial apoptotic pathway, which as already introduced above, is also called intrinsic or Bcl-2 controlled pathway. The death receptor-mediated extrinsic and the mitochondrial intrinsic apoptosis pathways are connected via the BH3-only protein Bid. Upon DISC formation Bid is cleaved into truncated Bid (tBid) by caspase-8 or caspase-10. Tbid translocates to the mitochondria and activates Bax and Bak, leading to the release of cytochrome c and other pro-apoptotic proteins from the mitochondria (Waterhouse, Ricci et al. 2002).

Cytochrome c, together with Apaf-1, dATP and caspase-9 forms the apoptosome, which serves as activation platform for caspase-9 which in turn activates downstream effector caspases. Additionally, Smac/DIABLO, which is also released from the mitochondria during apoptosis, counteracts the function of XIAP allowing for full activation of caspases 3, 7 and 9 (Shi 2004). A schematic view of the human TRAIL signalling network is shown in FIG. 1.

### TRAIL-Induced Apoptosis: Resistance Versus Sensitivity

Around 50% of all tumor cell lines and most primary tumors are resistant to apoptosis induction by TRAIL. Yet many tumors can be sensitized to TRAIL-induced apoptosis by various chemotherapeutic agents (Zisman, Ng et al. 2001; Munshi, McDonnell et al. 2002; Belyanskaya, Marti et al. 2007), cytokines (Fulda and Debatin 2006; Micali, Cheung et al. 2007), proteasome inhibitors (Ganten, Koschny et al. 2005), histone deacetylase (HDAC) inhibitors (Dzieran, Beck et al. 2008) or γ-irradiation (Maduro, de Vries et al. 2008). Several mechanisms that determine sensitivity versus resistance have been proposed for various drugs. However, these mechanisms are often poorly understood. Treatment with chemotherapeutic agents has been shown to upregulate TRAIL-R1 and TRAIL-R2 in a p53-dependent or -independent manner (Sheikh, Burns et al. 1998; Wu, Kim et al. 2000; Ganten, Haas et al. 2004). Although the upregulation of apoptosis-inducing TRAIL-Rs can contribute to the killing, it might alone not be sufficient to sensitise cells to TRAIL-induced apoptosis. For example, TRAIL-R expression and sensitivity to TRAIL-induced apoptosis showed poor correlation (Belyanskaya, Marti et al. 2007). Moreover, colon cancer cell lines could be sensitized to TRAIL-induced apoptosis independent of receptor upregulation (Lacour, Hamman et al. 2001). In line with these results, Ganten et al. reported that upregulation of TRAIL-R1 and TRAIL-R2 after treatment with 5-Fluorouracil (5-FU) is not essential for sensitisation to TRAIL-induced apoptosis (Ganten, Haas et al. 2004).

Furthermore, the same authors showed that caspase-8 recruitment to, and activation at, the DISC is facilitated by the downregulation of cFLIP while FADD levels remained unchanged. Consequently, the ratio of caspase-8 to cFLIP at the DISC is shifted enabling efficient caspase-8 activation which in turn leads to effector caspase activation and finally cell death (Ganten, Haas et al. 2004). Similarly, proteasome inhibitors have also been shown to sensitise cells to TRAIL-induced apoptosis by shifting the ratio of cFLIP, caspase-8 and FADD at the TRAIL-DISC leading to an increased DISC formation and apoptotic signal transduction (Ganten, Koschny et al. 2005).

Thus intracellular mechanisms, for example the expression levels of pro- and anti-apoptotic proteins are probably critical for apoptosis induction by TRAIL. Concerning the initial signalling events at the TRAIL DISC, expression of the anti-apoptotic death domain containing proteins cFLIP and PED/PEA-15 (protein containing a DED/phosphoprotein enriched in astrocytes 15) is critical. They can compete with caspase-8 for FADD binding and can therefore block caspase-8 activation (Walczak and Haas 2008). In neural stem and progenitor cells, high expression levels of PED/PEA-15 were shown to inhibit apoptosis induced by TRAIL and inflammatory cytokines (Ricci-Vitiani, Pedini et al. 2004). Inflammatory cytokines, especially IL-4 and IL-10, in turn were shown to be intrinsically produced by primary epithelial cancer cells from colon, breast and lung carcinomas, thereby regulating the expression of anti-apoptotic proteins PED/PEA-15, cFLIP, Bcl-X_{L} and Bcl-2 (Todaro, Lombardo et al. 2008).

The balance of Bcl-2 family members plays an important role in determining sensitivity versus resistance to apoptosis. High expression of anti-apoptotic proteins, especially Bcl-2 and Bcl-X_{L}, has been observed in several cancers (Reed 1995; Olopade, Adeyanju et al. 1997). The ratio of pro-apoptotic to anti-apoptotic Bcl-2 family members determines whether mitochondrial outer membrane permeabilisation (MOMP) can occur after apoptotic stimuli.

Overexpression of Bcl-2 or Bcl-X_{L} can block the release of cytochrome c, Smac/DIABLO and other pro-apoptotic factors from the mitochondria. Cytochrome c is essential for the formation of the apoptosome whereas Smac/DIABLO counteracts the function of IAPs that inhibit full activation of caspases 3, 7 and 9 (Shi et al., 2004). Therefore, resistance to TRAIL can be caused by high levels of anti-apoptotic Bcl-2 family members and high levels of IAPs.

The multikinase inhibitor sorafenib, which sensitizes various cancer cells to TRAIL-induced apoptosis, has been shown to downregulate the Bcl-2 family member Mcl-1 as well as clAP2 by blocking NF-κB (Ricci, Kim et al. 2007). Moreover, the BH3 mimetic ABT-737, a recently described compound from Abbott Laboratories (Oltersdorf, Elmore et al. 2005), was reported to selectively target certain anti-apoptotic Bcl-2 proteins and to efficiently induce apoptosis via Bak/Bax if Mcl-1 is neutralised (van Delft, Wei et al. 2006). In addition, ABT-737 enhances TRAIL-induced apoptosis by releasing Bim and Bak and enhancing the conformational change of Bax (Huang and Sinicrope 2008). Compounds called “Smac mimetics” have also been shown to sensitise cells to TRAIL-induced apoptosis by sequestering IAPs, in particular XIAP (Zobel, Wang et al. 2006; Petrucci, Pasquini et al. 2007; Nikolovska-Coleska, Meagher et al. 2008).

Although many drugs have been shown to sensitise cancer cells to TRAIL-induced apoptosis, little is known about the exact biochemical mechanism. Changes at the DISC, the level of proapoptotic Bcl-2 family members and the down-regulation of IAPs can favour apoptosis induction by TRAIL. However, the interplay of many factors is required for TRAIL-induced apoptosis and some of these factors might not have been identified yet.

### Non-Apoptotic Signalling by TRAIL

Intriguingly, TRAIL signaling does not only lead to apoptosis, but can also induce non-apoptotic pathways, which include the activation of NF-κB, proteine kinase B (PKB/AKT) and mitogen-activated kinases (MAPKs). It has been shown that NF-κB activation is mediated by TRAIL-R1, TRAIL-R2 and interestingly also by TRAIL-R4 (Degli-Esposti, Dougall et al. 1997; MacFarlane 2003). Furthermore, receptor (TNFRSF)-interacting serine/threonine protein kinase 1 (RIP1) has been detected in the TRAIL DISC (Harper, Farrow et al. 2001) and seems to mediate TRAIL-induced I kappa B kinase (IKK) activation. RIP1-29 deficient fibroblasts show no IKK and very little JNK activation upon TRAIL stimulation (Lin, Devin et al. 2000). JNK activation by TRAIL occurs in a caspase-dependent fashion and is mediated by TRAIL-R1 and TRAIL-R2 (Hu, Johnson et al. 1999). Although JNK activation in the TNF pathway has been associated with apoptosis induction, it is not required for TRAIL-induced apoptosis (MacFarlane, Cohen et al. 2000). TRADD has also been shown to be recruited to TRAIL-R1 and TRAIL-R2 (Chaudhary, Eby et al. 1997; Schneider, Thome et al. 1997). However, these studies were performed under overexpression conditions and may not resemble the native complex. It was shown that TRADD recruitment to TNF-R1 leads to the recruitment of several signaling proteins including TRAF2 (Hsu, Huang et al. 1996). TRAF2 has been implicated to play a role in TRAIL-induced NF-κB activation. Yet, this function of TRAF2 is controversial. It has been demonstrated that a TRAF2 mutant abrogated TRAIL-induced NF-κB activation (Hu, Johnson et al. 1999). However, TRAF2 deficient MEFs showed no differential NF-κB signalling after TRAIL treatment (Lin, Devin et al. 2000).

Activation of protein kinase C (PKC) has been reported to inhibit the recruitment of FADD to the TRAIL DISC, thereby modulating TRAIL sensitivity (Harper, Hughes et al. 2003). Furthermore, PKB/AKT was implicated in the stabilisation of cFLIP and Mcl-1 in TRAIL-resistant cells (Wang, Chen et al. 2008). MAPKs have also been found to affect TRAIL sensitivity (Frese, Pirnia et al. 2003). Recent studies by Song et al. suggest that mammalian sterile 20-like kinase 1 (Mst1) is needed for caspase-dependent MAPKs activation after TRAIL treatment (Song and Lee 2008). Interestingly, caspases 3 and 7 were shown to cleave Mst1 at different sites leading to differential signalling outcome. Caspase-3 seems to be a critical mediator of JNK, p38 MAP kinase and extracellular-signal regulated kinase (ERK) phosphorylation. Downregulation or absence of caspase-3 increased JNK and p38 phosphorylation while ERK phosphorylation was decreased (Song and Lee 2008).

Non-apoptotic TRAIL signalling can lead to increased proliferation or migration. Increased proliferation has been observed in various cancer cell lines of lymphoid and non-lymphoid origin after stimulation with TRAIL (Ehrhardt, Fulda et al. 2003). Enhanced migration after TRAIL treatment was shown for pancreatic ductal adenocarcinoma and cholangiocarcinoma cells (Ishimura, Isomoto et al. 2006; Trauzold, Siegmund et al. 2006). These studies demonstrated that TRAIL can promote cell migration and invasion via an NF-κB dependent pathway. It is important to bear in mind that this function of TRAIL under certain conditions might alter the outcome of TRAIL-based anti-cancer therapies.

It is important to note that some factors involved in apoptosis induction by TRAIL are also involved in non-apoptotic signal transduction by TRAIL, not the least the TRAIL receptors. Hence, novel factors identified as important for TRAIL-induced apoptosis may also play important roles in TRAIL-induced cell survival, proliferation, and/or migration. In addition, since there is substantive overlap between the molecules known to be important for non-apoptotic and apoptotic signal transduction mediated by the various death receptors, novel factors identified as important for TRAIL-induced apoptosis may also be important for non-apoptotic or apoptotic signal transduction mediated by other apoptosis factors such as other ligands of death receptors including, but not limited to, CD95L (FasL/APO-1 L), TNF, or TLIA.

### Physiological Role of the TRAIL/TRAIL-R System

Many studies proposed that TRAIL has immuno-suppressive, immunoregulatory, and immune effector functions (Lunemann, Waiczies et al. 2002; Opferman and Korsmeyer 2003). To date, several TRAIL and TRAIL-R knockout mice have been created to study the function of the TRAIL/TRAIL-R system in vivo (Sedger, Glaccum et al. 2002; Takeda, Smyth et al. 2002; Finnberg, Gruber et al. 2005; Yue, Diehl et al. 2005; Grosse-Wilde, Voloshanenko et al. 2008). Both TRAIL and TRAIL-R knockout mice are viable, fertile and do not show obvious phenotypic defects except for an enlarged thymus. Therefore, a prominent role for the TRAIL/TRAIL-R system in development can be excluded.

### TRAIL and Tumor Immunity

The first indication that endogenous TRAIL may suppress tumor growth arose when Sedger et al. reported that a syngeneic tumor transplant of a B cell lymphoma line displayed enhanced tumor growth in TRAIL_{-/-} mice (Sedger, Shows et al. 1999). Moreover, endogenous TRAIL on NK cells that were stimulated by IFN-γ or IL-12 was able to effectively kill even disseminated tumor cells in the liver but not in the lung after implantation of metastasising breast and renal carcinoma cells (Smyth, Cretney et al. 2001; Takeda, Hayakawa et al. 2001; Seki, Hayakawa et al. 2003). Furthermore, NKT cells stimulated by α-galactosylceramide (α-GalCer) showed an effective TRAIL-mediated antitumor effect (Takeda, Smyth et al. 2001; Cretney, Takeda et al. 2002).

Using xenografts of TRAIL-sensitive human cell lines and recombinant TRAIL or TRAIL-R agonists, it was shown that application of TRAIL reduced the tumor growth in vivo (Ashkenazi, Pai et al. 1999; Walczak, Miller et al. 1999). These results were confirmed in TRAIL knockout mice using syngeneic TRAIL-sensitive cell lines (Cretney, Takeda et al. 2002; Takeda, Smyth et al. 2002). In addition, TRAIL knockout mice showed increased experimental metastasis and an enhanced frequency of fibrosarcomas after treatment with the chemical carcinogen methylcholanthrene (MCA) (Cretney, Takeda et al. 2002). Furthermore, TRAIL suppressed the initiation and development of lymphomas and sarcomas in the context of the loss of at least one p53 allele (Zerafa, Westwood et al. 2005).

In TRAIL-R knockout mice Ep-myc-induced lymphomas and diethylnitrosamine (DEN)-induced hepatocarcinogenesis were enhanced (Finnberg, Klein-Szanto et al. 2008). In contrast, no role for the TRAIL/TRAIL-R system could be found in the formation of thymic or intestinal tumors (Yue, Diehl et al. 2005), in Her2/neu oncogene-driven mammary epithelial cancer (Zerafa, Westwood et al. 2005) or in DMBA/TPA-induced skin tumors (Grosse-Wilde, Voloshanenko et al. 2008).

The first indication of a metastasis-specific surveillance function of TRAIL was shown in an autochthonous multistep model of skin tumorigenesis. When TRAIL-R_{-/-} mice were treated with the tumor-initiating and -promoting agents DMBA (7,12-dimethylbenz[α]anthracene and TPA (12-O-tetradecanoylphorbol-13-acetate) papillomas and carcinomas developed without the influence of the TRAIL/TRAIL-R system. Surprisingly, lymph node metastases were greatly enhanced in the absence of TRAIL-R, which was explained by the fact that tumor cells gained TRAIL sensitivity by loss of adhesion (Grosse-Wilde, Voloshanenko et al. 2008). However, whether this specific metastasis suppressor function of TRAIL-R is confined to metastases in lymphoid organs, and which type(s) of cells are responsible for the TRAIL-mediated effect is still under investigation.

### Expression and Function of TRAIL in the Innate and Adaptive Immune System

Another hint at understanding the function of the TRAIL/TRAIL-R came when it was discovered that TRAIL is expressed on a variety of cells of the innate and adaptive immune system. Yet, the expression of TRAIL was found to be stimulation-dependent. TRAIL is upregulated on monocytes and macrophages after LPS and interferon-β (IFN-β) stimulation (Halaas, Vik et al. 2000; Ehrlich, Infante-Duarte et al. 2003). IFN-γ in turn can induce surface expression of TRAIL on monocytes, dendritic cells and natural killer (NK) cells (Fanger, Maliszewski et al. 1999; Griffith, Wiley et al. 1999).

Surface-bound TRAIL is one of the effector mechanisms of NK cells, as it has been shown that only combined neutralisation of TRAIL, CD95L and perforin can block NK cell-mediated killing of tumor cell lines in vitro (Kayagaki, Yamaguchi et al. 1999; Takeda, Smyth et al. 2001). This was also confirmed in vivo by Smyth et al. where it was demonstrated that IFNγ treatment induces TRAIL on NK cells and so prevents formation of primary tumors and experimental metastases (Smyth, Cretney et al. 2001).

High TRAIL expression on NK cells can be detected during development in fetal and neonatal mice (Takeda, Cretney et al. 2005). Some of these immature TRAIL-expressing NK cells remain in the liver of adult mice and their retention is dependent on IFNγ, but not on IL-12, IL-18 or host pathogens (Takeda, Cretney et al. 2005). Thus, a subpopulation of NK cells in the adult liver constitutively expresses TRAIL due to autocrine production of IFNγ (Takeda, Smyth et al. 2001; Takeda, Cretney et al. 2005).

In addition to NK cells, cytotoxic T lymphocytes (CD8₊ T cells) are involved in eliminating target cells by recognising tumor peptides presented by the major histocompatibility complex (MHC) on the surface of antigen presenting cells (APCs) (Lee, Bar-Haim et al. 2004). Membrane-bound TRAIL has not only been detected on NK cells, but also on activated CD8₊ T cells (Mirandola, Ponti et al. 2004). Furthermore, stimulation with anti-CD3 antibodies in combination with type I interferons (Kayagaki, Yamaguchi et al. 1999) led to upregulation of TRAIL on CD4₊ and CD8₊ human peripheral blood T cells. In contrast to CD95L, TRAIL protein expression on the cell surface was not strongly induced by TCR/CD3 stimulation alone (Kayagaki, Yamaguchi et al. 1999). The enhancement of TRAIL expression could be attributed to the costimulation with IFNα or IFNβ (Kayagaki, Yamaguchi et al. 1999). LPS in combination with pytohaemagglutinin (PHA) and IL-2 also led to upregulation of TRAIL in a type I interferon-dependent fashion (Ehrlich, Infante-Duarte et al. 2003). These results suggest that type I interferons can regulate TRAIL-mediated T cell cytotoxicity.

CD4⁺ or CD8⁺ T cells can be further subdivided according to their cytokine production. T-helper 1 (T_{H}1) cells predominantly produce IFNγ, IL-2 and IL-12 that stimulate a cellular immune response (Agaugue, Marcenaro et al. 2008). In contrast, T-helper 2 (T_{H}2) cells predominantly produce IL-4, IL-5 and IL-10. These cytokines boost an IgE-mediated humoral response and cause inflammation (Zimmer, Pollard et al. 1996). TRAIL has been implicated in the regulation of T_{H}1 and T_{H}2 responses. After anti-CD3 stimulation in vitro differentiated T_{H}1 cells upregulate CD95L, whereas T_{H}2 cells express TRAIL. Yet, T_{H}1 cells are more sensitive to TRAIL-induced apoptosis than T_{H}2 cells, possibly due to CD3-induced upregulation of c-FLIP in T_{H}2 cells (Roberts, Devadas et al. 2003; Zhang, Zhang et al. 2003). Furthermore, inhibition of TRAIL in mice with allergic airway disease, either by gene disruption or by RNA interference, inhibited the production of the chemokine CCL20 and the homing of DCs and T_{H}2 cells to the airways (Weckmann, Collison et al. 2007). As a result, less T_{H}2 cytokines were released and inflammation was reduced in TRAIL-deficient mice.

TRAIL has also been implicated in playing a role during viral and bacterial infections. It is known that many viruses are able to induce immunosuppression, but the mechanism is poorly understood. Vidalain et al. proposed that TRAIL plays an essential role. During measle virus infection, activated T cells were killed by monocyte-derived dendritic cells via TRAIL, thereby downregulating antiviral immune responses (Vidalain, Azocar et al. 2000).

TRAIL-R knockout mice were shown to have enhanced cytokine production after stimulation with Mycobacterium bovis and increased clearance of murine cytomegalovirus (MCMV) (Diehl, Yue et al. 2004). As the clearance of MCMV correlated with increased levels of IL-12, IFNα and IFNγ, the authors suggested that TRAIL-R negatively regulates innate immune responses to certain infections by influencing APCs that produce these cytokines.

It has also been shown that CD8′ T cells can kill virally infected cells via TRAIL (Mirandola, Ponti et al. 2004). Using TRAIL knockout mice, Brincks et al. demonstrated that TRAIL deficiency leads to more severe influenza infections by decreasing CD8₊ T cell-mediated cytotoxicity (Brincks, Katewa et al. 2008).

Recent reports suggest that TRAIL is involved in the killing of bystander T cells during HIV infection. HIV infection causes the production of type I interferons by plasmacytoid Dcs. This in turn leads to expression of membrane-bound TRAIL on T cells as well as to the production of soluble TRAIL by monocytes. As binding of HIV to CD4₊ T cells upregulates TRAIL-R2, these cells can be killed selectively via TRAIL-induced apoptosis (Hansjee, Kaufmann et al. 2004; Lichtner, Maranon et al. 2004; Herbeuval, Boasso et al. 2005; Herbeuval, Grivel et al. 2005; Herbeuval, Nilsson et al. 2006).

The TRAIL/TRAIL-R system may also play a role in the homeostasis of a particular subset of CD8₊ T cells. “Helpless” CD8₊ T cells are primed in the absence of CD4₊ T cells—and therefore in the absence of help—are unable to undergo a second round of clonal expansion upon restimulation with their cognate antigen (Shedlock, Whitmire et al. 2003). As TRAIL-deficient “helpless” CD8⁺ T cells can still expand a second time, this effect is thought to be mediated via TRAIL. Therefore, a mechanism was suggested in which non-helped T cells are eliminated via TRAIL by an activation-dependent killing upon antigen re-challenge (Janssen, Droin et al. 2005). More recently, IL-15 was identified to be a mediator of this effect by rendering “helped” CD8⁺ T cells resistant to TRAIL-induced apoptosis (Oh, Perera et al. 2008).

An immuno-suppressive function of so called “T suppressor cells” was described over 30 years ago (Gershon and Kondo 1971; Sy, Miller et al. 1977; Greene and Benacerraf 1980). In the original papers, the factor responsible for the observed suppression effect remained elusive. By performing some of the original experiments that were used to study suppressor T cells, but now employing TRAIL, mice and recombinant TRAIL, Griffith et al. have now provided quite compelling evidence that TRAIL may be the long sought-after suppressor factor (Griffith, Kazama et al. 2007).

### TRAIL and Autoimmunity

TRAIL has also been implicated to play a role in autoimmune diseases. TRAIL^{-/-} and TRAILR−/− mice do not show signs of spontaneous autoimmunity, but TRAIL was shown to inhibit autoimmune diseases in a number of animal models including collagen-induced arthritis (Song, Chen et al. 2000), diabetes (Lamhamedi-Chemadi, Zheng et al. 2003), experimental autoimmune encephalomyelitis (EAE) (Hilliard, Wilmen et al. 2001; Cretney, McQualter et al. 2005) and experimental autoimmune tyroiditis (EAT) (Wang, Cao et al. 2005).

TRAIL's influence on autoimmunity was at first attributed to its supposed role in thymic negative selection. However, a function of the TRAIL/TRAIL-R system in central tolerance is, to say the least, highly contested. Although an initial study showed that negative selection of human and mouse thymocytes is independent of TRAIL signalling (Simon, Williams et al. 2001), two subsequent reports contradicted the previous study by suggesting that TRAIL was necessary for intrathymic selection (Lamhamedi-Chemadi, Zheng et al. 2003; Corazza, Brumatti et al. 2004). However, TRAIL expression has not been detected on thymic dendritic and epithelial cells which are the most important mediators of negative selection in the thymus (Tanaka, Mamalaki et al. 1993; Sprent and Webb 1995). Finally, elegant studies in various different model systems for the study of negative selection by Cretney et al. (Cretney, Uldrich et al. 2003) using TRAIL_{-/-} mice and a neutralising anti-mouse TRAIL monoclonal antibody proved that negative selection in the thymus did not involve the TRAIL system. Additionally, negative selection was also reported to be normal in TRAIL-R_{-/-} mice (Diehl, Yue et al. 2004). In conclusion, it is rather unlikely that the TRAIL/TRAIL-R system plays a role in thymic negative selection under physiological conditions. TRAIL has also been shown to bind to OPG, an osteoblast-secreted decoy receptor that functions as a negative regulator of bone resorption (Emery, McDonnell et al. 1998; Boyce and Xing 2008). As TRAIL- and TRAIL-R-deficient mice do not show a bone phenotype, the physiological importance of the TRAIL-OPG interaction is still elusive.

### TRAIL as a Therapeutic Agent

TRAIL has been shown to selectively induce apoptosis in a variety of tumor cells while normal cells are resistant to TRAIL treatment (Ashkenazi, Pai et al. 1999; Walczak, Miller et al. 1999). Therefore TRAIL, as well as agonistic antibodies to TRAIL-R1 and TRAIL-R2, represent very promising novel biotherapeutic agents for cancer therapy.

Conventional anticancer therapies are often associated with damage of normal tissue and the development of drug resistance. The use of TRAIL and TRAIL-R agonists has two big advantages. Firstly, no severe toxicity to normal tissue is observed and secondly, apoptosis triggering via the extrinsic TRAIL receptor pathway is independent of p53. Deletion or mutation of p53 is a common feature of many cancers which can lead to resistance to conventional chemotherapy (Hollstein, Rice et al. 1994; Lee and Bernstein 1995; Igney and Krammer 2002). TRAIL has been shown to overcome cancer cell resistance to chemotherapy (Mitsiades, Treon et al. 2001) and to synergise with chemotherapy even in p53-deficient cells (Ravi, Jain et al. 2004; Wissink, Verbrugge et al. 2006).

However, most primary tumors are resistant to TRAIL (Todaro, Lombardo et al. 2008), but can be sensitized to TRAIL by cotreatment with various chemotherapeutic drugs or irradiation (Zisman, Ng et al. 2001; Munshi, McDonnell et al. 2002; Nyormoi, Mills et al. 2003; Ganten, Koschny et al. 2005; Fulda and Debatin 2006; Belyanskaya, Marti et al. 2007; Micali, Cheung et al. 2007; Dzieran, Beck et al. 2008; Maduro, de Vries et al. 2008). Recombinant versions of TRAIL as well as agonistic antibodies targeting TRAIL-R1 or TRAIL-R2 are currently in clinical development.

Several recombinant forms of TRAIL and TRAIL-R agonistic antibodies have been tested in various preclinical cell and animal models. Many soluble versions of TRAIL contain an N-terminal motif, for instance a polyhistidine tag (HIS-TRAIL) (Pitti, Marsters et al. 1996), a leucine zipper motif (LZ-TRAIL) (Walczak, Miller et al. 1999), an isoleucine zipper motif (IZTRAIL) (Ganten, Koschny et al. 2006), or a FLAG tag (FLAG-TRAIL) (Schneider 2000).

HIS-TRAIL was shown to exhibit considerable toxicity on freshly isolated human hepatocytes (Jo, Kim et al. 2000). Therefore, an untagged version of recombinant soluble TRAIL (rhApo2L/TRAIL) has been entered into clinical studies. rhApo2L/TRAIL is being co-developed by Genentech and Amgen as a targeted therapy for solid tumors and hematological malignancies (Ashkenazi and Herbst 2008). So far, patients receiving rhApo2L/TRAIL as a single agent showed no dose-limiting toxicity (DLT) or severe adverse effects (SAEs). A phase Ib study of rhApo2L/TRAIL in combination with rituximab was performed in patients with low-grade Non-Hodgkin Lymphoma (NHL) who had previously failed a rituximab-containing regimen. The combined administration seems to be safe and shows evidence of activity. So far, eight patients have undergone tumor response assessment in which two showed complete response, one a partial response and five stable disease (Yee 2007). However, two SAEs possibly related to the combination of rhApo2L/TRAIL and rituximab have been reported. These included pneumonia, septic shock and ileus, but the patient could continue the therapy.

Besides soluble TRAIL that targets both TRAIL-R1 and TRAIL-R2, TRAIL-R-binding agonistic antibodies specific for TRAIL-R1 or TRAIL-R2 have been developed. Human Genome Sciences (HGS) was the first company to test TRAIL receptor agonists in clinical trials. HGS is currently investigating fully humanised agonistic antibodies against TRAIL-R1 (Mapatumumab/HGS-ETR1) and TRAIL-R2 (Lexatumumab/HGS-ETR2) as a therapy for NHL, colorectal cancer, non-small cell lung cancer (NSCLC) and advanced solid tumors. HGS has already completed three Phase II clinical trials of Mapatumumab as monotherapy in heavily pretreated patients with NHL, colorectal cancer and NSCLC. The results of these trials show that Mapatumumab is well tolerated and that it could be safely and repetitively administered.

Preclinical studies with novel agents that sensitize cancer cells to TRAIL-induced apoptosis are ongoing. Among them are histone deacetylase (HDAC) inhibitors, IAP antagonists (Smac mimetics), BH3 mimetics (e.g. ABT-737) and kinase inhibitors (e.g. Sorafenib).

In summary, the clinical trials performed so far demonstrate that TRAIL receptor agonists are well tolerated in humans. Given as a single agent, they induce partial response or stable disease in a high percentage of patients. In combination with various chemotherapeutic drugs partial or complete regression of the tumors could be observed in some patients. It is important to consider that patients enrolled in phase I/II clinical studies mostly have advanced tumors that were heavily pretreated and did not respond to other treatments before.

By using TRAIL in combination with conventional therapy, the hope is that conventional therapies can be reduced to sub-toxic concentrations thus increasing the likelihood that TRAIL selectivity for tumor over normal cells will be retained. Therefore, TRAIL receptor agonists in combination with established chemotherapeutic drugs or novel agents (e.g. HDACI, Sorafenib or IAP antagonists), represent a promising therapeutic option for many cancer patients in the future.

### RNA Interference

RNA interference (RNAi) is a sequence-specific, post-transcriptional silencing process that is mediated by double-stranded RNA (dsRNA) molecules (Fire, Xu et al. 1998). This mechanism has evolved as a defense mechanism to target dsRNA from viruses and transposons, but also plays a role in regulating gene expression and genome maintenance. Long dsRNAs are processed by an enzyme called DICER into smaller fragments named small interfering RNAs (siRNAs). The siRNAs are then incorporated into the RNA-induced silencing complex (RISC) where they serve as templates to recognise, pair to, and cleave their complementary mRNAs (Elbashir, Harborth et al. 2001). After cleavage, mRNAs are degraded very rapidly and translation cannot occur.

Nowadays, RNAi is widely used as a powerful technique to downregulate the expression of specific genes. RNAi libraries that target most genes in plants, worms, flies and humans have been created and are now used in genome-wide screens (Boutros, Kiger et al. 2004; Boutros and Ahringer 2008). These approaches provided important insights into gene functions and revealed novel functions for already identified genes. In worm, fly or plant models, long dsRNAs (400-700 bp) can be used to induce gene silencing. However, in mammalian cells long dsRNA induces a strong interferon response (Manche, Green et al. 1992; Stark, Kerr et al. 1998). Therefore, short hairpin RNAs (shRNAs) that are introduced in retroviral, adenoviral or lentiviral vectors or chemically synthesized siRNAs have to be used for gene-silencing approaches.

It was the problem underlying the present invention to provide novel modulators of apoptosis-factor-associated cell death, apoptosis, cell survival, migration and/or proliferation, in particular TRAIL-induced apoptosis. Correspondingly, another object of the present invention was to provide methods for genome-wide screenings, in particular genome-wide RNAi screenings, capable of displaying markers of cellular growth and survival.

The identification of modulators of cellular factors that are responsible for the induction or inhibition of cell death, apoptosis, cell survival, migration and/or proliferation, in particular TRAIL-induced apoptosis, or the expression of which results in an increase or decrease of cell death, apoptosis, cell survival, migration and/or proliferation, in particular TRAIL-induced apoptosis, provides novel biomarkers and therapeutic targets for the diagnosis and treatment of apoptosis-related disorders, particularly TRAIL-related diseases.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure. Using a genome-wide RNAi screening, the inventors identified surprisingly a multitude of genes encoding proteins that are involved in cell death induction by TRAIL and were previously not known to be involved in this process. The newly identified genes are as depicted in Table 1.

TABLE 1
Genes involved in apoptosis-factor-associated cell death,
apoptosis, cell survival, migration and/or proliferation,
in particular TRAIL-associated apoptosis

| RefSeq. ID | GeneID | Description |
|---|---|---|
| NM_153345 | FLJ90586 | Homo sapiens hypothetical |
| | | protein FLJ90586 (FLJ90586), |
| | | mRNA |
| NM_019086 | FLJ20674 | Homo sapiens hypothetical |
| | | protein FLJ20674 (FLJ20674), |
| | | mRNA |
| NM_152687 | FLJ33641 | Homo sapiens hypothetical |
| | | protein FLJ33641 (FLJ33641), |
| | | mRNA |
| XM_379324 | LOC340113 | PREDICTED: Homo sapiens |
| | | hypothetical protein LOC340113 |
| | | (LOC340113), mRNA |
| NM_020350 | AGTRAP | Homo sapiens angiotensin II |
| | | receptor-associated protein |
| | | (AGTRAP), transcript variant 1, |
| | | mRNA. |
| NM_024735 | FBXO31 | Homo sapiens F-box protein 31 |
| | | (FBXO31), mRNA |
| XM_375375 | KIAA0431 | ATMIN, ATM interactor |
| NM_002268 | KPNA4 | Homo sapiens karyopherin |
| | | alpha 4 (importin alpha 3) |
| | | (KPNA4), mRNA |
| NM_016069 | MAGMAS | Homo sapiens mitochondria- |
| | | associated protein involved in |
| | | granulocyte-macrophage colony- |
| | | stimulating factor signal |
| | | transduction (Magmas), nuclear |
| | | gene encoding mitochondrial |
| | | protein, mRNA |
| NM_004991 | MDS1 | Homo sapiens myelodysplasia |
| | | syndrome 1 (MDS1), mRNA |
| NM_022731 | NUCKS | Homo sapiens nuclear casein |
| | | kinase and cyclin-dependent |
| | | kinase substrate 1 (NUCKS1), |
| | | mRNA |
| NM_001005284 | OR9G4 | Homo sapiens olfactory |
| | | receptor, family 9, subfamily G, |
| | | member 4 (OR9G4), mRNA |
| | | G-protein coupled receptor |
| NM_017730 | FLJ20259 | Homo sapiens glutamine-rich 1 |
| | | (QRICH1), transcript variant 1, |
| | | mRNA |
| NM_006913 | RNF5 | Ring finger protein 5, transcript |
| | | variant 1, mRNA |
| NM_005273 | GNB2 | Homo sapiens guanine |
| | | nucleotide binding protein (G |
| | | protein), beta polypeptide 2 |
| | | (GNB2), mRNA |
| NM_032368 | LZIC | Homo sapiens leucine zipper |
| | | and CTNNBIP1 domain |
| | | containing (LZIC), mRNA |
| NM_018075 | FLJ10375 | Homo sapiens transmembrane |
| | | protein 16K (TMEM16K), mRNA |
| | | PMID: 19513534 |
| NM_005096 | ZNF261 | Homo sapiens zinc finger, MYM- |
| | | type 3 (ZMYM3), transcript |
| | | variant 1, mRNA |
| NM_002201 | ISG20 | Homo sapiens interferon |
| | | stimulated exonuclease gene |
| | | 20 kDa (ISG20), mRNA |
| NM_012367 | OR2B6 | Homo sapiens olfactory |
| | | receptor, family 2, subfamily B, |
| | | member 6 (OR2B6), mRNA |
| NM_018052 | FLJ10305 | Homo sapiens Vac14 homolog |
| | | (S. cerevisiae) (VAC14), mRNA |
| NM_080740 | SUHW1 | Homo sapiens suppressor of |
| | | hairy wing homolog 1 |
| | | (Drosophila) (SUHW1), mRNA |
| NM_022486 | SUSD1 | Homo sapiens sushi domain |
| | | containing 1 (SUSD1), mRNA |
| NM_024518 | ULBP3 | Homo sapiens UL16 binding |
| | | protein 3 (ULBP3), mRNA |
| NM_017901 | TPCN1 | Homo sapiens two pore |
| | | segment channel 1 (TPCN1), |
| | | mRNA |
| NM_006752 | SURF5 | Homo sapiens surfeit 5 |
| | | (SURF5), transcript variant a, |
| | | mRNA |
| NM_004261 | SEP15 | Homo sapiens 15 kDa |
| | | selenoprotein (SEP15), |
| | | transcript variant 1, mRNA |
| NM_003134 | SRP14 | Homo sapiens signal recognition |
| | | particle 14 kDa (homologous Alu |
| | | RNA binding protein) (SRP14), |
| | | mRNA |
| NM_024644 | C14ORF169 | Homo sapiens chromosome 14 |
| | | open reading frame 169 |
| | | (C14orf169), mRNA |
| NM_004910 | P1TPNM1 | Homo sapiens |
| | | phosphatidylinositol transfer |
| | | protein, membrane-associated 1 |
| | | (PITPNM1), mRNA |
| NM_015945 | SLC35C2 | Homo sapiens solute carrier |
| | | family 35, member C2 |
| | | (SLC35C2), transcript variant 2, |
| | | mRNA |
| NM_018189 | DPPA4 | Homo sapiens developmental |
| | | pluripotency associated 4 |
| | | (DPPA4), mRNA |
| NM_207373 | C10ORF99 | Homo sapiens chromosome 10 |
| | | open reading frame 99 |
| | | (C10orf99), mRNA |
| NM_001311 | CRIP1 | Homo sapiens cysteine-rich |
| | | protein 1 (intestinal) |
| NM_015957 | MMRP19 | Homo sapiens APAF1 |
| | | interacting protein (APIP), |
| | | mRNA |
| NM_018235 | CNDP2 | Homo sapiens CNDP |
| | | dipeptidase 2 |
| | | (metallopeptidase M20 family) |
| | | (CNDP2), mRNA |
| NM_173623 | FLJ35808 | Homo sapiens tubulin tyrosine |
| | | ligase-like family, member 6 |
| | | (TTLL6), mRNA |
| NM_016076 | PNAS-4 | Homo sapiens chromosome 1 |
| | | open reading frame 121 |
| | | (C1orf121), mRNA |
| NM_016233 | PADI3 | Homo sapiens peptidyl |
| | | arginine deiminase, type III |
| | | (PADI3), mRNA |
| NM_025099 | FLJ22170 | Homo sapiens chromosome 17 |
| | | open reading frame 68 |
| | | (C17orf68), mRNA |
| XM_035299 | ZSWIM6 | Homo sapiens zinc finger, |
| | | SWIM domain containing 6 |
| | | (ZSWIM6), mRNA |

The present invention thus discloses an agent selected from a nucleic acid molecule as identified in Table 1, a homologue thereof, a polypeptide encoded by said nucleic acid molecule or homologue thereof or an effector of said nucleic acid molecule or of said polypeptide for use as a modulator of apoptosis-factor-associated cell death, apoptosis, cell survival, migration and/or proliferation, in particular TRAIL-associated apoptosis. Accordingly one preferred embodiment relates to an agent selected from a nucleic acid molecule as identified in Table 1, a homologue thereof, a polypeptide encoded by said nucleic acid molecule or homologue thereof or an effector of said nucleic acid molecule or of said polypeptide for use as a modulator of apoptosis-factor-associated cell death and/or apoptosis. Another embodiment relates to an agent selected from a nucleic acid molecule as identified in Table 1, a homologue thereof, a polypeptide encoded by said nucleic acid molecule or homologue thereof or an effector of said nucleic acid molecule or of said polypeptide for use as a modulator of apoptosis-factor-associated cell survival, migration and/or proliferation.

“Apoptosis-factor” according to the invention means any factor to be known to the person skilled in the art which is known to be associated with cell death and/or apoptosis such as any factors which induce, promote, inhibit or prevent apoptosis and/or cell death. Moreover, according to the invention the term “apoptosis-factor-associated” comprises endogenous agents, i.e. agents provided or produced by a subject to be treated itself, as well as exogenous agents, i.e. agents which may be administered to a subject to be treated, such as chemotherapeutic agents.

“Modulator” comprises any agent which affects, effects and/or mediates any kind of induction, inhibition, prevention and/or promotion. Thus, “modulation” according to the invention also comprises the use of agents which drive the expression of the nucleic acid molecules listed in Table 1, thereby, for example, rendering previously apoptosis-resistant tumor cells, in particular TRAIL-apoptosis-resistant tumor cells, sensitive to cell death induction by agents such as TRAIL or other TRAIL receptor agonists.

According to the invention, the term “cell death” comprises apoptotic cell death induced by apoptotic factors or non-apoptotic cell death, preferably apoptosis-factor-induced non-apoptotic cell death. The terms “apoptotic cell death” and “apoptosis” may be used interchangeable herein.

However, an apoptosis-factor according to the invention is preferably selected from the group consisting of TRAIL, CD95L, TNF, such as TNF-alpha or TNF-beta, TL1A and any combination thereof. According to an especially preferred embodiment the apoptosis-factor is CD95L. According to the most preferred embodiment the apoptosis-factor is TRAIL.

The specific triggering of cell survival, migration, proliferation, non-apoptotic cell death and/or apoptosis associated with only one apoptosis factor can have an advantage in that a possible toxicity or possible side effects, respectively, which are connected with further expressing or induced apoptosis factors, is minimized. For example, the specific triggering of TRAIL-induced cell death or TRAIL-induced apoptosis can be advantageous in comparison with CD95L-induced cell death in that such an agent may for example increase an antitumor-effect based on TRAIL-induced cell death or TRAIL-induced apoptosis without the appearance of a possible CD95L-dependent toxicity.

The invention also relates to the use of these compounds, i.e. nucleic acid molecules as identified in Table 1, homologues thereof, polypeptides encoded by said nucleic acid molecules or homologues thereof and effectors thereof, e.g. antibodies, biologically active nucleic acids, such as antisense molecules, RNAi molecules or ribozymes, aptamers, peptides or low-molecular weight organic compounds recognizing said polynucleotides or polypeptides, in the diagnosis, study, prevention, and treatment of medical conditions and disorders related with apoptosis-factor-associated cell survival, migration, proliferation, non-apoptotic cell death and/or apoptosis, in particular TRAIL-induced apoptosis.

In terms of the invention, an agent for use as a modulator of cell survival, migration, proliferation, non-apoptotic cell death and/or apoptosis, in particular TRAIL-induced apoptosis, may be a nucleic acid molecule as identified in Table 1, a homologue thereof, a polypeptide encoded by said nucleic acid molecule or a homologue thereof or an effector of said nucleic acid molecule or of said polypeptide.

It will be appreciated by those skilled in the art that as a result of the degeneracy of the genetic code, a multitude of nucleotide sequences, some bearing minimal homology to the nucleotide sequences presented in table 1, may be produced. The invention contemplates each and every possible variation of nucleotide sequence that can be made by selecting combinations based on possible codon choices. Also encompassed by the invention are polynucleotide sequences that are capable of hybridizing to the nucleic acid molecules of table 1 under various conditions of stringency. Hybridization conditions are based on the melting temperature (Tm) of the nucleic acid binding complex or probe, as described in Wahl G. M. et al. (1987: Methods Enzymol. 152: 399-407) and Kimmel A. R. (1987; Methods Enzymol. 152: 507-511), and may be used at a defined stringency. Preferably, hybridization under stringent conditions means that after washing for 1 h with 1*SSC and 0.1% SDS at 50° C., preferably at 55° C., more preferably at 62° C. and most preferably at 65° C., particularly for 1 h in 0.2*SSC and 0.1% SDS at 50° C., preferably at 55° C., more preferably at 62° C. and most preferably at 65° C., a positive hybridization signal is observed. Altered nucleic acid sequences of the nucleic acid molecules of table 1 include deletions, insertions or substitutions of different nucleotides resulting in a polynucleotide that encodes the same or a functionally equivalent protein.

In terms of the invention, the nucleic acid molecule may for example be a DNA or RNA molecule. The DNA and RNA molecules may also comprise modified nucleotides known to the person skilled in the art or a modified phosphate-backbone such as PNA molecules. The nucleic acid molecule preferably encodes a mammalian, particularly a human polypeptide or a variant thereof.

The polypeptide encoded by an above described nucleic acid molecule preferably is a mammalian, in particular a human polypeptide or a variant thereof.

The encoded polypeptide may also be mutated, i.e. may contain deletions, insertions or substitutions of amino acid residues, which produce a silent change and/or result in functionally equivalent polypeptides. Deliberate amino acid substitutions may be made on the basis of similarity in polarity, charge, solubility, hydrophobicity, hydrophilicity, and/or the amphipathic nature of the residues as long as the biological activity of the polypeptide is retained. Furthermore, the invention relates to fragments of the polypeptides, i.e. the polypeptides may be truncated, or derivatives thereof such as cyclic peptides, retro-inverso peptides or peptide mimetics having a length of at least 4, preferably at least 6, more preferably 10, even more preferably 20 and up to 50, more preferably 60 and even more preferably 75 amino acids. Preferably polypeptide fragments or truncated forms of the polypeptide are also functionally equivalent to the corresponding non-fragmented or non-truncated peptide.

Effectors of the nucleic acid molecules identified in Table 1 or of the encoded polypeptides can act on the nucleic acid and/or protein level, i.e. the nucleic acid molecules themselves and/or their transcription and/or their translation may be affected. Effectors of the nucleic acid molecules identified in Table 1 or of the encoded polypeptides are for example antibodies, biologically active nucleic acids, such as antisense molecules, small-interfering RNA molecules, small hairpin RNAs and other effector molecules for small-interfering RNA molecules, short hairpin RNAs and other effector molecules for RNAi (reviewed in Boutros and Ahringer, (2008) Nature Reviews Genetics, 9:554-566 and references therein) or ribozymes, aptamers, peptides or low-molecular weight organic compounds recognizing said polynucleotides or polypeptides. Preferred examples of effectors in terms of the invention are (i) antibodies directed against the polypeptide, (ii) truncated or mutated fragments of the polypeptide, (iii) nucleic acid effector molecules such as aptamers, ribozymes, antisense molecules, siRNAs etc. or (iv) low-molecular weight compounds. Antibodies or antibody-fragments effecting the transcription and/or translation of the nucleic acid molecules identified in Table 1 represent another preferred embodiment of effectors of said nucleic acid molecules.

For the production of antibodies, various hosts including goats, rabbits, rats, mice, humans, and others, may be immunized by injection with the polypeptide or any fragment or oligopeptide thereof which has immunogenic properties. Depending on the host species, various adjuvants may be used to increase immunological response. It is preferred that the polypeptides, fragments or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Köhler, G. and Milstein C., (1975) Nature 256: 495-497; Kozbor, D. et al., (1985) J. Immunol. Methods 81: 31-42; Cote, R. J. et al., (1983) Proc. Natl. Acad. Sci. 80: 2026-2030; Cole, S. P. et al., (1984) Mol. Cell. Biochem. 62: 109-120).

In addition, techniques developed for the production of ‘chimeric antibodies’, the splicing of mouse antibody genes to human antibody genes to obtain a molecule with appropriate antigen specificity and biological activity can be used (Morrison, S. L. et al., (1984) Proc. Natl. Acad. Sci. USA 81: 6851-6855; Neuberger, M. S. et al., (1984) Nature 312: 604-608; Takeda, S. et al., (1985) Nature 314: 452-454). Alternatively, techniques described for the production of single chain antibodies may be adapted, using methods known in the art, to produce single chain antibodies specific for the proteins of the invention and homologous proteins. Antibodies with related specificity, but of distinct idiotypic composition, may be generated by chain shuffling from random combinatorial immunoglobulin libraries (Kang, A S. et al., (1991) Proc. Natl. Acad. Sci. USA 88: 11120-11123). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. USA 86: 3833-3837; Winter, G. and Milstein C., (1991) Nature 349: 293-299).

Antibody fragments which contain specific binding sites for the proteins may also be generated. For example, such fragments include, but are not limited to, the F(ab′)2 fragments which can be produced by Pepsin digestion of the antibody molecule and the Fab fragments which can be generated by reducing the disulfide bridges of F(ab′)2 fragments. Alternatively, Fab expression libraries may be constructed to allow rapid and easy identification of monoclonal Fab fragments with the desired specificity (Huse W. D. et al. (1989) Science 254:1275-1281). According to the invention, antibody fragments show preferably almost the same antigen binding specificity as the corresponding complete antibody.

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering protein epitopes are preferred, but a competitive binding assay may also be employed (Maddox, supra).

Aptamers, i.e. nucleic acid molecules, which are capable of binding to a polypeptide of the invention and modulating its activity, may be generated by a screening and selection procedure involving the use of combinatorial nucleic acid libraries.

Antisense molecules are suitable for use in situations in which it would be desirable to block the transcription of the mRNA. In particular, cells may be transformed with sequences complementary to polynucleotides encoding the proteins of the invention and homologous proteins. Thus, antisense molecules may be used to modulate protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the nucleic acid molecules the invention. These techniques are described both in Sambrook et al. (supra) and in Ausubel et al. (supra). As mentioned above, modifications of gene expression can be obtained by designing antisense molecules, e.g. DNA, RNA or nucleic acid analogues such as PNA, to the control regions of the nucleic acid molecules of the invention and genes encoding homologous proteins, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee J. E. et al., (1994) Gene 149: 109-114; Huber B. E. and Carr B. I., Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the proteins of the invention and homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector molecules, e.g. aptamers, antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ and/or 3′ ends of the molecule or modifications in the nucleobase, sugar and/or phosphate moieties, e.g. the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

The agents according to the invention may be used to treat a wide variety of different disorders, diseases and conditions. A key role of apoptosis-factor-associated cell death, apoptosis, cell survival, migration and/or proliferation, in particular TRAIL-induced apoptosis, with respect to a wide range of diseases is known to the person skilled in the art.

In several models of autoimmune diseases the TRAIL/TRAIL-R system has been shown to be involved in the regulation of auto-reactive T cells. When TRAIL is absent, collagen-induced arthritis (animal model of rheumatoid arthritis), streptozotocin-induced diabetes (animal model of human type I diabetes), (Lamhamedi-Chemadi et al., 2003), experimental autoimmune thyroiditis (EAT) (animal model of thyroiditis) (Wang et al., 2005) and experimental autoimmune encephalitis (EAE) (animal model of multiple sclerosis) (Cretney et al., 2005) were increased.

Intriguingly, in EAE, systemic TRAIL blockage led to a higher degree of inflammation in the central nervous system (CNS) and a more severe disease which was also confirmed by studies using TRAIL−/− mice. However, the degree of apoptosis of inflammatory cells in the CNS was not affected by the blockage of TRAIL, suggesting that TRAIL does not regulate apoptosis of inflammatory cells but prevents the activation of auto-reactive T cells (Cretney et al., 2005; Hilliard et al, 2001). In another study, in which TRAIL-blocking TRAIL-R2-Fc was injected into the CNS, the exact opposite effect of TRAIL blockage was observed. The clinical severity of EAE as well as the neural apoptosis in brainstem motor areas was significantly reduced. This was due to less TRAIL-induced apoptosis of neuronal cells by TRAIL-expressing encephalitogenic T cells (Aktas et al, 2005). Therefore, TRAIL does not only have an immunoregulatory role in the periphery, but also contributes to neural damage in the inflamed brain.

In a model of EAT, treatment with recombinant TRAIL led to a milder form of the disease with a significant decrease in mononuclear cell infiltration in the thyroid and less thyroid follicular destruction (Wang et al, 2005).

A dual role for TRAIL was also suggested in a model of rheumatoid arthritis which is characterised by the expansion of fibroblast-like synoviocytes (FLSs). It was shown that TRAIL can induce apoptosis as well as proliferation of FLSs (Morel et al., 2005).

In mice with allergic airway disease, inhibition of TRAIL either by gene disruption or by RNA interference, inhibited the production of the chemokine CCL20 and the homing of DCs and TH2 cells to the airways (Weckmann et al., 2007). As a result, less TH2 cytokines were released and inflammation was reduced in TRAIL-deficient mice.

Haematopoietic progenitor cells and mature erythroblasts are resistant to TRAIL-induced apoptosis, in contrast to immature erythroblasts (Zauli et al., 2006; Secchiero et al, 2004). In patients with aplastic anemia, TRAIL expression in the bone marrow was increased (Kakagianni et al., 2006) which could cause the death of immature erythroblasts. Furthermore, enhanced release of TRAIL was reported in Fanconi anemia (Pigullo et al., 2007) and myelodysplastic syndrome (Campioni et al., 2005). On the contrary, in patients with multiple myeloma, erythropoiesis is stimulated by the decreased expression of TRAIL-R1, TRAIL-R2 and TRAIL (Grzasko et al., 2006).

Measle virus infection led to TRAIL-mediated killing of activated T cells by monocyte-derived dendritic cells, thereby downregulating antiviral immune responses (Vidalain et al., 2000). HIV-1 infection causes the production of type I IFNs by plasmacytoid dendritic cells which in turn leads to the expression of membrane-bound TRAIL on CD4+ T cells and TRAIL production by monocytes. Binding of HIV-1 to CD4+ T cells upregulates TRAIL-R2 and this has been suggested to facilitate selective apoptosis of CD4+ T cells (Hansjee et al., 2004; Herbeuval et al., 2005, 2006; Lichtner et al., 2004).

Sedger et al. demonstrated that TRAIL-resistant fibroblasts could be sensitised to TRAIL-induced apoptosis by infection with human cytomegalovirus (HCMV) (Sedger et al., 1999). The infection caused upregulation of TRAIL-R1 and TRAIL-R2 on infected fibroblasts, whereas IFN-γ, that is produced by T and B lymphocytes, NK cells, monocytes and macrophages, induced expression of TRAIL and downregulated the expression of TRAIL-Rs on uninfected fibroblasts. Hence, TRAIL selectively kills virus-infected cells while leaving uninfected cells intact.

An anti-viral response against encephalomyocarditis virus (ECMV) mediated by TRAIL-expressing NK cells was shown to be dependent on IFN-α and IFN-β, which is produced by virus-infected cells. Blocking of NK cell derived TRAIL resulted in higher viral titres and earlier death of infected mice (Sato et al., 2001).

Influenza virus-infected cells can be killed via TRAIL-expressing CD8+ T cells (Mirandola et al., 2004). Using TRAIL−/− mice, Brincks et al., showed that TRAIL deficiency leads to increased influenza virus titres and disease severity (Brincks et al., 2008).

Human cell lines infected with respiratory syncytialvirus (RSV) upregulate TRAIL-R1 and -R2 and become highly sensitive to TRAIL (Kotelkin et al., 2003). These results suggest that RSV-infected cells could be eliminated by TRAIL-expressing immune cells in vivo.

Thus, the agents according to the invention may be used, for example, for the treatment of hyperproliferative disorders, e.g. cancer, such as papilloma, carcinoma, solid tumors, colon cancer colorectal cancer, breast cancer, lung cancer (non-small lung cancer as well as small cell lung cancer (NSCLC and SCLC)), thyroid cancer, prostate cancer, liver cancer, any other type of cancer and metastases such as lymph node metastases and distant organ metastases derived from any type of cancer, and/or degenerative disorders, acute or chronic, such as neurodegenerative disorders including, but not limited to, Parkinson's disease, Alzheimer's disease, Huntington's Disease, ALS, stroke, myocardial infarction, aplastic anemia, Fanconi anemia, myelodysplastic myeloma, inflammation, autoimmune disorders including, but not limited to, rheumatoid arthritis, diabetes, in particular type I diabetes, thyroiditis, psoriasis, and multiple sclerosis, bacterial and/or viral infections e.g. by HIV, CMV, influenza virus, respiratory syncytialvirus etc.

According to one preferred embodiment of the invention an above defined agent acts as a stimulator of apoptosis-factor-associated cell death and/or apoptosis, preferably TRAIL-induced cell death, in particular TRAIL-induced apoptosis. Thus, an agent according to the invention may be used for the induction or promotion of apoptosis-factor induced non-apoptotic cell-death and/or apoptosis, in particular TRAIL-induced apoptosis.

Such an agent is in particular suitable for use in the treatment of disorders wherein an induction or promotion of non-apoptotic cell-death and/or apoptosis may be helpful, such as in the treatment of inflammation, rheumatoid arthritis, multiple sclerosis, viral infection, e.g. by CMV, influenza virus, respiratory syncytial virus, and/or hyperproliferative disorders, such as cancer. Such an agent is particularly suitable for use in the treatment of such disorders when used in combination with recombinant TRAIL and/other TRAIL-receptor agonists, in particular agonistic antibodies directed against TRAIL-R1 and/or TRAIL-R2, capable of inducing cell death.

According to another preferred embodiment of the invention, an above defined agent acts as inhibitor of apoptosis-factor-associated cell death and/or apoptosis, preferably TRAIL-induced cell death, in particular TRAIL-induced apoptosis. Thus, an agent according to the invention may be used for the inhibition or prevention of apoptosis-factor induced non-apoptotic cell-death and/or apoptosis, in particular for the inhibition or prevention of TRAIL-induced apoptosis. Such an agent is suitable for use in the treatment of disorders wherein an inhibition or prevention of non-apoptotic cell death and/or apoptosis may be helpful, such as in the treatment of degenerative disorder. Examples of disorders wherein an inhibition or prevention of apoptosis-factor induced non-apoptotic cell-death and/or apoptosis, in particular TRAIL-induced apoptosis, may be helpful include, but are not limited to acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis, and/or viral infections e.g. by HIV.

According to another preferred embodiment of the invention an above defined agent acts as a stimulator of cell survival, migration and/or proliferation, preferably TRAIL-induced cell survival, migration and/or proliferation. Thus, an agent according to the invention may be used for the induction of apoptosis-factor induced cell survival, migration and/or proliferation, in particular TRAIL-induced cell survival, migration and/or proliferation. Examples of disorders to be treated with an agent acting as a stimulator of cell survival, migration and/or proliferation comprise, but are not limited to acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis and/or viral infections e.g. by HIV.

A further embodiment relates to an above defined agent acts as an inhibitor of cell survival, migration and/or proliferation, preferably TRAIL-induced cell survival, migration and/or proliferation. Thus, an agent according to the invention may be used for the inhibition or prevention of apoptosis-factor induced cell survival, migration and/or proliferation, in particular TRAIL-induced cell survival, migration and/or proliferation. Examples of disorders to be treated with an agent acting as an inhibitor of cell survival, migration and/or proliferation comprise, but are not limited to, inflammation, rheumatoid arthritis, multiple sclerosis, hyperproliferative disorders, such as cancer and/or viral infections such as by CMV, influenza virus, respiratory syncytial virus etc.

For example, nucleic acid molecules as identified in table 1 or cDNAs encoding the polypeptides of the invention and particularly their human homologues may be useful in gene therapy. The polypeptides of the invention and particularly their human homologues may be useful when administered to a subject in need thereof. By way of non-limiting example, the agents of the present invention will have efficacy for treatment of patients suffering from, for example, but not limited to, hyperproliferative disorders or degenerative disorders as described above.

For example, an agent of the invention may be used directly as a modulator of apoptosis-factor-associated cell death, apoptosis, cell survival, migration and/or proliferation, in particular TRAIL-induced cell death or TRAIL-induced apoptosis, or indirectly as a targeting or delivery mechanism for bringing a pharmaceutical agent to cells or tissue showing apoptosis-factor-associated cell survival and/or proliferation, in particular TRAIL-induced cell death or TRAIL-induced apoptosis.

The agents according to the invention may be administered to a subject to be treated by any route of administration known to the person skilled in the art. The agent may be supplied in a liquid or solvent form. Of course, the agent may be supplied by systemic and/or topic ways. The administration can be intermittent or continuous.

For example, the agent may be applied intravenously to a person in need thereof. Of course, other forms of administration are also within the scope of the present invention. For example, capsules containing the agent or syrup may be used for oral administration.

According to a further preferred embodiment the agent may be encapsulated. Corresponding encapsulation materials are known to the person skilled in the art. In particular the use of tumour targeting agents or materials coupled with tumour targeting agents as encapsulation material is preferred.

According to a further preferred embodiment the agent of the present invention is used in combination with at least one further therapeutic compound such as chemotherapeutics, such as alkylating agents, antimetabolites, anthracyclines, plant alkaloids, topoisomerase inhibitors, and other antitumour agents, blockers of apoptosis, targeted drugs and/or even irradiation therapy.

According to the invention, targeted drugs comprise, but are not limited to, anti-VEGF, anti-EGF-R, anti-Her2, anti-CD20, kinase inhibitors with anti-neoplastic activity known to the person skilled in the art and preferably currently in clinical development, histone deacetylase (HDAC) inhibitors, proteasome inhibitors, DNA-methyl transferase inhibitors, etc., and most preferably recombinant TRAIL, anti-TRAIL-R1, anti-TRAIL-R2, as well as blockers of death ligands such as TNF-R2-Fc(Enbrel), anti-TNF, CD95-Fc, anti-CD95L (anti-FasL), steroid and non-steroid anti-inflammatory drugs and any combination thereof.

Especially preferred is a combination with a TRAIL-receptor (TRAIL-R) agonist. The term “TRAIL-R agonist” comprises any TRAIL-R agonist known to the person skilled in the art, cf. for example review articles by Falschlehner et al. (“Therapeutic Targets of the TNF Superfamily”, Chapter “TRAIL and Other TRAIL receptor Agonists as Novel Cancer Therapeutics”, 2009, Landes Bioscience and Springer Science Buisness Media) or Papenfuss et al. (“TRAIL-Rezeptor-Agonisten, eine neue Klasse pro-apoptotischer Krebstherapeutika”, Onkopipiline, not published yet), which are herein incorporated by reference, or which will be identified.

Preferred TRAIL-R agonists are selected from the group consisting of, but not limited to, TRAIL, preferably exogenous TRAIL such as any kind of recombinant TRAIL, and/or anti-TRAIL-receptor antibodies such as anti-TRAIL-R1 or anti-TRAIL-R2. In particular preferred TRAIL agonists are rhApo2L/TRAIL (PRO1762, AMG-951), i.e. recombinant TRAIL binding to TRAIL-R1 and TRAIL-R2, Mapatumumab (HGS-ETR1), i.e. a human monoclonal antibody against TRAIL-R1, Lexatumumab (HGS-ETR2), i.e. a human monoclonal antibody against TRAIL-R2, CS-1008, i.e. a monoclonal antibody, humanized form of the murine anti-TRAIL-R antibody TRA-8, LBY135, i.e. chimeric monoclonal antibody against TRAIL-R2, Apomab, i.e. human monoclonal antibody against TRAIL-R2, AMG-655, i.e. human monoclonal antibody against TRAIL-R2, and/or Ad5-TRAIL, recombinant TRAIL overexpressed by adenovirus, binding to TRAIL-R1 and TRAIL-R2. However, endogenous. TRAIL, the expression of which has been stimulated, enabled, induced and/or improved may be also a TRAIL-R agonist in terms of the present invention. According to a further embodiment, a therapeutic approach using TRAIL-R agonists may comprise the use of further chemotherapeutic agents.

Of course, it is also possible that re-expression of proteins encoded by nucleic acids listed in table 1 is achieved by e.g. an inhibitor of EGF-receptor, any other targeted drug, chemo- and/or radiotherapy and that this may predict sensitivity of a given tumor to EGF-receptor, any other targeted drug, chemo- and/or radiotherapy with or even without the need for addition of TRAIL or a TRAIL receptor agonist. The latter could be related to the tumor cells being sensitised to EGF-receptor, any other targeted drug, chemo- and/or radiotherapy and/or to endogenous TRAIL or to other endogenous mechanisms of cell death induction, e.g. by CD95L, TNF, TL1A and/or by other means.

Blockers of apoptosis-factors comprise, for example, blockers of TNF, e.g. TNF-R2-Fc (Enbrel), or anti-TNF antibodies (like Remicade and Humira), blockers of CD95L, e.g. CD95-Fc or any antibody which blocks CD95L (FasL), and/or blockers of TRAIL, e.g. TRAIL-R2-Fc or a fusion protein of any other TRAIL-R capable of binding to and thereby blocking TRAIL, or any antibody which blocks TRAIL (Apo2L). These inhibitors could also be combined with other current anti-inflammatory treatments e.g. steroid or non-steroid anti-inflammatory drugs.

According to a further especially preferred embodiment one agent according to the invention is used in combination with at least one further agent as defined by the present invention.

In some cases it may be particularly advantageous to combine a blocker of one apoptosis-factor, e.g. a blocker of TNF, with an agonist of another death receptor, e.g. a TRAIL-R agonist. In other cases it may be advantageous to combine one, two or three or more blockers of different apoptosis-factors as defined above. The therapeutic value of such drug combinations may be predictable or enhanced by combination with an agent or with combinations of agents according to the invention.

Preferably the agent of the invention is for use in human or veterinary medicine. The agent of the invention may be applied to any suitable subject including, for example, mammals such as dogs, cats, cows, horses, rabbits, monkeys, and most preferably, humans.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art.

Another aspect of the present invention is a method of diagnosing or monitoring a condition or disorder, preferably an apoptosis-factor-associated condition or disorder, in particular a TRAIL-associated condition or disorder, in a cell or an organism, comprising determining in a sample from said cell or organism the amount and/or activity of at least one nucleic acid molecule as identified in Table 1, a homologue thereof or a polypeptide encoded by said nucleic acid.

An alteration in the amount or activity relative to a sample from a corresponding unaffected cell or organism, is an indication that a condition or disorder to be diagnosed or monitored such as TRAIL-associated condition or disorder is present. The apoptosis-factor-associated condition or disorder is preferably caused by dysregulated cell survival, migration, proliferation and/or non-apoptotic or apoptotic cell-death, i.e. cell survival, migration, proliferation and/or non-apoptotic or apoptotic cell-death which differs from a corresponding unaffected cell or organism.

According to a preferred embodiment of the method of the present invention the apoptosis-factor is selected from the group consisting of TRAIL, CD95L, TNF, TL1A and any combination thereof, and is most preferably TRAIL.

Of course, at least one further condition- or disorder-associated factor may be determined. Thus, the inventive method may be combined with the use of further biomarkers, e.g. biomarkers of cancer if monitoring a cancer-associated condition or disorder.

The presence of a nucleic acid molecule of the invention in a sample can be detected by DNA-DNA or DNA-RNA hybridization and/or amplification using probes or portions or fragments of said nucleic acid molecules. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplifier.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of RNA probes, PCR amplification using a nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

The presence of a polypeptide of the invention in a sample can be determined by immunological methods or activity measurement. A variety of protocols for detecting and measuring the expression of proteins, using either polyclonal or monoclonal antibodies specific for the protein or reagents for determining protein activity are known in the art Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158: 1211-1226).

Suitable reporter molecules or labels, which may be used, include radionuclides, enzymes, fluorescent, chemiluminescent or chromogenic agents as well as substrates, co-factors, inhibitors, magnetic particles, and the like.

The invention further provides the use of a nucleic acid molecule as identified in Table 1, a homologue thereof or a polypeptide encoded by said nucleic acid as a diagnostic marker for TRAIL-, CD95L-, TNF- and/or TL1A-associated cell death, preferably TRAIL-induced apoptosis. The diagnostic markers according to the invention represent a class of predictive markers which can in particular be used for individualized tumor therapy, in particular using TRAIL-R agonists, possibly in combination with other drugs.

Another embodiment of the invention is a diagnostic tool or agent for TRAIL-, CD95L-, TNF- and/or TL1A-associated cell survival, migration, proliferation, non-apoptotic cell-death and/or apoptosis, preferably TRAIL-associated apoptosis, comprising at least one reagent for determining the amount and/or activity of a nucleic acid molecule as identified in Table 1, a homologue thereof or a polypeptide encoded by said nucleic acid. The reagents may be selected from nucleic acid molecules, polypeptides, antibodies, or other chemical compounds.

According to a preferred embodiment of the invention, the diagnostic tool comprises a plurality of reagents. For example, the diagnostic tools in terms of the invention may comprise a panel of at least two reagents for determining the amount and/or activity of a nucleic acid molecule as identified in Table 1, a homologue thereof or a polypeptide encoded by said nucleic acid.

The diagnostic tool may additionally comprise at least one further reagent for determining the amount and/or activity of further TRAIL-, CD95L-, TNF- and/or TL1A-associated nucleic acid molecules or polypeptides, in particular TRAIL-associated nucleic acid molecules or polypeptides such as FADD, cFLIP, Caspase-8, Caspase-10, TRAIL-R1, TRAIL-R2, TRAIL-R3, TRAIL-R4, OPG, RIP1, and Axin-1. The further reagents may be selected from nucleic acid molecules, polypeptides, antibodies, or other chemical compounds.

A preferred embodiment of a diagnostic tool for TRAIL-, CD95L-, TNF- and/or TL1A-associated cell survival, migration, proliferation, non-apoptotic cell-death and/or apoptosis, in particular TRAIL-associated apoptosis, is a microarray. A microarray has molecules distributed over, and stably associated with, the surface of a solid support. The term “microarray” refers to an arrangement of a plurality of reagents. The reagents may the provided for determining e.g. the amount and/or activity of a nucleic acid molecule as identified in Table 1, a homologueue thereof or a polypeptide encoded by said nucleic acid. Alternatively and/or additionally the reagents may be provided for determining the amount and/or activity of further TRAIL-associated nucleic acid molecules or polypeptides such as FADD, cFLIP, Caspase-8, Caspase-10, TRAIL-R1, TRAIL-R2, TRAIL-R3, TRAIL-R4, OPG, RIP1, and Axin-1. The reagents may be selected from nucleic acid molecules, polypeptides, antibodies, or other chemical compounds.

Microarrays may be prepared, used, and analyzed using methods known in the art (see for example, Brennan T. M., (1995) U.S. Pat. No. 5,474,796; Schena M. et al., (1996) Proc. Natl. Acad. Sci. USA 93: 10614-10619; Baldeschwieler et al., (1995) PCT application WO9525116; Shalon T. D. and Brown P. O., (1995) PCT application WO9535505; Heller R. A et al., (1997) Proc. Natl. Acad. Sci. USA 94: 2150-2155; Heller M. J. and Tu E., (1997) U.S. Pat. No. 5,605,662). Various types of microarrays are well known and thoroughly described in Schena M., ed. (1999); DNA Microarrays: A Practical Approach, Oxford University Press, London.

Oligonucleotides or longer fragments derived from any of the polypeptides described herein may be used as elements on a microarray. The microarray can be used to monitor the relative expression levels of a plurality of nucleic acid molecules identified in table 1 and/or of further TRAIL-, CD95L-, TNF- and/or TL1A-associated nucleic acid molecules, in particular TRAIL-associated nucleic acid molecules, simultaneously. The microarray may also be used to identify genetic variants, mutations, and polymorphisms. This information may be used to diagnose a disorder, to monitor progression/regression of disease as a function of gene expression, and to develop and monitor the activities of therapeutic agents in the treatment of disease.

In another embodiment, the invention relates to a method of identifying a modulator of TRAIL-, CD95L-, TNF- and/or TL1A-associated cell survival, migration, proliferation, non-apoptotic cell-death and/or apoptosis, in particular TRAIL-associated apoptosis, comprising evaluating or screening if a test compound has the ability to modulate the amount and/or activity of at least one nucleic acid molecule as identified in Table 1, a homologue thereof or a polypeptide encoded by said nucleic acid.

According to a preferred embodiment the test compound induces a nucleic acid molecule as identified in Table 1, a homologue thereof or a polypeptide encoded by said nucleic acid with the test compound and evaluating, if the amount and/or activity thereof is altered in the presence of the test compound. For example, the test compound could be a molecule that induces one of the nucleic acid molecules according to Table 1, or even a few of them simultaneously. For example, the test compound may be an inhibitor of histone deacetylases and such a compound leads to the re-expression of caspase-8 and two or three proteins encoded by nucleic acid molecules according to Table 1. It putatively does so in 30% of lung cancer, e.g. in lung cancer not having mutations in B-RAF. It also putatively does so in 25% of breast cancers, e.g. in non-Her2-expressing breast cancers. Then this test compound, the HDAC inhibitor, could be used in combination with TRAIL or another TRAIL-Receptor agonist to treat tumour patients, especially tumour patients in which we could observe the upregulation of these tumour markers following use of the HDAC inhibitor.

Accordingly, another preferred embodiment relates to the identification of a test compound to be used in combination with a TRAIL-R agonist as defined above, such as TRAIL, preferably exogenous TRAIL, e.g. recombinant TRAIL, and/or an anti-TRAIL-receptor antibody such as anti-TRAIL-R1 or anti-TRAI L-R2.

The method may for example comprise contacting a cell or an organism including a nucleic acid molecule as identified in Table 1, a homologue thereof or a polypeptide encoded by said nucleic acid with the test compound and evaluating, if the amount and/or activity thereof is altered in the presence of the test compound.

The result of evaluation may provide information, whether the test compound stimulates, enables, inhibits or prevents TRAIL-, CD95L-, TNF- and/or TL1A-associated cell survival, migration, proliferation, non-apoptotic cell-death and/or apoptosis, in particular TRAIL-associated apoptosis.

If the test compound stimulates the TRAIL-, CD95L-, TNF- and/or TL1A-associated non-apoptotic cell-death and/or apoptosis, in particular TRAIL-associated apoptosis, the test compound is a candidate agent for the treatment of inflammation, rheumatoid arthritis, multiple sclerosis, hyperproliferative disorders, such as cancer and/or viral infections such as by CMV, influenza virus, respiratory syncytial virus etc. In an especially preferred embodiment the test compound is used in combination with TRAIL and/or another stimulator of TRAIL-induced cell death.

If the test compound stimulates the TRAIL-, CD95L-, TNF- and/or TL1A-associated cell survival, migration and/or proliferation, the test compound is a candidate agent for the treatment of acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis and/or viral infections e.g. by HIV.

If, on the other hand, the test compound inhibits non-apoptotic cell-death and/or apoptosis, in particular TRAIL-associated apoptosis, the test compound is a candidate agent for the treatment of cancer, acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis and/or viral infections e.g. by HIV.

If the test compound inhibits the TRAIL-, CD95L-, TNF- and/or TL1A-associated cell survival, migration and/or proliferation, the test compound is a candidate agent for the treatment of inflammation, rheumatoid arthritis, multiple sclerosis and/or hyperproliferative disorders, such as cancer and/or viral infections such as by CMV, influenzy virus, respiratory syncytial virus, etc.

Of particular interest are screening assays for candidate agents that have a low toxicity for mammalian cells.

The term “test compound” as used herein describes any molecule, e.g. protein or pharmaceutical, with the capability of altering or modulating the physiological function of one or more of the nucleic acid molecules identified in table 1 or homologues thereof, or of the polypeptides of the invention of homologueues thereof. Test compounds encompass numerous chemical classes, though typically they are organic molecules, preferably small organic compounds having a molecular weight of more than 50 and less than about 2,500 Daltons.

The test compound may for example be (i) an antibody directed against the polypeptide, (ii) a truncated or mutated fragment of the polypeptide, (iii) a nucleic acid effector molecule or (iv) a low-molecular weight compound.

Test compounds are also found among biomolecules including peptides, saccharides, fatty acids, steroids, purines, pyrimidines, nucleic acids and derivatives, structural analogs or combinations thereof. Test compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

The invention will now be described in more detail with reference to the figures and experimental examples.

### FIGURES

FIG. 1: Schematic representation of the TRAIL signalling network. Binding of TRAIL to TRAIL-R1 or -R2 leads to receptor trimerisation and formation of the death-inducing signalling complex (DISC). The adaptor protein FADD is recruited to the DISC where the death domains (DD) of both proteins interact. Subsequently, procaspases 8 and 10 are recruited to the protein complex where they interact with FADD via the death effector domains (DEDs). cFLIP can compete with caspase-8 for the binding to FADD. Therefore, high levels of cFLIP can abrogate caspase-8 activation at the DISC. DISC-activated caspases 8 and 10 trigger a caspase cascade by cleavage of caspase-3. In addition, Bid is cleaved into tBid which initiates the mitochondrial apoptosis pathway leading to release of cytochrome c (CytC) and Smac/DIABLO from the mitochondria. CytC, together with Apaf-1 forms the apoptosome, an activation platform for caspase-9. Smac/DIABLO conteracts the inhibitory function of XIAP thereby allowing for full activation of caspases 3 and 9, ultimately leading to cell death.

FIG. 2: Titration curve for TRAIL-induced apoptosis. HeLa cells were stimulated with 0.001 to 1000 ng/ml TRAIL, followed by analysis of cell viability by the quantification of ATP (CelliterGlo® assay).

FIG. 3. Detection of RNA1-mediated viability phenotypes. An ATP-quantification assay was used to detect changes in viability upon transfection of HeLa cells with siRNAs targeting ubiquitin B (UBB), ubiquitin C (UBC) and polo-like kinase 1 (PLK1). Compared to a negative control siRNA targeting Renilla luciferase (RLUC), all three siRNAs resulted in severe cell death after 72 hours of mRNA depletion.

FIG. 4. Genome-wide survey of viability phenotypes in cultured human cells. (A) 21,115 siRNA-pools, prealiquoted in 384-well cell culture plates, were used to conduct a genomewide RNAi-survey of cell viability phenotypes. The screen was done in duplicates, divided into three subbatches. At day 0, HeLa cells were reversely transfected with the siRNA-library. After 72 hours of incubation, the viability was measured by quantifying the cellular ATP-content of each well. The data sets were analyzed using the R/Bioconductor software package cellHTS2 (http://www.bioconductor.org) (Boutros 2006). (B) The normalized value of each knockdown is plotted for both replicates. The replicate screens showed highly reproducible phenotypes of various strengths.

FIG. 5. TRAIL-induced cell death in HeLa cells can be rescued by siRNAs. (A) TRAIL induces apoptosis in HeLa cells. Membrane blebbing was observed after 1-2 hours and a complete loss of viable cells was detected after 24 hours. (B) The killing activity of TRAIL in HeLa cells is concentration dependent. (C) Silencing the TRAIL-R1 receptor and caspase-8 with siRNAs, rescued the apoptotic effect to 70% and 100% viability of untreated cells, respectively.

FIG. 6. Genome-wide siRNA screens for mediators of TRAIL-induced apoptosis. The genome-covering siRNA-library was reversely transfected into human HeLa cells according to the viability screen protocol. 48 hours after the transfection procedure, cells were treated with 100 ng/ml TRAIL to induce apoptosis. Cells were incubated for further 24 hours with the ligand to ensure a proper decay of cellular ATP. Finally, the ATP-levels were quantified and the data sets were analyzed.

FIG. 7. The TRAIL screen replicates were highly reproducible. The genome-wide TRAIL screen was done in five replicates over a time period of 14 months to monitor the technical reproducibility of genome-wide screens. The normalized values were plotted to demonstrate the distribution of the values (green plots on the diagonal) and plotted in pairwise combinations to show the reproducibility (lower dotplots). The relating Pearson's correlation coefficients are represented as numbers (upper right). The correlation coefficients of 0.83 to 0.95 (1.0=identical) demonstrate the high reproducibility of the genome-wide screens.

FIG. 8. Genome-wide siRNA screens reveal novel regulators of TRAIL-mediated apoptosis. The five TRAIL screen data sets were normalized (see text for details) and plotted against the theoretical distribution of quantiles (A). The stringent hitlist ‘A’ (red) was defined to contain the 48 top scoring siRNAs. With the help of a randomization approach, hitlist ‘B’ (blue) was created including 665 genes, which significantly deviate from a random distribution. (B) A heatmap was constructed using the 48 top scoring candidates from hitlist ‘A’ (TRAIL screen) and the 13 top scoring genes from the viability screen. The values for each replicate are shown in red meaning cell death in the viability screen and a rescue phenotype in the TRAIL screen and vice versa a proliferation phenotype upon gene knockdown (viability screen) and cell death after TRAIL treatment for blue.

FIG. 9. Systematic retests of TRAIL screen candidates. (A) A robust hitlist was generated from the five TRAIL screen replicates. The candidates were retested in two different approaches to exclude sequence-dependent off-targets. (B) 175 candidates were retested with independent siRNA-pools (Qiagen). Of those, 36 genes could be confirmed having a rescue phenotype when silenced. (C) For 24 candidates, the siRNA-pools used in the screen (Dharmacon) were deconvoluted and tested as single siRNA-sequences in addition to a self-mixed pool. 19 candidates were confirmed showing a rescue phenotype with 2 or more single sequences and the pool.

FIG. 10: Validation of novel TRAIL modulators in HeLa cells. Hela cells were transfected with the respective siRNA pools for 48 h. Then TRAIL was added in a concentration range from 0 to 500 ng/ml for 24 h, followed by the quantification of cell viability by a mitochondrial activity assay (MTT assay). Data are shown as the mean percent viability +/− range (one experiment, three wells per condition).

FIG. 11: Validation of novel TRAIL modulators in the breast cancer cell line MDA-MB-231. MDA-MB-231 cells were transfected with the respective siRNA pools for 48 h. Then TRAIL was added in a concentration range from 0 to 500 ng/ml for 24 h, followed by the quantification of cell viability by a mitochondrial activity assay (MTT assay). Data are shown as the mean percent viability +/− range (one experiment, three wells per condition).

FIG. 12: Validation of novel TRAIL modulators in the colon cancer cell line DKO4. DKO4 cells were transfected with the respective siRNA pools for 48 h, followed by TRAIL application in a concentration range from 0 to 500 ng/ml for 24 h. Cell viability was quantified by a mitochondrial activity assay (MTT assay). Data are shown as the mean percent viability +/− range (one experiment, three wells per condition).

FIG. 13: KD of novel TRAIL modulators enhances long-term survival. HeLa cells were cultured in the presence of the respective siRNA pools for 48 h followed by TRAIL treatment [500 ng/ml] or medium without TRAIL for 48 h and subsequent incubation with fresh medium for 5 days. Afterwards, dead cells were washed away and remaining, i.e. living cells, were stained with crystal violet.

FIG. 14: Western Blot analysis of Agtrap and control KD cells after TRAIL stimulation. (A) HeLa cells were cultured in the presence of Agtrap and Rluc (control) siRNAs, respectively, for 48 h followed by cell lysis, SDS-page and Western blot analysis. Western blot analysis of caspase-8, FADD, c-Flip, Bid, caspase-9 and XIAP after addition of TRAIL [100 ng/m1] for the indicated times (0, 0.5, 1, 2, 4 h) is shown. An antibody against β-actin was used as loading control. (B) In parallel to the Western blot analysis, part of the transfected Agtrap KD and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 15: KD of CRIP1 influences caspase-8 cleavage after TRAIL treatment. A) HeLa cells were cultured in the presence of CRIP1 and Rluc (control) siRNAs, respectively, for 48 h followed by cell lysis, SDS-page and Western blot analysis. Western blot analysis of caspase-8, FADD, Bid, Caspase-9, XIAP and Bcl-2 after addition of TRAIL [100 ng/m1] is shown for the indicated time period (0, 0.5, 1, 2, 4 h). An antibody against α-actin was used as loading control. B) In parallel to the Western blot analysis, part of the CRIP1 KD and Rluc KD HeLa cells were transferred to 96-well plates and used for viability quantification after stimulation with TRAIL (100 ng/ml) for 24 h. Viability was measured by the quantification of mitochondrial activity (MU assay). Data are shown as the mean percent viability +/− range (three wells per condition).

FIG. 16: KD of FBXO31 influences caspase cleavage. A) HeLa cells were cultured in the presence of FBXO31 and Rluc (control) siRNAs, respectively, for 48 h followed by cell lysis, SDS-page and Western blot analysis. Western blot analysis of caspase-8, FADD, caspase-3, Bid, TRAF2, PARP and Ubiquitin after addition of TRAIL [200 ng/ml] is shown for the indicated time period (0, 30, 60, 120, 240 min) in FBXO31 KD and Rluc KD HeLa cells. An antibody against β-actin was used as loading control. B) In parallel to the Western blot analysis, part of the transfected FBXO31 KD and Rluc KD HeLa cells were transferred to 96-well plates and used for viability quantification after stimulation with TRAIL (200 ng/ml) for 24 h. Viability was measured by the quantification of mitochondrial activity (MU assay). Data are shown as the mean percent viability +/− range (three wells per condition).

FIG. 17: KD of KIAA0431 influences caspase cleavage. A) HeLa cells were cultured in the presence of KIAA0431 and Rluc (control) siRNAs, respectively, for 48 h followed by cell lysis, SDS-page and Western blot analysis. Western blot analysis of caspase-8, FADD, Bid, Caspase-9, XIAP and Bcl-2 after addition of TRAIL [100 ng/m1] is shown for the indicated time period (0, 0.5, 1, 2, 4 h). An antibody against β-actin was used as loading control. B) In parallel to the Western blot analysis, part of the KIAA0431 KD and Rluc KD HeLa cells were transferred to 96-well plates and used for viability quantification after stimulation with TRAIL (100 ng/ml) for 24 h. Viability was measured by the quantification of mitochondrial activity (MU assay). Data are shown as the mean percent viability +/− range (three wells per condition).

FIG. 18: KD of KPNA4 only slightly influences caspase cleavage. (A) HeLa cells were cultured in the presence of KPNA4 and Rluc (control) siRNAs, respectively, for 48 h followed by cell lysis, SDS-page and Western blot analysis. Western blot analysis of caspase-8, FADD, Bid, Caspase-9, XIAP and Bcl-2 after addition of TRAIL [100 ng/m1] is shown for the indicated time period (0, 0.5, 1, 2, 4 h). An antibody against β-actin was used as loading control. (B) In parallel to the Western blot analysis, part of the KPNA4 KD and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 19: Western Blot analysis of Magmas and control KD cells after TRAIL stimulation. (A) HeLa cells were cultured in the presence of Magmas and Rluc (control) siRNAs, respectively, for 48 h followed by cell lysis, SDS-page and Western blot analysis. Western blot analysis of caspase-8, FADD, c-Flip, Bid, caspase-9 and XIAP after addition of TRAIL [100 ng/ml] is shown for the indicated time period (0, 0.5, 1, 2, 4 h). An antibody against β-actin was used as loading control. (B) In parallel to the Western blot analysis, part of the transfected Magmas KD and Rluc KD HeLa cells were transferred to 96-well plates and used for viability quantification after stimulation with TRAIL (100 ng/ml) for 24 h. Viability was measured by the quantification of mitochondrial activity (MIT assay). Data are shown as the mean percent viability +/− range (three wells per condition).

FIG. 20: Western Blot analysis of MAPK9 KD and control KD cells after TRAIL stimulation. A) HeLa cells were cultured in the presence of MAPK9 and Rluc (control) siRNAs, respectively, for 48 h followed by cell lysis, SDS-page and Western blot analysis. Western blot analysis of caspase-8, FADD, c-Flip, Bid, Caspase-9, and XIAP after addition of TRAIL [100 ng/ml] is shown for the indicated times (0, 0.5, 1, 2, 4 h). An antibody against β-actin was used as loading control. B) In parallel to the Western blot analysis, part of the MAPK9 KD and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 21: Western Blot analysis of MDS1 and control KD cells after TRAIL stimulation. A) HeLa cells were cultured in the presence of MDS1 and Rluc (control) siRNAs, respectively, for 48 h followed by cell lysis, SDS-page and Western blot analysis. Western blot analysis of caspase-8, FADD, Bid, Caspase-9, XIAP and Bcl-2 after addition of TRAIL [100 ng/m1] is shown for the indicated time period (0, 0.5, 1, 2, 4 h). An antibody against β-actin was used as loading control. B) In parallel to the Western blot analysis, part of the MDS1 KD and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 22: Western Blot analysis of MMRP19 KD and control KD cells after TRAIL stimulation. A) HeLa cells were cultured in the presence of MDS1 and Rluc (control) siRNAs, respectively, for 48 h followed by cell lysis, SDS-page and Western blot analysis. Western blot analysis of caspase-8, FADD, Bid, Caspase-9, XIAP and Bcl-2 after addition of TRAIL [100 ng/ml] is shown for the indicated time period (0, 0.5, 1, 2, 4 h). An antibody against β-actin was used as loading control. B) In parallel to the Western blot analysis, part of the MMRP19 KD and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 23: Western Blot analysis of NUCKS KD and control KD cells after TRAIL stimulation. A) HeLa cells were cultured in the presence of NUCKS and Rluc (control) siRNAs, respectively, for 48 h followed by cell lysis, SDS-page and Western blot analysis. Western blot analysis of caspase-8, FADD, Bid, Caspase-9, XIAP and Bcl-2 after addition of TRAIL [100 ng/m1] is shown for the indicated time period (0, 0.5, 1, 2, 4 h). An antibody against 11-actin was used as loading control. B) In parallel to the Western blot analysis, part of the NUCKS KD and Rluc KD HeLa cells were transferred to 96-well plates and used for viability quantification after stimulation with TRAIL (100 ng/ml) for 24 h. Viability was measured by the quantification of mitochondrial activity (MU assay). Data are shown as the mean percent viability +/− range (three wells per condition).

FIG. 24: Western Blot analysis of OR9G4 KD and control KD HeLa cells after TRAIL stimulation. A) HeLa cells were cultured in the presence of OR9G4 and Rluc (control) siRNAs, respectively, for 48 h followed by cell lysis, SDS-page and Western blot analysis. Western blot analysis of caspase-8, FADD, Bid, Caspase-9, XIAP and Bcl-2 after addition of TRAIL [100 ng/ml] is shown for the indicated time period (0, 0.5, 1, 2, 4 h). An antibody against β-actin was used as loading control. B) In parallel to the Western blot analysis, part of the Or9G4 KD and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 25: Western Blot analysis of PNAS-4 KD and control KD cells after TRAIL stimulation. (A) HeLa cells were cultured in the presence of PNAS-4 and Rluc (control) siRNAs, respectively, for 48 h followed by cell lysis, SDS-page and Western blot analysis. Western blot analysis of caspase-8, FADD, Bid and Caspase-9 after addition of TRAIL [100 ng/ml] is shown for the indicated time period (0, 0.5, 1, 2, 4 h). An antibody against β-actin was used as loading control. (B) In parallel to the Western blot analysis, part of the PNAS-4 KD and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 26: Western Blot analysis of Qrich1 KD and control KD cells after TRAIL stimulation. A) HeLa cells were cultured in the presence of Qrich1 and Rluc (control) siRNAs, respectively, for 48 h followed by cell lysis, SDS-page and Western blot analysis. Western blot analysis of caspase-8, FADD, c-Flip, Bid, and caspase-9 after addition of TRAIL [100 ng/ml] is shown for the indicated time periods (0, 0.5, 1, 2, 4 h). An antibody against β-actin was used as loading control. B) In parallel to the Western blot analysis, part of the transfected Qrich1 and Rluc KD HeLa cells were transferred to 96-well plates and used for viability quantification after stimulation with TRAIL (100 ng/ml) for 24 h. Viability was measured by the quantification of mitochondrial activity (MTT assay). Data are shown as the mean percent viability +/− range (three wells per condition).

FIG. 27: Western Blot analysis in RNF5 KD and control cells after TRAIL stimulation. (A) HeLa cells were cultured in the presence of RNF5 and Rluc (control) siRNAs, respectively, for 48 h followed by cell lysis, SDS-page and Western blot analysis. Western blot analysis of caspase-8, FADD, Bid, caspase-9, XIAP and Bcl2 after addition of TRAIL [100 ng/ml] is shown for the indicated time period (0, 0.5, 1, 2, 4 h) in RNF5 KD and Rluc KD HeLa cells. An antibody against β-actin was used as loading control. (B) In parallel to the Western blot analysis, part of the transfected RNF5 KD and Rluc KD HeLa cells were transferred to 96-well plates and used for viability quantification after stimulation with TRAIL (100 ng/ml) for 24 h. Viability was measured by the quantification of mitochondrial activity (MU assay). Data are shown as the mean percent viability +/− range (three wells per condition).

FIG. 28: Western Blot analysis of SEP15 KD and control KD cells after TRAIL stimulation. (A) HeLa cells were cultured in the presence of SEP15 and Rluc (control) siRNAs, respectively, for 48 h followed by cell lysis, SDS-page and Western blot analysis. Western blot analysis of caspase-8, FADD, c-Flip, Bid, caspase-9 and XIAP after addition of TRAIL [100 ng/ml] is shown for the indicated time period (0, 0.5, 1, 2, 4 h). An antibody against β-actin was used as loading control. (B) In parallel to the Western blot analysis, part of the transfected SEP15 and Rluc KD HeLa cells were transferred to 96-well plates and used for viability quantification after stimulation with TRAIL (100 ng/ml) for 24 h. Viability was measured by the quantification of mitochondrial activity (MU assay). Data are shown as the mean percent viability +/− range (three wells per condition).

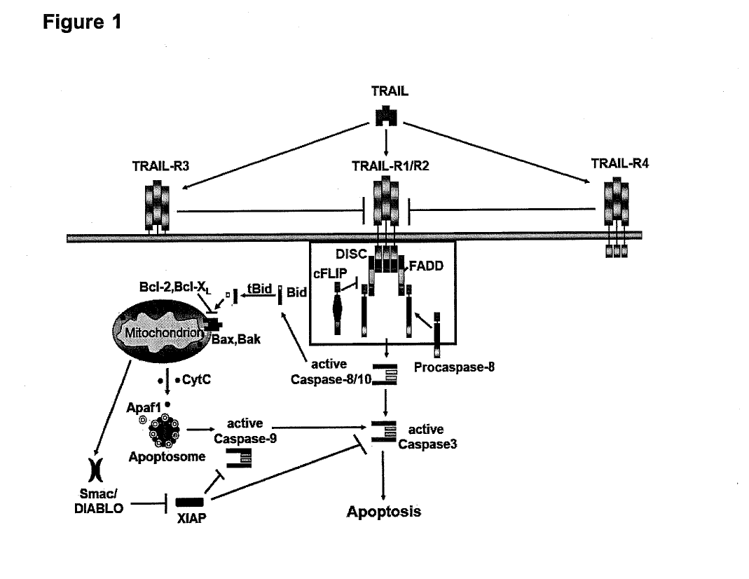

### EXAMPLES

### Methods

### Cell Biological Methods

### Cell Culture and Passaging of Adherent Cells

All cell lines were cultured in 75 cm² or 150 cm² flasks (TPP, Helena Bioscience) in D-MEM medium+Glutamax (Gibco/Invitrogen, Karlsruhe, Germany) supplemented with 10% FCS (Biochrom AG, Berlin, Germany) at 37° C. in a humidified atmosphere with 5% CO₂. At cell densities around 5×10⁶ cells (75 cm² flask) or 1×10⁷ cells (150 cm² flask), cells were washed with 1×PBS followed by incubation with 5-10 ml 1×Trypsin/EDTA for 1-5 minutes. Afterwards, fresh medium containing 10% FCS (5-10 ml) was added to stop the action of trypsin. Detached cells were transferred to a falcon tube, centrifuged and resuspended in fresh medium containing 10% FCS. 1/10 of the resuspended cell solution was transferred to a new flask and fresh medium containing 10% FCS was added to a volume of 10 ml (75 cm² flask) or 20 ml (150 cm² flask). As cells can change in long-term cultures, cells were discarded after a certain passage number (10-15 passages) and a vial of original cells (stored in liquid nitrogen) was taken into culture.

### Counting of Cells

To determine the exact number of cells per ml, adherent cells were detached with trypsin and resuspended in fresh medium containing 10% FCS as described before. 25 μl of this cell suspension was diluted with 25 μl trypane blue and applied to a “Neubauer counting chamber”. Trypane blue penetrates the cell wall of dead cells which is visible in the Neubauer counting chamber under the microscope. All trypane blue negative cells in the four outher big squares were counted and divided by four (=mean cell number/big square). This number is then multiplied by two, as the cell suspension was diluted 1:2 by trypane blue.

Therefore, the cell number per ml is calculated by following formula:

(2×mean trypane negative cell number per big square)×10⁴=cells/ml

### Freezing and Thawing of Cells

For long-term storage, cells were kept in liquid nitrogen (−196° C.). To freeze eukaryotic cell lines, adherent cells were detached from the flasks as described before. After centrifugation, cells were resuspended in precooled (+4° C.) FCS containing 10% DMSO and aliquoted into cyrotubes (5×10⁶-1×10⁷ cells/ml). DMSO was used as a cryoprotectant because it prevents the formation of ice crystals which otherwise would lyse the cells during thawing. The cells were slowly cooled to −80° C. and then transferred to the liquid nitrogen tank where they were kept for long-term storage at −196° C. To take frozen cells into culture, cells were thawed at 37° C. and rapidly transferred to a cell culture flask and 15 ml prewarmed (37° C.) medium containing 10% FCS was added. After attachment of the cells the medium was replaced by prewarmed fresh medium containing 10% FCS and cells were cultured at 37° C. in a humidified atmosphere with 5% CO₂.

### Molecular Methods

### DNA Amplification by Polymerase Chain Reaction (PCR)

For amplification of plasmid or cDNA, polymerase chain reactions (PCRs) were performed. Depending on the intended purpose, different polymerases were used. Taq polymerase (Fermentas Life Sciences) was used for analytic PCRs while proof-reading polymerases Pfu (Fermentas Life Sciences) and KapaHiFi (KAPA Biosystems) were used for preparative PCRs. For one PCR reaction primers, DNA template, polymerase buffer, nucleotides and DNA polymerase were mixed as followed:

Primer 1 (10 pmol/μl) 1 μl
Primer 2 (10 pmol/μl) 1 μl
10× polymerase buffer 5 μl
dNTP Mix (each 10 mM) 1 μl
Template DNA (plasmid, cDNA) 5 μl (10-100 ng)

### Polymerase 1 μl (2.5 U)

### H20 ad 50 μl

The melting temperature of the primers used for the PCR was calculated according following formula Tm=[(A+T)×2]+[(C+G)×4]. In an ideal situation, the GC content should be 50%. The annealing temperature ranged from 50° C. to 60° C. according to the used primers. The elongation time was calculated according to the length of the amplicon (60 sec/1000 bp). The scheme of the PCR is shown below.

Denaturation 95° C. 3 min; Denaturation 95° C. 35 sec; Annealing 50-60° C. (according to Primer Tm) 35 sec, 30×; Elongation 72° C. (68° C. for Pfu polymerase) 60 sec/1000 bp; Final elongation 72° C. 10 min; Cool-down to 4° C.

### DNA Digestion and Restriction Analysis

Plasmid DNA or amplified PCR fragments were digested with specific enzymes for restriction analysis or subsequent cloning into defined vectors. After DNA digestion, plasmid fragments were supplemented with DNA loading buffer, loaded onto a 1% agarose gel in 1×TAE buffer and subjected to gel electrophoresis. A marker (GeneRuler™ DNA 1 kb Ladder) which allows the determination of the molecular weight size was loaded in parallel to the DNA samples. Afterwards, the DNA molecules were stained with ethidium bromide to make them visible under ultra-violet light.

### Gel Extraction of DNA Fragments

DNA fragments were loaded onto a 1 agarose gel in 1×TAE buffer and subjected to gel electrophoresis as described before. For the isolation of the respective DNA fragment(s), the QIAquick Gel Extraction Kit from Qiagen was used.

### DNA Ligation

For the cloning of a DNA fragment into a vector that was cut at a single restriction site (e.g. pcDNA3.1 cut with BamHI), the linearised and purified vector had to be subjected to Shrimp Alkaline Phosphatase (SAP) treatment. Therefore, approximately 1 μg of the linearised vector DNA was incubated with 5 U SAP in 1×SAP buffer at 37° C. for 30 min. Afterwards the phosphatase was inactivated at 65° C. for 20 min. The cut and purified insert and the dephosphorylated vector used in a ration 5:1 (insert:vector) together with 2 μl T4 ligation buffer containing 10 mM ATP and 2 U T4 ligase for DNA ligation. H₂0 was added to the reaction to a final volume of 20 μl.

### TOPO® PCR Cloning

For instant cloning of PCR fragment without restriction digest, the TOPO® TA cloning kit (Invitrogen, K4800-01), or the Directional TOPO® cloning kit (Invitrogen, K4900-01) were used. The TOPO® TA vector is supplied with single 3′ thymidine overhangs and topoisomerase I covalently bound to the vector. For successful cloning, the PCR had to be performed with Taq polymerase with adds 3′ A-overhangs to the PCR product. For directional TOPO® cloning, the four bases (CACC) had to be added to the forward primer to allow site-directed (GTGG) integration into the TOPO® vector. For the PCR proof-reading Pfu Polymerase or KapaHiFi that create blunt-end PCR products were used. The PCR products were incorporated into the vector following manufacturer's instruction.

Preparation of Competent E. coli and Transformation Thereof.

Methods to generate competent E. coli as well as methods to transform such competent E. coli are known in the art, e.g., Sambrook, Fritsch & Maniatis, Molecular Cloning, A Laboratory Manual, 2′ edition, Cold Spring Harbor Laboratory Press 1989, Vols. I, II, III.

mRNA Quantification by Quantitative Real-Time PCR (qPCR)

### Total RNA Isolation

Total RNA was isolated from cells using TRIZOL (Invitrogen). Briefly, 5×105-1×10⁶ siRNA transfected or untransfected cells were detached from the plates as described before. The cell pellet was transferred into a 1.5 ml Eppendorf tube, centrifuged and the supernatant was removed. The cell pellet was then thoroughly resuspended in 1 ml TRIZOL and incubated for 5 min at RT under the fume hood. Afterwards, 200 μl chloroform were added and the solution was mixed by shaking for 15 sec followed by incubation for 3 min at RT and centrifugation at 13 000 rpm (4° C.) for 15 min. After centrifugation a phase separation can be observed. The upper aqueous phase containing the RNA was transferred to a new eppendorf tube and 200 μl isopropanol were added followed by incubation at RT for 10 min to precipitate the RNA. After a centrifugation step (13 000 rpm, 4° C., 15 min), the supernatant was removed and 300 μl 70% Ethanol were added to wash the RNA pellet. After centrifugation at 13 000 rpm (4° C.) for 10 min, the supernatant was removed and the RNA pellet was air-dried for approximately 5 min and subsequently dissolved in RNAse-free water.

### Reverse Transcription

After isolation, the RNA was reverse transcribed into cDNA using RevertAid™ cDNA Synthesis Kit according to manufacturer instruction (Fermentas Life Sciences).

Quantitative Real-Time PCR (qPCR)

The amount of gene specific mRNA was quantified using the ABI PRISM 7900 HT Sequence Detection System (Applied Biosystems) System and the Universal Probe Library (Roche Diagnostics). The Universal Probe library Assay Design Centre (https://www.roche-appliedscience.com) was used to create primers and probes specific for each gene of interest. The Mastermix ABsolute qPCR ROX Mix (ABgene) was used for the amplification of DNA. For each (qPCR) reaction 4.4 μl of the previously transcribed cDNA was used in a total volume of 13 μl. The qPCR reaction was performed as followed:

### Initiation 50° C. for 2 min

Enzyme activation 95° C. for 15 min

### Denaturation 95° C. for 15 sec

### Annealing/Extension 60° C. for 60 sec

The cycle threshold C(t) values were recorded and analysed using the PE Biosystems ABI 7900 sequencer software. After normalization on basis of the housekeeping genes GAPDH and HPRT, relative differences in mRNA levels were assessed based on the C(t) values.

siRNA-Mediated Knock-Down (KD) of Target Genes

Genome-wide siRNA knock-down experiments were done in two different studies A) and B).

### A)

For the genome-wide KD experiments a synthetic siRNA library (Dharmacon) covering all unique genes annotated in the human RefSeq database V5.0 was used. Each gene was targeted by a pool of 4 single siRNA sequences. A siRNA sequence targeting Renilla Luciferase (Rluc) was used as control (Elbashier et al., 2001). The Axin-1 phenotype was further confirmed with single siRNA sequences from Dharmacon (MU-009625-01), Ambion/Applied Biosystems (121445) and Qiagen (1027415). For high-throughput RNAi-approaches, lyophilized siRNAs were reconstituted in 1×siRNA buffer (Dharmacon) and further diluted with nuclease-free water (Acros Organics) to a concentration of 500 nM. 5 μl of the dilutions were aliquoted in 384-well cell culture plates (Greiner) and finally stored ready-to-use at −20° C. At the day of transfection, the siRNAs were incubated with 0.05 μl/well Dharmafect 1 transfection reagent (Dharmacon), which was prediluted with RPMI (Gibco/Invitrogen) according to manufacturer's instruction. After incubating the mixture for half an hour, a HeLa-cell suspension in complete medium was prepared in a way that 1000 cells were added to each well. The final siRNA-concentration per well was always 50 nM. After incubation of the cells for 48 h at 37° C. in a humidified atmosphere with 5% CO2, medium alone or medium containing TRAIL [100 ng/ml] was added. 24 h later, CellTiterGlo® (Promega) was added to the cells according to manufacturer's instructions. The resulting luminescence was recorded using Mithras LB940 plate reader (Berthold Technologies).

### Analysis of Screen Data Sets—384-Well Plates:

The screen data sets were normalized to remove systematic biases using the R/Bioconductor software package celIHTS2 (Boutros, Bras et al. 2006). Thereby quantile normalization was applied which normalized the distribution of intensities in each screen to the pooled distribution of probes in all screens. Afterwards a sigmoid transformation was applied to the normalised values to characterise whether a value significantly deviates from the normal distribution. The transformation mapped the values to the range 0-1 and the obtained values were called “calls”. For this transformation, the centre of the sigmoid curve was set to 1.5, corresponding approximately to the 95% quantile of the distribution of values. To define hits from the five TRAIL-screen replicates, the average call value for each gene was determined. Hits with a call equal or higher 0.9 were included into a stringent hitlist ‘A’ containing 48 genes. A less stringent hitlist ‘B’ was generated based on a randomisation approach. Therefore, the average rank of each gene was determined across the replicates, based on the matrix of normalized values. The quantile distribution of the combined TRAILscreens was plotted against the theoretical distribution and both hitlists ‘A’ and ‘B’ were colour-coded in red and blue, respectively.

Further siRNA transfections in larger scale (96-well up to 15 cm² plates) were done using single sequences or siRNA pools (indicated for each experiment) and Dharmafect Reagent 1, both purchased from Dharmacon.

### Preparation of cell lysates

HeLa cells were detached from the plates by Trypsin treatment. Cells were harvested by centrifugation at 300×g for 5 min at 4° C., washed twice with 1×PBS and lysates were prepared by resuspending the resulting cell pellets in 50 μl lysis buffer (30 mM Tris-HCl pH 7.5, 150 mM NaCl, 10% glycerol 1% Triton X-100) supplemented with Complete™ protease inhibitors (Roche Diagnostics, Mannheim, Germany) according to the manufacturer's instructions. After incubation for 30 min on ice, lysates were centrifuged at 15 000×g for 30 min at 4° C. to remove nuclei. The supernatant was transferred to a new eppendorf tube and stored at −20° C.

### BCA Assay—Determination of Protein Content

To determine the protein concentration of cell lysates, the bicinchoninic acid (BCA)-containing protein assay was applied (Pierce, Rockford, Ill., USA). A standard curve was created according to manufacturer's instruction and the protein content in the cell lysates was calculated.

### One-Dimensional SDS-Page

Cell lysates were supplemented with two-fold concentrated standard reducing sample buffer (2×RSB) and incubated at 75° C. for 15 min. Subsequently, the reduced lysate was separated on 4-12% Bis-Tris-NuPAGE gradient gels (Novex, San Diego, Calif., USA) in 1×MES or 1×MOPS running buffer at 200 V (120 mA) for 35-60 min. For the separation of proteins smaller than 40 kDa, 1×MES running buffer was used while 1×MOPS running buffer was applied to separate larger proteins.

### Western Blot Analysis

After separation of proteins by SDS-Page, the proteins were transferred to a nitrocellulose membrane (Amersham Pharmacia Biotech, Freiburg, Germany) by electroblotting at 30 V (160 mA) for 2 h. Afterwards, the membrane was shortly washed with deionised H₂0 and stained with Ponceau-S to control for equal blotting. The membrane was then washed with PBST (PBS containing 0.05% Tween-20), followed by blocking with 5% nonfat dry milk in PBST for at least 2 h. One-time washing with PBST served to remove the blocking solution. The membrane was then incubated with the primary antibody solution (0.1-5 μg antibody, 1×PBS, 0.2% milk powder, 0.01% Tween) for 1 h at RT or over-night at 4° C. under constant shaking. Afterwards, the primary antibody solution was transferred to a 50 ml Falcon tube and stored at 4° C. The primary antibody solution was reused 10-20 times. The membrane was washed 5 times for 3 min with PBST, followed by incubation with HRP-conjugated isotypespecific secondary antibody diluted 1:20 000 in PBST for 1 h. After 5 washing steps for 3 min with PBST, the blots were developed by enhanced chemoluminescence following the manufacturer's protocol (Amersham Pharmacia Biotech, Uppsala, Sweden). For weak signals, SuperSignal West Dura (Pierce/Thermo Scientific) or SuperSignal West Femto (Pierce/Thermo Scientific) was used as detection agent, while ECL Western Blotting substrate (Pierce/Thermo Scientific) was applied when strong signals were expected. After film development, blots were incubated in “stripping buffer” (50 mM glycine HCl pH 2.3) for 20 min at room temperature. Subsequently, blots were washed 3 times for 5 min in PBST followed by incubation with blocking solution for at least 30 min. Then, the next primary antibody solution was applied and the procedure repeated.

### Expression of CD95L

For expression of Fc-CD95L the coding sequence of human IgG1 (aa 247-472) was N-terminally fused to the extracellular portion of the CD95L (aa 117-281) and subcloned in a pcDNA3.1 vector with an N-terminal Ig-leader sequence. The vector was amplified in E. coli Top 10 F′ and purified using Qiagen Plasmid Maxi Kit (Qiagen, Germany). 5-9×10⁶ cells per 150 cm2 flask HEK 293 T cells were then transiently transfected with the vector (40 μg DNA per 150 cm² flask). 1 h prior to transfection, the culture medium of HEK 293 T cells was removed and 18 ml medium (D-MEM+10% FCS) containing 25 μM chloroquine were added. For transfection, 100 μl 2.5 M CaCl₂, 40 μg DNA and sterile H₂O were mixed in a total volume of 1 ml. Then, 1 ml 2×HBS was slowly added and the solution was incubated for 15-30 min to allow for formation of DNA/calcium-phosphate complexes. The transfection mix was then slowly applied to the cell culture flask and incubated over night. On the next day, the medium was changed and left on the cells for 48 h to allow for protein production and secretion into the supernatant. The supernatant containing Fc-CD95L was aliquoted, stored at −20° C. and analysed for killing efficiency. In addition, the concentration of Fc-CD95L in the supernatant was compared to known concentrations of purified TNF-R2-Fc. Consequently, the concentration of Fc-CD95L in the supernatant was estimated to be around 100 ng/10 μl (10 μg/ml). To ensure that cell death after application of the Fc-CD95L-containing supernatant is CD95L-mediated, CD95-Fc (Apogenix, Germany) was added to different concentrations of Fc-CD95L (1:10, 1:20, 1:50 dilutions of Fc-CD95L supernatant in culture medium). The mixture was preincubated for 1 h, applied to HeLa cells, and cell viability was quantified 24 h later using CellTiterGlo® assay (Promega).

Expression and Purification of moTAP-TRAIL

For immunoprecipitation, a recombinant form of human TRAIL was produced comprising the extracellular domain (ECD) and a modified Tandem Affinity Purification (moTAP) tag. The moTAP tag consists of a 3×Flag-tag, followed by a prescision site (PreSci) and an AviTag. This allows a two-step purification resulting in reduced contaminations and a very pure receptor signalling complex. The E. coli strain AVB 101 (Avidity, Colo.; USA) was used for expression of moTAPTRAIL. This strain contains a plasmid (paCYC, Chloramphenicol (Cam) resistance) encoding the enzyme BirA allowing for direct biotinylation of moTAP-TRAIL. E. coli AVB 101 were transformed with the vector encoding moTAP-TRAIL (pQE30-moTAP-TRAIL, Amplicillin (Amp) resistance) as described before (chapter 3.2.2.7). Fur production of moTAP-TRAIL, 4 l of bacterial culture were grown under antibiotic selection pressure (Cam 30 μg/ml, Amp100 μg/ml) until the OD600 reached 0.6. Subsequently, IPTG [100 μM] and Biotin [50 μM] were added to induce the production of biotinylated moTAP-TRAIL followed by incubation overnight at 18° C. On the next day, cells were harvested by centrifugation at 4600 rpm for 30 min at 4° C. and bacteria were lysed using Bacteria lysis buffer supplemented with lysozyme [50 μg/ml] and benzonase [5 U/ml]. Three freeze and thaw cycles, in liquid nitrogen and at 42° C. as well as sonification steps (3-5 times, 30 sec, duty cycle 30, output control 40) were performed to further disrupt the bacteria. To pellet still unlysed bacteria and cell debris, the solution was first centrifuged at 4600 rpm for 30 min at 4° C. and afterwards at 15 000 rpm for 30 min at 4° C. to remove the inclusion bodies. The supernatant was then filtered using 0.45 μm syringe filters and applied to a Ni-NTA Sepharose (QIAGEN) containing column (100 ml volume).

Before application of the lysate, the Ni-NTA column was equilibrated with bacteria lysis buffer. The filtered lysate was then applied followed by two washing steps with bacteria lysis buffer and column wash buffer. Subsequently, moTAP-TRAIL was eluted using the column elution buffer and collecting 30 fractions à 10 ml. A maximum flow rate was 3 ml/min and the maximum pressure limit was 0.3. The samples were subjected to SDSPAGE and western blot for purification analysis. After analysis of the elution fractions, moTAP-TRAIL-containing fractions were pooled and subjected to two rounds of dialysis. The protein was first dialysed in 5 l of maintenance buffer w/o L-Arginin over night and then in 3 l of maintenance buffer with L-Arginine over night. Aliquots of purified moTAP-TRAIL were stored at −80° C.

### Immunoprecipitation of Receptor Signalling Complexes

For the precipitation of receptor signalling complexes, 2×10⁶ cells were transfected with respective siRNAs in a 15 cm diameter plate, followed by incubation at 37° C. in a humidified atmosphere with 5% CO2. Afterwards, the supernatant was removed and 10 ml prewarmed (37° C.) D-MEM containing 10% FCS and 0.5 ml purified moTAPTRAIL per 10 ml medium was added to the cells. After incubation for 10 min (or otherwise indicated durations) the supernatant was removed and cells were immediately washed with ice-cold PBS. Cells were then scraped from the plates at 4° C. and transferred to a 15 ml Falcon tube with ice-cold PBS followed by a centrifugation step at 1300 rpm (4° C.) for 3 min. Afterwards, the supernatant was removed and the cells were resuspended in 900 μl ice-cold lysisbuffer (without Triton) and transferred to a 1.5 ml Eppendorf tube. 100 μl 10% Triton-X-100 (4° C.) were added, the tube was mixed and incubated on ice for 45 min. Afterwards, the lysate was centrifuged at 13 000 rpm (4° C.) for 20 min to remove nuclei and cell debris. The supernatant was transferred to a new 1.5 ml Eppendorf tube, the protein content was determined by the BCA assay and the cell lysates were adjusted to contain the same protein amount per ml. 30 μl of the adjusted cell lysates was removed and stored at −20° C. (=lysated before IP). M2 beads (15 μl bead volume) were added to adjusted cell lysates followed by over-night incubation at 4° C. in an overhead shaker. On the next day, the tubes were centrifuged at 7 000 rpm (4° C.) for 3 min, the supernatant was removed and the beads were washed with ice-cold lysis buffer. This procedure was repeated 5 times. Afterwards, 30 μl 2×RSB was added followed by an incubation at 80° C. for 10 min. The tubes were shortly chilled on ice, centrifuged and loaded onto a 4-12% Bis-Tris-NuPAGE gradient gel where the proteins were subsequently separated by SDS page.

### TRAIL Receptor Surface Staining by Fluorescence-Activated Cell Sorting (FACS) Analysis

For the analysis of surface-expressed receptors, cells were detached from the plates and washed with ice-cold FACS-buffer (1×PBS, 5% FCS). After centrifugation (3 min, 1200 rpm, 4° C.) 1-3×10⁵ cells were incubated with 100 μl of the respective solution containing antibody (TRAIL-R1 (HS101), TRAIL-R2 (HS201), TRAIL-R3 (HS301), TRAIL-R4 (HS402), control mIgG1) or recombinant LZ-TRAIL [each 5 μg/ml in ice-cold FACS-buffer] for 30 min on ice. Afterwards, cells were centrifuged and washed with 200 μl ice-cold FACSbuffer. This procedure was repeated 3 times, followed by addition of 100 μl biotinylated secondary goat anti-mouse antibodies or LZ-Antibody [each 5 μg/ml in ice-cold FACS buffer] and incubation for 20 min on ice. Subsequently, cells were centrifuged and washed 3 times with 200 μl ice-cold FACS-buffer. 100 μl biotinylated secondary goat anti-mouse antibodies were added to the cells where LZ-TRAIL and LZ-Antibody were applied, followed by an incubation step for 20 min on ice and 3 washing steps. Finally, cells were incubated with Streptavidin-PE (1:200 in FACS-buffer) for 20 min on ice. Subsequently, cells were centrifuged and washed 3 times with 200 μl ice-cold FACS-buffer, followed by fluorescenceactivated cell sorting (FACS) analysis.

### Phase Contrast Microscopy

Phase contrast microscopy was used to generate high-contrast pictures of living cells. As the speed of light is differently reduced by the medium, the cell, the cytoplasm, the nucleus etc., small phase shifts of the light are converted into contrast changes in the image. Cells were seeded in 96-, 12-, 24- or 6-well plates and pictures were taken after the respective treatment (e.g. after 4 h stimulation with 100 ng/ml TRAIL). For the generation of phase-contrast pictures, the microscope Axiovert 25 (Zeiss, Gottingen; Germany) with the objective LD-APlan, 20×/0.30 Ph1, the camera ProgResC10 (Jenoptik, Jena; Germany) and the Software ProgresC10 were used.

### Cell Viability Assays, Quantification of Apoptosis, and Long-Term Survival Assays

Cell viability was quantified by the measurement of ATP (CeIlTiterGlo® Luminescent Cell Viability Assay) or the quantification of mitochondrial activity (MTT assay). CellTiterGlo® Luminescent Cell Viability Assay was performed according to manufacturer's instructions. Briefly, 24 h after TRAIL treatment, CellTiterGlo® reagent was added to the medium. After mixing and shaking for 2 min, the luminescent signal was measured using Mithras LB 940 (Berthold Technologies, Bad Wildbad, Germany).

MTT (3-(4,5-Dimethylthiazol-2-yl)-2,5-diphenyltetrazolium bromide) is a water-soluble tetrazolium salt. The MTT viability assay is based on the conversion of MTT to purple coloured formazan by mitochondrial enyzmes which are only active in living cells (Mosmann 1983). After application of the respective death stimulus (TRAIL, CD95L, staurosporine etc.) for 24 h, 25 μl MTT solution (2.5 mg/ml in 1×PBS) were added to each well (96-well plate) and incubated for 3 h at 37° C. Subsequently, the MTT-containing medium was removed using a vacuum pump and 100 μl of isopropanol and acetic acid (95:5/v:v) were added. Plates were incubated at RT under constant shaking for 30 min to solve the purple formazan crystals. Subsequently, the absorption was measured at 540 nm using the Photometer Ultrospec 3100 pro (Amersham, Freiburg; Germany) or Multiskan Ascent (Thermo Labsystems, Vantaa; Finnland).

As a direct measurement of apoptotic cell death, DNA fragmentation was quantified as described (Nicoletti et al., 1991). Briefly, 1.5×105 cells were incubated in 12-well plates (Costar, Cambridge, Mass., USA) with or without apoptotic stimuli in 1 ml medium at 37° C. Cells were trypsinised and then collected by centrifugation at 300×g for 5 min at 4° C., washed twice with PBS and then resuspended in 80 μl “nicoletti buffer” containing 0.1% (v/v) Triton X-100, 0.1% (w/v) sodium citrate and 50 μg/ml propidium iodide (PI). Apoptosis was quantitatively determined by flow cytometry after incubation at 4° C. in the dark for at least 24 h as cells containing nuclei with subdiploid DNA content.

For the quantification of cellular caspase activity, the Z-DEVD-based Apo-One assay (Promega) was used. Cells were transfected and treated with TRAIL as described earlier. 24 h after the death stimuli, the assay was carried out according to manufacturer instruction. Fluorescence was measured after 3 hours incubation in the dark on a Mithras LB940 plate reader (Berthold Technologies).

For long-term survival assays, HeLa cells, treated with respective siRNAs for 48 h, were left untreated or treated with 100 ng/ml TRAIL. Dead cells were washed off with PBS after 24 h. Surviving cells were cultured for additional 5 days in the same dish with medium being replaced every 2 days without any further death stimulus. At the indicated time point, cells were washed 2× with PBS, fixed with 10% formaldehyde in PBS for 30 min at room temperature and stained with crystal violet (1% in 50% ethanol).

### Wnt Reporter Assay

Wnt-activity was measured by using a reporter assay consisting of the vectors pRL-CMV (Promega) and TopFlash (van de Wetering, 1997). Firefly and Renilla luciferase activity was measured 48 h after plasmid transfection on a Mithras LB940 plate reader (Berthold Technologies).

### Expression Profiling

Illumina bead chip technology was used to monitor changes in gene expression in response to RNAi treatment and TRAIL stimuli. Therefore, HeLa cells were harvested 72 hours after siRNA transfection and subjected to RNA preparation, cDNA synthesis and qPCR-analysis to verify the knockdown efficiency. The RNA was further checked for concentration and DNA or protein contaminations with a Nanodrop spectrophotometer (Thermo Fisher Scientific). 500 ng of each RNA-sample were used for further processing into more stable cDNA molecules. The cDNA was than transcribed back into complementary RNA (cRNA) in an in vitro transcription reaction, which simultaneously labelled the molecules with Biotin. The labelled probes were hybridized onto HumanRef-8 v2 bead chips (Illumina). They bind 50 bases long gene-specific oligonucleotides, which are coupled to beads immobilized on the array surface (Gunderson 2004; Steinberg 2004). The beads are covered with >1×10⁵ identical oligonucleotides and each bead type has an average 30×representation on the chip to provide the statistical accuracy of multiple measurements. The HumanRef-8 v2 (Illumina) bead chip covers 23,000 transcript probes based on RefSeq release 17 (NCBI). To quantify the expression levels in the different samples, the attached cRNAs were finally visualized with a fluorescent molecule binding Biotin and the array was scanned with a laser to read the signals.

The output files were analysed using BeadStudio software (IIlumina). The resulting data set was normalized across replicates and differences in expression levels were calculated based on control siRNA and medium (without TRAIL) samples. The technical and biological stability of the arrays was determined by calculating the bead standard error difference (BSED) and the array standard deviation difference (ASDD), respectively. Since every bead is represented about 30× on the chip, the BSED is used to describe the error in between beads targeting the same gene. When comparing a treated and an untreated sample, the BSED is calculated by subtracting the mean of the untreated samples from the mean of the treated samples and dividing the result by the sum of the standard errors of both samples. By contrast, the ASDD is used to compare biological replicates hybridized to two independent arrays. Therefore, the mean of one replicate is subtracted from the mean of the other one, and the resulting value is divided by the sum of both standard deviations (personal communication Bernd Korn). The transcription of the RNA samples into cDNA and further cRNA, the hybridisation and the analysis of the chip was done at the Genomics and Proteomics Core Facility (GPCF) of the German Cancer Research Center (DKFZ).

### RNAi Screen Data Analysis

### Data Processing and Normalization

In total, seven genome-wide siRNA screens (replicates) were done in this project. The effect of siRNA-knockdowns without additional treatments on cellular viability was quantified twice (viability screens). Furthermore, the genome-wide screens were conducted in combination with an apoptosis-inducing dose TRAIL, to identify knockdowns rescuing the apoptosis phenotype (TRAIL screens). The high-throughput screen data sets were normalized to remove systematic biases and effects using the R/Bioconductor software package cellHTS2 (http://www.bioconductor.org) (Boutros 2006). The viability screen replicates were adjusted for plate effects by dividing each measurement within a plate by the midpoint of the shorth of all sample values on the plate. The TRAIL screens were preprocessed in a similar fashion, except that plate effects were first log 2 transformed and then each value was subtracted by the midpoint of the shorth of all sample values within the corresponding plate. After normalization, the variance of each replicate screen was centered and adjusted in a per-replicate fashion. For the viability screens, the symmetric of the normalized values was used, in order to have viability defect phenotypes represented by positive score values.

To be able to compare the candidates of the five TRAIL screen replicates, the dynamic range of each screen had to be adapted. Therefore, a quantile normalization was applied, which normalized the distribution of intensities in each screen to a reference screen. The reference screen is estimated as the pooled distribution of probes in all screens. The original quantile of each value in the distribution of intensities within each screen is transformed to the quantile's value in the reference screen. Thus, as a result of quantile normalization, screens are transformed to have identical intensity distributions. In the case of the two viability screens, no quantile transformation was applied, since the screens correspond to technical replicates with comparable intensity distributions.

The relative distribution of the values in each of the five TRAIL screens was compact. To decide whether a value significantly deviates from the normal distribution or not, a sigmoid transformation to the normalized values was applied. This transformation expands the range of intermediate values and shrinks the more extreme ones. The transformation mapped the values to the range 0-1 and the obtained values were called “calls”. For this transformation, the centre of the sigmoid curve was set to 1.5, corresponding approximately to the 95% quantile of the distribution of values. The parameter lambda, which controls the smoothness of the transition from low values to higher values, was set to 3. The same sigmoid transformation was applied to the normalized values of the viability screen.

### Defining Hitlists

To define hits from the five TRAIL screen replicates, the average call value for each gene was determined. Hits with a call equal or higher 0.9 were included into a stringent hitlist ‘A’ containing 48 genes. A less stringent hitlist ‘B’ was generated based on a randomization approach. Therefore, the average rank of each gene was determined across the replicates, based on the matrix of normalized values. Using the observed normalized values for each screen, 500 randomizations of each replicate were performed and the average rank of each probe across each group of randomized screens was determined. For each probe, the number of times that the probe had a rank in the randomized data set lower or equal to the observed rank was counted and divided by the number of randomizations performed. 665 genes were identified to significantly differ from a rank caused by coincidence and were therefore grouped into the relaxed hitlist ‘B’.

### Results

Two studies A) and B) were performed.

### Study A)

To gain insight into the importance of different components of the TRAIL signalling pathway and to identify new mediators of this pathway, RNAi was used to individually silence the expression of different genes. The results of the genome-wide RNAi screens for TRAIL-induced apoptosis and the role of several factors newly identified as being required for TRAIL-induced apoptosis will be explained in the following.

### RNAi Screen Setup and Validation

### TRAIL-Sensitivity of HeLa Cells

Before performing genome-wide siRNA screens for TRAIL-induced apoptosis, the sensitivity of HeLa cells to TRAIL-induced apoptosis had to be determined. The cervix carcinoma cell line Hela was chosen for the screening experiments because it is easily transfectable with siRNAs and sensitive to TRAIL-induced apoptosis.

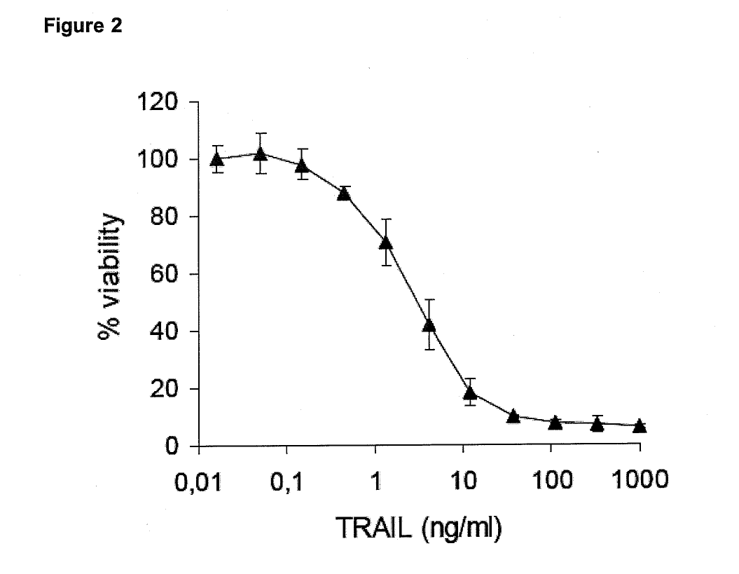

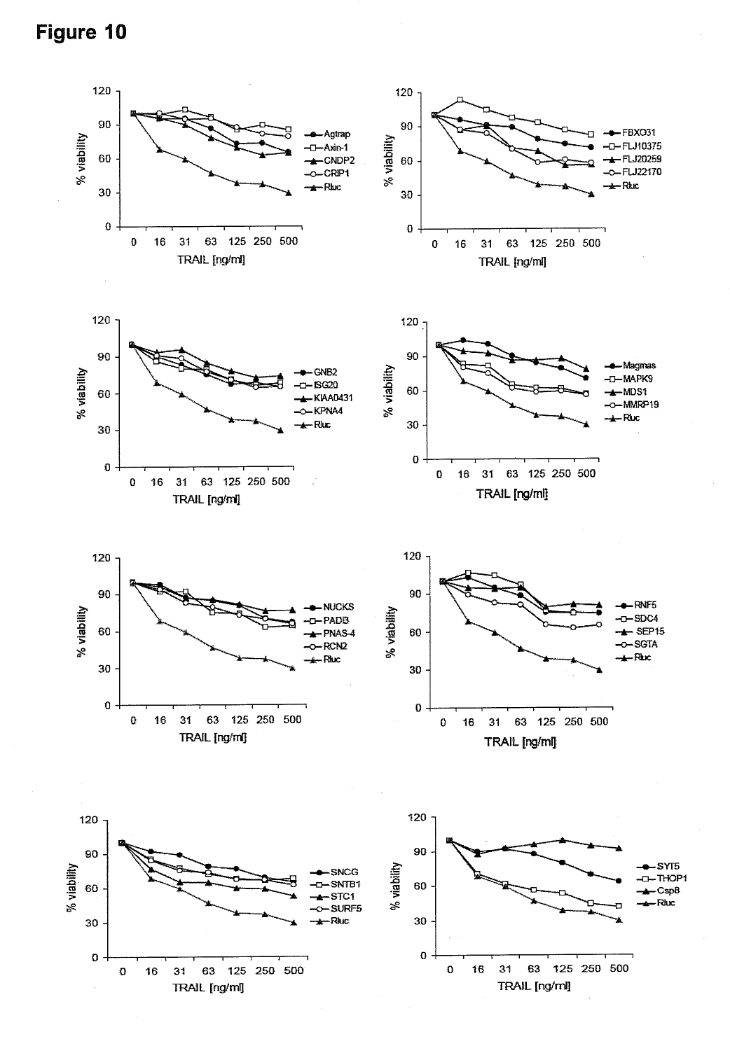

To determine the exact sensitivity to TRAIL-induced apoptosis, a dose-response curve ranging from 0.01 to 1000 ng/ml TRAIL was performed. Cell viability was quantified 24 h later by the CellTiterGlo® assay as this method is very sensitive, reproducible and applicable for high-throughput approaches. FIG. 10 shows that HeLa cells die in a dose-dependent manner following application of TRAIL. Maximum cell death is reached at TRAIL concentrations around 100 ng/ml (cf. FIG. 2).

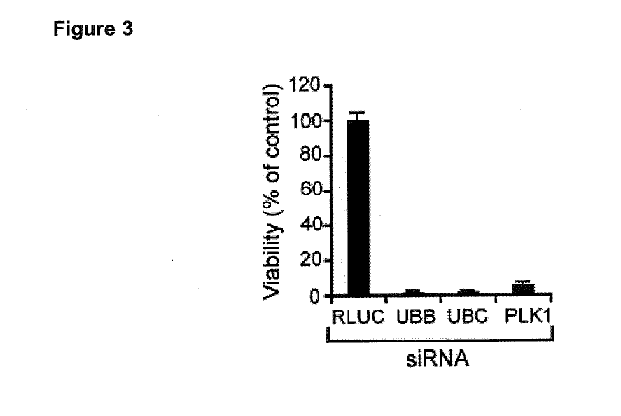

This result was also confirmed by microscopic inspection. After addition of TRAIL, HeLa cells rapidly undergo apoptosis, characterised by membrane blebbing, shrinkage of cells and chromatin condensation (FIG. 3).

### Set-Up of Genome Wide RNA1 Screens

To ensure efficient killing, a concentration of 100 ng/ml was used for the following genomewide RNAi screens for TRAIL-induced apoptosis. The TRAIL screens were also named “resistance screens” because KD of a factor required for TRAIL-induced apoptosis, for example caspase-8, would result in apoptosis resistance. Hence, “hits” in the “resistance screen” represent factors whose KD caused resistance to TRAIL-induced apoptosis.

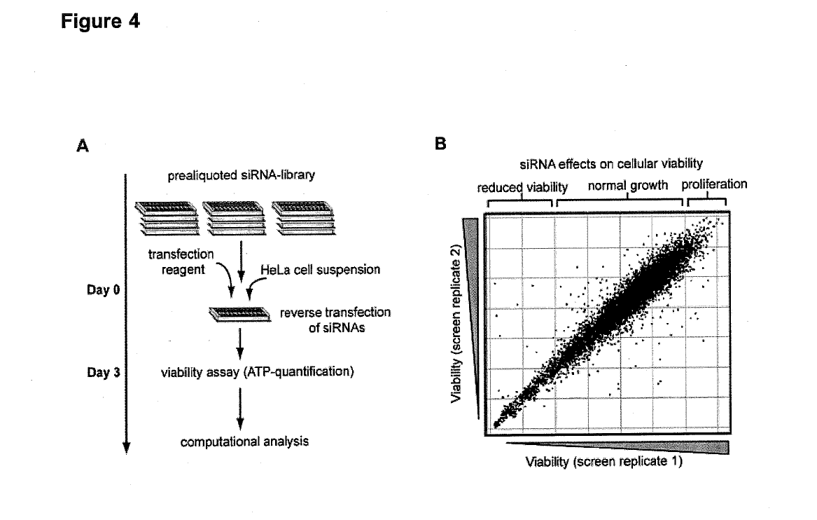

An overview of the genome-wide RNAi screen is shown in FIG. 4. For genome-wide RNAi screens, HeLa cells were reverse transfected with siRNA pools targeting individual mRNAs in 384-well plates. Thereby 21115 open-reading frames (ORFs) of the human genome were targeted with pools of four independent siRNAs to identify genes necessary for TRAIL-induced apoptosis.

### Identification of Novel Factors Involved in TRAIL-Induced Apoptosis by Genome-Wide RNAi

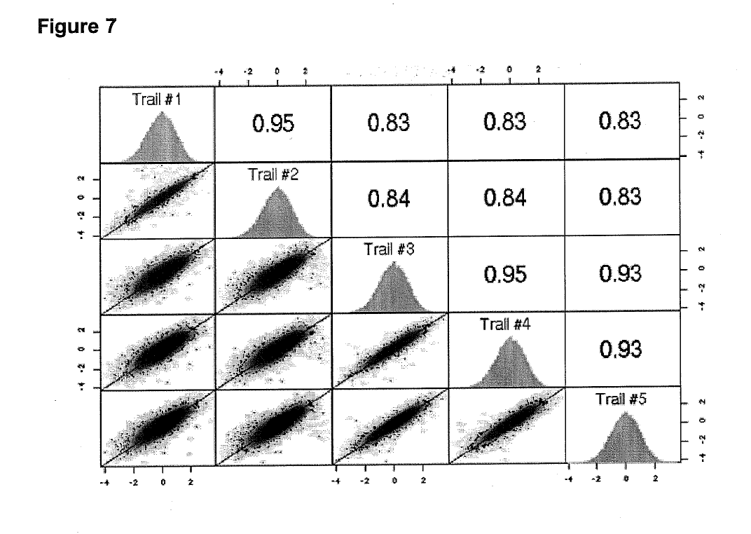

To ensure reliable and reproducible results, the entire siRNA library was searched in five replicates over a period of 14 months for novel TRAIL pathway components (TRAIL screens #1-#5). In addition to the five TRAIL siRNA screens, two screens were performed without the addition of TRAIL (Viability screens #1, #2) (FIG. 7).

KD of Axin-1, a rescue hit in the TRAIL RNAi screens, protects HeLa cells from TRAIL-induced apoptosis almost as good as caspase-8 which served as positive control. As HeLa cells express both, TRAIL-R1 and TRAIL-R2, KD of either of the receptors only led to a partial rescue phenotype indicated in the blue coloured “hitlist B”. KD of FADD was expected to result in a TRAIL rescue phenotype similar to KD of caspase-8. However, the application of FADD siRNAs for 48 h did not lead to an efficient KD of FADD protein and therefore FADD was not a hit in the TRAIL screen.

The viability screens (Viability screens #1, #2) reveal whether application of the respective siRNA leads to enhanced or diminished proliferation. This is important because the CellTiterGlo® assay used for the screen readout measures viability of cells by the quantification of ATP. Therefore, an enhanced viability in the TRAIL screen could be due to increased proliferation and not due to diminished cell death mediated by TRAIL. For this reason, it is important to determine whether the siRNAs that are classified as “hits” in the TRAIL apoptosis screens, alone exert a proliferation phenotype. For example, KD of TFF2 led to higher cell proliferation, but the rescue effect was not as strong as for other hits (e.g. KPNA4). Therefore, hits that already show strongly increased values in the cell viability assay without application of TRAIL may have to be considered as false positive in the TRAIL apoptosis screens.

The analysis of five consecutive RNAi screens for novel TRAIL apoptosis modulators revealed that the screens are highly reproducible (FIG. 7). Calculation of the Pearson's correlation coefficient (Williams 1996) shows that all five screens are very similar. The Pearson's correlation coefficient reflects the degree of linear relationship between two screening replicates. A correlation coefficient of +1 means perfect similarity whereas 0 means no linear correlation. Thus, Pearson's correlation coefficients of 0.83 to 0.95 as calculated for all five screens demonstrate high data reproducibility and a low technical failure rate during the screening procedure.

Furthermore, statistical analysis shows that the top 50 hits in screen 1 are also the top 50 hits in screens 2, 3, 4 and 5.

### Validation of Novel Factors Involved in TRAIL-Induced Apoptosis

The statistical analysis of five TRAIL-RNAi screens revealed that the screens show technical stability and that the data are highly reproducible. The use of siRNA pools (4 single siRNA sequences targeting one gene) increases the possibility that the KD of the gene of interest is very efficient, but it can also increase offtarget effects. To minimise the possibility that off-target effects play a role in the observed TRAIL phenotype, the siRNA pools were deconvoluted into single sequences. The term “off-target effect” means that application of the siRNA does not only downregulate the mRNA of interest, but also affects other genes.

Deconvolution of siRNA Pools

Deconvolution of siRNA pools into four single siRNA sequences targeting the gene of interest showed that in almost all cases several single siRNA sequences could confirm the TRAIL rescue phenotype (see FIG. 10). Furthermore, all four single siRNA sequences of the siRNA pool target the respective mRNA at different sites. Consequently, if more than two single siRNAs result in resistance to TRAIL-induced apoptosis off-target effects are rather unlikely.

### Examination of Novel Factors in Different Cell Lines

All newly identified factors that were shown to be required for TRAIL-induced apoptosis were retested in HeLa cells over a broad concentration range of TRAIL. Therefore, Hela cells were transfected with the respective siRNA pools and cell viability was determined by measuring mitochondrial activity (MTT assay; see FIG. 10). To determine whether the function of the newly identified TRAIL apoptosis modulators is restricted to the cervix carcinoma cell line HeLa or can also be observed in cell lines derived from other types of cancer, the TRAIL-sensitive breast cancer cell line MDA-MB-231 and the TRAIL-sensitive colon carcinoma cell line DKO4 were transfected with the respective siRNA pools and cell viability was measured after TRAIL treatment. Additionally, several controls for transfection and KD efficiency were performed for each experiment. KD of caspase-8 served as positive control because it conferred nearly completely resistance to TRAIL-induced apoptosis. siRNAs against several housekeeping genes (UBB, UBC and PLK1) were applied, resulting in highly reduced cell viability (negative control) which was confirmed by microscopic inspection and MTT staining (data not shown).

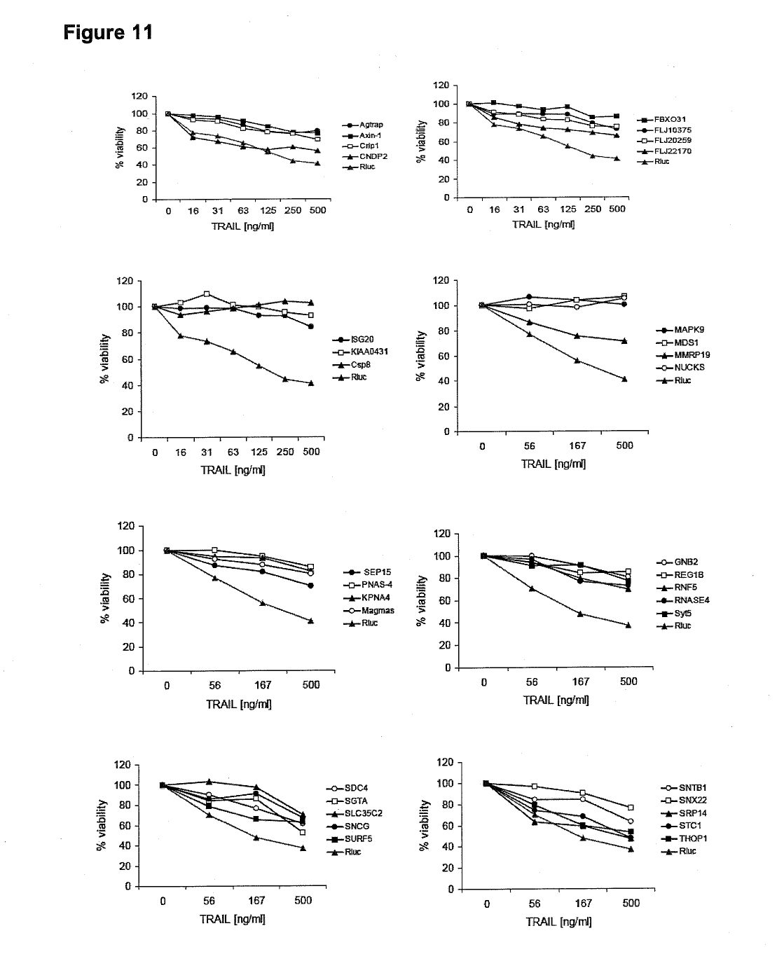

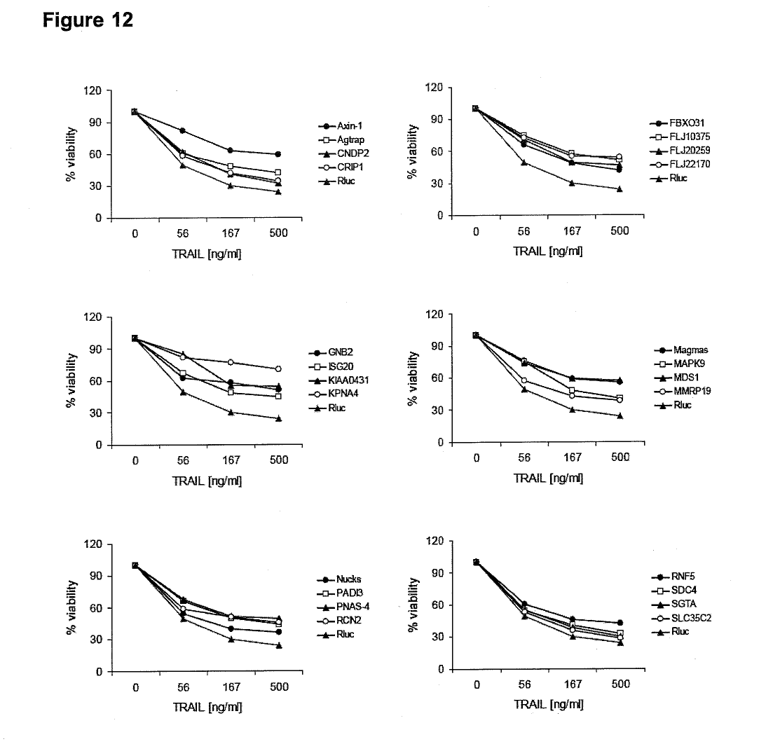

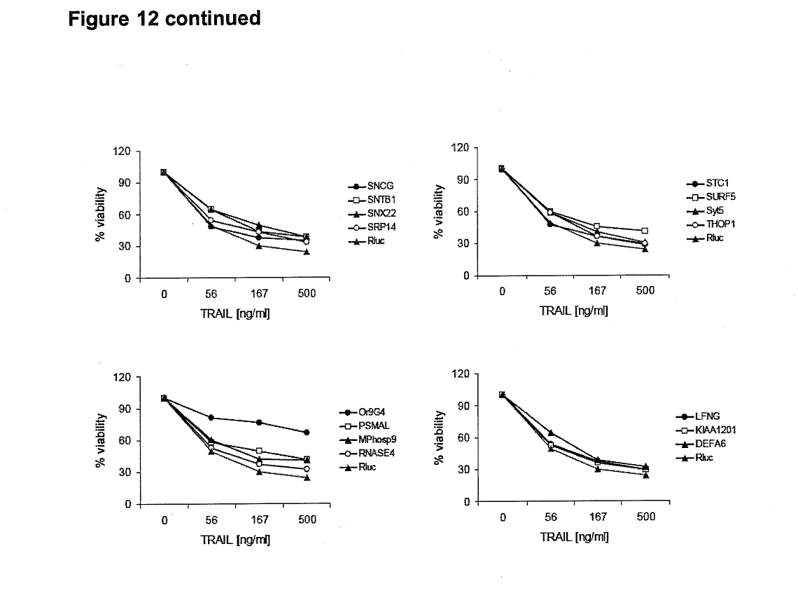

Most of the factors that were found to be required for TRAIL-induced apoptosis in HeLa cells are also required for apoptosis induction by TRAIL in MDA-MB-231 cells (FIG. 11). However, in contrast to HeLa cells, little or no rescue from TRAIL-induced apoptosis was observed after KD of SGTA, SNCG, SDC4, SURFS, FLJ22170, CNDP2, THOP1, SRP14 and STC1, respectively, in MDA-MB-231 cells.

The newly identified factors that were shown to influence TRAIL-induced apoptosis in HeLa and MDA-MB-231 cells were also evaluated in the colon cancer cell line DKO4 (FIG. 11). In general, KD of most of the factors protected DKO4 cells much weaker from TRAIL induced apoptosis than HeLa or MDA-MB-231 cells although the transfection efficiency was similar (microscopic inspection, data not shown). However, some siRNA pools strongly affected TRAIL-induced apoptosis in DKO4 cells including Axin-1, KPNA4, Magmas, MDS1 and Or9G4.

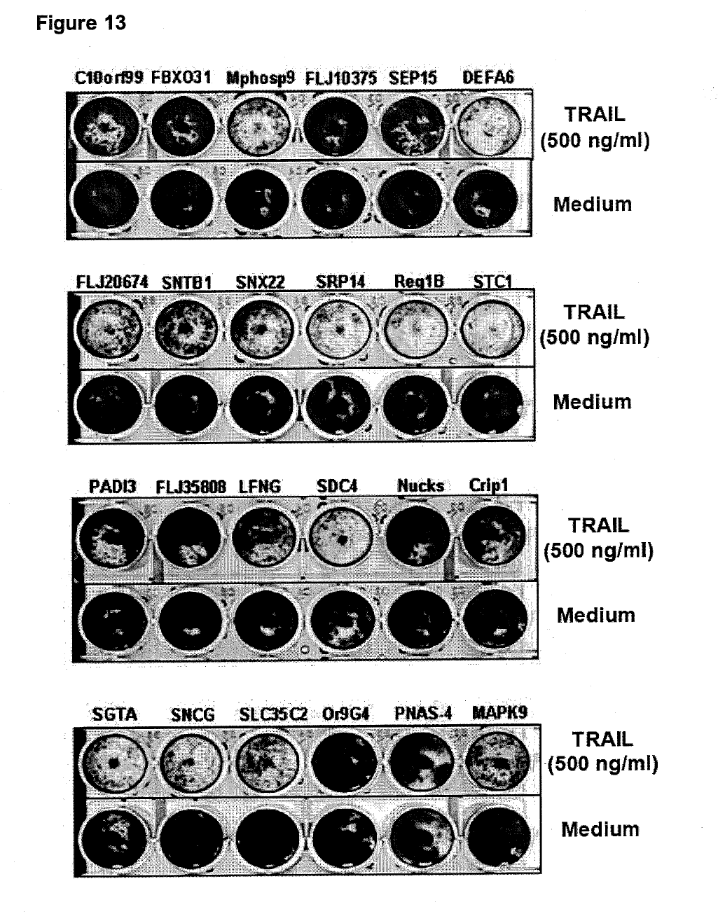

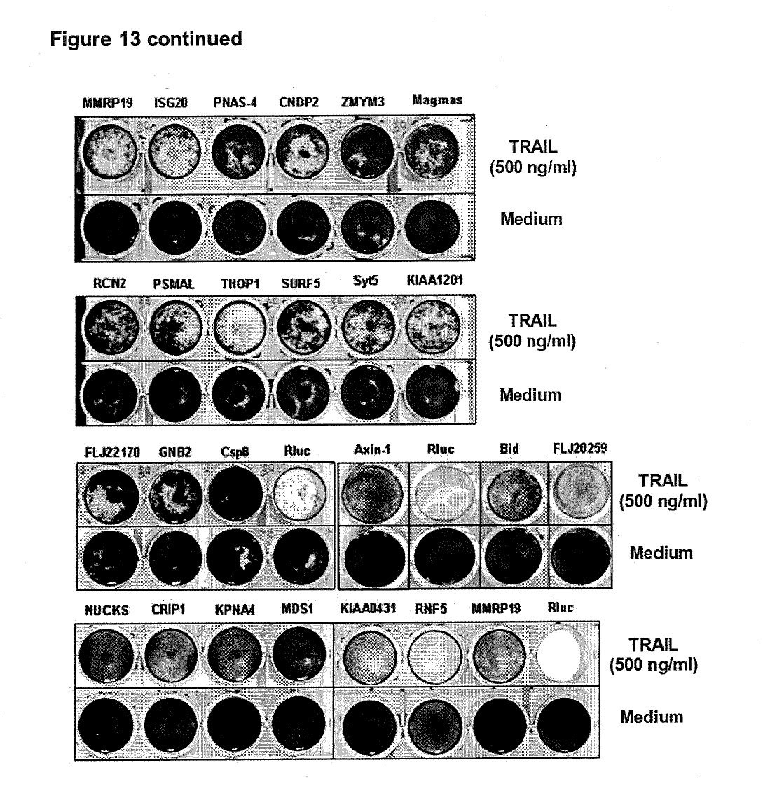

In all previous experiments, cell death after TRAIL stimulation was confirmed by cell viability measurements based on the quantification of ATP (CellTiterGlo®) or mitochondrial activity (MTT assay). These methods were used because they are very sensitive and applicable for high-throughput approaches. However, viability was always measured after TRAIL treatment for 24 h. Hence, it cannot be excluded that KD of the newly identified factors only leads to a delay in death induction and not to enhanced long-term survival. Therefore, long-term survival assays that measure long-term long-term survival after TRAIL treatment were performed. FIG. 13 clearly shows that the KD of almost all factors confers resistance to TRAIL-induced apoptosis and enhances long-term survival. However, the long-term survival capability is not equal among the newly identified factors. This observation is in line with previous experiments showing that KD of some factors can almost completely rescue TRAIL-induced apoptosis whereas other factors only marginally affect cell viability. The strongest long-term survival was observed after KD of Axin-1, Or9G4, FBXO31, FLJ10375, SEP15, PNAS-4, KPNA4, MDS1, ZYMYM3, C10orf99, NUCKS, and FLJ35808 (see FIG. 13).

### Novel Factors in TRAIL-Induced Apoptosis

After the identification and validation of proteins that are required for TRAIL-induced apoptosis, the function of these proteins in the TRAIL apoptosis pathway was investigated. Among the novel factors, Axin-1 was already known to play a key role in another cellular signalling pathway, the wnt pathway and is mutated in many cancers, especially in hepatocellular carcinoma. Therefore, Axin-1 was intensively inspected and the results are presented herein.

### AGTRAP

The type-1 angiotensin II receptor-associated protein (Agtrap) has been implicated in the negative regulation of type-1 angiotensin II receptor-mediated signalling by regulating receptor internalisation as well as receptor phosphorylation. Agtrap is a multi-pass membrane protein and appears to be mainly localised to the ER and the plasma membrane (Wang, Huang et al. 2002; Kamada, Tamura et al. 2003; Lopez-llasaca, Liu et al. 2003; lhara, Egashira et al. 2007).

A role for Agtrap in TRAIL-induced apoptosis has not been described so far. To gain insight into the regulation mechanisms after Agtrap KD, TRAIL was applied for distinct time periods and intracellular proteins involved in TRAIL apoptosis signalling were monitored. However, western blot analysis of these proteins did not lead to a clear conclusion concerning how Agtrap affects the TRAIL pathway (FIG. 13).

The analysis revealed no differential cleavage of caspase-8 and caspase-9 in Agtrap KD compared to Rluc KD cells. Surprinsingly, Bid seemed to be slightly upregulated while cFLIP was slightly downregulated in Agtrap KD cells which would rather favour apoptosis induction by TRAIL than resistance.

### CRIP1

The cysteine-rich protein 1 (CRIP1) is a member of the LIM/double zinc finger protein family and has been implicated in intestinal zinc transport (Hempe and Cousins 1991). Furthermore, it has been shown that ectopic expression of CRIP1 is able to suppress cell proliferation and to protect cells from UV- and staurosporine-induced apoptosis (Latonen, Jarvinen et al. 2008). In addition, CRIP1 is often hypomethylated in prostate cancer (Wang, Williamson et al. 2007).

CRIP1 has so far not been reported to play a role in TRAIL-induced apoptosis. However, KD of CRIP1 rescued cells from TRAIL-induced apoptosis (see FIG. 10 and FIG. 13). Interestingly, not the depletion but the overexpression of CRIP1 has been published to exert a protective effect for apoptosis induction by staurosporine and UV irradiation (Latonen, Jarvinen et al. 2008).

To get more insight into the regulatory mechanisms after CRIP1 KD, TRAIL was applied for different times and intracellular proteins involved in TRAIL apoptosis signalling were monitored. Caspase-8 cleavage is fully abrogated in CRIP1 KD cells. Furthermore, downstream events like Bid cleavage and caspase-9 activation do not take place in the first 4 h after TRAIL stimulation. Changes in the anti-apoptotic proteins XIAP and Bcl-2 could not be observed. These results suggest that CRIP1 may be crucial for efficient DISC formation that allows for recruitment and activation of caspase-8.

### FBXO31

The F-box only protein 31 (FBXO31) has been shown to recognise and bind to some phosphorylated proteins thereby promoting their ubiquitination and degradation. Furthermore, FBXO31 is a candidate breast tumour suppressor (Kumar, Neilsen et al. 2005).

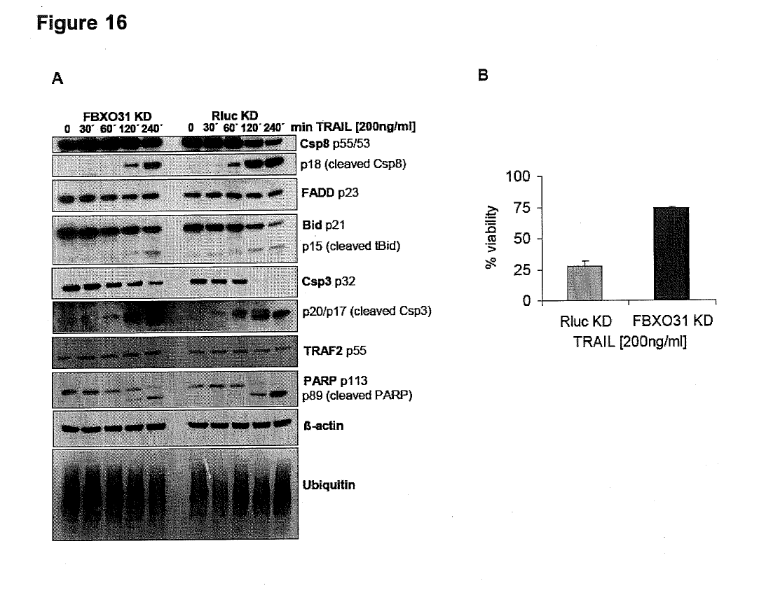

KD of FBXO31 causes resistance to TRAIL-induced apoptosis (see FIG. 10 and FIG. 13). Consequently, FBXO31 is required for apoptosis induction by TRAIL. To further evaluate the function of FBXO31 in the TRAIL apoptosis pathway, caspase cleavage events and known TRAIL pathway components were monitored after TRAIL application. HeLa cells were transfected with FBXO31 and Rluc siRNA pools, respectively and cells were then subjected to TRAIL treatment for different times followed by the preparation of cell lysates and western blot analysis (FIG. 16).

As FBXO31 was reported to be involved in the ubiquitination of proteins, an antibody recognising total ubiquitin was applied. However, the overall ubiquitination pattern of the cell is not changed by FBXO31 KD. This is not really surprising as it is unlikely that KD of a single E3 ligase has a big impact on the overall ubiquitination of the cell.

### KIAA0431

The protein KIAA0431, also named ATM IN (ATM interactor) has been shown to be involved in ATM signalling. ATM (ataxia telangiectasia mutated) is a checkpoint kinase which is activated in response to DNA damage. KIAA0431/ATMIN can interact with ATM through a C-terminal motif, which is also present in Nijmegen breakage syndrome (NBS)1 (Kanu and Behrens 2007).

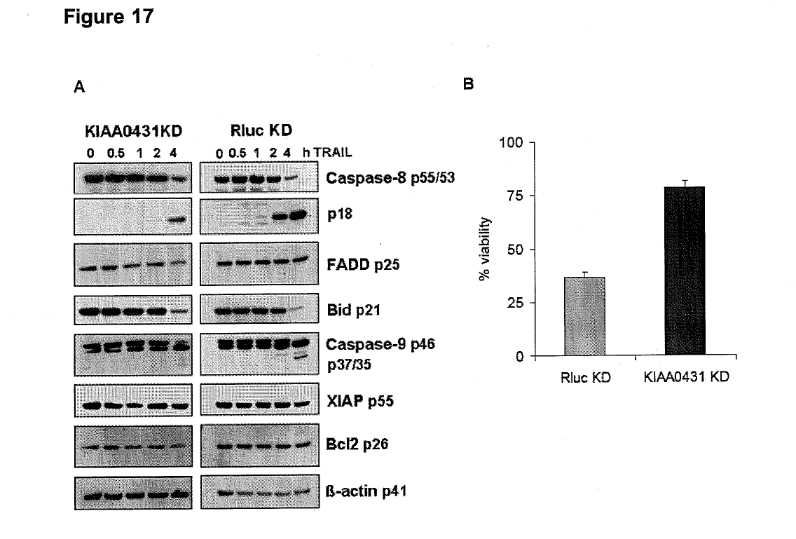

KD of KIAA0431 causes resistance to TRAIL-induced apoptosis (see FIG. 10 and FIG. 13). To get an insight into the function of KIAA0431 in the TRAIL apoptosis pathway, caspase cleavage events and known TRAIL pathway components were monitored after TRAIL application. Therefore, HeLa cells were transfected with KIAA0431 and Rluc siRNA pools, respectively. Afterwards, the cells were subjected to TRAIL treatment for different times followed by the preparation of cell lysates and western blot analysis. In parallel, a viability assay was performed to control for transfection efficiency and the degree of apoptosis induction by TRAIL. Western blot analysis revealed that caspase-8 cleavage into the p18 fragment is delayed in KIAA0431 KD cells (see FIG. 17). FADD levels seem to be slightly decreased in KIAA0431 cells which could lead to less caspase-8 recruitment, binding and activation of the initiator caspase at the TRAIL-DISC. However, cleavage of Bid is not strongly altered. Interestingly, caspase-9 cleavage can be detected in control (Rluc) KD cells while cleavage fragments are hardly seen in KIAA0431 KD cells. Yet, levels of anti-apoptotic proteins XIAP and Bcl-2 are unchanged.

### KPNA4

The karyopherin subunit alpha-4 (KPNA4) has been described to have a role in nuclear protein import as an adapter protein for nuclear receptor KPNB1. In vitro, KPNA4 mediates the nuclear import of human cytomegalovirus UL84 by recognizing a non-classical nuclear localisation signal (NLS) (Ayala-Madrigal, Doerr et al. 2000; Fagerlund, Kinnunen et al. 2005; Grundt, Haga et al. 2007).

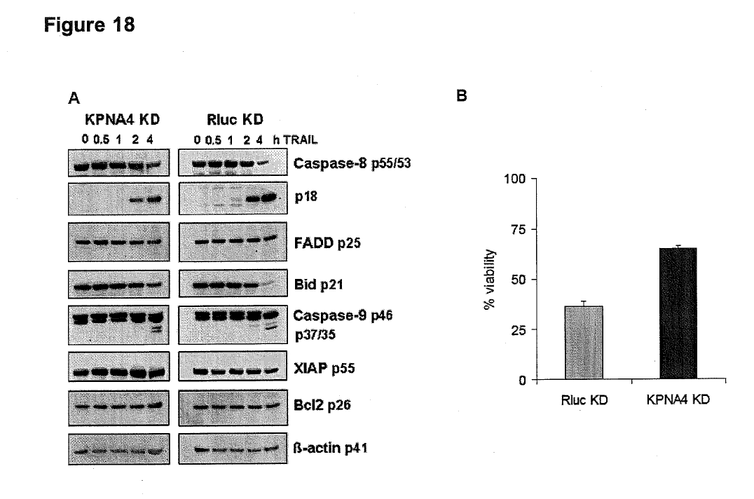

KPNA4 is required for apoptosis induction by TRAIL, as KD of this protein leads to TRAIL resistance (see FIG. 10 and FIG. 13). To unravel the regulation mechanisms after KPNA4 KD, TRAIL was applied for different times and intracellular proteins involved in TRAIL apoptosis signalling were monitored. However, western blot analysis of these proteins did not lead to a clear conclusion how KPNA4 affects the TRAIL pathway. Cleavage of the initiator caspase-8 was slightly decreased, but all other monitored proteins were unchanged (see FIG. 18).

### MAGMAS

The Mitochondria-associated granulocyte macrophage CSF-signaling molecule (Magmas) has been shown to be induced by granulocyte-macrophage-colony stimulating factor (GM-CSF) in hematopoietic cells (Jubinsky, Messer et al. 2001; Peng, Huang et al. 2005). The protein is also called mitochondrial import inner membrane translocase subunit (TIM16) because it is a component of the presequence translocase-associated motor (PAM) complex, which is required for the translocation of transit peptide-containing proteins from the inner membrane into the mitochondrial matrix in an ATP-dependent manner (Kozany, Mokranjac et al. 2004; losefson, Levy et al. 2007; Mokranjac, Berg et al. 2007).

During TRAIL-induced apoptosis, mitochondria are depolarised and pro-apoptotic factors are released from the mitochondria.

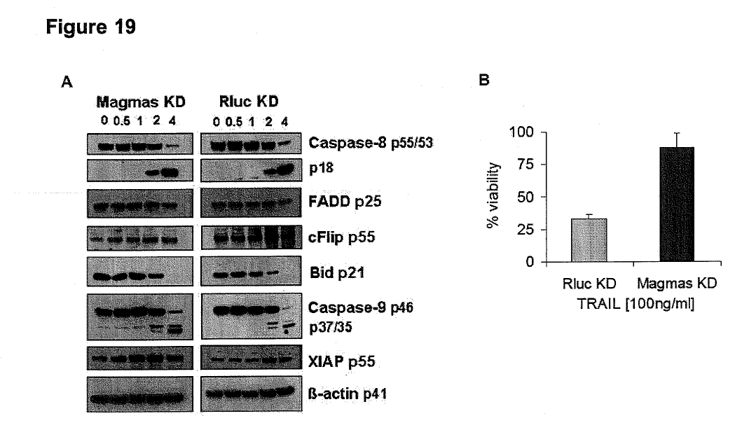

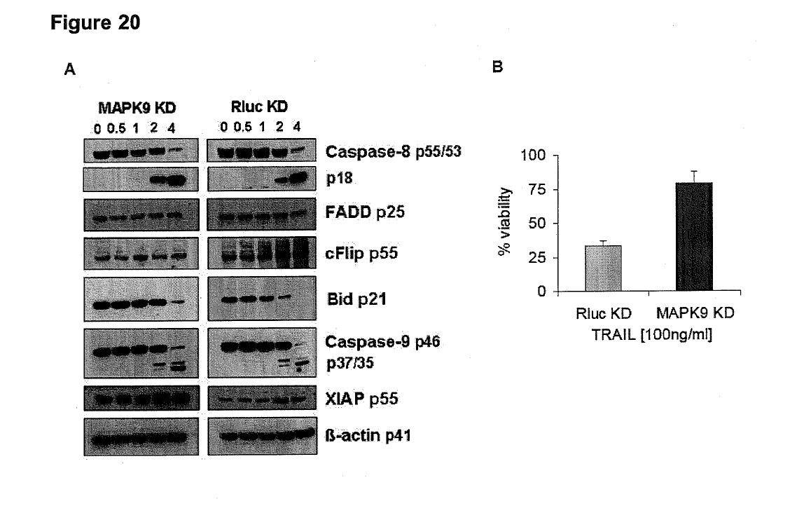

To investigate the role of Magmas in TRAIL-induced apoptosis, intracellular proteins known to be involved in the TRAIL apoptosis pathway were monitored. Interestingly, FADD expression was increased in Magmas KD cells while expression of cFLIP was decreased which would rather favour apoptosis induction. However, caspase-8 and Bid cleavage were not strongly altered while caspase-9 cleavage was slightly reduced and XIAP slightly upregulated in Magmas KD cells (see FIG. 19).

### MAPK9

The mitogen-activated protein kinase 9 (MAPK9), also called c-Jun N-terminal kinase 2 (JNK2) or stress-activated protein kinase 2 (SAPK2) has been reported to be involved in a wide variety of cellular processes such as proliferation, differentiation, transcriptional regulation and development (Sluss, Barrett et al. 1994; Gupta, Barrett et al. 1996). It is most closely related to MAPK8, which is involved in UV-irradiation-induced apoptosis and thought to be related to cytochrome c-mediated cell death pathways. Furthermore, MAPK9/JNK2 has been implicated to play a role in T cell differentiation (Jaeschke, Rincon et al. 2005).

KD of MAPK9 causes resistance to TRAIL-induced apoptosis (FIGS. 10, 11, 13). To get an insight into the biochemical events after TRAIL triggering in MAPK9-deficient cells, intracellular proteins involved in the TRAIL apoptosis pathway were monitored.

### MDS1

A chromosomal aberration (t3;21) involving the protein Myelodysplasia syndrome 1 (MDS1) is found in a form of acute myeloid leukemia (AML). MDS1 can be produced either as a separate transcript and as a normal fusion transcript with EVI1 (ecotropic viral integration site 1) (Metais and Dunbar 2008). Furthermore, high-risk myelodysplastic syndrome (MDS) has been associated with reduced NK cell function (Epling-Burnette, Bai et al. 2007).

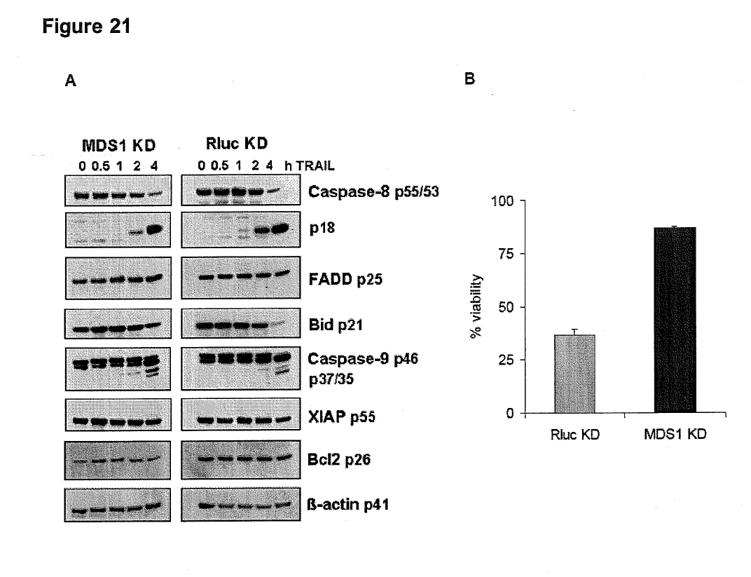

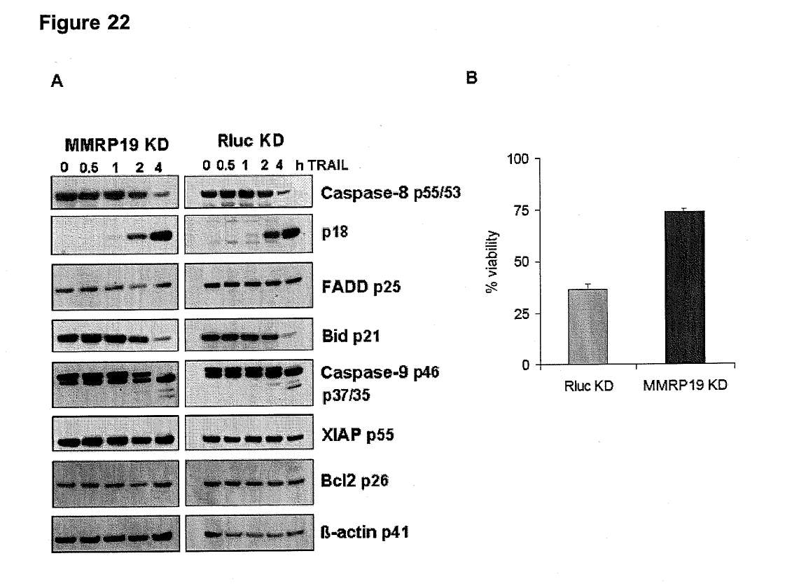

KD of MDS1 leads to TRAIL resistance (see FIG. 10 and FIG. 13). To unravel the regulation mechanisms after MDS1 KD, TRAIL was applied for different times and intracellular proteins involved in TRAIL apoptosis signalling were monitored (see FIG. 21). However, western blot analysis of these proteins did not lead to a clear conclusion how MDS1 affects the TRAIL pathway. Caspase-8 cleavage was slightly reduced in MDS1 KD cells, which correlates with slightly decreased cleavage of Bid and caspase-9. Bcl-2 levels were slightly lower in MDS1 KD cells, but XIAP levels were not affected.

### MMRP19

MMRP19, also called APIP (APAF1 interacting protein) has been shown to interact with APAF1, a protein that is required for apoptosome formation. Overexpression of MMRP19 has been demonstrated to inhibit cytochrome c-induced activation of caspase-9 and to suppress cell death triggered by mitochondrial apoptotic stimuli (Cho, Hong et al. 2004). Interestingly, in the mentioned study the overexpression of MMRP19 exerted a protective effect on intrinsically induced apoptosis while the KD of MMRP19 rescues cells from TRAIL-induced apoptosis (see FIG. 10 and FIG. 13).

### NUCKS

The Nuclear ubiquitous casein and cyclin-dependent kinases substrate (NUCKS) has been shown to be phosphorylated upon DNA damage, probably by CDK1, casein kinase, ATM or ATR (Ostvold, Norum et al. 2001; Grundt, Skjeldal et al. 2002; Grundt, Haga et al. 2004). Moreover, NUCKS possesses two nuclear localisation signals and a DNA binding domain (Grundt, Haga et al. 2007).

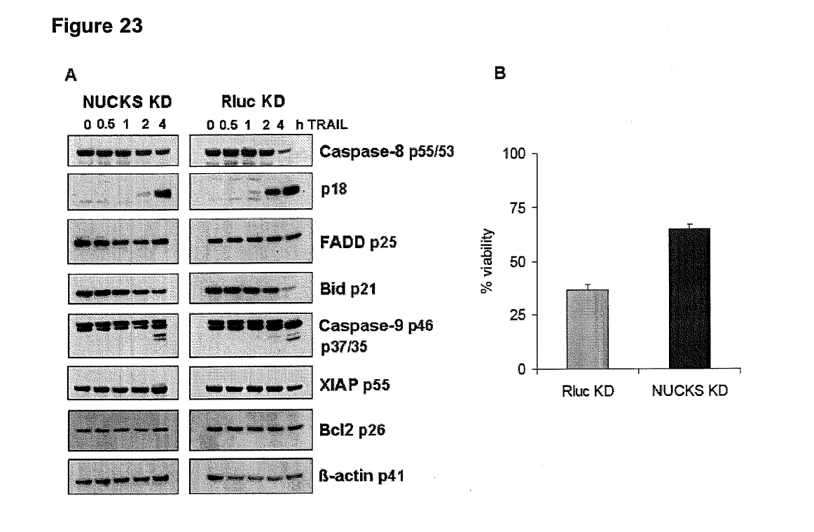

As KD of NUCKS leads to resistance to TRAIL-induced apoptosis (see FIG. 10 and FIG. 13), intracellular proteins involved in the TRAIL apoptosis pathway were monitored. Interestingly, already caspase-8 cleavage was affected by NUCKS KD (see FIG. 23). Diminished caspase-8 activation is also reflected in less Bid cleavage. However, FADD levels were not significantly altered. As expression of XIAP and Bcl-2 were not strongly changed in NUCKS KD cells, it is unlikely that these two proteins contribute to the TRAIL rescue effect induced by NUCKS KD.

### OR9G4

The olfactory receptor OR9G4 (Olfactory receptor, family 9, subfamily G, member 4) belongs to a large family of G-protein-coupled receptors (GPCR). Olfactory receptors share a 7-transmembrane domain structure with many neurotransmitter and hormone receptors and are responsible for recognition and G-protein-mediated transduction of odorant signals. However, little is known about the expression and function of OR9G4.

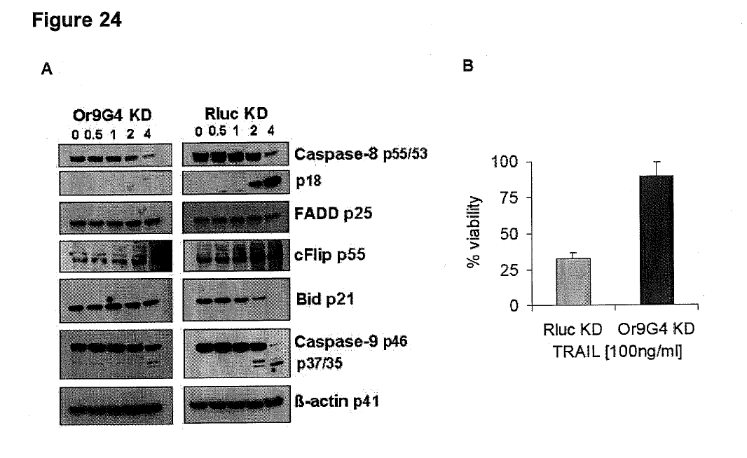

KD of OR9G4 causes resistance to TRAIL-induced apoptosis (see FIG. 10 and FIG. 13). To investigate how Or9G4 influences TRAIL-induced apoptosis, intracellular signalling events after ORG4 KD and TRAIL stimulation were monitored (see FIG. 24). Strikingly, caspase-8 expression was markedly reduced and caspase-8 cleavage did not occur after TRAIL stimulation. Furthermore, downstream events like cleavage of Bid and caspase-9 were impaired in OR9G4 KD cells as a consequence of diminished caspase-8 activation.

### PNAS-4

PNAS-4 (novel proapoptosis protein) has recently been identified as a pro-apoptotic protein in Xenopus laevis (Yan, Qian et al. 2007) and in mouse (Zhang, Wang et al. 2008). Human PNAS-4 has 96% identity with mouse PNAS-4 in primary sequence and has been reported to be involved in the apoptotic response to DNA damage.

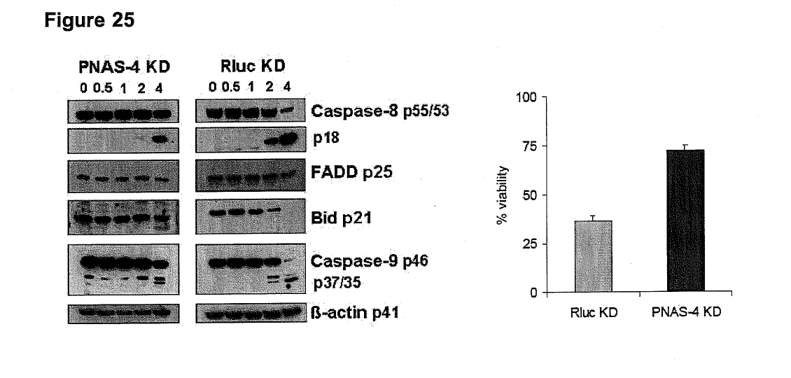

PNAS-4 has so far not been implicated to play a role in TRAIL-induced apoptosis. PNAS-4 is required for TRAIL-induced apoptosis as KD of PNAS-4 leads to TRAIL resistance (see FIG. 10 and FIG. 13). To gain insight on the regulatory mechanisms after PNAS-4 KD, intracellular proteins involved in TRAIL apoptosis signalling were monitored. FIG. 25 shows that caspase-8 cleavage was strongly delayed and reduced in PNAS-4 KD cells while FADD levels were not significantly changed. Furthermore, downstream events like cleavage of Bid and caspase-9 were markedly decreased after TRAIL stimulation.

### QRICH1

The glutamine-rich protein 1 (Qrich1/FLJ20259) has recently been annotated as a novel gene transcript located on chromosome 3 (3p21.31) (Gerhard, Wagner et al. 2004; Ota, Suzuki et al. 2004). Interestingly, the protein contains a CARD domain. Via this domain it could interact with other CARD domain-containing proteins, for example caspase-9 or Apaf-1.

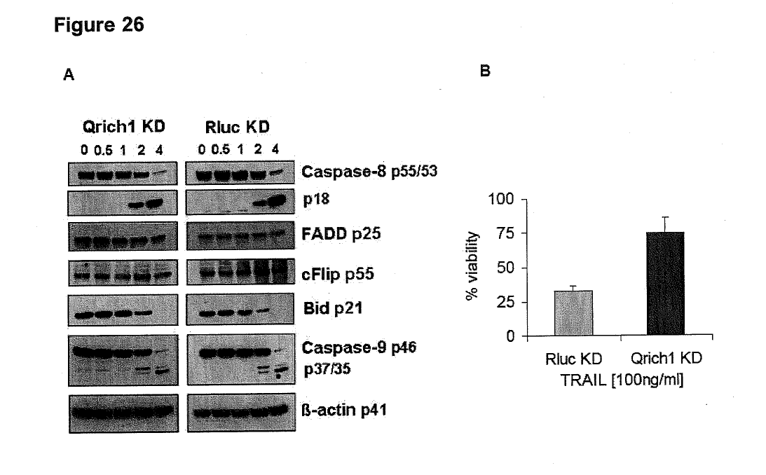

To test whether Qrich1 KD affects caspase-9 activation, intracellular caspase cleavage events were monitored. Although the viability assay that was done in parallel to the western blot analysis confirmed the rescue phenotype, caspase-8, caspase-9 and Bid cleavage were not altered (see FIG. 26).

### RNF5

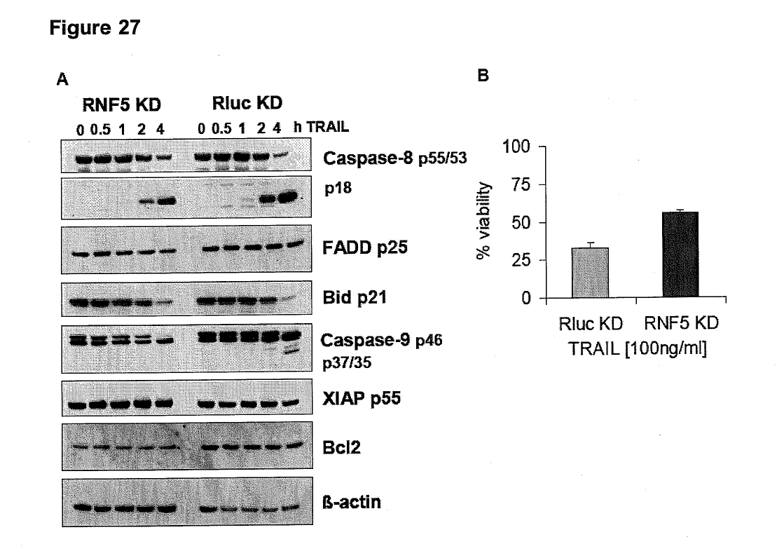

As the name already implies, the Ring-finger protein 5 (RNF5) contains a ring finger domain and it has been shown to mediate the ubiquitination of paxillin and Salmonella type III secreted protein sopA (Kyushiki, Kuga et al. 1997; Didier, Broday et al. 2003). Furthermore, increased expression of RNF5 has been associated with decreased survival in breast cancer (Bromberg, Kluger et al. 2007). For additional information on RNF5 please refer to chapter 7. To unravel the role of RNF5 in TRAIL-induced apoptosis, intracellular levels of pro- and anti-apoptotic proteins as well as caspase cleavage events after TRAIL treatment were monitored (see FIG. 27). Cleavage of the initiator caspase-8 into the p18 fragment which is a hallmark of caspase-8 activation is slightly diminished after KD of RNF5. FADD levels are slightly decreased in RNF5 KD cells which could result in less caspase-8 binding to its adaptor protein FADD at the TRAIL DISC. However, cleavage of the BH3-only protein Bid is not significantly changed. The biggest difference between RNF5 KD and control cells can be seen in the cleavage of caspase-9. While caspase-9 is already cleaved 2 h after TRAIL application in control cells, caspase-9 expression is slightly decreased and no caspase-9 cleavage can be observed in RNF5 KD cells.

### SEP15

The 15 kD selenoprotein (SEP15) has been implicated in disulfide bond assisted protein folding in the ER where it can bind to UDP-glucose:glycoprotein glucosyltransferase (GT), an essential regulator of quality control mechanisms within the ER (Labunskyy, Ferguson et al. 2005; Labunskyy, Hatfield et al. 2007). Furthermore, malignant mesothelioma cells that lack SEP15 expression were shown to be more resistant to growth inhibition and apoptosis induction by selenium (Apostolou, Klein et al. 2004).

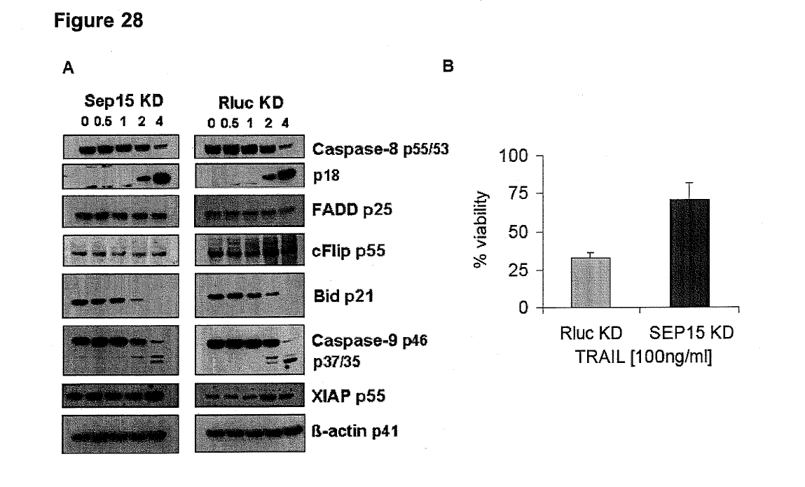

KD of SEP15 renders cells resistant to TRAIL-induced apoptosis (see FIG. 10 and FIG. 13). To further evaluate the role of SEP15 in the TRAIL signalling pathway, intracellular events after the addition of TRAIL were monitored. Interestingly, caspase-8 and Bid cleavage are similar but caspase-9 cleavage is delayed in SEP15 deficient cells (see FIG. 28). In addition, XIAP levels are higher in SEP15 KD cells. The cell viability assay performed in parallel to the western blot analysis shows that these SEP15 KD cells are protected from TRAIL-induced apoptosis.

### Summary—Novel Factors

The genome-wide TRAIL RNAi screens revealed several novel factors that are required for TRAIL-induced apoptosis. The results presented in this chapter implicate an early contribution of CRIP1, FBXO31, KIAA0431, NUCKS, OR9G4 and PNAS-4 in the TRAIL pathway as already caspase-8 cleavage after TRAIL treatment was strongly diminished in the respective KD cells. KD of KPNA4, MDS1 or RNF5 also affected caspase-8 processing although to a lesser degree, indicating that those proteins may not be crucial for, but may contribute to the recruitment of caspase-8 to the TRAIL DISC and its subsequent activation. Diminished caspase-8 cleavage after TRAIL treatment mostly correlated with less processing of the downstream effectors Bid, caspase-3 and caspase-9. Interestingly, KD of RNF5 only slightly influenced caspase-8 cleavage while caspase-9 processing was strongly affected. Moreover, in SEP15 KD cells normal processing of Bid, but diminished caspase-9 cleavage could be observed after TRAIL treatment. XIAP upregulation could be observed after KD of MAPK9, Magmas and SEP15. A strong upregulation of cFLIP or Bcl-2 which could contribute to TRAIL resistance was not detected in the investigated KD cells. Furthermore, FADD levels were not strongly changed. A summary of the different regulation mechanisms in the respective KD cells is shown in Table 2.

TABLE 2
Summary - novel factors in TRAIL-induced apoptosis

| Protein | | | | | | | |
|---|---|---|---|---|---|---|---|
| KD | Caspase-8 | Caspase-9 | FADD | cFLIP | XIAP | Bcl-2 | BID |
| AGTRAP | Cleavage not | Cleavage not | Unchanged | Slightly | Unchanged | n.d. | Slightly |
| | affected | affected | | reduced | | | upregulated |
| CRIP1 | Cleavage | Cleavage | Unchanged | n.d. | Unchanged | Unchanged | No |
| | nearly | nearly | | | | | cleavage to |
| | abolished | abolished | | | | | tBID |
| FBXO31 | Cleavage | n.d. | Unchanged | n.d. | n.d. | n.d. | Cleavage |
| | strongly | | | | | | almost |
| | delayed and | | | | | | abrogated |
| | diminished | | | | | | |
| KIAA0431 | Cleavage | Cleavage | Slightly | n.d. | Unchanged | Unchanged | Less |
| | strongly | nearly | decreased | | | | cleavage |
| | delayed and | abolished | | | | | |
| | diminished | | | | | | |
| KPNA4 | Cleavage | Not | Unchanged | n.d. | Unchanged | Unchanged | Slightly less |
| | slightly | significantly | | | | | cleavage |
| | delayed and | changed | | | | | |
| | diminished | | | | | | |
| MAGMAS | Cleavage not | Cleavage | Slightly | Slightly | Slightly | n.d. | Cleavage |
| | affected | slightly | increased | increased | upregulated | | not affected |
| | | reduced | | | | | |
| MAPK9 | Cleavage not | Cleavage not | Unchanged | Slightly | Upregulated | n.d. | Slightly |
| | affected | affected | | reduced | | | upregulated |
| MDS1 | Cleavage | Cleavage | Unchanged | n.d. | Unchanged | Slightly | Cleavage |
| | delayed and | slightly | | | | down regulated | slightly |
| | diminished | reduced | | | | | reduced |
| MMRP19 | Cleavage not | Cleavage not | Slightly | n.d. | Unchanged | Unchanged | Unchanged |
| | affected | affected | decreased | | | | |
| NUCKS | Cleavage | Cleavage | Unchanged | n.d. | Unchanged | Unchanged | Cleavage |
| | delayed and | slightly | | | | | slightly |
| | diminished | reduced | | | | | reduced |
| OR9G4 | Lower | Cleavage | Unchanged | Slightly | n.d. | n.d. | No |
| | expression, | strongly | | reduced | | | cleavage |
| | cleavage | reduced | | | | | observed |
| | nearly | | | | | | |
| | abolished | | | | | | |
| PNAS-4 | Cleavage | Cleavage | Slightly | n.d | n.d. | n.d. | Cleavage |
| | strongly | strongly | reduced | | | | almost |
| | delayed and | reduced | | | | | abrogated |
| | diminished | | | | | | |
| QRICH1 | Cleavage not | Cleavage not | Slightly | Slightly | n.d. | n.d. | Cleavage |
| | affected | affected | increased | reduced | | | not affected |
| RNF5 | Cleavage | Expression | Unchanged | n.d. | Unchanged | Unchanged | Cleavage |
| | slightly | slightly | | | | | slightly |
| | delayed and | decreased, | | | | | reduced |
| | diminished | cleavage | | | | | |
| | | nearly | | | | | |
| | | abolished | | | | | |
| SEP15 | Cleavage not | Cleavage | Unchanged | Slightly | Upregulated | n.d. | Cleavage |
| | affected | reduced | | reduced | | | not affected |

Many kinases identified in this approach are known cell cycle regulators, such as the family of cyclin-dependent kinases (CDK3, 5, 6, 9, 10 and 11) and cell division cycle proteins (CDC2 and 7) (reviewed in Bloom 2007). Furthermore, the list contains proteins known to regulate chromosome movement and segregation (aurora kinase B) and cell cycle checkpoints (CHEK1, BUB1 and PLK1, 2, 3, 4) (Xie 2005; Tang 2006; Logarinho 2008; Vas 2008). The finding of known regulators as top scoring candidates in screens generally demonstrates a robust assay system. The identification of several known cell cycle components in the siRNA kinome screen showed that the systematic characterization of gene function with RNAi in human cells is feasible. In the next step, the technique was employed to develop highly miniaturized assays for the use in largescale genome-wide RNAi approaches in human cells.

Genome-Wide siRNA Screens for Markers of Cell Death and Resistance

Cellular growth and survival is tightly regulated by a complex network of mediatorssuch as kinases and many other types of regulators. To better understand these basic processes, a detailed characterization of all genes involved is needed. Genome-wide RNAi screens can be used as a tool to identify pathway components by systematically characterizing gene functions. However, due to their size and complexity, genome-wide RNAi screens require specific screening protocols as well as fast and stable read-out techniques to deliver high-quality data sets.

The inventors established protocols to characterize the regulation of cell growth and survival on a genome-wide scale. My aim was to monitor the gene silencing effect of 21,115 siRNA-pools on cellular viability in order to get a fingerprint of genes, which are essential for HeLa cells to grow and survive.

In the following step, the inventor expanded this approach and induced apoptosis in the RNAi-treated cells to identify markers of cellular resistance. Therefore, a deathinducing dose TRAIL was used to trigger receptor-mediated apoptosis in a genomewide siRNA screen in HeLa cells. The inventors identified genes, which mediate TRAIL-induced apoptosis, as siRNA-knockdowns leading to an apoptosis resistance.

Processing of Genome-Covering siRNA Libraries

The genome-wide screening approaches were done with a siRNA library containing pools of four single siRNA sequences per gene (Dharmacon) to target 21,115 genes of the human genome. The library was delivered in a 96-well format as lyophilized substance. To reduce the number of plates handled during a genome-wide screen and to limit the cost of reagents, methodologies to screen in 384-well cell culture plates had to be established. The first step before applying the siRNAs in an experiment was the rehydration of the lyophilized siRNA-pellets and the aliquoting into different assay and storage plates. To automate the process, save time and increase the accuracy of the aliquoting, protocols for a liquid handling robot were established and applied. The rehydrated siRNA was either kept as stock solution for long-term storage or was further diluted and distributed into ready-to-use cell culture plates. All plates were stored at −20° C., carefully sealed with adhesive aluminium foil or heat seals to avoid evaporation. During the whole process precautions were taken to avoid contaminations of plates and stocks with RNases or bacterial and fungal infections. This was ensured by aseptic working conditions and the use of RNase-free disposables and liquids.

### Cell Based Assays for the Quantification of Cell Viability Phenotypes

For the detection of cell viability phenotypes in high-throughput RNAi screens a sensitive assay system, capable of distinguishing between a wide range of cell numbers, had to be established. Such a system needs to give stable signals with small standard deviations from low to very high cell numbers, to enable the assembly of reliable and reproducible screening data sets. Furthermore, the assay has to meet the requirements of high-throughput experiments: the signal needs to be measurable even in high-density cell culture plates and the handling should be automatable. Frequently used assays to assess cellular viability rely on the determination of cellular redox and metabolic statuses, which can be correlated to cell number and cell health. When comparing an ATP-quantification assay (CellTiter-Glo, Promega) to a resazurin-based dye that detects metabolic capacity (CellTiter-Blue, Promega) the ATPquantification method showed lower variation and a higher detection range between low and high cell numbers. Furthermore, the ATP-quantification revealed more stable values across many replicates in a high-density format, especially in the case of evaporation of medium in wells at the border of a plate (data not shown).

To test the ATP-quantification assay for its capability to detect RNAi-mediated changes in cellular viability, siRNAs were used to silence genes essential for cell survival. For this purpose, a liposomal siRNA-transfection protocol was established in 384-well high-density plates. The knockdown of ubiquitin B (UBB), ubiquitin C (UBC) and polo-like kinase 1 (PLK1) resulted in severe viability defects represented by decreased levels of cellular ATP (FIG. 3). The ATP-quantification assay reliably and accurately detected cellular viability phenotypes upon siRNA treatment, thus it is a good cell based assay for the high-throughput detection of RNAi-induced viability phenotypes. Therefore, the assay was further used to establish and apply genome-wide RNAi screens for viability and apoptosis phenotypes.

Genome-Wide siRNA Screens for Markers of Cell Viability

To systematically dissect the mechanisms underlying the regulation of growth and survival in human cells, the inventor established high-throughput screening techniques for genome-wide RNAi surveys. The challenge was to establish protocols and procedures, which lead to reliable and reproducible screening data sets and avoid false-positive and false-negative hits. These false signals can occur in case RNAi reagents show off-target effects or the assay system is inappropriate for distinguishing between true and background signals (Jackson 2003; Judge 2005; Birmingham 2006). To avoid these false signals, the ATP-quantification assay was used to establish and conduct the siRNA screens, since the assay showed high sensitivity and stability. In addition, the assay was miniaturized into 384-well format and the protocol was optimized for batch sizes allowing stable conditions in each plate throughout the transfection procedure (FIG. 4 A). Screens were done in duplicates on the same day to produce a complete and reliable data set. An automated transfection protocol was applied to facilitate high transfection efficiencies of siRNAs into the cells. Thereby, the cells were added on top of the preincubated liposome-siRNA complex instead of being seeded on the day before (‘reverse transfection’). After 72 hours of incubation with the siRNAs, the cellular ATP-content was quantified in the samples (FIG. 4 A).

### Data Analysis and Results

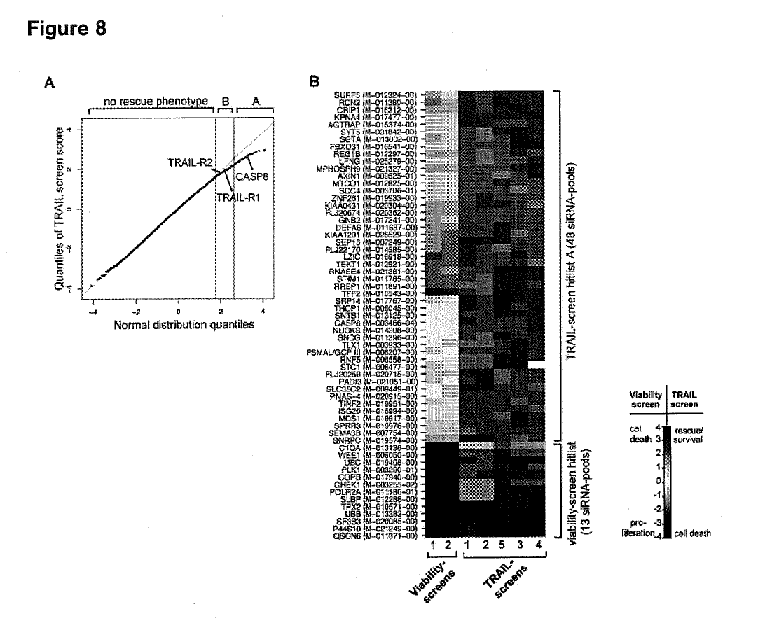

All ATP-quantification values were analysed using the R/Bioconductor software package celIHTS2 (http://www.bioconductor.org) (Boutros 2006). The data sets were normalized across plates and batches to account for differences resulting from the screening procedure. Both screen replicates were compared in a scatterplot to demonstrate the reproducibility of the screen (FIG. 4B). Viability phenotypes of different strength could be visualized ranging from reduced viability to normal growth and proliferation effects. The siRNAs targeting UBB, UBC and PLK1, that had been previously used to establish the ATP-quantification assay, resulted in top scoring viability phenotypes in the screen (FIG. 8 B). These genes were used as internal controls to evaluate the screening results. Furthermore, in the list ofstrongest phenotypes, the inventor identified the cell cycle checkpoint regulators CHEK1 and WEE1 and genes that had not been linked to survival functions before, such as the complement subcomponent C1QA and the histone pre-mRNA-binding protein SLBP (FIG. 8 B).

The viability RNAi-approach generated a genome-wide overview of genes involved in cell growth and survival regulation in HeLa cells. In addition, the data set provides a resource for the alignment of screens involving a compound treatment such as the TRAIL-approach. Thereby the knowledge of the siRNA effects on cellular viability is essential for the interpretation of the data.

Genome-Wide siRNA Screens Identify Novel Mediators of TRAIL-Induced Apoptosis

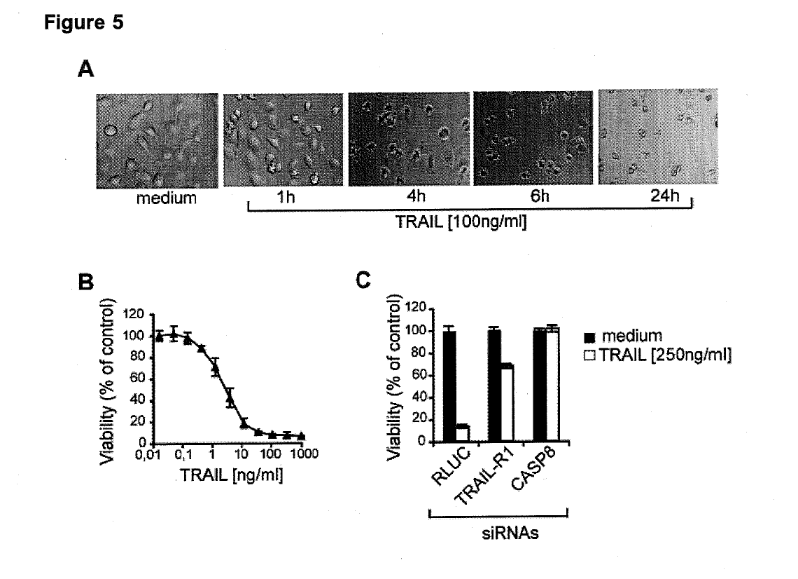

The death-inducing ligand TRAIL triggers apoptosis in HeLa cells shortly after application (FIG. 5 A). During this process, the cytoplasm shrinks, the DNA condensates and the cell membrane starts blebbing, releasing fragments of the cell in a controlled manner (reviewed in Yasuhara 2007). 24 hours after the treatment with 100 ng/ml recombinant TRAIL no viable cells were left (FIG. 5 A). This process was also monitored with a cell-based viability assay, quantifying cellular ATP-levels. TRAIL induced cell death in HeLa cells in a dose-dependant manner (FIG. 5 B). As a control, siRNAs targeting the TRAIL-R1 receptor and caspase-8 inhibited apoptosis and resulted in so-called ‘rescue phenotypes’ (FIG. 5 C).

### Genome-Wide TRAIL Screens

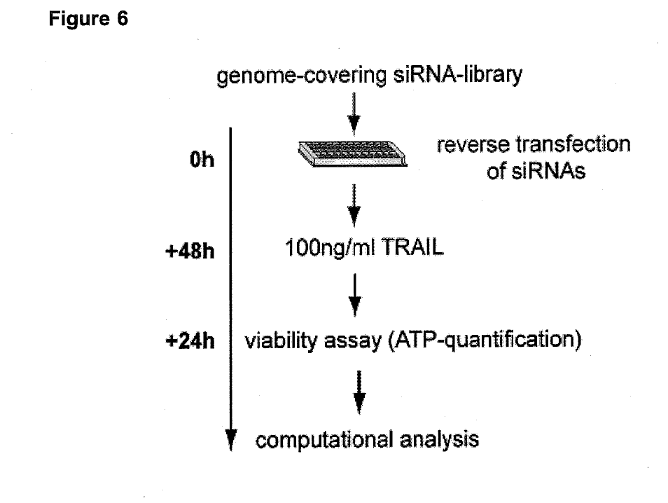

The ATP-quantification assay successfully identified the phenotype of siRNAs targeting known TRAIL-pathway members, such as TRAIL-R1 and caspase-8. This showed that the established protocols could be used for genome-wide RNAi screens to identify novel mediators of the TRAIL pathway. The inventor adapted the protocols used for the viability screens to conduct the TRAIL screens with the exception that 48 hours after the transfection procedure, 100 ng/ml TRAIL were added to the cells (FIG. 6). After further 24 hours incubation with the death ligand, the ATP content of the residual cells was quantified.

### Data Analysis

The TRAIL screen was repeated five times within a timeframe of 14 months to determine the technical reproducibility. The resulting raw data sets were normalized for plate and batch differences using the cellHTS2 software package (http://www.bioconductor.org). The normalized data sets were compared after adjusting the dynamic range of each replicate using quantile normalization). Additionally, all replicates were subjected to a sigmoid transformation to improve the separation of hits from the background signal). The similarity of all five replicates was demonstrated by plotting a pairwise comparison of each replicate and calculating the Pearson's correlation coefficient (FIG. 7) (Moore 2006). The Pearson's correlation reflects the degree of linear relationship between two screening replicates. It ranges from +1 meaning perfect correlation to 0 (no linear correlation) and −1 (perfect negative linear relationship). Correlation coefficients of 0.83 to 0.95 between screen replicates therefore reflect high data reproducibility and a low technical failure rate during the screening procedure (FIG. 7).

The five TRAIL screen replicates were combined and two different hitlists were defined: a stringent one ‘A’ and a relaxed one ‘B’. The stringent hitlist ‘A’ contained the top scoring 48 candidates whereas the relaxed hitlist ‘B’ was computed to contain all knockdowns showing a phenotype differing from a random distribution. The quantile distribution of the combined TRAIL screens was plotted against the theoretical distribution and both hitlists ‘A’ and ‘B’ were colourcoded in red and blue, respectively (FIG. 8 A). The internal control siRNA targeting caspase-8 appeared in the stringent hitlist ‘A’, demonstrating the ability of the approach to identify known pathway components (FIG. 8 A). Furthermore, the knockdowns of TRAIL-receptor TRAIL-R1 and -R2 were identified in hitlist ‘B’. The 48 genes from the stringent TRAIL-hitlist ‘A’ were compared to the top 13 candidates from the viability screen in a heatmap showing the values from each screen replicate (FIG. 8 B). Thereby a hit in the TRAIL assay comprises a siRNA abrogating apoptosis and leading to cellular resistance, whereas a knockdown-candidate from the viability approach results in severe cell death without death stimulus. The heatmap revealed the TRAIL hits of the stringent hitlist ‘A’ as high scoring and reproducible across the replicates. In this list a variety of genes were identified, which have not been connected with TRAIL-induced apoptosis signalling before. Several disease-related genes were identified such as the peroxisome proliferative activated receptor (PPARGCIA), the UL16 binding protein 3 (ULBP3), the AT-binding transcription factor (ATBF1) and the programmed cell death 10 protein (PDCD10) (Guclu 2005; Sutherland 2006; Sun 2007; Lai 2008). Furthermore, the list of the 200 top-scoring knockdowns contains several potential tumour suppressor genes such as the F-box protein 31 (FBXO31), the suppressor of tumorigenicity 18 (ST18) and the tumor suppressor candidate 3 (TUSC3) (MacGrogan 1996; Jandrig 2004; Kumar 2005).

### Secondary Validation of Candidate Genes

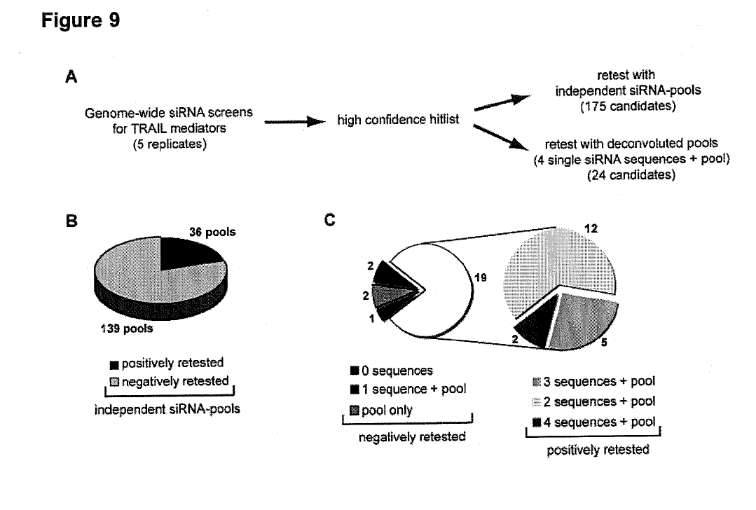

The five TRAIL screen replicates resulted in reproducible hitlists. Nevertheless, besides known and novel regulators, screening hitlists often contain false positive signals (Echeverri 2006). False positives can arise from RNAi reagents having offtarget effects or from measurement artefacts. Since the TRAIL screens were repeated five times and the candidates were calculated across the replicates, variances in screening and microbial contaminations producing high ATP-signals could be excluded. To further exclude false-positives resulting from sequence-dependent off-targets, the inventor intended to reproduce the screening results with independent siRNA-pools (FIG. 9A). Additionally, the siRNA-pools used in the screen were deconvoluted into the four single sequences to monitor the individual gene silencing effects. Thereby the reproducibility of the phenotype with single siRNAs was tested, since a phenotype is more likely to be true if it can be shown with several independent siRNAs. First, to evaluate the candidates from the initial TRAIL screens (Dharmacon siRNA library) 175 independent siRNA-pools from Qiagen were used. The pools were tested with the screening protocol for their ability to rescue TRAIL-induced apoptosis. The transfection protocol was optimized to minimize concentration-dependent off-target effects by using 25 nM siRNA instead of 50 nM. The assay was repeated four times and the resulting values were normalized by the negative control siRNA on each plate. After adding up the values for each gene, 36 siRNA-pools (˜21% of total) were found to reproduce the TRAIL rescue-phenotype according to stringent criteria (on average 2.5 times higher than the TRAIL-treated control siRNA) (FIG. 9 B).

In the second approach, further 24 siRNA-pools (Dharmacon) were deconvoluted and retested as single sequences. The siRNAs were transfected according to the TRAIL screen protocol as single sequences and in combination as pools of four sequences with a constant total concentration of 50 nM. The cellular ATP-levels were quantified for each knockdown and the z-score was calculated. A z-score >4 was considered to be a rescue phenotype. To be positively retested, a gene knockdown experiment needed to show a rescue phenotype in the TRAIL assay with at least the pool and two of its four single sequences. Applying these criteria, 19 out of 24 targeted genes were confirmed (FIG. 9 C). Some candidates were confirmed with single siRNAs as well as with independent sequences, e.g. KPNA4, FBXO31 and Axin1. These genes therefore very likely play a role in TRAIL-induced apoptosis signalling. A second group of candidates, such as FLJ20259, MDS1 and DFFB, were successfully retested with one of the assays and are as well potentially interesting. To further confirm and evaluate their function in apoptosis signalling additional retests are needed.

### Discussion

The TNF family members regulate cellular functions through binding to membrane-bound receptors belonging to the TNF-R family. A subgroup of the TNF-R family, the death receptors transmit an apoptotic signal via their death domains by recruiting intracellular proteins that get activated at the DISC. Amongst the death receptors, the TRAIL/TRAIL-R system is distinct due to its complexity and tumour-specific killing activity. However, many primary tumours are resistant to TRAIL-induced apoptosis. Yet most resistant tumour cells can be sensitised to TRAIL-induced apoptosis by chemo- and radiotherapy whereas normal cells usually remain resistant to TRAIL also in combination with sensitising agents. While the basic machinery that drives TRAIL-induced apoptosis is known, the molecular mechanisms of the regulation of apoptosis induction by TRAIL, especially with respect to tumour-cell-specific sensitisation to TRAIL are poorly understood. Therefore, genome-wide RNAi studies were performed with the aim to identify novel factors that are required for apoptosis induction by TRAIL. A number of such factors was identified. The effect of their absence on TRAIL-induced apoptosis will be discussed. One of these factors, Axin-1, which has extensively been studied in the presented context, will be discussed in most detail.

### Genome-Wide RNAi Screens

The genome-wide RNAi approach proved to be highly stable and reproducible as five consecutive screens over a period of 14 months led to very similar results (FIG. 7). As off-target effects, meaning that the application of an siRNA did not only downregulate the mRNA of interest but also a different mRNA, cannot be excluded when using RNAi-based approaches (Jackson, Bartz et al. 2003), the top hits of the five TRAIL RNAi screens were further evaluated.

### Novel Modulators of the TRAIL Apoptosis Pathway

The genome-wide RNAi screens led to the identification of several factors previously not known to be required for efficient apoptosis induction by TRAIL. Strikingly, KD of many of these factors already affected caspase-8 cleavage (see Table 2). Caspase-8 recruitment to and activation at the TRAIL DISC is crucial for the transduction of the apoptotic signal. It is well established that procaspase-8 is recruited to the TRAIL DISC in a stimulation-dependent manner. At the TRAIL DISC caspase-8 interacts with FADD via its DED and is thereby activated (Walczak and Haas 2008). Other DED-containing proteins for example cFLIP or PED/PEA-15 can compete with caspase-8 for FADD binding which hampers its activation. However, the exact biochemical mechanism and requirements of procaspase-8 recruitment and activation are still unclear. It was recently reported that caspase-8 can be phosphorylated by SRC kinase and that this phosphorylation blocks autocatalytic cleavage of caspase-8 resulting in the suppression of FasL-induced apoptosis (Cursi, Rufini et al. 2006). Some of the factors identified in the genome-wide TRAIL RNAi screens may directly or indirectly, via other signalling pathways, be involved in efficient recruitment and activation of caspase-8.

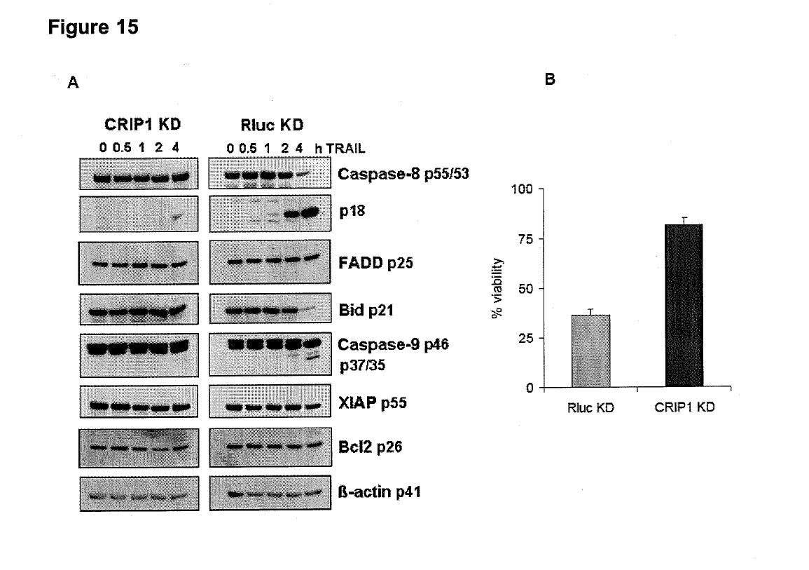

One of these novel factors whose KD almost abolished caspase-8 cleavage and led to TRAIL resistance is the cysteine-rich protein 1 (CRIP1) (FIG. 7, FIG. 13, FIG. 15). CRIP1 belongs to the LIM/double zinc finger protein family CRIP1 and has been implicated in intestinal zinc transport (Hempe and Cousins 1991). Furthermore, CRIP1 is often hypomethylated in prostate cancer (Wang, Williamson et al. 2007). CRIP1 has been identified as a prognostic marker for early detection of cancers and peptide ligands to CRIP1 are currently designed as novel biomarkers for cancers (Hao, Serohijos et al. 2008).

Another protein that strongly affected caspase-8 cleavage at the TRAIL-DISC is the Olfactory receptor, family 9, subfamily G, member 4 (OR9G4). OR9G4 belongs to a large family of Gprotein-coupled receptors (GPCR) which are responsible for recognition and G-proteinmediated transduction of odorant signals. However, little is known about the expression and function of OR9G4. OR9G4 is required for TRAIL-induced apoptosis (FIG. 2) and its function is most likely to regulate caspase-8 expression (FIG. 24). OR9G4 KD leads to a decrease in caspase-8 expression and therefore caspase-8 cannot be efficiently recruited and activated at the TRAIL DISC (FIG. 24). It can now be investigated whether the regulation of caspase-8 happens at the mRNA or the protein level. Furthermore, it should be examined which signalling pathway(s) are affected by OR9G4 and whether OR9G4 can be triggered by activating antibodies or small molecules that bind to OR9G4. Such treatment renders cancer cells more susceptible to TRAIL-induced apoptosis.

The nuclear ubiquitous casein and cyclin-dependent kinases substrate (NUCKS) and KIAA0431 are also novel factors that are required for TRAIL-induced apoptosis and whose KD affects caspase-8 cleavage (FIG. 7, FIG. 13, FIG. 18, FIG. 23). Interestingly, both proteins have been shown to be localised in the nucleus and to interact with ATM (ataxia telangiectasia mutated), a kinase known to play a role in DNA repair after double-strandbreaks (Grundt, Naga et al. 2004; Kanu and Behrens 2007).

KIAA0431 is also named ATMIN for ATM interactor. ATMIN can interact with ATM through a C-terminal motif, which is also present in Nijmegen breakage syndrome (NBS)1 (Kanu and Behrens 2007; Kanu and Behrens 2008). NBS1 is characterised by short stature, progressive microcephaly with loss of cognitive skills, premature ovarian failure in females, recurrent sinopulmonary infections, and an increased risk for cancer, particularly lymphoma (Demuth and Digweed 2007).

As mentioned before, KD of ATMIN delayed and diminished caspase-8 cleavage after TRAIL treatment (FIG. 17). In addition FADD levels were slightly decreased in ATM 1N KD cells. At the moment, it is unclear whether the lower FADD levels in ATMIN KD cells led to less FADD recruitment and therefore less caspase-8 activation at the TRAIL DISC or whether other mechanism that regulate caspase-8 cleavage are affected by ATMIN KD. Furthermore, Bid cleavage was markedly decreased which is most likely due to less caspase-8 activity in ATMIN KD cells.

NUCKS was shown to be phosphorylated upon DNA damage, most likely by ATM, ATR or CDK1 (Ostvold, Norum et al. 2001; Grundt, Skjeldal et al. 2002; Grundt, Haga et al. 2004). KD of NUCKS led to pronounced resistance to TRAIL-induced apoptosis (FIG. 7, FIG. 13). Delayed and decreased caspase-8 cleavage could be observed in NUCKS KD cells upon TRAIL stimulation (FIG. 17). In addition, Bid cleavage was markedly decreased which can most likely be attributed to decreased activity of caspase-8 in NUCKS KD cells. NUCKS possesses two nuclear localization signals and a DNA binding domain. It is mainly localised in the nucleus (Grundt, Haga et al. 2007). At this point it is unclear how nuclear kinases and their activity to phosphorylate various substrates contributes to caspase-8 activation. Interestingly, nuclear caspase-8 staining has been observed in primary tumour samples from various tissues (Sykora, Walczak; unpublished data).

Furthermore, Yao et al. proposed that the DED domain of caspase-8 translocates to the nucleus by binding to ERK1/2 contributing to caspase-8-dependent apoptosis (Yao, Duan et al. 2007).

TRAIL has been shown to induce a DNA damage response causing the phosphorylation of H2AX (Histone 2AX), Chk2 (checkpoint kinase 2), ATM and DNA-PK (DNA-dependent protein kinase) (Solier, Sordet et al. 2008). In addition, Bid has been shown to be phosphorylated upon DNA double-stand breaks in an ATM-dependent manner (Kamer, Sarig et al. 2005). However, whether Bid phosphorylation alters its pro-apoptotic function in the TRAIL apoptosis pathway is still contested.

The F-box only protein 31 (FBXO31) was another protein whose KD strongly delayed and diminished caspase-8 cleavage after TRAIL treatment (FIG. 7, FIG. 13, FIG. 16). In addition, direct downstream effector events like cleavage of Bid and caspase-3 were markedly reduced.

There are indications that FBXO31 functions as a senescence and tumour suppressor gene. Furthermore, FBXO31 was shown to be downregulated in various breast cancer cell lines as well as primary mammary tumours (Kumar, Neilsen et al. 2005). FBXO31 has been implicated in the ubiquitination of proteins (Jin, Cardozo et al. 2004). FBXO31 probably exerts its function by generating SCF(FBXO31) complexes that target particular substrates for ubiquitination and subsequent degradation. In the study by Kumar et al., the proposed targets of SCF(FBXO31) complexes were several proteins which are critical for cell cycle progression (Kumar, Neilsen et al. 2005).

Another protein whose KD led to markedly decreased caspase-8 cleavage after TRAIL treatment is PNAS-4 (putative apoptosis-related protein in human acute promyelocytic leukemia cell line NB4) (FIG. 7, FIG. 13, FIG. 25). Furthermore, downstream effector events like Bid and caspase-9 cleavage were strongly reduced in PNAS-4 KD cells.

PNAS-4 is a highly conserved protein and it was recently identified as a pro-apoptotic protein in Xenopus laevis (Yan, Qian et al. 2007) and mouse (Zhang, Wang et al. 2008). PNAS-4 is up-regulated in human papillomavirus-infected invasive cervical cancer and androgen-independent prostate cancer (Best, Gillespie et al. 2005; Santin, Zhan et al. 2005). Furthermore, it was demonstrated that transfer of PNAS-4 plasmid/liposome complexes induced apoptosis in vivo in a nude mice xenograft model and enhanced sensitivity to gemcitabine in lung cancer (Hou, Zhao et al. 2008).

PNAS-4 has also been reported to be a novel regulator for convergence and extension during vertebrate gastrulation (Yao, Xie et al. 2008). In zebrafish, KD of PNAS-4 caused gastrulation defects with a shorter and broader axis. In addition, the authors proposed that PNAS-4 might act in parallel with non-canonical Wnt signalling in the regulation of cell movement. Human PNAS-4 has 96% identity with mouse PNAS-4 in primary sequence and has also been reported to be involved in the apoptotic response to DNA damage (Filippov, Filippova et al. 2005). As mentioned earlier, DNA damage causes the activation of ATM which has been reported to interact with ATMIN and NUCKS that are required for TRAIL-induced apoptosis. These proteins as well as PNAS-4 already affected the processing of caspase-8 after TRAIL treatment.

KD of KPNA4 (karyopherin subunit alpha-4), MDS1 (Myelodysplastic syndrome 1) or RNF5 (Ring-finger protein 5), three proteins identified in the genome-wide TRAIL RNAi screens, slightly affected the cleavage of caspase-8 after TRAIL treatment. Therefore, these proteins may not be crucial for recruitment of caspase-8 to and its activation at the TRAIL DISC but may influence signalling pathways contributing to efficient caspase-8 processing.

KPNA4 has been described to play a role in nuclear protein import as an adapter protein for the nuclear receptor KPNB1. In vitro, KPNA4 has been shown to mediate the nuclear import of the human cytomegalovirus protein UL84 by recognizing a non-classical NLS (Ayala-Madrigal, Doerr et al. 2000; Fagerlund, Kinnunen et al. 2005; Grundt, Naga et al. 2007). It is possible that KPNA4 regulates the nuclear import of (an)other protein(s) that facilitate(s) TRAIL-induced apoptosis.

KD of MDS1 led to pronounced resistance to TRAIL-induced apoptosis (FIG. 2). A chromosomal aberration (t3;21) involving the MDS1 protein is found in a form of acute myeloid leukemia (AML). MDS1 can be produced either as a separate transcript and as a normal fusion transcript with EVI1 (ecotropic viral integration site 1), resulting in overrepresentation of MDS1 (Metais and Dunbar 2008). As KD of MDS1 conferred resistance to TRAIL-induced apoptosis, an overexpression could make cells more sensitive to TRAIL treatment. Interestingly, however high-risk myelodysplastic syndrome (MDS) has been associated with reduced NK cell function (Epling-Burnette, Bai et al. 2007). The reduced NK cell function in turn correlated with downregulation of activating receptors NKp30 and NKG2D. TRAIL expression is upregulated on NK cells after activation (Kayagaki, Yamaguchi et al. 1999) and surfacebound TRAIL is one of the main effector mechanisms of NK cells (Kayagaki, Yamaguchi et al. 1999). Therefore, MDS1-overexpressing cells could have developed a mechanism to regulate the activation of NK cells, thereby preventing TRAIL-mediated killing by NK cells.

KD of RNF5 also reduced caspase-8 processing. Yet, interestingly, the effect on caspase-9 cleavage was even more pronounced (FIG. 27). RNF5 is predominantly located at the plasma membrane and possesses a RING-type zinc finger domain which is required for its ubiquitin ligase activity. RNF5 has been shown to mediate the K63-linked ubiquitination of paxillin and subsequent relocalisation of paxillin (Didier, Broday et al. 2003). It is possible that RNF5 interplays with GNB2 (guanine nucleotide binding protein beta polypeptide 2) and AGTRAP (type-1 angiotensin II receptor-associated protein) two other hits of the TRAIL RNAi screens which will be discussed in more detail below.

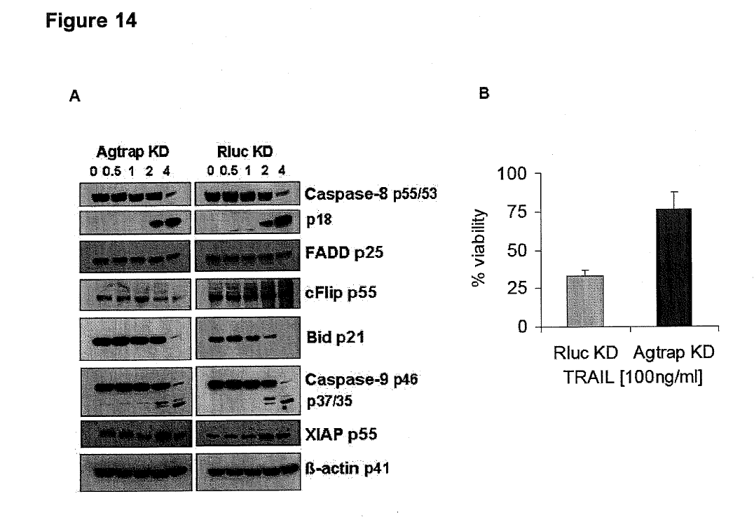

AGTRAP has been implicated in the negative regulation of type-1 angiotensin II receptormediated signaling by regulating receptor internalisation as well as receptor phosphorylation. Interestingly, a yeast two-hybrid screen revealed RACK1 (Receptor of Activated Protein C Kinase) as an interaction partner of human AGTRAP (Wang, Huang et al. 2002). RACK1 is also known as guanine nucleotide binding protein beta polypeptide 2-like 1 (GNB2L1) a protein that shares high similarity with GNB2 which was also a hit in the TRAIL screen (FIG. 7 and FIG. 13). Both proteins, GNB2 and GNB2L1, belong to the WD repeat G protein beta family. RACK1/GNB2L1 has recently been suggested to play a role in protecting cancer cells from paclitaxel-induced apoptosis by regulating the degradation of BimEL (Zhang, Cheng et al. 2008). Furthermore, a recent paper by Parent et al., showed that inhibition of RACK1/GNB2L1 affected cell surface expression of the G protein-coupled receptor for thromboxane A(2), CXCR4 and the angiotensin II type 1 receptor that is associated with AGTRAP. It could be possible that the mainly ER- and membrane-bound protein AGTRAP (Wang, Huang et al. 2002; Kamada, Tamura et al. 2003; Lopez-Ilasaca, Liu et al. 2003; Ihara, Egashira et al. 2007) acts together with RACK1/GNB2L1 to transport TRAIL receptors from the ER to the cell surface. However, caspase-8 cleavage was not affected after TRAIL treatment in AGTRAP KD cells, speaking against a role for regulation at the TRAIL receptor surface expression level (FIG. 14). This can, however, easily be tested by determining the surface expression of the different TRAIL receptors with and without AGTRAP KD. If RACK1/GNB2L1 and AGTRAP caused degradation of pro-apoptotic BimEL after TRAIL stimulation, as it was shown after paclitaxel treatment, the changed ratio of pro- to antiapoptotic Bcl-2 family members could prevent mitochondrial depolarisation and so contribute to TRAIL resistance. However, under these circumstances, a decreased activation of caspase-9 would be expected. As caspase-9 processing was not affected by AGTRAP KD, a role together with RACK1/GNB2L1 in regulating BimEL and this being the cause for the observed resistance to TRAIL-induced apoptosis after AGTRAP KD is rather unlikely.

Moreover, RACK1/GNB2L1 has been shown to form a ternary complex with TRIM63 (tripartite motif-containing 63) and PRKCE (protein kinase C, epsilon). PRKCE in turn has been shown to protect MCF-7 breast cancer cells from TRAIL- and TNF-induced apoptosis (Lu, Sivaprasad et al. 2007; Shankar, Sivaprasad et al. 2008).

RACK1/GNB2L1 is also an adaptor protein that regulates signalling via SRC and PKCdependent pathways and has been implicated in cell migration (Doan and Huttenlocher 2007). KD of RACK1/GNB2L1 reduced the dynamics of paxillin and talin at focal complexes whereby RACK1/GNB2L1 functioned to regulate SRC-mediated paxillin phosphorylation. As mentioned before another hit of the TRAIL screen, RNF5, has been shown to mediate the K63-linked ubiquitination of paxillin and subsequent relocalisation of paxillin (Didier, Broday et al. 2003). Furthermore, it has been shown that ectopic expression of RNF5 increased the cytoplasmic distribution of paxillin while its localization within focal adhesions was decreased (Didier, Broday et al. 2003). Yet, interestingly, according to these data KD of RNF5 should increase paxillin at focal adhesion points by blocking K63-ubiquitination that would lead to translocation of paxillin to the cytoplasm, while KD of RACK1/GNB2L should decrease the number of focal adhesion points (Doan and Huttenlocher 2007) and thereby decrease migration. Concerning TRAIL-induced apoptosis, the KD of either of the proteins, RNF5 or GNB2 conferred resistance to apoptosis induction by TRAIL (FIG. 7).

TRAIL itself has been implicated to play a role in cell migration (Secchiero, Melloni et al. 2008). Recombinant TRAIL was shown to stimulate migration of TRAIL-resistant mesenchymal stem cells (Secchiero, Melloni et al. 2008). Furthermore, it was demonstrated that certain colorectal cancer cells started migrating upon TRAIL treatment (Hoogwater et al., personal communication). Interestingly, only TRAIL-resistant cells were shown to migrate upon TRAIL stimulation while TRAIL-sensitive cells obviously die upon TRAIL treatment. However, it is unlikely that all TRAIL-resistant cells will migrate upon TRAIL stimulation.

Initiator caspases that are activated at the TRAIL DISC cannot only trigger a caspase cascade but also cleave the BH3-only protein Bid. Pro-apoptotic tBid then translocates to the mitochondria where it interacts with other Bcl-2 family members, leading to the activation of Bax and Bak and ultimately to mitochondrial membrane depolarisation and the release of cytochrome c. Cytochrome c, together with dATP, APAF1 and caspase-9 forms the apoptosome where caspase-9 is autocatalytically activated. Interestingly, the genome-wide RNAi screen for TRAIL apoptosis modulators revealed a protein named APIP (APAF1 interacting protein) that has been shown to interact with APAF1. This protein is also called MMPR19 (monocyte macrophage protein 19) because it was first identified as a novel cDNA sequence from murine monocyte and macrophage tissue. Overexpression of MMRP19/APIP has been shown to inhibit cytochrome c-induced activation of caspase-9 and to suppress cell death triggered by mitochondrial apoptotic stimuli (Cho, Hong et al. 2004). A recent paper described a novel Apaf-1-independent anti-apoptotic role for MMRP19/APIP where MMRP19/APIP leads to sustained activation of AKT and ERK1/2 and thereby prevents hypoxic cell death (Cho, Lee et al. 2007).

Besides ERK1/2, other MAPKs have been implicated in TRAIL signalling. JNK activation by TRAIL has been reported to occur in a caspase-dependent fashion and can be mediated by TRAIL-R1 and TRAIL-R2 (Hu, Johnson et al. 1999). Although JNK activation in the TNF pathway has been associated with apoptosis induction (Wullaert, Heyninck et al. 2006), JNK activation is not required for TRAIL-induced apoptosis (MacFarlane, Cohen et al. 2000).

Interestingly, the genome-wide RNAi screen revealed that KD of JNK2 (MAPK9), but not JNK1, confers resistance to TRAIL-induced apoptosis (FIG. 10, FIG. 13). JNK2/MAPK9 deficiency has been shown to protect mice from hypercholesterolemia-induced endothelial dysfunction and oxidative stress (Osto, Matter et al. 2008). Furthermore, differential roles of JNK1 and JNK2/MAPK9 have been described in murine steatohepatitis and insulin resistance. In mice with established steatohepatitis, KD of JNK1 decreased the amount of steatohepatitis together with a normalization of insulin sensitivity while KD of JNK2/MAPK9 improved insulin sensitivity but had no effect on hepatic steatosis. Furthermore, JNK2/MAPK9 KD increased hepatic expression of the proapoptotic Bcl-2 family members Bim and Bax and the increase in liver injury resulted in part from a Bim-dependent activation of the mitochondrial death pathway (Singh, Wang et al. 2008). Yet, JNK2/MAPK9 KD protected cells from TRAIL-induced apoptosis (FIG. 13, FIG. 19). Therefore, it is unlikely that in our case the pro-apoptotic Bcl-2 family members Bim and Bax are upregulated because they would rather contribute to apoptosis induction by TRAIL. An upregulation of XIAP has been observed after MAPK9/JNK2 KD which could contribute to the TRAIL resistance.

Another protein that is required for TRAIL-induced apoptosis is the glutamine-rich protein 1 (Qrich1/FLJ20259) (FIG. 7, FIG. 26) which has been annotated in 2004 as a novel gene transcript located on chromosome 3 (3p21.31) (Gerhard, Wagner et al. 2004; Ota, Suzuki et al. 2004). Domain structure analysis showed that Qrich1 contains a CARD domain (UniProtKB/Swiss-Prot). So far, this protein has not been described to interact with any particular CARD-containing protein, but theoretically it could interact with the CARD domain-containing caspase-9 or Apaf-1. However, the results presented speak against a crucial role of Qrich1 for the activation of caspase-9, as the cleavage of this caspase is not altered by Qrich1 KD (FIG. 26). The Mitochondria-associated granulocyte macrophage CSF-signaling molecule (Magmas) has been shown to be induced by granulocyte-macrophage-colony stimulating factor (GM-CSF) in hematopoietic cells (Jubinsky, Messer et al. 2001; Peng, Huang et al. 2005). The protein is also called mitochondrial import inner membrane translocase subunit (TIM16) because it is a component of the presequence translocase-associated motor (PAM) complex, which is required for the translocation of transit peptide-containing proteins from the inner membrane into the mitochondrial matrix in an ATP-dependent manner (Kozany, Mokranjac et al. 2004; Iosefson, Levy et al. 2007; Mokranjac, Berg et al. 2007). During TRAIL-induced apoptosis, mitochondria are depolarized and pro-apoptotic factors are released from the mitochondria.

The 15 kD selenoprotein (SEP15) which was also required for TRAIL-induced apoptosis (FIG. 7, FIG. 13) has been implicated in disulfide bond assisted protein folding in the ER where it can bind to UDP-glucose:glycoprotein glucosyltransferase (GT), an essential regulator of quality control mechanisms within the ER (Labunskyy, Ferguson et al. 2005; Labunskyy, Hatfield et al. 2007).

Therefore, SEP15 could be important for the correct folding and subsequent surface expression of TRAIL receptors. However, as caspase-8 cleavage was not affected by SEP15 KD, this is rather unlikely. Interestingly, Bid cleavage was normal while caspase-9 cleavage was delayed in SEP15 KD cells (FIG. 28), indicating a role of SEP15 between Bid cleavage and caspase-9 activation, maybe at the level of mitochondrial depolarisation controlled by Bcl-2 family members.

Furthermore, SEP15 binds selenium and is involved in selenium-mediated apoptosis. Apostolou et al. showed that malignant mesothelioma cells that lack SEP15 expression are more resistant to growth inhibition and apoptosis induction by selenium (Apostolou, Klein et al. 2004).

1. A method of modulating apoptosis-factor-associated cell death and/or apoptosis in a patient in need of such modulation, comprising administering to said patient an effective amount of an agent selected from a nucleic acid molecule as identified in Table 1, a homologue thereof, a polypeptide encoded by said nucleic acid molecule or homologue thereof or an effector of said nucleic acid molecule or of said polypeptide.

2. The method according to claim 1, wherein the modulator of apoptosis-factor-associated cell-death modulates apoptosis-factor-induced apoptotic cell death and/or apoptosis-factor-induced non-apoptotic cell death.

3. The method according to claim 1, wherein apoptosis-factor-associated cell death and/or apoptosis is TRAIL-induced cell death, in particular TRAIL-induced non-apoptotic cell death and/or TRAIL-induced apoptosis.

4. Agent selected from a nucleic acid molecule as identified in Table 1, a homologue thereof, a polypeptide encoded by said nucleic acid molecule or homologue thereof or an effector of said nucleic acid molecule or of said polypeptide for use as a modulator of apoptosis-factor-associated cell survival, migration and/or proliferation.

5. The agent for use according to claim 4, wherein the modulator of apoptosis-factor-associated cell survival, migration and/or proliferation modulates TRAIL-induced cell survival, migration and/or proliferation.

6. The method according to claim 1, wherein the apoptosis-factor is selected from the group consisting of TRAIL, CD95L, TNF, TL1A and any combination thereof, preferably TRAIL.

7. The method according to claim 1, wherein the nucleic acid molecule is a DNA or RNA encoding a mammalian, particularly human polypeptide, or a variant thereof.

8. The method according to claim 1, wherein the polypeptide is a mammalian, particularly human polypeptide or a variant thereof.

9. The method according to claim 1, wherein the effector is
(i) an antibody directed against the polypeptide,
(ii) a truncated or mutated fragment of the polypeptide,
(iii) a nucleic acid effector molecule or
(iv) a low-molecular weight compound.

10. The method according to claim 1, which is a stimulator of apoptosis-factor-associated cell death and/or apoptosis-factor-associated apoptosis.

11. The method according to claim 1, which is a stimulator of TRAIL-induced cell death and/or TRAIL-induced apoptosis.

12. The method according to claim 10 for the treatment of inflammation, rheumatoid arthritis, multiple sclerosis, hyperproliferative disorders, such as cancer and/or viral infections such as by CMV, influenza virus, respiratory syncytial virus.

13. The method according to claim 1, which is an inhibitor of apoptosis-factor-associated cell death and/or apoptosis-factor-associated apoptosis.

14. The method according to claim 1, which is an inhibitor of TRAIL-induced cell death and/or TRAIL-induced apoptosis.

15. The method according to claim 13 for the treatment of cancer, acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis and/or viral infections e.g. by HIV.

16. The agent for use according to claim 4, which is a stimulator of apoptosis-factor-associated cell survival, migration and/or proliferation.

17. The agent for use according to claim 4 which is a stimulator of TRAIL-induced cell survival, migration and/or proliferation.

18. The agent for use according to claim 16 for the treatment of cancer, acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis and/or viral infections e.g. by HIV.

19. The agent for use according to claim 4, which is an inhibitor of apoptosis-factor-associated cell survival, migration and/or proliferation.

20. The agent for use according to claim 4, which is an inhibitor of TRAIL-induced cell survival, migration and/or proliferation.

21. The agent for use according to claim 19 for the treatment of inflammation, rheumatoid arthritis, multiple sclerosis, hyperproliferative disorders, such as cancer and/or viral infections such as by CMV, influenza virus, respiratory syncytial virus.

22. The method according to claim 1 in combination with at least one further therapeutic compound such as an agent as defined in claim 1, chemotherapeutics, blockers or inducers of apoptosis-factors, targeted drugs and/or irradiation therapy.

23. The method according to claim 22, wherein the targeted drug is a TRAIL-R agonist.

24. The method according to claim 23, wherein the TRAIL-R agonist is TRAIL, preferably exogenous TRAIL such as recombinant TRAIL, and/or an anti-TRAIL-receptor antibody such as anti-TRAIL-R1 or anti-TRAIL-R2.

25. The method according to claim 1 when used in human or in veterinary medicine.

26. Method of diagnosing or monitoring a condition or disorder in a cell or an organism, comprising determining in a sample from said cell or organism the amount and/or activity of at least one nucleic acid molecule as identified in Table 1, a homologue thereof or polypeptide encoded by said nucleic acid.

27. The method of claim 26, wherein the condition or disorder to be monitored can be treated by a TRAIL-R agonist.

28. The method of claim 26, wherein the TRAIL-R agonist is TRAIL, preferably exogenous TRAIL such as recombinant TRAIL, and/or an anti-TRAIL-receptor antibody such as anti-TRAIL-R1 or anti-TRAIL-R2.

29. The method of claim 26, wherein the condition or disorder is an apoptosis-factor associated condition or disorder.

30. The method of claim 29, wherein the apoptosis-factor is selected from the group consisting of TRAIL, CD95L, TNF, TL1A and any combination thereof, preferably TRAIL.

31. The method according to claim 26, wherein the apoptosis-factor-associated condition is related to dysregulated cell survival, migration, proliferation and/or non-apoptotic or apoptotic cell-death.

32. The method according to claim 26, wherein at least one further condition or disorder-associated factor is determined, such as a biomarker for e.g. cancer.

33. Use of a nucleic acid molecule as identified in Table 1, a homologue thereof or polypeptide encoded by said nucleic acid as a diagnostic marker for TRAIL-, CD95L-, TNF- and/or TL1A-associated cell survival, migration, proliferation, non-apoptotic cell-death and/or apoptosis, preferably TRAIL-induced apoptosis.

34. Diagnostic tool for TRAIL-, CD95L-, TNF- and/or TL1A-associated cell survival, migration, proliferation, non-apoptotic cell-death or apoptosis, preferably TRAIL-induced apoptosis, comprising at least one reagent for determining the amount and/or activity of at least one nucleic acid molecule as identified in Table 1, at least one homologue thereof or polypeptide encoded by said nucleic acid.

35. The diagnostic tool of claim 34 comprising a panel of at least two reagents, preferably at least two reagents for determining the amount and/or activity of at least one nucleic acid molecule as identified in Table 1, at least one homologue thereof or polypeptide encoded by said nucleic acid.

36. The diagnostic tool of claim 34, further comprising at least one reagent for determining the amount and/or activity of further TRAIL-, CD95L-, TNF- and/or TL1A-associated nucleic acids or polypeptides, in particular TRAIL-associated nucleic acid molecules or polypeptides such as FADD, cFLIP, Caspase 8, Caspase 10, TRAIL-R1, TRAIL-R2, TRAIL-R3, TRAIL-R4, OPG, Axin-1, RIP-1.

37. The diagnostic tool of claim 34, wherein the reagent is selected from antibodies or nucleic acid molecules.

38. Method of identifying a modulator of TRAIL-, CD95L-, TNF- and/or TL1A-associated cell survival, migration, proliferation, non-apoptotic cell-death and/or apoptosis, in particular, TRAIL-induced apoptosis, comprising evaluating or screening whether a test compound has the ability to modulate the amount and/or activity of at least one nucleic acid molecule as identified in Table 1, a homologue thereof or polypeptide encoded by said nucleic acid.

39. The method of claim 38, wherein the test compound induces at least one nucleic acid molecule as identified in Table 1, a homologue thereof or polypeptide encoded by said nucleic acid.

40. The method of claim 38, wherein the test compound is to be used in combination with a TRAIL-R agonist, such as TRAIL, preferably exogenous TRAIL such as recombinant TRAIL, and/or an anti-TRAIL-receptor antibody such as anti-TRAIL-R1 or anti-TRAIL-R2.

41. The method of claim 38, wherein the test compound stimulates or enables TRAIL-, CD95L-, TNF- and/or TL1A-associated non-apoptotic cell-death and/or apoptosis, in particular TRAIL-induced apoptosis.

42. The method of claim 41, wherein the test compound is a candidate agent for the treatment of inflammation, rheumatoid arthritis, multiple sclerosis, hyperproliferative disorders, such as cancer and/or viral infections, e.g. by CMV, influenza virus, respiratory syncytial virus etc.

43. The method of claim 38, wherein the test compound stimulates or enables TRAIL-, CD95L-, TNF- and/or TL1A-associated cell survival, migration and/or proliferation.

44. The method of claim 43, wherein the test compound is a candidate agent for the treatment of acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis and/or viral infections e.g. by HIV.

45. The method of claim 38, wherein the test compound inhibits TRAIL-, non-apoptotic cell-death and/or apoptosis, in particular TRAIL-induced apoptosis.

46. The method of claim 45, wherein the test compound is a candidate agent for the treatment of acute or chronic degenerative disorders, such as neurodegenerative disorders, spinal cord injury, autoimmune disorders, stroke, myocardial infarction, aplastic anemia, Fanconi anemia, myelodysplastic myeloma, diabetes, in particular type I diabetes, thyroiditis, multiple sclerosis and/or viral infections e.g. by HIV.

47. The method of any claim 38, wherein the test compound inhibits TRAIL-, CD95L-, TNF- and/or TL1A-associated cell survival, migration and/or proliferation.

48. The method of claim 47, wherein the test compound is a candidate agent for the treatment of inflammation, rheumatoid arthritis, multiple sclerosis and/or hyperproliferative disorders, such as cancer and/or viral infections such as by CMV, influenza virus, respiratory syncytial virus.

49. The method of claim 40, wherein the test compound is (i) an antibody directed against the polypeptide,
(ii) the polypeptide or variant therefrom,
(iii) a nucleic acid effector molecule, or
(iv) a low-molecular weight compound.


SEQUENCE LISTING (41 sequences)

SEQ ID NO: 1 - DNA, 2400 bp, Homo sapiens
  mRNA (1)..(2400): NM_153345
gacaggtgagcgacgaacttctgagacaggtgtgggtgcgagggtcgggagggtcatgggattgggaccgaggtgtgaggagggaatctgcaattccttgctacacagagcgctggcaacttctgacaggctgtttctggggtatgggctgcctcgggttgttgctgttacaaggaaagaaaagagttcccctgcccaccgcctcccagccactgggctacctcctggcaggaaatttgcaaactgagtttaacaagttaggatcagcagagggtagaggagggccctggcagatgtggggtctagaagaggacaggagttatcagggcctccggccattgtgctgggcctttgcctgtacaattgtttctcaagcagttgtgtccctgtggctttggtgcgcctgtgtgcactttctccctccacctggagcatgggctaacaccggaggaaaggaaaagacagagtcagacagggagcctggggaggggccatggtgccaatgcacttactggggagactggagaagccgcttctcctcctgtgctgcgcctccttcctactggggctggctttgctgggcataaagacggacatcacccccgttgcttatttctttctcacattgggtggcttcttcttgtttgcctatctcctggtccggtttctggaatgggggcttcggtcccagctccaatcaatgcagactgagagcccagggccctcaggcaatgcacgggacaatgaagcctttgaagtgccagtctatgaagaggccgtggtgggactagaatcccagtgccgcccccaagagttggaccaaccacccccctacagcactgttgtgatacccccagcacctgaggaggaacaacctagccatccagaggggtccaggagagccaaactggaacagaggcgaatggcctcagaggggtccatggcccaggaaggaagccctggaagagctccaatcaaccttcggcttcggggaccacgggctgtgtccactgctcctgatctgcagagcttggcggcagtccccacattagagcctctgactccaccccctgcctatgatgtctgctttggtcaccctgatgatgatagtgttttttatgaggacaactgggcacccccttaaatgactctcccaagatttctcttctctccacaccagacctcgttcatttgactaacattttccagcgcctactatgtgtcagaaacaagtgtttctgcctggacatcataaatggggacttggaccctgaggagagtcaggccacggtaagcccttcccagctgagatatgggtggcataatttgagtcttctggcaacatttggtgacctaccccatatccaatatttccagcgttagattgaggatgaggtagggaggtgatccagagaaggcggagaaggaagaagtaacctctgagtggcggctattgcttctgttccaggtgctgttcgagctgttagaacccttaggcttgacagctttgtgagttattattgaaaaatgaggattccaagagtcagaggagtttgataatgtgcacgagggcacactgctagtaaataacattaaaataactggaatgaactcctgatcccaaagcctattgtgttttcaacacaaggtaataggaagccaagcaacttgcatgtctcccaatccctgtctagggacagctgctggtgcaggatggaagacttcctataaacagaatgggcgaccaattgtgtctacagagggggtgggctgagcatgggcacgcatgtccccagcctcctggacgagcaaagtcagtcaaagcgctggtgatccctgctccgcgtgcgtagcagtgtctgtgcctctcctgcccaggggctagagagcagtctccagtgcagggtccccatcctatctgaaaacagtggagtcagtgaccccaggtggagggacttccagttttaacaatgggttggtatgaaggccagaaggagtgaaacatgtgaactttctggcgaggagactccctcttgaatcatccatatgtagccccggggtcacttgcgaagagtctctaacagcttctgcctcactcctaagctctgaccgctaggctttcaccccagccgccggtcctgattcctgagatcccaatattgagcaccaggtttcctggaattgtgtgctgcggctgtgatgtaggtttcggtcgctggaagctgctaaaccatagctgacgccccctcctaagccagccttccttccccgcgcgggcatctgtccagggccttgtcgcgctctcagtctccttcgcagcctggccccaaccgttcaacttcaataaagcaaaactccagccaaaaaaaaaaaaaaaaaaaaaaaaaaaaaaaaaaaaaa

SEQ ID NO: 2 - DNA, 5003 bp, Homo sapiens
  mRNA (1)..(5003): NM_019086
agttgggctcccgcctggctgggaggcgggagggatcccgctcctgttgttttccgccggcaggagtaggctggcgggcgcagggggcggggtgcgccctccctccccggccagggcgctcgggagcggggacccgagcctgcagccgagctccgctgccggccctggacactcggctcagccaagcatccttcctgggggccgaggaagtggggccactctgccgttccgaggacctgggaggagccctcggtaccccgggccccggggccctggggcacacacgtccagcccagcccgagcctgcgtttcctgagccgggatctggggcgagatggccgcaggcggcagtgcgcccgagccccgcgtcctcgtctgcctcggggcgctcctggccggctgggtcgccgtaggattggaggctgttgtcattggagaagttcatgagaatgttactctgcactgtggcaacatctcgggactgaggggccaggtgacctggtaccggaacaactcggagcctgtcttccttctctcgtccaactctagcctccggccagctgagcctcgcttctctctagtggatgccacctccctgcacattgaatcgctgagcctgggagatgagggaatctacacctgccaggagatcctgaatgtgactcagtggttccaagtgtggctgcaggtggccagcggcccctatcagattgaggtccacatcgtggccaccggcacactccccaacggcaccctctacgcagccaggggctcccaggtggacttcagctgcaacagcagctccaggccaccacccgtggttgaatggtggttccaggccctgaattccagcagcgagtcctttggccacaacctgacagtcaactttttctcactgttactgatatcgccaaacctccaagggaactacacctgtttagccttgaatcagctcagcaagagacatcgaaaggtgaccaccgagctcctggtctactatccccctccatcagctccccagtgctgggcacagatggcatcaggatcgttcatgttgcagcttacctgtcgctgggatgggggataccctgaccctgacttcctgtggatagaagagccaggaggtgtaatcgtggggaagtcaaagctgggggtggaaatgctgagcgagtcccagctgtcggatggcaagaagttcaagtgtgttacaagccacatagttgggccagagtcgggcgccagctgcatggtgcagatcaggggtccctcccttctctctgagcccatgaagacttgcttcactgggggcaatgtgacgcttacatgccaggtgtctggggcctacccccctgccaagatcctgtggctgaggaaccttacccagcccgaggtgatcatccagcctagcagccgccatctcattacccaggatggccagaactccaccctcactatccacaactgctcccaggacctggatgagggctactacatctgccgagctgacagccctgtaggggtgagggagatggaaatctggctgagtgtgaaagaacctttaaatatcggggggattgtgggaaccattgtgagcctccttctgctgggactggccattatctcagggcttctgttgcattatagccctgtgttctgctggaaagtaggaaacacttccaggggacaaaacatggatgatgtcatggttttggtggattcagaagaggaagaggaggaggaggaggaggaggaggaagatgctgcagtaggggaacaggagggagcacgtgagagagaggagttgccaaaagaaatacctaagcaggaccacattcacagagtgaccgccttggtgaatgggaacatagaacagatgggaaatggattccaggatcttcaagatgacagcagtgaggagcaaagtgacattgttcaagaagaagacaggccagtctgaagaagaggatggtccatggttgtcttgctctgaaagcttggagagctacattgaagacgagctcttcattcagctttgactccacctgcacccctggcgggggcttgcactaacaatgtttgggtctcagcaaaaaacaaaaccaagcacacacatctttccttccatgtattgaaaaacattggtttgatttgctctaagttttcccaatgatgtttaaaagctttgagaaggaaagctgctttggtgtctgaggtgccacttctgctgtgaatcctggctttatccaggttgatctactgtgatagatgctgatttagagggaacagaggtcagggaagcactgggtcttggtgccttttgccgcttttttttttttttttttttttttttgagacggagtctccctctattgcccaggctggagtgcaatggcacgatcttggctcaccacaacttctgcctcccaggttcaagcgattctcctgcctcagccacggcaccttgcaaatatcagctccttggaacaggtgaagttccaggtaccaatgccaatcagaggaaggcagtttggttcaggctttggagttagaaacacctgaagttgaatctgggctctgttgcttccttctttcatgggctagagcacgactctttacctctctcttggcctcaatttcctcacctgtaaaatagatgaggaagctgctcacttattattgtctcgttctgaaagcttggaaagctacatcgaagatgagctcttcattcagctttgatttgacctgtacccctggtgggggattgcactggcaacatttgggtctcagcaaaaaaaccaagcacacacatctttccttccatttattgaaaaacatctttgtaagatccattcattgaaaaacataatccatttattgaaaaatatctttgtaagatcacctgctaaatatgaaaatctgacttgaatttgtactctttaaagttgcgtatctgctctagtgggcaggacctagggcttaaaggggaacttcctttctccatttctaagaactgggactctaaaatgagaagctggttgtctgaagtaaccctgcaggtgtggttggggaaggtctgttttcttggatgaaggaactaaactaagcatatcagagcactgtcttaaccagttttatttccctggagatagaattcttttaaaaagagttagggagctggtaataggaagtgcctttcattataactacattttgcagagcttcatatttatatacaagcctcctaggtgatacactgttagcttgcagactttcctatgcttcatttctcctgttgctttcaaagaaggcaggagacacgtttaataacggagtatctggtgataagaattgcttgggcaaaccagctcatctggactctttctcagtcttggaagtgggaagaggaaaacttgtttccttcctgcttcttaaggatattctgagggtacactgatcaataacactaaatttggaatgaaaataccatgtgatgagtttagcctgctgatgcttccagtagatccttgtatagtttcaagatttaagttttccgatttcatataaatttcttaaagtcgaggaccttataagggtgcaatggatgtttgctaaatatgaaaaactgacttgaatttgcactctttaatgttgcgtatctgctctagtgggcaggacctagggcttaaaggggaacttcctttctccaattctaggaactgggactctaaaatgagaagctggttgtctgaagtaaccctgcaggtgtggttggggaaggtctgttttcttggatgaagggattaaactgagcaaatcactagaagtatgccctgtcccctgctcagaacactggggagctcaagagtgggctgcaatgtgcacccctcaggaatagctgtgaattgcaggtctactggctttttgctttttgtcttttgctgcaaggtaccccacgtacttaaccattctcaacagtgtaaatcagtgtcattttagaatgagatactcagcttgcttctaaagtcactgaattactgagtgagtctctcctttagagtcttcggcaaccaaattccagaattgaagagtctactactcagaggcaacaagattaaaaaaagaaaacacaaaaactgttgaggtgaaaaaaaaaaaaaaccctagctaggaacacagagaatgttttgtaggatcactgggatattttccacaacttcctcttctctagcacacacatctgttgataggaaatatttgagggtttttccactaccaaatgggagcttcatggtcctggtgtcaaacactataaacctttgaccagctgagctgtgactgctgtcacatatctgagtcctgtgtgcacagtaatatcctgggtcaggtaaaatccaggtcttcaagttttaaggattttttgaagaattcgggcttctttaagacgatccatgcccaaatccacaagcttgttgacagtggattacagtttgtgtggcaaagtccaagttgttacactgtgctttaaaaaaaatcttatctgcatgtattgttaacttagagaccatgagatctatttatcaggaccaggaagatacacacttcaggtccattgcaactgacttttttcttgtttttctcaaaccctggtggagcctgggaagggggcctccacaattctgtggctttgatattagccccaattctcacaagcacatacaagccccataattgccgcaggaaaacacaagatggaaaattgcaataacccatgcactgagacttagaaaatcatccttactaggcaaaatgtattatgatgcaataagtgccaactgatatttctcacgttgggactggccaggaactgctgcaaagaaaaataagcagctccttctccattatttacattttaagatgtggtggggggaggttgggagaaattagttctgaggttatcatatgccttttttaaaagataatggaataaagctatttttaagtaa

SEQ ID NO: 3 - DNA, 2216 bp, Homo sapiens
  mRNA (1)..(2216): NM_152687
aaaagaacaaaagagagagcttgacacaccaatcttgaggtgttgtgccattctctgggaacatacagatgaaatgacctgaaccctgcccaccatttaaccaaaaaaggaagtggaatggaataaaatcccccaatacagtacaattatacattaatggctgtaatgtgaagagcatcacacacgaagagagccatcttccagaaataagtttatacactctctcctctaattgcatcaggactttaccagataatgttcttccagatctgaaaaggaaaatgcttaaaagagcttccaaactcatttttgaataatactaggctacaaagaattacactgtgaattcattaagggtaacaccaaaccactaaacagcactgtttgtacagaaatgtcgaaaagctgtggaaataatttagcggccatttctgtaggaatttcgcttcttttactcttagtggtttgtggaattgggtgtgtttggcactggaaacaccgtgttgccacacgatttaccttaccgaggtttttacaaaggagaagcagcaggagaaaagtctgtactaaaacattcttgggcccccgcatcattggcttaaggcatgaaatctcagttgaaacccaagaccacaaatctgctgtcaggggaaataacacacacgacaactatgaaaatgtggaagcaggtcctcccaaagctaaaggaaaaaccgataaggaactatatgaaaacacagggcagtctaatttcgaggagcatatctatggaaatgagacatcttctgactattataacttccagaagcctcgtccttctgaagttcctcaagatgaagatatatacattcttccagattcatattagcttttcaaaatattgacttttgttattgggtgataaatattcactgtaatttttcaacagcaaagacaaggaatcaaactaaatgttgatcaactgtagactggataaagaaaatgtggtacacatacaccatagaatattatgcagccgtaaaaaaagaacaaaactaacatgggaacagaaaatcaaataccacatattctcacttaaaagtgggagctaaataataagaacacatggagagaaggagaggaacaacagacactggggcctacttgagggaggacagtggaaggagggagaggttcagggaaaaaaaaaatatcaggtactatgcttagtacacacatgatgaaataatctgtacaccaaacccccaagtcacaagtgttcctacataacaaacctgaacatgtacccctgaacataaaattataattaaaatattaaaaataattcactgtgatttttatggtactgatgccattcttaatcaagttctgataagtggatggtctctgcctatctccacctttctgaatcctatgtgtatcactgtggattaattctagatatcttctccaccctccttgcaccagactaaatctgtattatgtgatattgattcttccttctaaatattacccgttatctctttcctttatttctaccattatctttatctggctcagaattattgtcataggctcctaactgttcctcctgcttctagtttctacccactcaatcaattaccgatggtgttgccagatttatcttcagaaaatattcctaacagccacattatttctttcacttaaaatgttttaatgccccctctttgcaaaagacataatacccataatttgaactccaaaatttatggttttccacaattggttccaattcacttttccagtgacttctcttactatctctcatttctttgccttcagcagaatcatcttaaaacctgccaaacttatccttccttcacagctttgcttttctgcctcttctctcaagcctgcttcagatcataagttcttccacacatctcctgaatcactccaaacccgcatttacctttttattttctgatataagctttgatgcctcttcaattcttaggacatttaaacatatgaatgttgccacagcattttattacctagcttcatatgaaaatgtcttaaattcccacctaaatgaaaagaaactgcccaaatgcctagaacatcacataaggcactaaatgcctcatgttttactgacgggaattgaattgtacattttgctgagtagttttgagaaaaaaatctaataaattcatctgttattcatccat

SEQ ID NO: 4 - DNA, 2008 bp, Homo Sapiens
  mRNA (1)..(2008): XM_379324
gcgagactccatctgaataaacaaaaaaaaaccccaaaaaacaaaaaaacaatgtcatggtattagttaaggcaagtgatattcgctaccataacaaaaatctctaaaaatcttatggcttaatataattagaatgtactatttgccccggagaattcagacagatgtttgtttctgctctagtcactttccatatagtcattccaagtccagtcttcttccatcctgtggctccgacttcttccaagaccttaaaatcctttctccttccagcagatgcagaaggagagaatgtgtggaggattatgtagaaggatttcacgagcctggcctggagaggacacatcacttccaactccattccattaccagagcttagtctatttccagaagattataagcttcatgggggtagatgctttacttcattcatcactgcatctccaacgcctagcacagagtcctgaattacaggctcaataactatttactgaatgtgaaaacagagctgcttgaagatatatgaggctaattgagcttgtgcatttgttggctattgctgctacagtggatgctgttgttcagtattcagatatcaagaaacaactagccaaagtaaggaggaacgaggggcccatgaagctcagtagagcttgtgataatatttgcaagagaagactacctgccaaactaacttcagaaaaggtgtttctggttgaaatcgtcgtgcccaacattccaccaggttcctcacatgttgaatacatcaaagcctactccacccatctggaatggacaagcagaaatgactcactgtgttttccaattgcactcaagacctgtctccatatgccctcctcctctgcttgagcagaaggtgaaagaggttgtgttgactgtggaatttggccattgaatcttgaagacaaatgctctttgacttcactacaacatcagagggcacttggccaaggaagactagttagaaccagcctttttgcaaacacagctgaggtttttatcctgtcttttcttcatcacaaaagtagcatggagaataatggacaatagagttcaagtggagcaagaactgtccagaatttggggaagaagtcacatatcagtcctggccatgataactggggcacatgtactatatccaagttgatatccttaatttggggaaagttttcttgcctttcccacagcctcccaagccattgacaattcccacattcaaaacattctcattcacatttctgattcactttgggctacaggaacaacagatgttgagtacttctagggagcaaatccattatagatggaggtaggtcttgcaaacagagaggtagagagctttctcgagacctttggaccaagcagataacatcacaaggtgtaccttatgctaagtgcctgatctctaagaccctccaccagcgtcagagagacagcatgctcacaacatgacagctggtcatacaggtgaagaagaacatgaagaccagcccacccctgaaaatgacagatcaaactcagcttcacaatgtctcttattccatcgctaatggtgtttaaaaacacactttgtcttaaggaacaaggggaactgaccacgtgttcatgacggttcatttcttaccctctgctttggcttgaccaaactttagtcaggtatctctcctccacaaagattcctagactttggctgtcccccaagtttaagcaagcactgaaacactaaggagcagaacatctgttaacagctcatcctgaaaatcaactgaccataggaaaaaacactccctattgagatatcctggttttgccacctgctcgcccacactctatgcccttctcctccacaaaggtctggctggttcttctccctccatacaaaggaaaagcttatttctgtttgattttgagacatttgcagatatctgaggttggcatgttcttcctattgcaatagtctttttgagtaaagtctctctttatctaa

SEQ ID NO: 5 - DNA, 1200 bp, Homo sapiens
  mRNA (1)..(1200): NM_020350
ctgcgctggggttggagtggccgcaacgggcggggcggggcggggccgggcaagtttgttccccgagttcggagcctaggagccccccgcggctgcggcgcaggtgccctcggcctgagtcgggatggagctgcctgctgtgaacctgaaggtgattctcctaggtcactggctgctgacaacctggggctgcattgtattctcaggctcctatgcctgggccaacttcaccatcctggccttgggcgtgtgggctgtggctcagcgggactccatcgacgccataagcatgtttctgggtggcttgctggccaccatcttcctggacatcgtgcacatcagcatcttctacccgcgggtcagcctcacggacacgggccgctttggcgtgggcatggccatcctcagcttgctgctcaagccgctctcctgctgcttcgtctaccacatgtaccgggagcgcgggggtgagctcctggtccacactggtttccttgggtcttctcaggaccgtagtgcctaccagacgattgactcagcagaggcgcccgcagatccctttgcagtcccagagggcaggagtcaagatgcccgagggtactgaagccagccacgctgcgcccggccctgccccgggccttcctcgtgcctgggaggtcgttctagggatgctcctgacctccgtctcttggacctaagatggaatgtgtccccagctcagggattgcctgaaccaagaggccaggagcccccatgggccgcccagtaccatgcacactcctgtcccgaactccctgaggcctcccctcccttcagggcacccactggttcccaggctggaaccagggtctctctttacctcctaccccatggtggcaccacagaggccctcagccgagtcctgcctgagtgttgcaagctcaggcctttaaggactgctgatgccccctcaggcctcccccaagtttgctgggctttggtggaagccctgagagcttcaggtcctgctcagcccgaggagcagtctggcatgggagtgaggccccgtccttctcactgcctggtcacatggtgcctagggatgcagggctggaggccagaggtgtcagcaacactgtgtcccaccacaacctccagcctcccttttcagagcacagcattaaagtttggggaattctgtagaaaaaaaaaaaaaaaaaa

SEQ ID NO: 6 - DNA, 3636 bp, Homo sapiens
  mRNA (1)..(3636): NM_024735
tggagcgtgcgcacaggcggcagcagtggccgtcactgggcggcatggcggtgtgtgctcgcctttgcggcgtgggcccgtcgcgcggatgtcggcgccgccagcagcgccggggcccggccgagacggcggcggccgacagcgagccggacacagaccccgaggaggagcgcatcgaggctagcgccggggtcgggggcggcttgtgcgcgggcccctcgccgccgcccccgcgctgctcgctgctggagctgccgcccgagctgctggtggagatcttcgcgtcgctgccgggcacggacctacccagcttggcccaggtctgcacgaagttccggcgcatcctccacaccgacaccatctggaggaggcgttgccgtgaggagtatggtgtttgcgaaaacttgcggaagctggagatcacaggcgtgtcttgtcgggacgtctatgcgaagctgcttcaccgatatagacacattttgggattgtggcagccagatatcgggccatacggaggactgctgaacgtggtggtggacggcctgttcatcatcgggtggatgtacctgcctccccatgacccccacgtcgatgaccctatgagattcaagcctctgttcaggatccacctgatggagaggaaggctgccacagtggagtgcatgtacggccacaaagggccccaccacggccacatccagattgtgaagaaggatgagttctccaccaagtgcaaccagacggaccaccacaggatgtccggcgggaggcaggaggagtttcggacgtggctgagggaggaatgggggcgcacgctggaggacatcttccacgagcacatgcaggagctcatcctgatgaagttcatctacaccagtcagtacgacaactgcctgacctaccgccgcatctacctgccgcccagccgccccgacgacctcatcaagcctggcctcttcaaaggtacctatggcagccacggcctggagattgtgatgctcagcttccacggccggcgtgccaggggcaccaagatcacgggcgaccccaacatccccgctgggcagcagacagtggagatcgacctgaggcatcggatccagctgcccgacctcgagaaccagcgcaacttcaatgagctctcccgcatcgtcctggaggtgcgcgagagggtgcgccaggagcagcaggaaggcgggcacgaggcgggcgagggtcgtggccggcagggcccccgggagtcccagccaagccctgcccagcccagggcagaggcgcccagcaagggcccagatgggacacctggtgaggatggtggcgagcctggggatgccgtagctgcggccgagcagcctgcccagtgtgggcaggggcagccgttcgtgctgcccgtgggcgtgagctccaggaatgaggactacccccgaacctgcaggatgtgtttttatggcacaggcctcatcgcgggccacggcttcaccagccctgaacgcacccccggggtcttcatcctcttcgatgaggaccgcttcgggttcgtctggctggagctgaaatccttcagcctgtacagccgggtccaggccaccttccggaacgcagatgcgccgtccccacaggccttcgatgagatgctcaagaacattcagtccctcacctcctgaccggccacatccttgccgccacatcccgggtggctctggggctctgaactctgacctgtgaatagaagcagcatgcactttggaaatccggccttttgaccagaacgcacacctcgtcggggggcccagtccagccaccccccagcactttatgtagagagtgtgacatagacctgcatatttgtcagtgccatgatggaagaagctgagcatgtcttaccaaaaacagagagaacgagcctgaatacagcagatgtaggggacagccgtgggaccgcgtgagaattgaagcggtggggttcccgcaccctgggctggctggtggttttctcgggaagcaggaccctcctgactggtgctcttcctgtgagcggatagagtgatagactgggtcgtgtgtgagacgcatgtgctccaccccactccttttgggggaagccaggcaacagtggcctctgggagggggtcaggaagaggcgaacagctcaggcagcgcaggtgtgatgggcacagtacgcagagcaagctcgggaagttggtaggatctcaggcttggggccgggactctggagtgaatccccatttctctaccggcttgcttggagtttggacagaagcatttcacctctgatctcagcttccccacctgtggagtgggtttagtgacctgagtcactagggaatgtcacctgaatgcacagcccagcccatgcacctgccccagcccctccagctttggagccaaggccatcgttccagccacttgactgtcctcgacggcctgttccagacagggcgtttgttttgtccatgccttcctccctgcacgcacacggcgtcaaaaccaagctgccggccactgtctccagaacgcaaggctccaggcccgtgtgtctgaagcagtgagtggtccacacaggtgccaggagtgcccatatgagatgacgaggaaacccctttgcaggtgaggggacagctttctagaaaagccacacctgcatctggggacacactttggaaagtgggaccctccagcctggagaccccatggactgatgcctccactgctgtgtgccccatgttgtgttaacacctgcgtgtggggaccccatctgaggtcttggctgaggttggcatctcctgaagaacagagagcacggtgtccagagctggcccttcccccagcccacagccagctccgtgcccgagtgggcgtccccagcgagccttccctctctgccgcttgtccttgtgtctgggctgctccaagtccttgtgctgggcaccctggacacgtcctgctggtgagggacctcgggaaggtgacagtctgtgtgccttggtgtggagaccaacctgaggatgtcctgggaaatgttttcctgatgaatttctccttgactggcctttaaagaacataagaattcccattgcccagcctcagtgcatttggcaaatgcttactttgcttcccagagtcagagaattggcaaaggttcctaaatggtaatctggccggcctgggagaaagactcacgagaaaagccagtggagaaagcgcccttccagggcggcagcagcgggagccacgcagaccccgaggcgcacctgctggctcttgtgtgtggccccagtttctagcggcttttgcagcattagcctacaagctttgtcactccctgccctctgtggtggtcactgtttttctctcttgccaaatgaggcagtctctgagtgacggtgactgtggccttgaagcctggaggactgttgggcatgtagactggcaccttgaagattcaccattgtttaaataaaatcaagcaaatgcttttttaccaagagcccgagcctcgctctaagggacgcagtcctagaggcgtgccctttggggcttgaagagcacactgtgggacgcacgtgcttctgattaaaggaatctcagatctcaaaaaaaaaaaaaaaaaaaaaa

SEQ ID NO: 7 - DNA, 4866 bp, Homo sapiens
  Unsure (1)..(4866): XM_375375
ggcggccgtgcgggagccatggcggcctcggaggcggcggcggcggcggggtccgcggctctggcggcgggtgcccgggccgtcccggcggccacgacaggagccgccgccgccgcctcgggcccgtgggtgcccccgggaccccgactgaggggcagccggccgcggcccgcgggggcgacgcagcagcccgctgtccccgcgccgccggcgggggagctgatccagccgtcggtgagcgagctgtcccgggccgtgcggaccaacatcctgtgcaccgtgcgcggctgcggcaagatcctgcccaacagccccgcgctcaacatgcacctagtcaagagccaccgcctgcaggatggcatagtcaatccaacaataagaaaagatttgaaaactggaccgaaattctactgctgtccaattgaaggctgccccagaggccctgagagaccgttttctcagttttctctcgtaaaacagcactttatgaaaatgcatgctgagaagaagcacaaatgtagtaagtgcagcaattcgtacggtacagaatgggacctgaaaagacatgcagaggactgtggcaagaccttccggtgcacatgcggctgtccctacgccagtagaacagcactgcagtctcacatctaccgaactgggcacgagatacctgcagaacacagggacccacctagtaagaaaaggaaaatggaaaactgtgcacaaaaccagaagttatccaacaagaccattgaatcattgaacaaccaaccaatccctagaccagacactcaagaactagaagcttcagaaataaagctagaaccatcttttgaagactcttgtggctctaacactgacaagcagactcttacaacaccaccgagatatcctcagaagttgcttttaccaaagcccaaagtggctttggttaaactacccgtgatgcagttttctgtcatgcctgtctttgtgcctacagccgactcctcagcccagcctgtggtgttaggtgttgatcagggctctgccacaggggctgtgcacttaatgcccttgtcagtaggaaccctgatcctcggcctagattcagaggcttgctctcttaaggagagcctacctcttttcaaaattgctaatcctattgctggtgagccaataagtactggtgttcaagtgaactttggtaaaagtccatctaatcctttacaagaactagggaacacgtgtcaaaagaatagcatttcttcaatcaacgtgcagacagatctgtcttatgcctcacaaaactttataccttctgcacagtgggccactgctgattcctctgtgtcgtcttgttctcaaactgatttgtcgtttgattctcaagtgtctcttcccattagtgttcacactcagacatttttgcccagctctaaggtaacttcatctatagctgctcagactgatgcatttatggacacctgtttccagtcaggtggggtctccagagaaactcaaaccagtgggatagaaagtccaacggatgaccatgtacagatggaccaagctggaatgtgcggagacatttttgagagtgttcattcatcatataatgttgctacaggtaacattataagcaacagtttagtagcagagacagtaactcatagtttgttacctcagaatgagcctaagactttaaatcaagatattgagaaatctgcaccaattataaatttcagtgcacagaatagtatgcttccttcacagaacatgacagataatcagacccaaaccatagatttattaagtgatttggaaaacatcttgtcaagtaatctgcctgctcagacattggatcatcgtagtcttttgtctgacacaaatcctggacctgacacccagctcccatctggcccagcccagaaccccggaatcgattttgatatcgaagagttcttttcggcctcaaatatccagactcaaactgaagagagtgaacttagcaccatgaccaccgagccagtcttggagtcactggacatagagactcaaacggacttcttactcgcagatacctctgctcagtcctatgggtgtaggggaaattctaacttcttaggccttgagatgtttgacacacagacacagacagacttaaactttttcttagacagtagccctcatctgcctctgggaagtattctgaaacactccagcttttccgtgagtactgattcatctgacacagagacccaaactgaaggagtctccactgctaaaaatatacctgctctagaaagcaaagttcagttgaacagtacagaaacacagaccatgagttctgggtttgaaaccctggggagcttgttcttcaccagcaacgaaactcagacagcaatggatgactttcttctggctgatctggcctggaacacgatggagtctcagttcagctctgtagaaacccagacttctgcggaaccacacacagtctccaacttctaaaactaacggtggagtccatgtgtgaaatggcatctaccatttcctctggattaaaactacggactggggacaacagtattaattcgattgaatgtggctgatgatgcagttgcttagcttctttgtgtttctttgccttttgtacttgtaaacagaaatttgcgtataaatgtgagtgtattataaagtttgagatgttgatctaaattgtttttgtgttgcctacatttgccttttcacagctagtcttttcatgttaaaaaaaaaaatgtatttcatatctataaaacctatatagccatttagctgaagcccagcttaccaggttcaagggtacaaacttctcaaatcttcaaaacattttagtcaaagtgtaatatacttaaactgcacctaaaatatctttggcactgcttgttagaaattcctgattcctgttactaatcactaaagaaaccggatgctgccaccgtaggatttaagcagtagtgcttccatgctcttaagactcctgctgcctggaccttcgtcagctttgacacctcttttctgatttaaagacaccaaggaaaactacaactgtctttagctttgaagcagttttcatgtaatcattgccacctcttcgctacatgaactactattgataccagcatacaagtgtacagcactttacacacaagaggtttattgatgtaaaattatcggctagggaagcagcagcgggccaggtgtggtggcttacccctgtaatcccagcactttgggaggccaaagcaggacgatcacttgagcccaggagttcaacaccagcttgggcaacgtaagaagaccgtgtctctggaatttttttttttttttaattagccaggcacagtggcatgcgcctgtgatcccagctacttggaaggctgaggtgagaggatcactcgaggagattggggctgccatgagccatggtcttggcactgtactccaacctgggtaacagggcaagaccctatctcaaaaaaaaaaaaagtcgccagcaacaagcacgtagtgtagtgttcctgctaaatgagcgtaggttatccaaaccttgggaacagggagttatggaaacatgcctatgacttcatcttggggtgtgtcctatgaagatcctttctggtctccacagtaggccagagttgggggctctggagctgtttccccaagtgcatccacaagctggatctgagttttgtcactctaaaattaaacaagaaaaaaagtgggaaaagggcatcccccattaggtttcaatactttgcacttctactaagcttgatagggcaggagtgcaatctacaattattttaaagtgaatttccttccattcaccattctttatcttttctttgaataagaaaaagtatctagcaaggatattacttgtgccttgaggctagcaattataggatagattcatctaaaatatggtattctgcattttggttttttttcttaagtgaataataccagtcttcaaagaaaacaaggtgaagacctattgcttcaataatcaagaatgctttgtgtgttttgaggtaggagcatgatcaagtatgctttggggattttctgtatttaggagatcctggattcttaattgttggctaagttccagtcaagtaggaatcagtgcagcctgtaagttctccacattgacacacacacacacacacacacacacacacgacatgctcctttctgtggcacatgcctgtattactgaaagctaaatcctcaaaacctagtaaggggactaatgattcattaaagtaaattgatggttttgctactaattcctatcccatacatttgacacaaaagaagtgttggtaatggataaataacatatcccgggcagatgagctcaacctagtaggtaagagtttggtttggtcacagttgcctatgagtgtgggtttcaaaagaaacataaagccttaacttagaatttcattatgttttagaatcatcactgccttaatattcaagcatctatttaagtcctaataaaggagaaatgcatgtttatggcttttttgtaaatataaatgcagtgatctatggcttaaaaaatttgtttctgtgacaatgtttgtaaatctagccaatagagtcatttacagaagaaaaatgagcatgtaataatacaagaactgtttccccctcaaaacctgaacctgaattatttgtaaaaactgaaatttaatgattaaagagaagccagaattgtacccttttttgtgaattcttgaacgtactcataaatatgacttattgtattgccttaagttttcactcattgtcttttgaaagccatatgataaaatgattttatttaat

SEQ ID NO: 8 - DNA, 3816 bp, Homo sapiens
  mRNA (1)..(3816): NM_002268
gcagcgccggggccctcagatcgaggctgcctccccctctcagccggcagcacattccgtcgccttgccgccgcccggccccacagttgtctttgccggagctgcagtcgcgctggggttagggcgggggggcgggcggggagaggagaaggccgcggcggaggcaggtgagaggaggaaggaagcggcggcgcggcgggcacagcagctcaggcggcggcccggttcctgggagtgtcggtgcggcgcgttggttggggcggatcccgcggggcccgggcgggggaaggagtcaccggcccggccatggcggacaacgagaaactggacaaccaacggctcaagaatttcaagaacaaaggccgcgacttggagactatgagaagacaacgaaatgaagttgtagttgaattaaggaagaataaaagagatgaacatctcttaaagagaaggaatgtaccacatgaagatatctgtgaagactctgatatagatggtgattatagagtgcaaaatacctctctagaagctattgttcaaaatgcttcaagtgataaccaaggaattcaattaagtgcagttcaagctgctaggaagcttttgtccagtgatcgaaatccaccaattgatgacttaataaaatctggaatattgcccattttagtccattgtcttgaaagagatgacaatccttctttacagtttgaagctgcatgggctttgacaaacattgcatctggaacttctgaacaaactcaagcagtagttcagtccaatgctgtgccacttttcctgaggcttctccattcaccccatcagaatgtctgtgagcaagcagtgtgggcattgggaaatatcataggtgatgggccccagtgtagagattatgtcataagtcttggagttgtgaaacctttactttccttcataagtccatctattcctataacattcttaagaaatgttacttgggttatggtcaacttatgtcgccacaaagacccaccaccaccaatggaaaccattcaggagattcttccagccctttgtgttttaattcatcacacagatgtaaatatactggtagacacagtctgggccctctcttaccttactgatgctggcaatgaacaaatacagatggtaatagactctggaatagttcctcatttggttcctctgctcagccaccaggaagttaaagttcagactgctgcacttagagctgtgggcaacattgttactggaactgatgagcaaacacaagtagttttgaactgtgatgctctttcacacttcccagcactcctgacacatcccaaagagaaaattaataaagaagcagtgtggttcctctccaacatcactgcaggaaatcagcagcaggtacaggcagtaattgatgccaatcttgtaccaatgataatacaccttttggataagggggattttggcactcaaaaagaagctgcttgggccataagtaacttaacaattagtggaaggaaagatcaagtggcttaccttatccaacaaaatgttatcccacctttttgcaacttgctgactgtaaaagatgcacaagttgtgcaagtagtactcgatggactaagtaatatattaaaaatggctgaagatgaggcagaaaccataggcaatcttatagaagaatgtggagggctggagaaaattgaacaacttcaaaatcatgaaaatgaagacatctacaaattggcctatgagatcattgatcagttcttctcttcagatgatattgatgaagaccctagccttgttccagaggcaattcaaggcggaacatttggtttcaattcatctgccaatgtaccaacagaagggttccagttttagaaagatgttgtggaagttaggtacaatgcagcactgagatatatatatatatatgtgtgtgtgtatatatatatatatatacatatatataaaaaggtttgatccatcaagcttggctcatgggatctgctgctgcattaaatcgggaaagaaaatgtgaagatttcatttggaatcacagaaaatgcccaaatgaggtcaagatggcgagtgggtgcgagtgagaatgagtggcaaaatgtaatgaaaactttacatgaatgcttatttaggttgttcaaagtaaaaagggctacaggtcacagatcgtcagtgcctgagaaagaacattgacttactctatatcaattgaggggaaagtgcagtaccgtcatcttcaagccttgtaagcataaaagagaataggctgcccatataagtcaaaggaaaatgagcccaggccttgctatgaagcagtgtgtgaatggacaatgttgaatgaatgtctggctcagtgatggagagccaggttcatctttgaaatctagggctcttcactcatgaagcagactcctagtcctggagtgactgtgtacgagagcgtggttgtggtgctgtatgtgaacgcatgcaagcttgattcaccttcagggggctgataacctagtaaatcatcaaaatgagatcataagtgttaatgtacactggacatgaaaacaaagactggtttagcagcagacattggtttactctgcagcctgtgttttctgtttccccctttcccacctccttccccccacccaatccttttttttttcttttttgcttttcttttctttttttttagtttttatttactttacctagtatgcctttttttagttgcttctcaagtcagaaaacttttcaggaaggtttccctgtgcatttgcaccagatgaatgtttgatgctatgaaaagctttccatatcatcaaaactaatttgtgtagatttttgcatgaaaaaaatcataaatttccctcaaaatagactgtgttgcagtacacaagttgccataatagtataaaacagtaaaatgtgcttaaaaggccatccttttcattttcagagataacataaagatctttgcatgaggtaaatctacagcatagttcatttttagattttgttgagtcctgtaaagaagaagaagaaaaaagtttcagttgtggtagaataccgtgctgtgtttaaatgttacttgttttcaaactttgttttctatgaaaatgatatggaaacttctaaaatggaatttggtgcatatgtactgctgaataaagaccgatgaagaggtttgagtagatgtacaaatcaagtaatggtttgaacacctttaataatatgcctaatctgttcaattgttttagaatctttttatcttagatgtaggcagccatgaacaatctattttgagccactttagggagaaaactttgtatttttaaaacttgcataaaagttatgcaagtggtttttataaattggaataatacctcagttttgaggttatgcacactaaattaaatgtgacataaattaatttgtacaaaaagaactctttataaggtggctcattgtaggaaatcctgtgccttcccctttgagcacaagtgttgcatgaacaacagtttgctataagaaacataccagattagccaccattagcatctatatctactttgtgtttaaaaatcaactggtaattctgaaacactgtagaatggataaaaattattttgtgatcataactctttgttgaactagagtatttttgcagcattccttgtcatcagaaacatggttaaagtttaaaactagaagcagcagaaaactagcttgtaaaatttatccaagtagagtgcaggctaggctgtcttggggaaataaacattaaaacttaaagcaaaaaaaaaaaaaaaa

SEQ ID NO: 9 - DNA, 600 bp, Homo sapiens
  mRNA (1)..(600): NM_018069
ggcaatgcgcatgcccagcgccgtatcgcgcacgcgctctctgcggctttccttgacctctgacccgccgaccacgcttgatccccggccgcggggccaggaagtcggagtttgagccccggaggcagagcggctgccatggccaagtacctggcccagatcattgtgatgggcgtgcaggtggtgggcagggcctttgcacgggccttgcggcaggagtttgcagccagccgggccgcagctgatgcccgaggacgcgctggacaccggtctgcagccgcttccaacctctccggcctcagcctccaggaggcacagcagattctcaacgtgtccaagctgagccctgaggaggtccagaagaactatgaacacttatttaaggtgaatgataaatccgtgggtggctccttctacctgcagtcaaaggtggtccgcgcaaaggagcgcctggatgaggaactcaaaatccaggcccaggaggacagagaaaaagggcagatgccccatacgtgactgctcggctccccccgcccaccccgccgcctctaatttatagcttggtaataaatttcttttctgcaaaaaaaaaaaaaaaaa

SEQ ID NO: 10 - DNA, 5533 bp, Homo sapiens
  mRNA (1)..(5533): NM_004991
gattgccatctgacaagatctccaaatcaaagtgataaatcgctccaaactttttttggcggcgctgagatgttggaggggcgtctagcgcgcatgtgcgaaggtgtccaaactgacaatgctggagagatagcgagtgtggattgagagaaagggagagagggagggagagagagtgaaagaagaaaatacagagagtgagtgtgtggaagagagagagaaacaggagagaaacaggagggagggagagagagagagagagagagagagagagagagagagagagagagagagagagagacaggagagagagggagggagcgagagggagagcaaaagaaggaaaggatccaagaaaaaaaagccccaaccacacaccagcggctgcaggactgggcacagcatgagatccaaaggcagggcaaggaaactggccacaaataatgagtgtgtatatggcaactaccctgaaatacctttggaagaaatgccagatgcagatggagtagccagcactccctccctcaatattcaagagccatgctctcctgccacatccagtgaagcattcactccaaaggagggttctccttacaaagcccccatctacatccctgatgatatccccattcctgctgagtttgaacttcgagagtcaaatatgcctggggcaggactaggaatatggaccaaaaggaagatcgaagtaggtgaaaagtttgggccttatgtgggagagcagaggtcaaacctgaaagaccccagttatggatgggagatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtattttt

SEQ ID NO: 11 - DNA, 6471 bp, Homo sapiens
  mRNA (1)..(6471): NM_022731
gcacaaaccctagtgggtttggttgcacggcggctttggcgcattttcggctggtttgattcatccattttgaagagacgggggagcggggggctcgtctgttccaggagccctgaaccaaagagcagcggagtttgagaagccagcagctcggggttcggcagcagcggtcccatcggctgaagttcggggggggtggggcgccgagcgcgcggggtggggggggtcctggtctttggcttctcgactcggtcctgtttcgacagcgaacatgtcgcggcctgtcagaaataggaaggttgttgattactcacagtttcaggaatctgatgatgcagatgaagattatggaagagattcgggccctcccactaagaaaattcgatcatctccccgagaagctaaaaataagaggcgatctggaaagaattcacaggaagatagtgaggactcagaagacaaagatgtgaagaccaagaaggatgattctcactcagcagaggatagtgaagatgaaaaagaagatcataaaaatgtgcgccaacaacggcaggcggcatctaaagcagcttctaaacagagagagatgctcatggaagatgtgggcagtgaggaagaacaagaagaggaggatgaggcaccattccaggagaaagattccggcagcgatgaagatttcctaatggaagatgatgacgatagtgactatggcagttcgaaaaagaaaaacaaaaagatggttaagaagtccaaacctgaaagaaaagaaaagaaaatgcccaaacccagactaaaggctacagtgacgccaagtccagtgaaaggcaaagggaaagtgggtcgccccacagcttcaaaggcatcaaaggaaaagactccttctcccaaagaagaagatgaggaaccggaaagcccgccagaaaagaaaacatctacaagccccccacccgagaaatctggggatgaagggtctgaagatgaagccccttctggggaggattaaaagtgatgatggtctggggagagattttattaaaaaaaaaaagaaaaaaaaagaaaaaagagggaggaaaaaaaagaacctacttaagatagaacatggttttggctatggcttgactcatgggctttcagtgcttttttccatttgttgaaagtaacatttctctctctctctctttttttttttttttttttaaagcaaaccattgtatgtgtaagtgtttaagttacctttttgtctattggtctctttgccagccctcccctttcccaatgaaagccatgtcaaattaatcactggattgactgcttcatctttttatttttaatgaaaggtgtaccacggttgtaaagcaataagatttgagatgaacactattgaaacttcgctttttgctaaaaaatagcaagttgaatagtaatcaaaaaacatagaaagattttagttcaaaatgattgctcctttctctacctggacttttaaaaaatcaattgtcatctaatatgagtttatttgtctatagacacaagtatcaatgtctaaaaaaaatcatgactttaaacttccaccgatgaggcaggtaggagataaagatgaattctgaactgttactaaaagtactcattttttaccttgtagggagggtgggcaatggggttacctgaccttatttgagggtatgggctttcttttttatttcatcacttgttatctcaaagagactcggagccagtgatccttttatcctgctacagtctttagggagctaaaaaaaaaaaaaaagcaggggctgccaaaactcttgatttcatatttccttctctaaatatatatgtatcctgttttttggataaaattttaccaagaatccaaaaaaaaaaaaaccctagaatttaatcaacaagatcagtctacaggtcacagtggatttcttttcaaactgacaatgtttaggttttaagcaaataaagttccagttaatgtgaaactcagtcacaaagagttgagatttttcctttatgaaatagaattgacattcttttatgctataaatgtgcattcaggtcccattaaccatgctctgcttttatttggggatagaacattttctttttcatatcccgatcttcccatttcttcatagaaatgtgataagaagtacatccctgtgatcctgctgcttcgtagagcaccactgcacaccctaccccgagtgccaaccacctctgctataggacactattttcctggccctattcttcacttacttcccatcctgtccttgactaggaatatgttaaatgctgctcccatacaattcagttagctcttgtctttttatttggtccaacccctgctttactgctcatgctgcttaaagcaggagggactagagaaacaaggcattttaggaggcctgtgtgcagttgaaaaccgacttttacacgccttataaaagcagtcaggagatagatccgtaggtttgatccttcacatctaataccaggcgctaatgggaacaaggtttaaagggtcctggtatgctaataaattgaaaaattagtgaaatttaaacttctgcctttttttcctgccttttaatctagatttgcttcctcaatatcctactttgtggtttactaggaacatgcttactctgatctttttttaaaaaacacacagtggcagagtcatttcactattgcactgtgtgttaaagaatgaataaggagttttcagttacatggccaaaaatacaggacttgaacataaatagcagttggatcattctctttcatgacggttaaattcagaggtgtgaactttgtaatgagggtgttaaagattaatctatttgcctaaatgggtttgttcaggtatccatttttaacaaagaagtttgtgttcatatagtaaaagacctatcagtgtttccaccatgcacttctattttttaggagtttataattttaagtcttacattcctagtaacatttgggcttttcttaggttatgtttcgtgaagatttggggggagggctcttttaaaacttcagcctcagttgtttaacagtctctttaatatattaatctgcactaacatctctgtgatatatgcacatattttagaggtaatcatgtcttctagattacttgtgtgcatttgattgggcttcttgtttagggtcccttttaaaattaattcattagattgaaaaatgtattctatatttctgatagactggacagaaggatctgtgtccccaagtgagacaggctctgaataacctttgttttctccactttttattgatgatttaaaacactctagtcttcccctcaaatcatgcatgcaaataggaggacagtggtggtgactcaactggatacaggtgctcaatagtcaggcttgatagtgatgtcaggacgcattacaagctgtaagccgatactgactggccattggcaccatccttgactaaccttcctctttttctctagtgtgcctatggtgaaatggcaatagcattcactgtcgtattttgcagtgctcaggaagtgggacgttaactttgaaggtgcttgtttgtattagctctgctaggtttacctctacaacgtagatttcagcagctatgctgactgacactacattctagttcttaagattttttttccagatccccccttccccagctagacatacgtagcatactttcatcttattcagtctttctgtaacctgctgctgcttttagtcctcctcacctcagatcggaatcaatggagtgggcccagaggatacattttaattccagtaatggtaggtagatttgtcctgctttctaaaacatctcctcatttcatatttccactccatattgattccataagggaaaattaatgggtgtttcctcctttagggaggtaatgcaaagagtgtggacatcttctaatcttgaggaacagtagttgatttcccttgaaggagcttacatattgactgttttcacaataacctgtttgccccagttcaatcctcattttaatacttaatttggtactggctcaaatagcattttcttacagataacaaatcaagagtgaaatttgaggttatactccagtaaagtttttaacacttgtgaatatggtcagctagactaaacttgactcttttttttaatggcttttttatctgtgaacattcagataagtggattttcaagtactggttggggatgggaatcgtgcttttctttaaacttcagtttacgagatgctttgagagcgttaggcaaaagcagaaataaatatcaggagcaacggggaaagctttataaaagatcatggtggccactgttgcagctttgaagaatgagtgctggcttgaacagttctttgcctgcatcattggtagctgcactgaaaggaaaaaactttcaccttaagaatttgaaaaggaagaaacctgggctctggtcttcatggcatttagactgagatgcttaaacagaacagaagtaatacgcatttcctgccataggatagggaaaatgtaacaagctggttgctcttgaggttagaaaattgtctgtttctctgtggatgaagctggatttacttgaaaatggagagttggcttattgtttgaatattgggacatcaagctatctatagccaagtttcagtcgcaaccagttttccctttgtctggggtaaattcgatacaaaatgattctttttgaatcctgaatccataaattacacttttttttttcaaattcacaaaattcacagtggtgctgactgtgtaataaccactattgggaaacatcccgtaaacctgcctgttgccatgccaatggagtgactgaactggtgacatctgtttgagcatgctttgtgtggctggtagaatgccaccgttgtgcatacactttgtacatcaggggtgaagggagggttttctagattattgggggagggtaaaattgggatttttttgttgttccttttttgatggggtgtgggggtatagtactcagcttatgccctaaaataacatgtataaaaacccctgaagtattgtgtgggtgtgtacgtgtgagtgtgtgtttgtatacatctggcaattaaagctttgtcttctggaacttagtgaattcttttctctttttcctccagaagtatttgttacaagatttgtaaataagagctctacttagtttgtttaccatgaacatgttgcagcaaaccttatgcatctaattcctacaaggttaaagaaaggcttttagacttgccaggttaagcaacagccaagttctcagtaattgtttgccttgatttatcttttagacttcattttgccagctctaaaactcccagtcttccttgattttagtccttaatcttttatgttctgagcaggaagggtaaaagacaggaacctgcttcactgtattaactagtccatgggctgagaccggggcatctcttttcttcatactgcaatgttgctagatacatgatcagacaccagagggttgggcattcttgcaataccttaacagtgctgaaatctgcagcatggtactaaggaagttaaagtttgaatgtaaccactttatttaaaaggtttttttctttaatttaaatgaaatggggttgaagtgaacatgattttgttgaccatgttcgtgaattacagatgcaacatgcattggtagaatcgtgtgatggtcttttgtgatacttaatttttacatatcccagtctctgtatgtatctgcatagacaaagaaaaaacaaactcctgctttgcttttattgaagggtttccaggactgcgtgtctgctcctgagctctgttttaagtatgtgtatcctttgcttgtattttgtattaaaaaaataagaaaaagaagcctttattgttgagcatgttggcattgtcccctttatttttttctctttttgggacatatgaagcaagttattctttttctgtatctttttttcttttgtaaactttttttttgttttgtttaaaaatggctttataaaagggcttttataacccagatgtgtgctctgtgtacttcttgtaataccttaaagcaaacacactaacttacacagctttgttaatcagctcccagttcagcctgactttgtgggaacttatttccctaataaattatttagaaactttaacagtgaccatccctgttgttggtaaaagataaacatgtcttggttcaaaagttaatacctcaggatggaatcaagaagcagtagacttaccagtttgattaattaagggagcactggactgggtgcgtaaggattcttaattagcattttttctcatgtttacatccaaggttatgaagtgttgtggaaagcaaagtttaacaatcatatataataaaagataatgtttatactcacagaaaaaaaaa

SEQ ID NO: 12 - DNA, 984 bp, Homo sapiens
  mRNA (1)..(984): NM_001005284
atgattttcccttctcatgatagtcaggctttcacctccgtggacatggaagtgggaaattgcaccatcctgactgaattcatcttgttgggtttctcagcagattcccagtggcagccgattctatttggagtgtttctgatgctctatttgataaccttgtcaggaaacatgaccttggttatcttaatccgaactgattcccacttgcatacacctatgtactttttcattggcaatctgtcttttttggatttctggtatacctctgtgtatacccccaaaatcctggccagttgtgtctcagaagataagcgcatttccttggctggatgtggggctcagctgtttttttcctgtgttgtagcctacactgaatgctatctcctggcagccatggcatatgaccgccatgcagcaatttgtaacccattgctttattcaggtaccatgtccaccgccctctgtactgggcttgttgctggctcctacataggaggatttttgaatgccatagcccatactgccaatacattccgcctgcatttttgtggtaaaaatatcattgaccactttttctgtgatgcaccaccattggtaaaaatgtcctgtacaaacaccagggtctacgaaaaagtcctgcttggtgtggtgggcttcacagtactctccagcattcttgctatcctgatttcctatgtcaacatcctcctggctatcctgagaatccactcagcttcaggaagacacaaggcattctccacctgtgcttcccacctcatctcagtcatgctcttctatggatcattgttgtttatgtattcaaggcctagttccacctactccctagagagggacaaagtagctgctctgttctacaccgtgatcaacccactgctcaaccctctcatctatagcctgagaaacaaagatatcaaagaggccttcaggaaagcaacacagactatacaaccacaaacatga

SEQ ID NO: 13 - DNA, 3331 bp, Homo sapiens
  mRNA (1)..(3331): NM_017730
attggctgtgagggttgaatgtaagatggcgcccagggagctgtgaggagaaaaccctgtcggtcttggagcgacgacggcagaaccagggtccctggcggtgcggcggggccggcgggtgcagcggaggcggcggcggcggcggcagtgacgtcgccggaatattagaagtcttaagaactcaggacaagcagcagaaatacatgcaacatggtgactggaaccctaaggactctgcaatatgaataattccctagagaacaccatctcctttgaagagtacatccgagtaaaggcacggtctgtcccgcaacacaggatgaaggaatttctggactcactggcctctaaggggccagaagcccttcaggagttccagcagacagccaccactaccatggtgtaccaacagggtgggaactgcatatacacagacagcactgaagtggctgggtctttgcttgaacttgcctgtccagtcaccaccagtgttcagccacaaacccagcaagaacagcagatccaggttcagcagccgcagcaggttcaggtccaggtgcaggtacagcagtctccgcaacaggtctcggctcagctctccccacaactcaccgttcaccagcctactgagcaacccatccaggtccaggtgcagatccaaggccaggcaccacagtcagcagccccctccattcagaccccgtctctgcagagtcccagtccctcgcagctgcaagcagctcagatccaggtgcagcacgtgcaagcagcccagcagatccaggctgcagaaatcccggaggagcacatcccacatcagcaaatccaggctcagctggtggctggccagtctcttgctggtggtcagcagatccaaatccagaccgtgggtgccctttccccaccaccatcccagcagggctcaccccgggaaggggagcggcgggttggcacggccagtgtcctccaaccagtgaagaagcgcaaagtggacatgcccatcactgtgtcctacgccatctcagggcagccggtggccaccgtgctggccattccacagggccagcagcagagttatgtgtctttgaggccagacttactgacagtagacagtgcccacctgtacagtgccactgggaccattactagccctacaggagaaacctggaccatccctgtttattctgcccagccccggggggaccctcagcagcagagcattacccacattgccattccccaggaagcctacaacgcagttcacgtcagtggctcacccacagccctggcagctgttaagctggaggatgacaaggagaagatggtgggcaccacatctgtagtgaaaaactcccatgaagaggtagtgcagacccttgcaaactctctctttccagcacagttcatgaatggcaacatccacattccagtggctgtgcaggctgtggcaggcacgtaccagaatacggctcaaactgtccatatatgggacccccaacagcagccgcagcagcaaactccccaggaacagacaccaccaccacagcagcagcagcagcaactccaagttacttgttcagctcaaactgtccaggttgctgaagttgaaccacagtcacagccacagccttccccagaacttctgcttccaaattctttgaagccagaagaagggcttgaagtatggaaaaactgggcccagaccaagaatgctgaactagagaaggatgctcagaacagattggcacccattgggaggcgccaactgctgcgattccaggaagatctcatctcctctgctgtggcagagttgaattatgggctctgtctaatgacacgggaagctcgaaatggagaaggtgaaccctatgacccagatgtgctctactatattttcctgtgtattcaaaagtatctttttgaaaatggaagggtagatgacattttctccgatctttattatgttcggttcacggagtggctacatgaagttctgaaggatgttcagccccgggtcactccacttggctatgtcttgcccagccacgtgactgaggagatgctatgggagtgcaagcagcttggggctcactccccctccaccttgctgaccaccctcatgttctttaataccaagtacttcctattgaagacagtggaccagcacatgaagctggccttctccaaggtcttgcgacagacaaagaagaacccctctaatcccaaggataaaagcacgagtatccggtacttgaaggcccttggaatacaccagactggccagaaagttacagatgacatgtatgcagaacagacggaaaatccagagaatccattgagatgtcccatcaagctctatgatttctacctcttcaaatgcccccagagtgtgaaaggccggaatgacaccttttacctgacacctgagccagtggtggcccccaacagcccaatctggtactcagtccagcctatcagcagagagcagatgggacaaatgctgacgcggatcctggtgataagagaaattcaggaggccatcgcagtggccaatgcaagcactatgcactgagatgccttggccatggcacaagagaaaccagccaggaaaaaccagacagactttcacactaaagaagaggcctccatttttttttttcttttttttattggtgtagttacgaagcctttcaggctgcttctgtttaaaatataaaagaaaactttgccccctttgcatcttcataaacctgctgcggcagactcctcagccgatggtggctctgggtttccttgagtgtcatatgtcctagaaagttgctggctgactcttttttgtctggggcctggggaaagggcttggactgtgaaaagaaatgtggcccctttccatcttcaagagagatggaattaatgatggatggaccctggagggaatctccccagccgacttccactgggctgacagactttgctgaccacaggggaacgatgttcttttctttcttcatgatcagacataaacttagcatcttaatggaagaaaaatgaggggaacttcaattatgatttattaaagacaatttctattacaccctcctttatgacaagtgacattttagatgtaaaagtaaaaactttaccatgccttttttttttttgttggcctaacattgaggccttaaaacctgaggctcctgtgcctgatggaattcttgtaacatacacttgtgtatcatataaagataccactctgtttctcttatgtattcttactctagttgtttattaagaatgacaagcacgtcttttcaaaaaaaaaaaaaaaaaa

SEQ ID NO: 14 - DNA, 1176 bp, Homo sapiens
  mRNA (1)..(1176): NM_006913
aatagtgattaggaaaccttgaagcctgcccaacgatcgtgggcaggaggtggtttctggtttgttggggcgtgtgtatgtgtatttggggggactgaagggtacgtggggcgaaacaaaaccggccatggcagcagcggaggaggaggacgggggccccgaagggccaaatcgcgagcggggcggggcgggcgcgaccttcgaatgtaatatatgtttggagactgctcgggaagctgtggtcagtgtgtgtggccacctgtactgttggccatgtcttcatcagtggctggagacacggccagaacggcaagagtgtccagtatgtaaagctgggatcagcagagagaaggttgtcccgctttatgggcgagggagccagaagccccaggatcccagattaaaaactccaccccgcccccagggccagagaccagctccggagagcagagggggattccagccatttggtgataccgggggcttccacttctcatttggtgttggtgcttttccctttggctttttcaccaccgtcttcaatgcccatgagcctttccgccggggtacaggtgtggatctgggacagggtcacccagcctccagctggcaggattccctcttcctgtttctcgccatcttcttctttttttggctgctcagtatttgagctatgtctgcttcctgcccacctccagccagagaagaatcagtattgagggtccctgctgacccttccgtactcctggacccccttgacccctctatttctgttggctaaggccagccctggacattgtccaggaaggcctggggaggaggagtgaagtctgtgcatagatgggagagccttctgctcagaggctcactcagtaacgttgtttaattctctgccctggggaaggaggatggattgagagaatgtctttctcctctcctaagtctttgctttccctgatttcttgatttgatcttcaaaggtgggcaaagttccctctgactcttcccccactccccatcttactgatttaatttaatttttcactccccagagtctaatatggattctgactcttaagtgcttccgccccctcactacctcctttaatacaaattcaataaaaaaggtgaaatataaaaaaaaaaaaaaaaaaaaaaaaaaaaaaaaaaaaaaaa

SEQ ID NO: 15 - DNA, 1681 bp, Homo sapiens
  mRNA (1)..(1681): NM_005273
actggcgggcgggatccctccgctctggggaggcagcgctggcggcggggctggggccactgaggaaatccatccgcgccgccgccgccgccgccgccgccgccgccgccgcctccgccgcggaggaagacagcgccgcccgcgcaccgccagcgacctccgccgcagagtcccaccgccacaggcctcgggccagcggccaggagctgcctcccccagcccccgtcccgcggcccccagccgcccccaaccctgccccacgggcccggcgccatgagtgagctggagcaactgagacaggaggccgagcagctccggaaccagatccgggatgcccgaaaagcatgtggggactcaacactgacccagatcacagctgggctggacccagtggggagaatccagatgaggacccggaggaccctccgtgggcacctggcaaagatctatgccatgcactgggggaccgactcaaggctgctggtcagcgcctcccaggatgggaagctcatcatctgggacagctacaccaccaacaaggtccacgccatcccgctgcgctcctcctgggtaatgacctgtgcctacgcgccctcagggaactttgtggcctgtggggggttggacaacatctgctccatctacagcctcaagacccgcgagggcaacgtcagggtcagccgggagctgcctggccacactgggtacctgtcgtgttgccgcttcctggatgacaaccaaatcatcaccagctctggggataccacctgtgccctgtgggacattgagacaggccagcagacagtgggttttgctggacacagtggggatgtgatgtccctgtccctggcccccgatggccgcacgtttgtgtcaggcgcctgtgatgcctctatcaagctgtgggacgtgcgggattccatgtgccgacagaccttcatcggccatgaatccgacatcaatgcagtggctttcttccccaacggctacgccttcaccaccggctctgacgacgccacgtgccgcctcttcgacctgcgggccgatcaggagctcctcatgtactcccatgacaacatcatctgtggcatcacctctgttgccttctcgcgcagcggacggctgctgctcgctggctacgacgacttcaactgcaacatctgggatgccatgaagggcgaccgtgcaggagtcctcgctggccacgacaaccgcgtgagctgcctcggggtcaccgacgatggcatggctgtggccacgggctcctgggactccttcctcaagatctggaactaatggccccacccccactgggcccaggccaggaggggccctgcccatgcccacactacaggccagggctgcggggctggcgcaatcccagcccccttccccgggccacggggccttgggtccctgccctcccacccaggtttggttcctcccggggcccccactgtggagataagaaggggatggaatgggggaagaggaggagcaggaggccctcatccttctgctgccctggggttggggcctcacccctctggagggccggaggcaggaggtggaaaccccaggggctggcttttttaaaactggttttattttaatttttattatattttcagtttttccataaaggagccaattccaactctgtaaaaaaaaaaaaaaaaaa

SEQ ID NO: 16 - DNA, 1484 bp, Homo sapiens
  mRNA (1)..(1484): NM_032368
ggtggtttgaactttgagccttttgtagtcctgatgaataatttcattttcctcaagtttatgacactcggaacgtcaagaactggaggtttgtgcaatttgagaccggtcggcactgtgcagagatcagagtactaagagacagagattaaaatggcttccagaggaaagacagagacaagcaaattaaagcagaatttagaagaacagttggatagactcatgcaacaattacaagatctggaggaatgcagagaggaacttgatacagatgaatatgaagaaaccaaaaaggaaactctggagcaactaagtgaatttaatgattcactaaagaaaattatgtctggaaatatgactttggtagatgaactaagtggaatgcagctggctattcaggcagctatcagccaggcctttaaaaccccagaggtcatcagattgtttgcaaagaaacaaccaggtcagcttcggacaaggttagcagagatggatagagatctgatggtaggaaagctggaaagagacctgtacactcaacagaaagtggagatactaacagctcttaggaaacttggagagaagctgactgcagatgatgaggccttcttgtcagcaaatgcaggtgctatactcagccagtttgagaaagtctctacagaccttggctctggagacaaaattcttgctctggcaagttttgaggttgaaaaaacaaaaaaatgacatggtgcagaagcttgtaacattgatcacattcttaatgtaaatggtgtctttcttctggggttttcagttattgcaaagaaatgaagagattctggaaatgcatcaataacctaagaaaaagcgacataaaaatatacttatggcttgtgtttatgctcttcatcattgtgcgttgtgtgcggttacctgcttgagtgatcctgaacttgttgcgacagagggactcactggactctgttcgttatgatttgtctgtttaagagagaaaacaaagtggacttgatttttattaaggctgtttgtttttaagtgttgatagtgaacgaaaagatgtgaagtaatgatatttttctgcttacaacttatccccactcattggagtgaacagtgacgcaagctcaatagacttcataagtgttcatagaattttacaattctgagtgatcttagaaatcatttctgtttttacaaacaaggaaactgaggtccagaaagagcaagcgactttgcttaaagtcgcatcagagagctgagggtaagactcaggtgtcctgactcccagtttagtatcttttgaattttatttctgtaccatttaaaaaaaataattaacactatttgtgcaagtcagtgtttttgaaaattcagtgtcccaataaaaagtggactgcacactaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 2743 bp, Homo sapiens
  mRNA (1)..(2743): NM_018075
gaccgcatctaggccccgccgcgtccgccacgcccagcgctcggcggcgcccgccccactggcctggcccgccccccgcaccgagcactctttcgcccggcctccggttctcgccggctctcggacccgcctccgaagacgtggagcgctgcggcggctgatttgtcaaagatgaaagtgaccttatcagctttggatacttctgagagttctttcacacctttggtggtcatagaacttgctcaggatgtcaaagaagaaaccaaagaatggctgaaaaacagaattatagctaaaaaaaaagatggaggtgcccagttgttgtttagaccattgttaaataaatatgaacaagaaacactagaaaatcagaacttatatcttgttggtgcctccaagattagaatgttactaggggcagaagcagtgggattggtaaaagagtgcaatgataacaccatgagagccttcacatacagaaccagacagaacttcaaaggttttgatgataacaatgatgatttcctgacaatggcagaatgtcaattcattatcaaacatgaacttgaaaatcttagagctaaagatgaaaaaatgatccctggttaccctcaggcaaagttgtatccaggaaaatcattgttgagaagattgctcacgtctggcatcgtgattcaggtgtttccactgcatgacagtgaagccctgaagaagcttgaggacacctggtacactcggtttgctttgaagtatcagcccatagacagtattcgtggctactttggggaaacaattgctctgtactttggatttttggagtatttcacttttgcattaatccccatggctgtcattgggttaccttactacttgtttgtgtgggaagactatgacaagtacgtgatctttgcctcgttcaacctcatctggtccacggtgattctggaactgtggaagcgtggctgtgccaacatgacctacaggtgggggacactgctcatgaagagaaagtttgaggagccccggccaggatttcatggtgtcttgggtatcaattccatcactgggaaggaggagcctctgtaccccagctacaagagacagttgcgcatttacctggtctccctgccattcgtgtgcctctgcctctatttctcactgtatgtcatgatgatttacttcgacatggaggtttgggccttgggtctacatgagaacagcgggtctgagtggaccagtgtcctgttgtatgtgcccagcatcatctatgccattgtgattgagatcatgaatcgtctctatcgatatgctgccgagtttttaacttcatgggagaatcacagattggaatctgcctatcagaaccatctaattctgaaagttttagtgttcaacttcctcaattgctttgcctcactcttctatattgcctttgtcttgaaagatatgaagcttttgcgccagagcttggccactctcctaattacctcccagatcctcaaccaaattatggaatcttttcttccttattggctccaaaggaagcatggtgtgcgggtgaagaggaaggtgcaggctttaaaggcagacattgatgctacattatatgaacaagtcatcctggaaaaagaaatgggaacttatttgggcacctttgatgattacttggagttattcctgcagtttggttatgtgagccttttctcctgtgtttacccattagcagctgcctttgctgtgttaaataacttcactgaagtaaattcagatgccttaaaaatgtgcagggtcttcaaacgtccattctcagaaccttcagccaatattggtgtgtggcagttggcttttgaaacgatgagtgttatatctgtggtcactaactgtgcgctgattggaatgtcaccacaagtgaatgcagtctttccagaatcaaaagcagacctcattttgattgtagtagcagtggagcacgcactcctggctttaaagtttatacttgcatttgccatacctgataagccacggcatatccagatgaaactagccagactggaatttgagtctttggaggcactcaagcagcagcaaatgaagctcgtgaccgagaacctgaaggaggaaccaatggaaagcgggaaggagaaggcaacctgagtgcccagcgtgcccagctgccctgttggcagaggcctgtgtctgtgccacacctgccacggtggcagggggggtacccggggcagcatcgtggctcctgaacccagacccaatgcttagccaaacgaagtggctcccatgtggcaagcacccttctcagtttcgcagtggcttggctcgggatccttggcagttcccccagccccaccctgtctgctccttcccagttccttcccgggccccacacgctgctccagctgccaactttgctgcagagccactgccgcccttgagcctctcaccatgagtgagccaccagctctccacgttcccctcatagcagtgtcactcccaaccccaccatggcccagggacccgtggacaggttggggatggggtgtgtgcccactgtgctcatcacaggagcctcagttgagagtgagcggggtacagtaaggcagtgcttcccacactggacctctttcctggttctcttttgcaatacattaacagaccctttatcaacataaacaatagtaactgagctattaaaggcaacctctctgactccttctgcctaaaaaaaaaa

SEQ ID NO: 18 - DNA, 5611 bp, Homo sapiens
  mRNA (1)..(5611): NM_005096
attgaggggggcggacgccgagggggtgaacacgagtgggaagctaagagagacacggggagggggaggggaccgggaaccatttgaatgagaggaggggatcacgggtagagtgggctccaggaggtagggcgagcagggtgtgacgggggccagactcttgagccagtacatatccagccatactcatggaccccagtgatttccccagtccatttgacccattgaccctgccagagaagcccctggctggagacctaccagtagacatggaatttggagaggatctactggaatcccagactgccccaactcgaggatgggccccccctggcccttctccatcctcgggagccctggacctgcttgatacccctgctggcctggaaaaagaccctggagtcctggatggagccactgagttgctggggctgggggggctgctctataaagccccctctcccccggaggtggaccacggtcctgagggaaccctggcatgggatgcaggagatcagaccctagagcctggaccagggggccagacccctgaggtggtaccacctgatccaggggctggggcaaattcctgttcacctgaggggctactagagcctttggctccagattctccaataacactgcagtccccacatattgaagaggaggagaccacctccatagctactgcaagaaggggctcccctgggcaggaggaggagcttccccaagggcagccacagagcccaaatgccccgcctagcccttcagtgggagagactctgggggatggaatcaacagttctcagaccaaacctgggggctctagcccccctgcacatccttccttgccaggagatggcctgactgcgaaggcgagtgagaagccgcctgaacggaagagaagcgagcgcgttaggagagcagaacctccaaaacctgaggttgtagattccactgagagcattccagtgtcagatgaggattctgatgccatggtagatgaccccaatgatgaggactttgtgccattccggccccggcgctctcctcgcatgtccctacgctcaagtgtgtcacaaagggccgggcgctctgcagtgggcaccaagatgacttgtgcacattgccggacaccactgcagaaggggcagactgcctatcagcgcaaggggctgcctcagctcttctgctcgtcatcctgcctcaccactttctccaagaagccctcgggcaaaaagacctgtaccttctgcaagaaggagatctggaacaccaaggactcggttgtggcgcagactggttctggaggctccttccatgagttctgcacatccgtctgtctctccctgtatgaggcccagcagcagcgcccgatcccccagtctggggatcccgccgacgctactcgctgcagcatatgccagaagactggagaggtcctgcacgaggtcagcaatggcagcgtggtacaccggctctgcagcgattcttgcttctccaaattccgggccaacaagggactgaaaaccaactgttgtgaccagtgtggggcttacatctacaccaagaccgggagtcctggccctgagctcctcttccacgagggccaacaaaagcggttctgcaacacaacctgcttgggggcgtacaagaagaaaaacacacgtgtgtacccatgtgtctggtgcaagaccctgtgtaagaactttgagatgctatcacatgtggatcgtaatggcaagaccagcttgttctgttccctgtgctgtaccacttcttacaaagtgaagcaggcagggctcactggccctccccgaccctgcagcttctgccgccgcagcctctctgacccctgttactacaacaaggttgaccgcacagtctaccagttctgcagccccagctgctggaccaagttccagcgcacaagccctgaggggggcattcacctgagctgtcactactgtcacagcctcttcagtggcaagcctgaggtcttggactggcaggaccaagtgttccagttctgctgccgtgattgctgtgaggacttcaagcggcttcggggtgtggtgtcccagtgtgagcactgtcggcaggagaaactcttgcatgagaaactccgattcagcggagtggagaaaagcttctgcagcgaaggctgtgtgctgctgtacaaacaggacttcactaagaagctgggcttgtgctgtatcacttgtacttactgctcccagacctgccagcgcggagtcaccgagcaactggatggcagcacctgggacttctgcagtgaggactgtaagagcaagtacctgctgtggtactgcaaggctgcccggtgccatgcgtgtaagcgccaggggaagctgctggagaccatccactggcgtgggcagatccgtcatttctgcaaccagcagtgtcttctgcgtttctatagccagcagaaccaacccaacctggatacccagagtgggcccgagagcctcctgaacagtcagtctcctgagtcaaaaccccagacaccctctcaaaccaaagtggagaacagcaacacagtgaggaccccagaggaaaatgggaatttgggcaagatccctgtgaagacccgatcagctcccactgctcccacccctccacccccaccacccccagcaacaccccgcaaaaacaaggctgccatgtgtaagccactgatgcagaatcggggcgtctcctgcaaggtggagatgaagtccaaaggaagtcaaacagaagagtggaagccacaggtgatcgtgctgcccatcccagtgcccatcttcgtgccagtgcctatgcatctgtactgccagaaagtcccggtgcctttctcgatgcctatcccggtgcctgtgcccatgttcttgcccactaccttggagagcacagacaagattgtagagaccattgaggagctgaaggtgaagatcccttccaaccccttggaggccgacatcctggctatggcagaaatgattgcagaggctgaggagttagacaaggcctcatctgacctttgtgatcttgtgagcaaccagagtgcagagggactcctggaagactgtgacctgtttgggcctgctcgagatgatgtcctggccatggcagtcaagatggccaatgtcttggatgagcctgggcaagacttggaggcagacttccctaagaatcctctggacattaatcccagtgtagacttcctctttgattgtggcctggtagggcctgaggatgtgtctactgaacaagaccttccccgaaccatgaggaagggtcaaaagcggctggtgctttccgaaagctgctcccgggactccatgagcagtcagcctagttgtaccgggctcaactattcatatggtgtcaatgcttggaagtgctgggtgcagtcaaaatatgccaatggagaaaccagcaagggtgatgagctgcgctttggccccaaacccatgcgtatcaaagaggatattctcgcctgctcagctgctgagctcaactacggtctggcccagtttgtgagagaaatcactcggcccaatggtgaacgatatgaacctgacagtatctactatttgtgtcttggcatccagcagtacttgctggaaaataaccggatggtgaacattttcacggacctttactacctgacttttgttcaagaactcaacaagtctctgagtacctggcagcccacactcctccccaacaatacggtgttctctcgagtggaggaggagcacctctgggagtgtaagcaactgggggtctactcgccctttgtcctcctcaacaccctcatgttcttcaacactaagttttttgggctgcagacagctgaggaacacatgcaactctccttcaccaatgtggtgcggcagtcccgcaagtgtaccacccctcggggcaccaccaaggtggtgagcatccgctactatgccccagtccgccagaggaaagggcgagacacgggtcctggaaaacggaagagagaagatgaagcccctatcttagagcagcgtgagaaccgcatgaatcccctccgctgccctgtcaagttctatgaattctatctctcaaaatgtcctgaaagcctccggactcgcaacgatgtgttctacctgcaacctgaacggtcctgcatcgccgagtcacctctctggtattctgtgatccccatggaccgcagcatgttggagagcatgctcaatcgcatcctggctgtgcgcgagatttatgaggaactgggtcgtcctggggaggaagacctggactgagctcgtgtgccatccatatccatctttcacatcaatgtctgtcctgtggccatgtccctcagggtgacaggcccaggaaccaatgctactcattctgaagggccctgactgctcctttccgctcacccattccctgccttctctaggaaccctggcttttatcttcttccgtaccacttgacaaccatggggccctggtcttctgtactcaggggctggtctcccagtgatgggcaaaagccagcttgcccgttttctttatgcttcagagtaaacccctccttctgggtccagactctgggtggagtgttaatagctctggtgatcctgttggctttgggtttcctgacccatcccgcataggtagagcctcttgttcctaggcatgacctagggaaaaacccagctgccttctctgccctgtgcccactcccttctctactcttccccagcaccatgccaaaaggtcttatctgaaaggtaagaaataaacaatgaaagcgatgaggggaccatttacataaaacacagagcttagacactcttcccctcctatgaaataattggttgttggcaccatctcaccaccgcatatccctcaccccctcggcaagcaccaatccttggtgctgccgtttttaaaatcttccaaatgcctttttttcctcagaggcagagaatgactaagtacggggagcagactcctgttgtgcagactcctgtccccttggtttctgtgtttgtctctctgccatcttaggttgccatgagccatggtgtcaacatgcttagccccctctgtaactgcctccctttagttcaatggacagacctcccaaggcaaaaactaccttctgacttgggttgaggctgggttcccctctattgttcccctatcataagagctaggccaagcctatgggaccttgagtcatgcaggatgggatctgtggtcaaaggacaggcgaggagctgtgggcgcagggcctgccgccactgcctacatcttctctcttccccatcttgcattggaggtcccagaaaacaattagcttctggcaaagggggtacccacttctttccctgttgactttgctgtttcccaggctcctttttgtgtttttataactgtcaccagttagccactgtttaaattgtatatattgttctgaggcgcctggcctgtcccttcagtgagccatgcccacccttgtgttgtagtgagaagctgttgtcacgactaaccttctgtctctgaaattgtttgtttcaaataaagagttaaaattgtcaaaaaaaaaaaaaaaaaa

SEQ ID NO: 19 - DNA, 974 bp, Homo sapiens
  mRNA (1)..(974): NM_002201
cctgacatggagcctgccagctccgtcagccctgactcggcccggagctgagctccccacctgccggtagcccaggagatggagcagcccagcccacgtgcccggccttccgcccctgacttcacttgataacaaactagaaactgaaacagggtcgggatgccgatgccggcttggagttagagatgagtcaccgctgagagcagctgcagtagctgagcagtggcagcagagaggcagacgtgagctgagggcgcagaggcaggcagcatctctgagggtccccaaggagcatggctgggagccgtgaggtggtggccatggactgcgagatggtggggctggggccccaccgggagagtggcctggctcgttgcagcctcgtgaacgtccacggtgctgtgctgtacgacaagttcatccggcctgagggagagatcaccgattacagaacccgggtcagcggggtcacccctcagcacatggtgggggccacaccatttgccgtggccaggctagagatcctgcagctcctgaaaggcaagctggtggtgggtcatgacctgaagcacgacttccaggcactgaaagaggacatgagcggctacacaatctacgacacgtccactgacaggctgttgtggcgtgaggccaagctggaccactgcaggcgtgtctccctgcgggtgctgagtgagcgcctcctgcacaagagcatccagaacagcctgcttggacacagctcggtggaagatgcgagggcaacgatggagctctatcaaatctcccagagaatccgagcccgccgagggctgccccgcctggctgtgtcagactgaagccccatccagcccgttccgcagggactagaggctttcggctttttgggacagcaactaccttgcttttggaaaatacatttttaatagtaaagtggctctatattttctctacgcaaaaaaaaaaaaaaaaaa

SEQ ID NO: 20 - DNA, 942 bp, Homo sapiens
  mRNA (1)..(942): NM_012367
atgaattgggtaaatgacagcatcatacaggagtttattctgctgggtttctcagatcgaccttggctggagtttccactccttgtggtcttcttgatttcttacactgtgaccatctttggcaatctgaccattattctagtgtcacgcctggacaccaaacttcatacccccatgtatttttttcttaccaatctatcactcctggatctttgttacaccacatgtacagtcccacaaatgctagtaaatttatgcagcatcaggaaagtaatcagttatcgtggctgtgtagcccagcttttcatatttctggccttgggggctactgaatatcttctcctggccgtcatgtcctttgataggtttgtagctatttgtcggcctctccattactcagttatcatgcaccagagactctgcctccagttggcagctgcatcctgggttactggttttagtaactcagtgtggttgtctaccctgactctccagctgccactctgtgacccctatgtgatagatcactttctctgtgaagtccctgcactgctcaagttatcttgtgttgagacaacagcaaatgaggctgaactattccttgtcagtgagctcttccatctaatacccctgacactcatccttatatcatatgcttttattgtccgagcagtattgaggatacagtctgctgaaggtcgacaaaaagcatttgggacatgtggttcccatctaattgtggtgtctcttttttatagtacagccgtctctgtgtacctgcaaccaccttcgcccagctccaaggaccaaggaaagatggtttctctcttctatggaatcattgcacccatgctgaatccccttatatatacacttaggaacaaggaggtaaaggaaggctttaaaaggttggttgcaagagtcttcttaatcaagaaataa

SEQ ID NO: 21 - DNA, 3107 bp, Homo sapiens
  mRNA (1)..(3107): NM_018052
gactcgagtaacatggccgctgtctcgtgagtcccgctagtgccgggcgggagttgttaagcggccagggtcaggtgtgctggagcggggtccgggcccgggttccagggcgaggcggcggagcgtggcaggcaagcctagagcggcgtggtccatgcgccggcgccgggggcagagcggagccgcagactcccctggccccggcgcggccccggcagccgcgggctaaggagtcgcgaggttcccccagctgccaccatgaaccccgagaaggatttcgcgccgctcacgcctaacatcgtgcgcgccctcaatgacaagctgtacgaaaagcggaaggtggcagcgctggagatcgagaagctggtccgggagttcgtggcccagaacaataccgtgcaaatcaagcatgtgatccagaccctgtcccaggagtttgccctgtctcagcacccccacagccggaaagggggcctcatcggcctggccgcctgctccatcgcactgggcaaggactcagggctctacctgaaggagctgatcgagccagtgctgacctgcttcaatgatgcagacagcaggctgcgctactatgcctgcgaggccctctacaacatcgtcaaggtggcccggggcgctgtgctgccccacttcaacgtgctctttgacgggctgagcaagctggcagccgacccagaccccaatgtgaaaagcggatctgagctcctagaccgccttttaaaggacattgtgactgagagcaacaagtttgacctggtgagcttcatccccttgttgcgagagaggatttactccaacaaccagtatgcccggcagttcatcatctcctggatcctggttctggagtcggtgccagacattaacctgctggattacctgccggagatcctggatggactcttccagatcctgggtgacaatggcaaagagattcgcaaaatgtgtgaggttgttcttggagaattcttaaaagaaattaagaagaacccctccagtgtgaagtttgctgagatggccaacatcctggtgatccactgccagacaacagatgacctcatccagctgacagccatgtgctggatgcgggagttcatccagctggcgggccgcgtcatgctgccttactcctccgggatcctgactgctgtcttgccctgcttggcctacgatgaccgcaagaaaagcatcaaagaagtggccaacgtgtgcaaccagagcctgatgaagctggtcacccccgaggacgacgagctggatgagctgagacctgggcagaggcaggcagagcccacccctgacgatgccctgccaaagcaggagggcacagccagtggaggtccagatggttcctgtgactccagcttcagtagcggcatcagtgtcttcactgcagccagcactgaaagagccccagtgacccttcacctcgacgggatcgtgcaggtcctaaactgccacctcagtgacacggccattgggatgatgaccaggattgcagttctcaagtggctctaccacctctacatcaaaactcctcggaagatgttccggcacacggacagcctctttcccatcctactgcagacgttatcggatgaatcggatgaggtgatcctgaaggacctggaggtgctggcagaaatcgcttcctcccccgcaggccagacggatgacccaggccccctcgatggccctgacctccaggccagccactcagagctccaggtgcccacccctggcagagccggcctactgaacacctctggtaccaaaggcttagaatgttctccttcaactcccaccatgaattcttacttttataagttcatgatcaaccttctcaagagattcagcagcgaacggaagctcctggaggtcagaggccctttcatcatcaggcagctgtgcctcctgctgaatgcggagaacatcttccactcaatggcagacatcctgctgcgggaggaggacctcaagttcgcctcgaccatggtccacgccctcaacaccatcctgctgacctccacagagctcttccagctaaggaaccagctgaaggacctgaagaccctggagagccagaacctgttctgctgcctgtaccgctcctggtgccacaacccagtcaccacggtgtccctctgcttcctcacccagaactaccggcacgcctatgacctcatccagaagtttggggacctggaggtcaccgtggacttcctcgcagaggtggacaagctggtgcagctgattgagtgccccatcttcacatatctgcgcctgcagctgctggacgtgaagaacaacccctacctgatcaaggccctctacggcctgctcatgctcctgccgcagagcagcgccttccagctgctctcgcaccggctccagtgcgtgcccaaccctgagctgctgcagaccgaagacagtctaaaggcagcccccaagtcccagaaagctgactcccctagcatcgactacgcagagctgctgcagcactttgagaaggtccagaacaagcacctggaagtgcggcaccagcggagcgggcgtggggaccacctggaccggagggttgtcctctgacaggcctggcacggaggagggcccaccgagtggtcccatgaaacactaagggtcgtcacgccctcccgaggagctcaaggacctgcctgtcaggaccagggctgggcctgccaacccagggcagtgttggggccggaggctgctgtgtctgcccaagctcctctcagagtccagtccccaggcctccagcgctgtcagctgcaccctggcattctcacagagctggctgcccacccagtggggggctatagcctcagagaccactcatcctctggaatcaacctctttctaataccctcttggaaaaagagcttgcccctcctccagcacactagagctctggccttgtgtgtatatgtatacatacgtgaacacatgcctgtgtgtgtgtgtgtgtgtgtgtacttgtatgcacgtaggcaccagcacaaagatctgaatgatgcaccccacccccaccccaataaagaaataacagaaaaccctcaaaaaaaaaaaaa

SEQ ID NO: 22 - DNA, 2180 bp, Homo sapiens
  mRNA (1)..(2180): NM_080740
agaaaggactttgaagagcaagcggcgttgttcgcctgcggaagcagtgctgccggtctctggacaaatttctcccggaggtggagacaaggaagctccagcttcgctttcccttgtggcccagagtggaacatactgagctgcactgaatcatctggatgcactcgcaactttaggaactggtggcaatttgtaaaaagacagctctggaagttgaagagaccagtggcttcaagcaaaaagtaaattgaaaatgggagatatctttttgtgtaagaaagtggaatcaccaaagaagaatttgagagaatccaaacaaagggaggaggatgatgaagatccagatctgatctatgttggggtggagcatgtacatagagatgctgaagttctctttgtcgggatgatttcaaattcaaaaccagtcgtttcaaacattttgaacagagtcaccccaggctcaaagtcaagaagaaagaaaggccacttccgtcaatatcctgctcacgtgtcgcagcctgcaaatcatgtgacctctatggcaaaagccatcatgcccgtttctctgtctgaggggcgatcgacagatagtcctgtcactatgaagtcttcatctgaacctggttataaaatgagctcaccacaagttgtttctcccaattactcagattcgctccccccagggactcagtgtctagttggagctatggtctctggaggaggcagaaatgagagttctcctgattcaaagcgactttccacttcagatataaacagcagagattccaaaagggttaaactcagggatggaatcccaggggtaccttctttagctgtggtcccttcagatatgtcttctacaataagcacaaatacaccctcacaggggatctgcaactcatcaaaccatgttcagaatggagtaacatttccttggcctgatgctaatggaaaggcacatttcaatcttacagatccagagagagcaaatgagtctggcctggcaatgacagacatttcaagtctagcaagtcaaaacaagacctttgatcccaagaaagaaaatcccatcgtgttacttagcaacttttactatggacagcataaaggagatgggcagccggaacagaagactcacaccacctttaaatgcctcagctgcgtgaaagttctaaaaaatattaagtttatgaatcacatgaagcatcatttggaatttgagaagcagaggaacgacagctgggaagaccacaccacctgccagcactgccaccggcagtttcccactcccttccagctacagtgtcacattgatagtgtacacatcgccatggggccctctgctgtctgtaaaatctgtgaattgtcatttgaaacagatcaggtcctcttacaacacatgaaggaccatcataagcctggcgaaatgccctacgtgtgccaggtttgccattacagatcgtcggtctttgctgatgtggaaacacattttagaacgtgccatgaaaacacaaagaatttgctttgtctgttttgtctcaaacttttcaaaactgcaataccatacatgaatcattgttggaggcacagcagaaggagggtccttcagtgttccaagtgccggctacagtttttgacgttgaaggaggaaatagagcacaaaaccaaggaccatcaaacatttaaaaagccggagcaactgcaagggttccctcgtgaaacaaaagttattattcaaacttcagttcagccaggatcaagtggtatggcttccgttattgttagcaacactgaccctcagtcttctcctgtaaaaactaaaaagaagacggctatgaacactagagattccagactcccttgcagcaaggattctagctgaaaatatgttcgactgacttccaggagttccgaaaggcagaagtgaggctaggccatatgtgcattgtggctgtagatgctagcaagcaatgtcatcaactgtcaggttgtttttcacagtgacatctcatcagctccctattgtcaagaagtagctccaaagtctttgagccctttcccagtcaaatgctcccaacaaggcatcccttattctgacctcagaggtttttcttgtttatgaactttaaataaatggaatcacacaaaaaaaaaaaaaaaaaaaaaaaaaaaaaaaaaaa

SEQ ID NO: 23 - DNA, 3010 bp, Homo sapiens
  mRNA (1)..(3010): NM_022486
cgggaggaggcgcgggcgcgccgggagggaccggcggcggcatgggccgggggccctgggatgcgggcccgtctcgccgcctgctgccgctgttgctgctgctcggcctggcccgcggcgccgcgggagcgccgggccccgacggtttagacgtctgtgccacttgccatgaacatgccacatgccagcaaagagaagggaagaagatctgtatttgcaactatggatttgtagggaacgggaggactcagtgtgttgataaaaatgagtgccagtttggagccactcttgtctgtgggaaccacacatcttgccacaacacccccgggggcttctattgcatttgcctggaaggatatcgagccacaaacaacaacaagacattcattcccaacgatggcaccttttgtacagacatagatgagtgtgaagtttctggcctgtgcaggcatggagggcgatgcgtgaacactcatgggagctttgaatgctactgtatggatggatacttgccaaggaatggacctgaacctttccacccgaccaccgatgccacatcatgcacagaaatagactgtggtacccctcctgaggttccagatggctatatcataggaaattatacgtctagtctgggcagccaggttcgttatgcttgcagagaaggattcttcagtgttccagaagatacagtttcaagctgcacaggcctgggcacatgggagtccccaaaattacattgccaagagatcaactgtggcaaccctccagaaatgcggcacgccatcttggtaggaaatcacagctccaggctgggcggtgtggctcgctatgtctgtcaagagggctttgagagccctggaggaaagatcacttctgtttgcacagagaaaggcacctggagagaaagtactttaacatgcacagaaattctgacaaagattaatgatgtatcactgtttaatgatacctgtgtgagatggcaaataaactcaagaagaataaaccccaagatctcatatgtgatatccataaaaggacaacggttggaccctatggaatcagttcgtgaggagacagtcaacttgaccacagacagcaggaccccagaagtgtgcctagccctgtacccaggcaccaactacaccgtgaacatctccacagcacctcccaggcgctcgatgccagccgtcatcggtttccagacagctgaagttgatctcttagaagatgatggaagtttcaatatttcaatatttaatgaaacttgtttgaaattgaacaggcgttctaggaaagttggatcagaacacatgtaccaatttaccgttctgggtcagaggtggtatctggctaacttttctcatgcaacatcgtttaacttcacaacgagggaacaagtgcctgtagtgtgtttggatctgtaccctacgactgattatacggtgaatgtgaccctgctgagatctcctaagcggcactcagtgcaaataacaatagcaactcccccagcagtaaaacagaccatcagtaacatttcaggatttaatgaaacctgcttgagatggagaagcatcaagacagctgatatggaggagatgtatttattccacatttggggccagagatggtatcagaaggaatttgcccaggaaatgacctttaatatcagtagcagcagccgagatcccgaggtgtgcttggacctacgtccgggtaccaactacaatgtcagtctccgggctctgtcttcggaacttcctgtggtcatctccctgacaacccagataacagagcctcccctcccggaagtagaattttttacggtgcacagaggacctctaccacgcctcagactgaggaaagccaaggagaaaaatggaccaatcagttcatatcaggtgttagtgcttcccctggccctccaaagcacattttcttgtgattctgaaggcgcttcctccttctttagcaacgcctctgatgctgatggatacgtggctgcagaactactggccaaagatgttccagatgatgccatggagatacctataggagacaggctgtactatggggaatattataatgcacccttgaaaagagggagtgattactgcattatattacgaatcacaagtgaatggaataaggtgagaagacactcctgtgcagtttgggctcaggtgaaagattcgtcactcatgctgctgcagatggcgggtgttggactgggttccctggctgttgtgatcattctcacattcctctccttctcagcggtgtgatggcagatggacactgagtggggaggatgcactgctgctgggcaggtgttctggcagcttctcaggtgcccgcacagaggctccgtgtgacttccgtccagggagcatgtgggcctgcaactttctccattcccagctgggccccattcctggatttaagatggtggctatccctgaggagtcaccataaggagaaaactcaggaattctgagtcttccctgctacaggaccagttctgtgcaatgaacttgagactcctgatgtacactgtgatattgaccgaaggctacatacagatctgtgaatcttggctgggacttcctctgagtgatgcctgagggtcagctcctctagacattgactgcaagagaatctctgcaacctcctatataaaagcatttctgttaattcattcagaatccattctttacaatatgcagtgagatgggcttaagtttgggctagagtttgactttatgaaggaggtcattgaaaaagagaacagtgacgtaggcaaatgtttcaagcactttagaaacagtacttttcctataattagttgatatactaatgagaaaatatactagcctggccatgccaataagtttcctgctgtgtctgttaggcagcattgctttgatgcaatttctattgtcctatatattcaaaagtaatgtctacattccagtaaaaatatcccgtaattaagaaaaaaaaaaaaaaa

SEQ ID NO: 24 - DNA, 735 bp, Homo sapiens
  mRNA (1)..(735): NM_024518
atggcagcggccgccagccccgcgatccttccgcgcctcgcgattcttccgtacctgctattcgactggtccgggacggggcgggccgacgctcactctctctggtataacttcaccatcattcatttgcccagacatgggcaacagtggtgtgaggtccagagccaggtggatcagaagaattttctctcctatgactgtggcagtgacaaggtcttatctatgggtcacctagaagagcagctgtatgccacagatgcctggggaaaacaactggaaatgctgagagaggtggggcagaggctcagactggaactggctgacactgagctggaggatttcacacccagtggacccctcacgctgcaggtcaggatgtcttgtgagtgtgaagccgatggatacatccgtggatcttggcagttcagcttcgatggacggaagttcctcctctttgactcaaacaacagaaagtggacagtggttcacgctggagccaggcggatgaaagagaagtgggagaaggatagcggactgaccaccttcttcaagatggtctcaatgagagactgcaagagctggcttagggacttcctgatgcacaggaagaagaggctggaacccacagcaccacccaccatggccccaggcttagctcaacccaaagccatagccaccaccctcagtccctggagcttcctcatcatcctctgcttcatcctccctggcatctga

SEQ ID NO: 25 - DNA, 5379 bp, Homo sapiens
  mRNA (1)..(5379): NM_017901
tggataggagagctggcgcagctgccctggtggcagtggctgaagtggcggcggcttcggcggctgcggcggctgcaacagcttcgggctcggggttttggcggcggcgccggcgggctaggctgcgcggtgcggaccccggcgcgcggtccgggttgctggggcggcgcgtaagatgcctctaatggaggagtttctgagcagcacccctggcccagtggctttgaaagggagctcaaaccagagactatttcaagccctggatatcatatcctgagggccacaggagaagagaacatggctgtgagtttggatgacgacgtgccgctcatcctgaccttggatgagggtggcagtgccccactggctccctccaacggcctgggccaagaagagctacctagcaaaaatggcggcagctatgccatccacgactcccaggcccccagtctcagctctgggggtgagagttccccctccagccccgcacacaactgggagatgaattaccaagaggcagcaatctacctccaggaaggcgagaacaacgacaagttcttcacccaccccaaggatgccaaggcgctggcggcctacctctttgcacacaatcacctcttctacctgatggagctggccacggccctgctgctgctgctgctctccctgtgcgaggcccccgccgtccccgcactccggcttggcatctatgtccacgccaccctggagctgtttgccctgatggtggtagtgtttgaactctgcatgaagttacgctggctgggcctccacaccttcatccggcacaagcggaccatggtcaagacctcggtgctggtggtgcagtttgtcgaggccatcgtggtgttggtacggcagatgtcccatgtgcgggtgacccgagcactgcgctgcattttcctggtggactgtcggtattgcggtggcgtccggcgcaacctgcggcagatcttccagtccctgccgcccttcatggacatcctcctgctgctgctgttcttcatgatcatctttgccatcctcggtttctacttgttctcccctaacccttcagacccctacttcagcaccctggagaacagcatcgtcagtctgtttgtccttctgaccacagccaatttcccagatgtgatgatgccctcctactcccggaacccctggtcctgcgtcttcttcatcgtgtacctctccatcgagctgtatttcatcatgaacctgcttctggctgtggtgttcgacaccttcaatgacattgagaaacgcaagttcaagtctttgctactgcacaagcgaaccgctatccagcatgcctaccgcctgctcatcagccagaggaggcctgccggcatctcctacaggcagtttgaaggcctcatgcgcttctacaagccccggatgagtgccagggagcgctatcttaccttcaaggccctgaatcagaacaacacacccctgctcagcctaaaggacttttacgatatctacgaagttgctgctttgaagtggaaggccaagaaaaacagagagcactggtttgatgagcttcccaggacggcgctcctcatcttcaaaggtattaatatccttgtgaagtccaaggccttccagtatttcatgtacttggtggtggcagtcaacggggtctggatcctcgtggagacatttatgctgaaaggtgggaacttcttctccaagcacgtgccctggagttacctcgtctttctaactatctatggggtggagctgttcctgaaggttgccggcctgggccctgtggagtacttgtcttccggatggaacttgtttgacttctccgtgacagtgttcgccttcctgggactgctggcgctggccctcaacatggagcccttctatttcatcgtggtcctgcgccccctccagctgctgaggttgtttaagttgaaggagcgctaccgcaacgtgctggacaccatgttcgagctgctgccccggatggccagcctgggcctcaccctgctcatcttttactactccttcgccatcgtgggcatggagttcttctgcgggatcgtcttccccaactgctgcaacacgagtacagtggcagatgcctaccgctggcgcaaccacaccgtgggcaacaggaccgtggtggaggaaggctactattatctcaataattttgacaacatcctcaacagctttgtgaccctgtttgagctcacagttgtcaacaactggtacatcatcatggaaggcgtcacctctcagacctcccactggagccgcctctacttcatgaccttttacattgtgaccatggtggtgatgacgatcattgtcgcctttatcctcgaggccttcgtcttccgaatgaactacagccgcaagaaccaggactcggaagttgatggtggcatcacccttgagaaggaaatctccaaagaagagctggttgccgtcctggagctctaccgggaggcacggggggcctcctcggatgtcaccaggctgctggagaccctctcccagatggagagataccagcaacattccatggtgtttctgggacggcgatcaaggaccaagagcgacctgagcctgaagatgtaccaggaggagatccaggagtggtatgaggagcatgccagggagcaagagcagcagcgacaactcagcagcagtgcagcccccgccgcccagcagcccccaggcagccgccagcgctcccagaccgttacctagcccagcgcccgaaagccgtctcttctatgcaataacacaatagtattactctactgcgatgtacggaactgcggtgtgtgtacacatactcacgtatatgcacatatttatatacaggaagaaaaaagacagacaagatggggcttggtttataaccaccttgccctgtcttccttaactccagaagccagtttggtgaggggtgggggtgcggccaccaggtctgagctcttcctactgtggaaggctccagaaggcccttcacaaggagacccctcacctggatccagtcgactgcggggcttgcccctcatgtgggctggcctccatcggccacgtccaaagctgtcactgctactgcttcaggctcacatccccccgacctgatggcgtgcccgccccctctccctgcggcccatgccacaggtttctgtgttttgctttagggacagaaccacttaggaaggaaagaactcccggtctccagggtggtatttcagtgtctgtgataatgtcacgcaacacctcttcggggaccagtgcccaggatctaatggaagcggaattggggcaactgggcccatgtggccagagctcagttagccagtgccgggcggccacagattacactgaccaatctcctcccttggctctgcaagcctcccacccagccttctctggcttaacccttgttggcgaaaactcttccacagtggcctccttggggacccagaacccggaggaaggggcatgaggcaggaagtggggccgatgtctgcaacccagaccacttcgtggaatgggctcttgaccaaatccctttttttgcgatttacccgttcaagcaaaacaacgttttggttaactaaggattgtgctaaagccgataccaggtccttcacacgtgtgcactaggaacaggagcgaacagcacagagagacgctccctgtgggacgcagcagccccgtggccccggcccagttcccagccaccctccctggctctgctcacaccagagatttccatagcaggagcggttggtgcagaagtaggttcagatgaacctcagttaacgtcgccacccctcctcccaccatggtaccctgtaggagccctgtatgacatctgagcgtggtggaggtaggagggttgccagctgcagtgaccctgccacagaggcagggtcagtgcagaggtcgctttggttccgcttccctgggccacagaacggaacacagcataggttctgcagcaggagccgcagtggcaggatggagggtgcgaagggcaaggagtgcactgctgggcattcctggccagccccggccctctggtgcctgcttcctgtgacttcagaaggcaggtggacagagcctccctctggccttgtcctcttcccagccacagaacgggcagggtggcacccgaccccaggggagcagtacctggtcccccaccccctcctcccaaccacctccaaggccaagctgggtcccatagccagcacggcatggttctccccttccccccttcccaggtcaggggagttggacaagtagcaggtgtttgtttttaaagcacagccctttgggaaagcaacacattattgagactcactgtgattcccccgggagtcagactggctttgtcctcttcctctctggagggccatgggccatcagcaggagctccacatcgagccccaggccagaaccccctccctttcacagagagggaactttattgcacaattgggtgccttttagcttttgtgtgttgaaatgggcgttttggaagcaagggtcaggggacagcttctaaaggtgtgagtctctgacctgagcatctgggcctcgcctgggcccttcttcctccccaggggtggaaacgtggaggggccagcagcaccccggggtctgcccaggggagtcaaggccccgaggtgggggggcctattccaggaggagtgggatctcggccctgtccagagcgagttaatgtgtccatctgcccaaccctgtcctgagaaaggacctggttttggccaggacctgacaaatacccaatggcagcagtgtcaacagacgggagtccagccagggtgggtgcccttgtcattgaggttagggacagctatccccaggttatgcctggccccacccagcagggagttggggtccccccacaggctgtgagctctgtgggccctgggatgtgatctagctcagatgccctctcatccttgatgtcatagttgagtgcacccaagtggcacccactggcggccaggggcacagcctggtggtggtggcaatagaactgtgcccctcctcagctcctcgcttcccctcccacagccccagccttactccaccatgcggacaatcattttgtacggatcacgggagcaatgctgtacggttttgtacactggtggtttgtttcctagaaaacccattgtgtctctggatttctagcacattactaaaagagcctctgctttgtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - DNA, 2748 bp, Homo sapiens
  mRNA (1)..(2748): NM_006752
aaaaatcgaggcggggctgagaggctgcggcggctgactaggttttcggaccgcggcgcggcggggaagagagactgcggcgggagggccaggccgtgggagagacgcgggaaagtgcgggtcgcagaggcacttgaccaccccgccctaggccgacccggcggacgcggcgtctggtgtgcgggcgctggggcgggtcccaggtctccccgctgcgctgcttgaggctcagccatggcccagcagagagccctgccccagagcaaggagacgctgctgcagtcctacaacaagcggctgaaggacgacattaagtccatcatggacaacttcaccgagatcatcaagaccgccaagattgaggacgagacgcaggtgtcacgggccactcagggtgaacaggacaattacgagatgcatgtgcgagccgccaacatcgtccgagccggcgagtccctgatgaagctggtgtccgacctcaagcagttcctgatcctcaatgacttcccctccgtgaacgaggccattgaccagcgcaaccagcagctgcgcacactgcaggaggagtgcgaccggaagctcatcacgctgcgagacgagatctccattgacctctacgagctggaggaggagtattactcgtccaggtataaatagcgctggactccccatgcagagcgggagcctgcctacctgggcctggccagcaggcagggctgccttctgctttttcaaattcttgctggtcttagcagtggagccatgcctgggtttcagagcagagctcctggccagagcgtttgaccgacagacaattcacatccatatgccagggccctgggcctttcccacagtgcaatgtgatgaaaaccacaggactcacgccagtcggataggccgagtctggagaagggaggcgcctggctgtatcccccgcaggccctcttccgagagccttcctcctcgggcagtgcgttctggggctgtgctgctcctgttaccttctgaatccatatgtagagatttcagccaaggctgggccagccttttttgggcagtcaggtccacacctatgtccagggcaccagggatgcaattccatgtggatgtcaccaaaccccagtgtggaggcagggacagtcatgggaatgtgggggatgaagcccaggcagggaatggccttgaaagccattggagctccaattcgtgacccactcagccttatccacggagctggagccaacctacgtgccaggccccgtgctgggtcccagggatgcagaagggtcaaaacccatcatcctgacccttgtggggctccgtaagaagctgaaaccttcgaccgtttgagctggaggggccctgagaaatcagagtctacgtatcatttacttagggggaaacttaggctggagacagggaggccttccactctgccccattagcttagaaaatcaagattcagtccagcagatgcagagtccatgtccatcttgtgccttctcctggacaaacctttccttcctggtggtggatttaaaatactcctttctgcccattggccatgctgggagccacagatatccagagccagcatgacctggggcttggtttccctgccctgggctcagtggcactgctgagctgcagcagtcctagagttttccagggggttctgagggaatctttggtccccagtactcattaactcagcagacatgaggcagcatttcctccacactagggtggctgagaggggtcctggggtgtttcagacccttctgggcatctccttccacagctgttcagtttgtcggtctctttgaggcagccaccgtccctgagggcccctgcacagagcagctgtgggcctgtaattcagcctgcctgccttgccttggggcagggagagagggaacctgctcacggccctgcagcagagcagggcgcaaacccaggacatctgtgccaggcttcccatgccctcccccaacagtccctcagcttcacccagcggggcttccaggccagcctgtgtcccctcccgcaggcctcctgtccacaccagcgccccctacaggtggcagcccctgcccactcccatgctggtggccctggccccactgagcacgcctgagcctccggggccacgcttcgttctcaggaacaaaacctgaggcagccctttggatgccctcacagccttgcttctctcagcctaggttcccatttggggacttcaggaccccagagccactaggacttccttgggaagcccgttagcccagggtgggtcccgccaggacagtagggaaacagttgtttccctagccatttccgaatagcccatcattccgagtcatcatctctgtttgctgccttcctggccagccaggtggaagaaagtttccaagctaggtctggcccgttggggatctcagcagtggggcaggagggtgcctgatttcggggagtcctgacccgagcctgttgtcagagttgggaggggctctgagcagtgttgggcaggccgggtctcccatcccgaggccagcgttcctgtgcagagccccatccactggttcttgccctgagccacatatgtctgtgccatgggctgagtgccacgacaggcccgtgtgacagctgctgcccacgcatgtggaagctaggtgggactcattcctaattctgccgttgtaatgagacttgattaaaacaccgccacttttttgcaaaaaaaaaaaa

SEQ ID NO: 27 - DNA, 1851 bp, Homo sapiens
  mRNA (1)..(1851): NM_004261
ggagaagaaggcggggctaaaactggcgaaggcgtggcttcttggctgcttgacgaagtgtcgtgaataaaagaaaggagaccgcagaagtaaagaagtggggagtttaggcaagtgcctgatttgggtaatcgaaagcacccagtgattgtatttgatgacttttaagctttcatatgccgttatttaatacctgtcacttccaaatgagagatgtaagggcaacggccgttagcgttctgttttggatcaggctctggagtggacgcccctagcttaggggtccttctaggcagccagaaacctgcggaaaatggtagcgatggcggctgggccgagtgggtgtctggtgccggcgtttgggctacggttgttgttggcgactgtgcttcaagcggtgtctgcttttggggcagagttttcatcggaggcatgcagagagttaggcttttctagcaacttgctttgcagctcttgtgatcttctcggacagttcaacctgcttcagctggatcctgattgcagaggatgctgtcaggaggaagcacaatttgaaaccaaaaagctgtatgcaggagctattcttgaagtttgtggatgaaaattgggaaggttccctcaagtccaagcttttgttaggagtgataaacccaaactgttcagaggactgcaaatcaagtatgtccgtggttcagaccctgtattaaagcttttggacgacaatgggaacattgctgaagaactgagcattctcaaatggaacacagacagtgtagaagaattcctgagtgaaaagttggaacgcatataaatcttgcttaaattttgtcctatccttttgttaccttatcaaatgaaatattacagcacctagaaaataatttagttttgcttgcttccattgatcagtcttttacttgaggcattaaatatctaattaaatcgtgaaatggcagtatagtccatgatatctaaggagttggcaagcttaacaaaacccattttttataaatgtccatcctcctgcatttgttgataccactaacaaaatgctttgtaacagacttgcggttaattatgcaaatgatagtttgtgataattggtccagttttacgaacaacagatttctaaattagagaggttaacaagacagatgattactatgcctcatgtgctgtgtgctctttgaaaggaatgacagcagactacaaagcaaataagatatactgagcctcaacagattgcctgctcctcagagtctctcctatttttgtattacccagctttctttttaatacaaatgttatttatagtttacaatgaatgcactgcataaaaactttgtagcttcattattgtaaaacatattcaagatcctacagtaagagtgaaacattcacaaagatttgcgttaatgaagactacacagaaaacctttctagggatttgtgtggatcagatacatacttggcaaatttttgagttttacattcttacagaaaagtccatttaaaagtgatcatttgtaagaccaaaatataaataaaaagtttcaaaaatctatctgaatttggaattcttctggtttgttctttcatgtttaaaaatgatgtttttcaatgcatttttttcatgtaagccctttttttagccaaaatgtaaaaatggctgtaatatttaaaacttataacatcttattgttggtaatagtgctttatatttgtctgattttatttttcaaagttttttcatttatgaacacattttcattggtatattatttaaggaatatctcttgatatagaatttttatattaaaaatgatttttctttgcttaaaaaaaaaaaaa

SEQ ID NO: 28 - DNA, 1126 bp, Homo sapiens
  mRNA (1)..(1126): NM_003134
ccagcctaggccgaacttccggctctcactgctaggggcttaagcggagggagtcgagccagcgtcgccgcgatggtgttgttggagagcgagcagttcctgacggagctgaccagacttttccagaagtgccggacgtcgggcagcgtctatatcaccttgaagaagtatgacggtcgaaccaaacccattccaaagaagggtactgtggagggctttgagcccgcagacaacaagtgtctgttaagagctaccgatgggaagaagaagatcagcactgtggtgagctccaaggaagtgaataagtttcagatggcttattcaaacctccttagagctaacatggatgggctgaagaagagagacaaaaagaacaaaactaagaagaccaaagcagcagcagcagcagcagcagcagcacctgccgcagcagcaacagcaccaacaacagcagcaacaacagcagcaacagcagcacagtaaagggcatacatttcctgctttcaccaattaaccactgaattgctattttttccttttggccagatagctaggtttctggttcccccacagtaggtgttttcacataagattagggtccttttggaaagaatagttgcagtgtttataggatagttgtggtaagaatctagtttattttgcatttggctaattggtctgtgctgcatggttatatactcctggattatagattaaaagtctctgtagacatctctgtgaagagcaagctatcattaaacatgtctgtttatcagcactgtctctttattcctttcccaacccattttaatagttctggcaataactactaaatctagaatgatgtgattaatgaataggctttagctctataatatcttctaggttattagaattgaaacctgacagttttataaaaagtcatgttatctcatgagctgcttcccacctggctgtataattttatcatcatggttccccagtttcgatgagttctcacagtcaaatgagagtttgtttaaccaccttaggagaaacatactacaaagtcatcaagaataaaggttccaaagtaattatgatttttggtttctttatgccctttggtttggatattttcatgtgc

SEQ ID NO: 29 - DNA, 2463 bp, Homo sapiens
  mRNA (1)..(2463): NM_024644
caccgcagccgctgcattcaggaaccgctttagcttcgcccccggccggccgggcggggaagactggtgtggtctggccatggatgggctccaggccagtgcagggccgttgaggcgcgggcggccgaagcgccggcgcaagccccagccacacagcgggtcggtcctggccctgcccttgaggtccaggaagatacgaaagcagctgcgaagtgttgtatcccgcatggcagcgctgaggacgcagacgctgcctagcgagaactcggaggaatcgagggtggagtcgacggccgacgacctgggggacgcgctacccggtggggcggcggtggcggccgtcccggacgcagcccggcgagagccatacggccacctggggcccgcagagctgctggaggcctcgcccgccgcgcgctccctgcagaccccgtcggcgcgcctggtgcccgcttccgcgccgcccgcgcgcctggtggaggtgcccgccgcgccggtccgggtggtggagacctcggccctgctgtgcaccgcgcaacacttagcggccgtccagtcgtccggggcccctgcgacggcgtcggggccgcaggtggataacacgggtggggagccggcctgggactccccgctgcggcgcgtcttggccgagctgaaccgcatccccagcagccggcggcgagcggcccgcctctttgagtggctcatcgcgcccatgccgccagatcacttttaccggcgcctatgggagcgcgaggcggtgctggtgcggcggcaggaccacacctactaccagggacttttctctaccgctgacctggattcgatgctgcgcaacgaggaggtgcagttcggccagcatttggacgccgctcgctacatcaacggacgacgcgagaccctgaacccacccggccgcgcgctgcccgccgccgcgtggtccctgtaccaggccggctgctccctgcgtctcctctgtccgcaggctttctctactactgtgtggcagtttttggctgtgcttcaagagcagtttggaagcatggcaggctccaacgtttacctcacgccccctaactcgcagggctttgccccccactacgacgacatcgaggccttcgtgctgcagctggaaggtaggaaactctggcgtgtataccgaccccgagtcccaaccgaggaactggctctgacatccagccccaacttcagtcaggacgacctcggtgagccggtgctgcagaccgtgctggaacctggagatttgctgtattttcctcggggcttcattcaccaagctgaatgccaggatggagtccactctctgcacctcaccttgtccacgtaccagcgcaatacctggggtgacttcttagaggccatactgcctctggcagtgcaggctgcaatggaagaaaatgtggagtttcggaggggtctgccccgagacttcatggattacatgggggcccagcattcagattctaaggatccgcgaagaaccgctttcatggagaaggtgcgggtcttggttgcccgcctgggacactttgctcctgttgatgctgtggccgaccagcgagccaaagacttcattcacgattctctgccccctgttttgactgatagggagagggcactaagtgtttacgggcttccaattcgctgggaggctggagaacctgtaaacgtgggggcccagttgacaacagaaacagaagtccatatgcttcaggatgggatagctcggctggtgggtgaggggggccatttgtttctctattacacagtggaaaactcccgtgtgtatcatctggaagaacccaagtgcttggaaatatacccccagcaagctgatgccatggaactgttgcttggttcttatccagagtttgtgagagtgggggacctgccctgtgacagtgtggaggaccagctgtccttggcaaccacgttgtatgataaggggctgctgctcactaagatgcctctagccctaaattagtttcttgttgattgctggaaacaaggcagtagtgattctccgctgccactgctaccttttttttttttttttccttaaactcacgttcttaccttgataagcatcagtgtgctcacatttacctttatcactgcttcagtgtcacaaacctcggaaggtcttctaggaagaaccatctcatctaggtacaaaaggaaaaggagaagttggaggtggaaaaaaaacccttgatccgtgatcatttcagagcaccaacttcatcaccttcaggcttcagtgtactgggtaacactgaccatgtcgttctgcttgagacagatattagattttttttggaatttggatctttcatctgagttctttttcatgggcgggtcggggtcagtatcctgtttgttattgttaaatttgtatgaaccttagaaaagttattaaagtgccaaagaatgtttcccttt

SEQ ID NO: 30 - DNA, 4248 bp, Homo sapiens
  mRNA (1)..(4248): NM_004910
agggactgcaggggcgccggcgggcggatcgagaggaaggtcggggccatgggccggcactgcgcctcgggagggtccggccaccgctggaacccgaggccggggctgggggcgctccgggctccgacccacgggccggccggccctgcccgggctgggtgaggggcgcccgcctcaagctagaggaggagcggaggccgcgcgcggcccgccgagcgccttcaggatgctcatcaaggaataccacattctgctgcccatgagcctggacgagtaccaggtggcccagctctacatgatccagaaaaagagccgggaggagtctagtggtgagggcagcggcgtggagatcctggccaaccggccctacacggatgggcccgggggcagcgggcaatacacacacaaggtgtaccacgtgggctcccacatcccaggctggttccgggcactgctgcccaaggctgccctgcaggtagaagaggaatcctggaatgcctacccctacacccgaacccggtacacctgccctttcgtggagaaattctccattgaaattgagacctattacctgcctgatggggggcagcagccaaacgtcttcaacctgagcggggccgagaggagacagcgcatcctggacaccatcgacatcgtgcgggatgcagtggccccaggcgagtacaaagcagaagaggacccccggctttatcactcggtcaagacgggccgagggccactgtctgatgactgggcacggacggcggcacagacggggccccttatgtgtgcctataagctgtgcaaggttgagttccgctactggggcatgcaagccaagatcgagcagttcatccatgatgtaggtctgcgtcgggtgatgctgcgggcccaccgccaggcctggtgctggcaggatgagtggacagagctgagcatggctgacatccgggcactggaagaggagactgctcgcatgctggcccagcgcatggccaagtgcaacacaggcagtgaggggtccgaggcccagccccccgggaaaccgagcaccgaggcccggtctgcggccagcaacactggcacccccgatgggcctgaggcccccccaggcccagatgcctcccccgatgccagctttgggaagcagtggtcctcatcctcccgttcctcctactcatcccaacatggaggggctgtgtctccccagagcttgtctgagtggcgcatgcagaacattgcccgagactctgagaacagctccgaggaagagttctttgatgcccacgaaggcttctcggacagtgaggaggtcttccccaaggagatgaccaagtggaactccaatgacttcattgatgcctttgcctccccagtggaggcagagggaacgccagagcctggagccgaggcagctaaaggcattgaggatggggcccaagcacccagggactcagagggcctggatggagccggggagctgggggctgaggcatgcgcagtccacgccctcttccttatcctgcacagcggcaacatcctggactcaggccctggagacgccaactccaagcaggcggatgtgcagacgctgagctccgccttcgaggccgtcacccgcatccacttccctgaggccttgggccacgtggcgctgcgactggtgccctgtccacccatctgcgccgccgcctatgcccttgtctccaacctgagcccttacagccacgatggggacagcctgtctcgctcccaagaccacattccactggctgccctgccactgctggccacctcatcctcccgctaccagggcgccgtggccaccgtcattgcccgcaccaaccaggcctactcagccttcctgcgctcacctgagggtgccggcttctgtgggcaggtcgcactgattggagatggtgttggtggcatcctgggctttgatgcactctgccacagtgctaacgcgggcaccgggagtcggggcagcagccgccgtgggagcatgaacaatgagctgctctctccggagtttggcccagtgcgggaccccctggcagatggtgtggaaggcctgggtcggggcagcccagaaccctcggccttgcctccccagcgcatccccagcgacatggccagtcctgagcccgagggctctcagaacagccttcaggcagcccccgcaaccacctcctcctgggagccccggcgggcaagcacggccttctgcccacccgctgccagttccgaggcacctgacggccccagcagcactgcccgccttgacttcaaggtctctggcttcttcctcttcggctccccactgggcctggtgctggctctgcgcaaaactgtgatgcccgccctggaggcagcccagatgcgcccagcctgtgaacagatctacaacctcttccacgcggctgacccctgcgcctcacgcctcgagcccctgctggccccgaagttccaggccatcgccccactgaccgtgccccgctaccagaagttccccctgggagatggctcatccctgctgctggccgacactctgcagacgcactccagcctctttctggaggagctggagatgctggtgccctcaacacccacctctactagcggtgccttctggaagggcagtgagttggccactgaccccccggcccagccagccgcccccagcaccaccagtgaggtggttaagatcctggagcgctggtgggggaccaagcggatcgactactcgctgtactgccccgaggcgctcaccgcctttcccaccgtcacgctgccccacctcttccacgccagctactgggagtccgccgacgtggtggcgttcatcctgcgccaggtgatcgagaaggagcggccacagctggcggaatgcgaggagccgtccatctacagcccggccttccccagggagaagtggcagcgaaaacgcacgcaggtcaagatccggaacgtcacttccaaccaccgggcgagcgacacggtggtgtgcgagggccgcccccaggtgctaagcgggcgcttcatgtacgggcccctggacgtcgtcacgctcactggagagaaggtggatgtctacatcatgacgcagccgctgtcgggcaagtggatccactttggcaccgaagtcaccaatagctcgggccgcctcaccttcccagttcccccagaacgcgcgctgggcattggtgtctaccccgtgcgcatggtggtcaggggcgaccacacctatgccgaatgctgcctgactgtggtggcccgcggcacggaggctgtggtcttcagcatcgacggctccttcaccgccagcgtctccatcatgggcagcgaccccaaggtgcgagctggcgccgtggacgtggtcaggcactggcaggactccggctacctgatcgtgtatgtcacaggccggccggatatgcagaagcaccgcgtggtggcatggctgtcgcagcacaacttcccccacggcgtcgtctccttctgcgacggcctcacccacgacccactacgccagaaggcaatgtttctgcagagcctggtgcaggaggtagaactgaacatcgtggccggttatgggtctcccaaagatgtggctgtatacgcggcgctggggctgtccccgagccagacctacatcgtgggccgtgccgtgcggaagctacaggcgcagtgccagttcctgtcagacggctatgtggcccacctgggccagctggaagcgggctcgcactcgcatgcctcctcgggacccccgagagctgccttgggcaagagcagctatggtgtggctgcccccgtggacttcctgcgcaaacagagccagctgcttcgctcgaggggccccagccaggcggagcgtgagggcccgggaacaccacccaccaccctggcacggggcaaagcacggagcatcagcctgaagctggacagcgaggagtgaggcccacaccagcctggacctgggttatttattgacacacccaaggggcccgaggggctgcgtgtgggaggctggggacccagacttttggccccagcgctggcccccccagccccacaccctatatctccgtgtgctcctcggtgttacttccctttcatatgaggggacccagcgccggggggagggaggagggcgtgggcatgggcgcagaggcttttccagtgtgtataaatccatgaaaataaacgccacctgcaccccaaaaaaaaaaaaaaaaaaaaaaa

SEQ ID NO: 31 - DNA, 2266 bp, Homo sapiens
  mRNA (1)..(2266): NM_015945
atccggcctcgcacttccggtggggagattccggcctggagctcccagggccgagcagaccttgggacctgtgagcgctgcatccaattaaccatgggaagggtcagcaccagccaccagccccttaggtgaggactctgcctggggctctgctgatggttccgaatcatggagctgcagagagctcctccagcctggagacgttcttggtgaaagctgtggtctaactccaccggctcttcctgcacattgtattcaagaggggtgcctgcccccgctgactcaggagctccggtgctgcagccgccacgaatggggaggtgggccctcgatgtggcctttttgtggaaggcggtgttgaccctggggctggtgcttctctactactgcttctccatcggcatcaccttctacaacaagtggctgacaaagagcttccatttccccctcttcatgacgatgctgcacctggccgtgatcttcctcttctccgccctgtccagggcgctggttcagtgctccagccacagggcccgtgtggtgctgagctgggccgactacctcagaagagtggctcccacagctctggcgacggcgcttgacgtgggcttgtccaactggagcttcctgtatgtcaccgtctcgctgtacacaatgaccaaatcctcagctgtcctcttcatcttgatcttctctctgatcttcaagctggaggagctgcgcgcggcactggtcctggtggtcctcctcatcgccgggggtctcttcatgttcacctacaagtccacacagttcaacgtggagggcttcgccttggtgctgggggcctcgttcatcggtggcattcgctggaccctcacccagatgctcctgcagaaggctgaactcggcctccagaatcccatcgacaccatgttccacctgcagccactcatgttcctggggctcttccctctctttgctgtatttgaaggtctccatttgtccacatctgagaaaatcttccgtttccaggacacagggctgctcctgcgggtacttgggagcctcttccttggcgggattctcgcctttggtttgggcttctctgagttcctcctggtctccagaacctccagcctcactctctccattgccggcatttttaaggaagtctgcactttgctgttggcagctcatctgctgggcgatcagatcagcctcctgaactggctgggcttcgccctctgcctctcgggaatatccctccacgttgccctcaaagccctgcattccagaggtgatggtggccccaaggccttgaaggggctgggctccagccccgacctggagctgctgctccggagcagccagcgggaggaaggtgacaatgaggaggaggagtactttgtggcccaggggcagcagtgaccagccagggcaaatggcttagaagcaggccactccccagcctgctgccagcactcactgtgctcaagccgccagggctcatcatggtagctgggagctgtggacgggagtcaccaggtggtggggccaagccagggactcatgacttttgcccctcccttcagagcctggtcacacaaggggcgagcaccaggccagcctgggactggccagagctgggcccaagctgcgctggaatcgcagcaggagaggggagtgggctggttcttcccaccacttcccaggctctgacagccgagactcatttccaaggcacagcagctttctaaagggactgagtttggactgggttttggacctccaggggctggagcttcatcacctgggcagtgtcttttctcagagagcaggtttctttatagtttggaaataaatggttcacggtccactggccgccttgtgttgctggagacgtgggggcagggaggggacagtgtgggcctggcctctcctttcctttccctgcctggagccttcttcaaatgtctggtcttaagccaggcctccttcattttctcgctcctgttagaacaccagtcccctccccagtggggccccactgcacctgctggcaggaaataaatgaatgtttactgagtactgcattctggagaccttacatgttttcacagcctagtttgaatactggctttgtcactagctgtgtgaccctaagcaaatgacctaacctgtctgtgccgtagttttttaatctgtgaaatggggataatgtctatctcagagtccttttgaagattgagtcattattagtaacagattaaatgttatataagca

SEQ ID NO: 32 - DNA, 2823 bp, Homo sapiens
  mRNA (1)..(2823): NM_018189
aagtgggaggagactttgcaaatagcaatcttggggcaggggccattttggaagcatgttgcgaggctccgcttcttctacaagtatggagaaggcaaaaggcaaggagtggacctccacagagaagtcgagggaagaggatcagcaggcttctaatcaaccaaattcaattgctttgccaggaacatcagcaaagagaaccaaagaaaaaatgtctatcaaaggcagtaaagtgctctgccctaagaaaaaggcagagcacactgacaaccccagacctcagaagaagataccaatccctccattaccttctaaactgccacctgttaatctgattcaccgggacattctgcgggcctggtgccaacaattgaagctgagctccaaaggccagaaattggatgcatataagcgcctgtgtgcctttgcctacccaaatcaaaaggattttcctagcacagcaaaagaggccaaaatccggaaatcattgcaaaaaaaattaaaggtggaaaagggggaaacgtccctgcaaagttctgagacacatcctcctgaagtggctcttcctcctgtgggggagccgcctgccctggaaaattccactgctctccttgagggagttaatacagttgtggtgacaacttctgccccagaggctttgctggcctcctgggcgagaatttcagccagggcgaggacaccagaggcagtggaatctccacaagaggcctctggtgtcaggtggtgtgtggtccatgggaaaagtctccctgcagacacagatggttgggttcacctgcagtttcatgctggtcaagcctgggttccagaaaagcaagaagggagagtgagtgcactcttcttgcttcctgcctccaattttccacccccgcaccttgaagacaatatgttgtgccccaaatgtgttcacaggaacaaggtcttaataaaaagcctccaatgggaatagaatatcaggaaaaaggccacatctatggtaattaatggcagaaaagctggagagttggattctgcggtgctgctgacaggtgaactctggtcctctgcacctgtttatgggccatgcagactggtggggtggcagatgttagcctaagacccctagcagtgcctgttgctttgtgagtggagatagagactcttacatttaaaaatggaaaaacatttcacaaattaccataaattgtagttaatatgtagaaaaactcattcatactacttttctaaaatagacatgacttcagcagcagcttttttttgttgtattttgagacagtgtctcactgttgcccaggctggagtgcagtggtgcaatctcagttcagtgcaatctccgcctcctgggttcaaatgattctcctgcctcagcctcctgagtagctaggtacaggcacctgccaccacacccagctaattttttgtatttttagtagagatggggtttcaccatgttggccaggttggtctcaaactcctggactcaagtgatcaccctcctcagcctcccaaaatgctgggactatgggcatgagcccctgcgcctgaccttcaacagctcttttaagtgagttcttcagctaagcattgtgatggacttgagtaaaatggtagttggctcttgtgctcaattttctcttcctctgaacactgactactttaggagctgcttcattccaattgcaatttcataaaacgtaaagtattttaaggcaaagaaaggctgttaattccctccctcccccaaacacatgatttttaatattctaaacaatatttttcaaagttctcttaataacctgagatttctatggtttgactccaggatcaaaacacaagggactttgtattatttcacttataattgttttgtatatttctggagtttaaaatgtttaaggttgcttcccgctcataaatacataatatattgaatttaaaatgtgtttattaaccgattctccataaataaaaataagatgtgtatgtaaaataattcatctgttgtatttagagaaccatattcattgcatgcaaattttattgttagtgttcttaactcaagtaggagtaaaccaaaaagtgtgatttttcttttgtatgactcgtttgttctttattagttggtggtatgggttggatcatttgtttttaaaactacttaggtatgattcacatacaaaaagctgcacatatttaatgtatcctattgtgtaattaatttttaatttttttgtgtacttcctaaacttatagtcctgcgagtctgggaacagatctgtttttcacttatcctgatttaatgacagtttccaacattgttttgttattacaagtaggggatctttttttttgcccgtttaatgaagatactaaaaataatgcactggaaggagtggaagagttggaaaatttgtaaccatcataatacaggtgtaataggtttgggaaagaatcctcaaaaatgttaaagcaagggaggaaagtttgttgagaagcaagatgttcttctctcctgcccgcccccgccgttggttgttggtggtcagaattattgtgtaataaataatagacattttttcttatactatgtgtattgttccttttgtttcctttttaaacttctcccctgctttatttggatgggtcaagtttctgttctgtttccttcctttctattaatttggaaatgtccttggctttacgattctgcttgtagatacttcccctgcttctaacacatttcaataaacttaaatttctctatatacaaaataaattaataattggagtctaccaaaaaaa

SEQ ID NO: 33 - DNA, 835 bp, Homo sapiens
  mRNA (1)..(835): NM_207373
aggacttctgcagcacagctcccttcccaggacgtgaaaatctgccttctcaccatgaggcttctagtcctttccagcctgctctgtatcctgcttctctgcttctccatcttctccacagaagggaagaggcgtcctgccaaggcctggtcaggcaggagaaccaggctctgctgccaccgagtccctagccccaactcaacaaacctgaaaggacatcatgtgaggctctgtaaaccatgcaagcttgagccagagccccgcctttgggtggtgcctggggcactcccacaggtgtagcactcccaaagcaagactccagacagcggagaacctcatgcctggcacctgaggtacccagcagcctcctgtctcccctttcagccttcacagcagtgagctgcaatgttggagggcttcatctcgggctgcaaggaccctgggaaagttccagaactccacgtccttgtctcaattgtgccatcaactttcagagctatcatgagccaacctcaccccacagggcctcagtcgccaccatgtgggcctctccagtgcaaaccaccgagcattccaccatgaccggtcacagctacaaatccagagaccatcaatcctgctagagtgcagggtggcaagcacccaagggtggctgaccaagactgcagagtctcctccatcttcaggtccattcagcctcctggcatttaactaccagcatccagtggtccccaaggaatcccttcctagcctcctgacatgagtctgctggaaagagcatccaaacaaacaagtaataaataaataaataaactcaatgcagacacaaaaaaaa

SEQ ID NO: 34 - DNA, 480 bp, Homo sapiens
  Unsure (1)..(480): NM_001311
agcgctgggctaggggcgcggcttgaactcgcctaaagagctgcgccctctcatctcgcgcctgcagcccgtgccgccccagccgctgccgcctgcaccggacccggagccgtcatgcccaagtgtcccaagtgcaacaaggaggtgtacttcgccgagagggtgacctctctgggcaaggactggcatcggccctgcctgaagtgcgagaaatgtgggaagacgctgacctctgggggccacgctgagcacgaaggcaaaccctactgcaaccacccctgctacgcagccatgtttgggcctaaaggctttgggcggggcggagccgagagccacactttcaagtaaaccaggtggtggagaccccatccttggctgcttgcagggccactgtccaggcaaatgccaggccttgtccccagatgcccagggctcccttgttgcccctaatgctctcagtaaacctgaacacttggaaaa

SEQ ID NO: 35 - DNA, 1266 bp, Homo sapiens
  mRNA (1)..(1266): NM_015957
ctcagcgccgcctgattgcatttgcggcctcgctgccgtatcccaggctaagcgccgcgcgcaaagccgtgcggagattggaggccgcgcgggtccctggtctgggccatgtctggctgtgatgctcgggagggagactgttgttcccggagatgcggcgcgcaggacaaggagcatccaagatacctgatcccagaactttgcaaacagttttaccatttaggctgggtcactgggactggaggaggaattagcttgaagcatggcgatgaaatctacattgctccttcaggagtgcaaaaggaacgaattcagcctgaagacatgtttgtttgtgatataaatgaaaaggacataagtggaccttcgccatcgaagaagctaaaaaaaagccagtgtactcctcttttcatgaatgcttacacaatgagaggagcaggtgcagtgattcatacccactctaaagctgctgtgatggccacacttctctttccaggacgggagtttaaaattacacatcaagagatgataaaaggaataaagaaatgtacttccggagggtattatagatatgatgatatgttagtggtacccattattgagaatacacctgaggagaaagacctcaaagatagaatggctcatgcaatgaatgaatacccagactcctgtgcagtactggtcagacgtcatggagtatatgtgtggggggaaacatgggagaaggccaaaaccatgtgtgagtgttatgactatttatttgatattgccgtatcaatgaagaaagtaggacttgatccttcacagctcccagttggagaaaatggaattgtctaagccaaaagaaagtctaattatatacagagataaagctaaacgtaattattatttaaatgaaagctatttttttaaatgaattgaaatttttcatgatgctactaatttgccactaaatactgcaaatggtcaccctgaatctcttctgacattggatgttatttgcttatattcttataattttaaatgagggcacagtgaaatgaaaattttatactctatgtttctgtttatttttaaatccttaacagcaaaatatttgcctttaatttcttttttatatatactctcagagaattcctcttaatttttaaagatgctggtgataataaaattcattagaaaatttcctcattgtggaatgagcattctcttgttttaatgttggtgtcagaaaataaatatgaaacattaagtccaaaaaaaaaa

SEQ ID NO: 36 - DNA, 5089 bp, Homo sapiens
  mRNA (1)..(5089): NM_018235
gtggcgggggcgtggcgatgcctgccctccggacaaggcggggttggtggccagggcgaccgcggggcgggccagcgccgggggtcgtggccgggacacgtggtacggaaccggcgccgcgcttgctgctggtaacagggccttgcctagtgggccttccttcccaggtcgcccctcagtctccactagagacaggactgaccagttgctcttccttccaagaaccttcgagatctgcggtctggggtctggttgaaagatggcggccctcactaccctgtttaagtacatagatgaaaatcaggatcgctacattaagaaactcgcaaaatgggtggctatccagagtgtgtctgcgtggccggagaagagaggcgaaatcaggaggatgatggaagttgctgctgcagatgttaagcagttggggggctctgtggaactggtggatatcggaaaacaaaagctccctgatggctcggagatcccgctccctcctattctgctcggcaggctgggctccgacccacagaagaagaccgtgtgcatttacgggcacctggatgtgcagcctgcagccctggaggacggctgggacagcgagcccttcaccctggtggagcgagacggcaagctgtatgggagaggttcgactgatgataagggcccggtggccggctggataaacgccctggaagcgtatcagaaaacaggccaggagattcctgtcaacgtccgattctgcctcgaaggcatggaggagtcaggctctgagggcctagacgagctgatttttgcccggaaagacacattctttaaggatgtggactatgtctgcatttctgacaattactggctgggaaagaagaagccctgcatcacctacggcctcaggggcatttgctactttttcatcgaggtggagtgcagcaacaaagacctccattctggggtgtacgggggctcggtgcatgaggccatgactgatctcattttgctgatgggctctttggtggacaagagggggaacatcctgatccccggcattaacgaggccgtggccgccgtcacggaagaggagcacaagctgtacgacgacatcgactttgacatagaggagtttgccaaggatgtgggggcgcagatcctcctgcacagccacaagaaagacatcctcatgcaccgatggcggtacccgtctctgtccctccatggcatcgaaggcgccttctctgggtctggggccaagaccgtgattcccaggaaggtggttggcaagttctccatcaggctcgtgccgaacatgactcctgaagtcgtcggcgagcaggtcacaagctacctaactaagaagtttgctgaactacgcagccccaatgagttcaaggtgtacatgggccacggtgggaagccctgggtctccgacttcagtcaccctcattacctggctgggagaagagccatgaagacagtttttggtgttgagccagacttgaccagggaaggcggcagtattcccgtgaccttgacctttcaggaggccacgggcaagaacgtcatgctgctgcctgtggggtcagcggatgacggagcccactcccagaatgaaaagctcaacaggtataactacatagagggaaccaagatgctggccgcgtacctgtatgaggtctcccagctgaaggactaggccaagccctctgtgtgccatctccaatgagaaggaatcctgccctcacctcacccttttccaacttgcccagggaagtggaggttccctctttcctttccctcttgtcaggtcatccatgactttagagaacagacacaagtgtatccagctgtccacgggtggagctacccgttgggcttatgagtgacctggagtgacagctgagtcaccctgggtaagttctcagagtggtcaggatggcttgacctgcagaagatacccaaggtccaaaagcacaaggtctgcggaaagttctggttgtcggccgggcaccacggctcacacctataatcgagcactttgggaggccaagacaggaggatcacttgaggccaggagtctgagacaagcctaggcaacaaaacaagactctgtctctacaaaaagtttaagaaatgagccagacatggtggtgtatgcctgtagtcccagccactcagaaggctgaggcaggaggatcgcttgagaccaagagtttgagcctgcggtgagctgtgaatgcaccacggcactcaagcctgggcaatgtagcaagatcctgtctctacaagaaattttttaaaaatgagccaagtgtggtggtgcatgcctgtagttccagctactcaggacactgacgtaggagggttgcttgagactgagagttggaggctgcgatgagccatgaatgccccactgcactccagcctgggcgacagaacgagaccccatctcaaaaaaaataagttctggttgtcattgaattgggataaacagagagcttgatgctttctgccttctgtctcaggtgatgcattgcacatttgggatatttggaaaggaaatgaggaaagaaattagggcctcctctgatctctcgctatctgcgggtcctgtccttttctcaagaccttcaccattactggcattttcctgtcttctctttagtatgatccctcaaaacctcactaactggaaggatgattttgtctcagtttgtactcctaaataaaaagtaaacatgacacctctaaaagaatcgctccatctcagttgctcgtcgcgtgcacaccatgcttattgagtggtgtctggttggtggtgtcagctcagggcaggggacagcaggcaggacccggaaaacagaacccaggtcttcccaggcgagcactccctccctcgtccacacaagtggaggctcactcgtggccccctgcatgttccgagaagtgtccctgtctctgctccttaacggctgtggggccacaggaagtccagctggcttaaacaaagaattgattggcccacttgacataaaagaacagagtaaactcaatgaaaggaaaaggctccttgaagtggttggttccagggctgggcagggaagatgcaagatgagtctggaccctctcacatcggaacgtaaggatgcgctcctgagggcgtggggctgcgtccaaggacacaggagccagcctggaagagtttgcaggggcccaggggagcaattggagtgagaaaatagcagtagtgttggatttttaccatggaataaacaacaatccatgagtccatactaatataaataaagtgacaaagtgggtgagaaggaccagtgcccctggatgaagaatctctgctcatgtgggaggaaggtgggagatcaaagctggagcacttgctgctgcaggtgagacccctgacacactctcaaattggtaggcaaaggtttaaagaacgggatatttgcttagcttaaaagcacctcccaaggggaaaacagtaacttcacagtggagagacctggcaggcaccaccctcagatcaaaatgaacctcagcagtaatgacacgtccacatcagcacccccaacaccactccctgaggaggacatgccagttctgggaacatcacactcatcgtgacaaaacatcagacaaacccaaacggatgaaatgtgaggaaaatctgtagttggttttacgtggatgttaatctcttagttttgatcattgtgctgtagctgcataagaggttagctgggtgaagtgtatctggggaccctctatggcttttgcatcttttctctaagtctcctgtcatttcaaagtaaaaacaaaaaaagaagacaagggtagcggcttcaggtacagctggacccagcactcagtgctatctgggcttgtggctccaactcaactcctccatcagaactgtgagaaatgcattctttcagcttctaatcctgcaaaaagaactgcatggtcttcccccagaaatcctaagagtagctgctggtgtctcatgcatgcgagctgcatatgggcccccgttagtcaggaacccatgctgagaggtgctatgctttgaatgtttggctcctcccaaactcgtgttgaaacttaatagccattgtaacagtattaagaggcagggccttcaagagctggtcaggccatgagggccctgtcctcctgactgagtgaatgctgttattgtaggagtgggcttgtttggcctcctcttgctctctctacccctctctgagcccttgtgttgtgggatgacacagccagaaggcccttgccaggttccggtccctccaccttggacctcccagcctcccgaacgttgagccaatacatctctgctcattgtaaattacctagtcttgggcattgtgttacagcagcaaaaagcagcctacggcagtcaccgtgctaacacctgcagcagcctgcggcggtcaccgtgctaacacgtgcagcagcctgtgtggctcaggccagtcagcttcccctggaagcaagtgtgaggtcgtcgcctctggaagtgctagcggagggtgatctcccggaggaaatcagagtggtgttctactagaaggccgatgactcctgggtaaagtgccaatgctcacctccatctctgcaagctcagtccctcgtggaatttccaggagcacaagggcaatctcgacagctccacgttcccagtgcccgacccaagcctggcccatgatcagataaatgcctaagtggggtggcatggacatttgcacacgtccagactgctttatgttagaagctgagctggcatgccactagctggttttgatgtaaacaacaattttgtaaaaccctggactgaatgggtctgactaacggctggttctgtgaacttgagcctcagcgtcctgtgtcagaagagcacaaaccactttcccagagaggttatgaagacagaaatgaccctgtgacccctttgcaaagtatgaagaacggttcccactcatgcatggagctggatcttctcaagtgggaaacaggcgtggtaaataaatactgttgtttcagctggtgtaaaaaaaaaaaaaaaaaa

SEQ ID NO: 37 - DNA, 2542 bp, Homo sapiens
  mRNA (1)..(2542): NM_173623
agtctaagggcacagaatggaaaggttctcattttcctcctccaatgagaattggcaccccaacagcaggccctgggccctcggctgtgatggaggggtgtctcggggtagcagaactaaggaagctctccaccttcagtgcatacttggaggaccacagctacaacgtggagcagatatggagggatattgaggacgtcatcatcaagaccctcatctcggcccaccccatcatcaggcataactaccacacctgcttccccaaccacacactcaacagcgcctgctttgagatcctgggctttgacattttgttggaccacaaactcaaaccctggctgctggaggtcaaccactctccaagcttctccaccgactctcggttggataaagaggtgaaagatggtctgctgtatgacaccttagtcctgatcaacctggaaagctgtgacaagaagaaagtcttggaggaggagagacaacgggggcagttcctgcagcagtgttgttctcgggagatgaggattgaggaagccaagggtttccgggccgtgcagttaaagaaaactgaaacgtatgagaaggaaaactgtggagggttccgactgatttatcccagtctgaattcggagaagtatgagaagtttttccaggacaacaactccctcttccagaatactgttgcttccagggctcgggaggagtatgcccggcaactgatccaggagctgagactaaaacgggagaaaaagcccttccaaatgaagaagaaggtagagatgcagggggaatcggcaggcgagcaagtgagaaagaagggcatgaggggctggcaacagaaacaacagcagaaagacaaggccgccacccaagcctccaaacagtacatccagccattgacattagtatcctacacacctgacttgctcttgagtgtcagaggtgaaaggaaaaatgaaacagacagcagcctcaaccaggaggctcccacggaggaggccagctctgttttccccaagctgacgtctgcgaagcccttcagttctctacccgatctgaggaatatcaatctcagcagctcgaagttggagcccagtaaacccaacttcagcatcaaggaggccaagtctgcctctgcagtgaacgtattcactggcactgtgcacttaacctccgtagaaaccaccccagaatccaccacccaactctcaatctccccaaagtctccgccaaccctggctgtgaccgccagctctgagtacagtggcccagagacggacagggtggtatcctttaaatgcaagaagcagcagacccctccacacttaacccagaagaaaatgttaaaatcttttctgcccacaaaatccaagagcttctgggagagtccgaacacaaactggactttgctaaagagtgacatgaacaagccacatttgatatccgagctactcaccaagcttcaactgagtgggaagctctccttcttcccagctcactacaaccccaagctggggatgaataacctgtcacaaaacccctccctgcctggggagtgccactcccgcagtgacagctctggcgagaagaggcagctggatgtgtcctccctcctcttgcagagtcctcagagctataatgttactctgagggacctgctggtgattgccactccagcccaactggatccaaggccttgtagaagccacgcaagtgctatgagggacccatgtatgcaggatcaagaagcatacagccattgcctgatctctggccaaaaaggatgtgagaggagctagaatcagactggatctctgccacttttcccttcctacctcttaacttgcaggaaacaatgtgtctcttccgacagtgtctccagtgttcacagaatcaactgaagttttgtttgctcatctagatatactggtcttccattagttcctagtagcagcaatggggccaacagccctgcctctgttccctcccagttagcttacagtgctacaggaccatgatttctggggttggattggtgctgccccaggacacactccagttgccgtaccagtggcacttctctgggttcttcacatggcaccatcatccagaatactcccccagataaagcaacttcgggagtagtgtttcagagaaggactcatgatttccaggagtcaaaagccctcagtccaccaacagaactatgccagttggtcttcgtctccaagataaaagatgaggtcagggatgggcatggtgcataactctgagccctgagtgagagaactgaggcagctgcaacttcacaaggtcaaggcccgacctccgcccatccccaccagcccaaagacttgccacctcagtgcccgagcatcctctggtgacctccctcaccagcccaacttaatctcaccagtgaaaacagaggtgaggcgagagtacccagcctgaagatgcttcttctatcagcctgcagaaatgttacactttcccttcaggcatccattaataaaagaaattgccatgcttactgccagga

SEQ ID NO: 38 - DNA, 4093 bp, Homo sapiens
  mRNA (1)..(4093): NM_016076
ctttccccacgtgtctgccgcctaggggaggtgcctcatccggagcgggccgccaacggtccggccccgtccgcacagacgctcctgtcggcggcgcccgggagcggctcggctgcccgatgcttccgccccggctgccgcgggccgggctgtacgcttagtgcccggctcaggccccctgaagcgcccgcgggggtgagagcggcctccggccccgcggagacggagcggcttgaggacgaggcggcggccgcggggaggaggatgggggctaaccagttagtggtgctcaacgtgtacgacatgtattggatgaacgaatatacctcatccattggaattggagtttttcattcaggaattgaagtctatggcagagaatttgcttatggtggccatccttaccccttttctggaatatttgaaatttccccaggaaatgcttctgaactaggagaaacatttaaatttaaagaagctgttgttttagggagcacggacttcctagaagatgatatagaaaaaattgtagaagaactgggaaaagaatacaaaggcaatgcttatcatttaatgcataaaaactgcaatcatttttcttcagctttatcagagattctttgtgggaaagagattcctcgctggatcaatcgacttgcctacttcagctcctgtataccctttctacagagttgcctcccgaaggagtggctcacgcccgcagccctgcagtctagtgtcagccaagaactccaggatgaactggaggaagcagaggatgctgccgcatccgcttccgtggcaagcactgcagcaggctccagacccgggcgccacactaaactataaatgtctccaaagtcacacattcagaactgtctctggcagtcgaatatcactagagaaaagtaaacagagaagcatcctttagatattttgtatgcaaagatggctctcccccaaatcccagtttttcagctcaggattatatttgtaatcaaaaaaaaaaaatcacttggcgcaggagggagaactttgtaagaagctgccctctgttttttttatccactcgtaaatctggatttatttcttctgttttatacaagctctgttaagttatgtttacagtatcttgtatcgctgtttacaaatcttgcatggactcctgccacagtgaaagaagaaaatcttcatgtcttcaaaaattaggcaggtaatctttgtatacttctgacaattgccagatctatggcataaataggcacacaaaaaggtacttaaacagttatagtcaccatcacctgcttcagaatggtcttttagatttgtgtttgttttgttaaagttgttggcaccaggatgcagagaatcagactggcctgaggtgaaggagcacacagccctgagggcttggaaccctgggtccagttcctcttcacacccccttccactctgagtagcacatctccccaggtgcccatggaacacctgctttcatcccaaatatccgtccacctaggcggggtggtatgttcttacgtctctctgactttgatgccactcattctatagtttagctggttttcgttcaagatattcttggtagtaactgacaagtatgttgcacatgtattgggggaggcgcttcatttttattttaatacacatgtatttcctccttgcacaggattttgatggtgtgggaatatcctaagtggtagccttccaagtagcagtgagttgacattcagctgcttttaactattcaggctaccttttatactaaaccttgaaaactagaatctaatgtctaccccaaaaaagtagttctttgatattttatactttttatgtaccatgtcagaaagagtatgttggcgtttgtcatgggactcatttcacataatagaatgcctagtctcattgaccaatcgttaaaaaatcatatttgtgtgtcttaagattcatatttatatgttctctcaaatgtatgtctcttacgtaacatactctaagaatgaaactgtcaccacaggtaaatccttgttagcaaggaatctgtctgctccagtctactcctagtttgatccttggatgtaagaaccaagtcattacatgtcaaattcaatttttctgccttaagaatgaatgtccttcataaaatattggatgcagtgtaacactatccaaggcagtgacttcagctttatatacatataaaatatagttagttttaaaattattgacatttatttaaacttttagattggattgtttgctattgctctgtgtgaggatacataatctttcagtaaactgtatttttaacttttccataagctgattttggttcattttatcaacgtaagcacaccctgttcatagggaaaataaaccttgggttataagcattagcctgaggacaatgaagccacttaacctaatttatgctttcgactgttctgtttccagagaggaaagcctttacaaattactctcagttctttaggggcagaaggcttgtttcaagaggtttgacagaagaaaggaatatatgaacttaatgagatgtcgacttggttcaggtctaaaaatgagggcaaaacactaaggctctagcagtgacttgttcactaaaaagagagagtcctgtccccagacggttagtacaaagccttggatacagtttgcttgtaatatttttaataatgtgaggagtacagtgttttctaattcattcaagtatatatgatttaaacctgggctactgacacacacacagtagccattagttagactcttcttagtgaatatcaggaacatcccatctgtgcttaaccagaatccagcaagtcagcacacaagtgattttattgttattttgttgtatttacttgcatttgttgtatttactttcatctgcagcatttggagtttaaaaataatgtaaagggttctagtagaaatagtgtcctaaggccaattacctaccatactaacaatcagcagataaaattctggacgtgagattccttataatctaattatacctgaggttgagcaagaaatgtcttcctttagaaaatctcattcaagtcaggttcttctctacagttcaaaattgagaatggatttaattaactagcatttagccagctttttcttgcccttggagaaaaagaatcattctcaacctgataatctgttaagaaaaatcccatatgaacaatctggtcattaacatacatatgatacggagtctctttgttgtcaccaagtgaacatacttctcatggtgggttggacagtaatacatgttagagggtcagaagcttctggtttctgctgtttgctttaaatacccttggggtttttttttaaacccttacaaggggagcatcagctttggaaagtgtgactctgtaggagtgtagaaggcagtggtgtatgatcttagcctcgtcctgatgcctgaatccagccagctgttgctctgacccacagcaatagagcaagttacccatcaccagcatttgtacagagcagggaattctggttttagtccattggtagcattgtgtgtatgaggagattcaacaccacagacagctgcaggactcgatatccatggcttctttccatcacaaaacgggtagaaacacattcactgcttcagggttctaatctgtgtgtctccttatgactccatttctgtaagctactctgtaactttgatatatgctgtattttctttctttaaaagatttagatgttttttcagcaagctagccatacaaccattgtatctctttctcttcagtatggtttagagcccagatcagttagtaggctttcgttgtcttctctttcaatacatgtacatctttactgtttgaaaagtgttacagctgtcaaagaatcttcatggacctgaagataatttcttgtgaagttgaatgcaagtgtactgtcattcatagtgtttatatcaaaataccaggaatcttcacttttgctaccttgatatagcattgggctatcatgttacaacattgaaatacattgatttattaaaaaatacttttataagaaa

SEQ ID NO: 39 - DNA, 3189 bp, Homo sapiens
  mRNA (1)..(3189): NM_016233
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 40 - DNA, 7089 bp, Homo sapiens
  mRNA (1)..(7089): NM_025099
ccccatccccacccccaagcgagcacctgcccctcccgggggcggagctccggcgcatcatggcggctggccgggcccaggtcccttcctccgaacaagcctggcttgaggatgctcaggtcttcatccaaaagaccctgtgtccagctgtcaaggagcctaatgtccagttgactccattggtaattgattgtgtgaagactgtctggttgtcccagggaaggaaccaaggttctacactgcccctcagctatagcttcgtctcagtacaggacctcaagactcaccagcgtctcccatgctgcagccacctgtcgtggagcagtagtgcataccaggcctgggcccaagaggctggaccaaatgggaaccccctgccccgagagcagctgttacttttagggacactaacagacctatcggcagacttggaacaagagtgcaggaacggaagcctctatgtgagagataacactggcgtcctgagctgtgagctcatagacctggacctttcttggttgggccatctttttctgttcccccgttggagttacctccctcctgccaggtggaattcctcaggggaagggcacttggagctgtgggatgcccctgtgccagtgtttcctttgaccatcagtcctggccccgtcacgcctatccctgtcctctacccagagagtgcttcctgcctgctcaggctcagaaacaagctcagaggtgtgcagcgaaacctggctgggagtctagttcgattgagtgctctggtgaaaagtaaacagaaagcttacttcatcctgtctcttggtagatcacacccagctgtcacccacgtgtccatcatcgtgcaggtccctgcccagctggtgtggcacagagcccttcggcctggtacagcctatgtgctgacagaactgcgagtgtccaagatccgtggtcagcgccagcatgtttggatgaccagtcagtcctcccgtctgttgctgctgaaaccagaatgtgtgcaggagctggaactggagctggaaggacccctcttagaggctgaccccaagccactccccatgcccagcaactcggaggacaagaaggatccagaaagtcttgtccggtattctagactcctatcctattcgggagcagtcactggcgtgttgaatgagcccgctggcctctatgagctggatgggcagctggggctctgccttgcctaccagcagttccgtggccttaggcgggtgatgcgacctggagtgtgtctgcagctccaggatgttcacctgctccagtcagtgggaggggggacaagaaggccagtgctcgccccctgcctccgtggcgccgttctgcttcaaagcttctctcgtcagaagcctggggctcactcatcccgtcaagcctacggggcctccctgtacgagcagctggtgtgggaacgtcagttaggacttcccctctacctgtgggctaccaaggccctggaggagctggcctgcaagctgtgtccccatgtgctgagacaccaccagttcctgcaacattcctctcctgggagccccagcctgggactgcaactcctggctcctaccctggatcttctagctccgccaggcagccctgttcggaatgcacacaatgagatccttgaagagccacatcactgtcccctccagaaatacactcggctgcagactccctcctccttccccactctggccaccctgaaagaagaaggacagcgtaaggcctgggcctcctttgaccctaaggcccttctgcccctcccggaggcctcctacctgcccagctgccaactcaatcgccgcctggcttggtcctggctctgtctgctgccctctgccttctgcccagcccaggttttacttggggttctggtggcttcatctcataaaggttgtctgcaacttcgggaccaaagtggttccctgccctgcctgctcctggccaagcactctcaacccctcagtgacccacggctgataggctgcctggtgcgggcagagaggtttcagttgatcgtagagagggacgtgagaagcagcttcccttcctggaaggagctgagcatgccaggcttcatccagaagcagcaggccagagtctatgtccagttctttctggctgatgccctgatcctgcctgtgcccagaccctgccttcattcagcaacaccctcaacacctcagacagatcccaccggcccagagggaccccacctaggacagagccggctcttcttgctctgccacaaggaggccctcatgaagcgtaatttttgtgtccccccaggagcaagtccagaggtgcccaagcccgccctcagtttctatgtgttggggagctggcttgggggcacccagaggaaggagggtactggatgggggctgcccgagccccagggaaatgacgacaatgatcagaaggttcacctcattttctttggctcttcagtccgctggtttgagttcttgcacccgggacaggtgtaccgactcatagctcctggccccgctacaccaatgttgtttgaaaaggatggttcatcctgcatatctcggcgtcctctggagttggctggctgtgcatcctgcctcactgtccaggacaactggactctggagcttgaaagctcccaggatatccaagatgtgctggatgcaaacaagtcattgcctgaatcctcactgaccgacctgctcagtgacaatttcacagattccttggtgtctttctccgctgagattttgtcacggacactatgtgaaccccttgtggcgtctctctggatgaaactggggaacacgggggccatgagaaggtgtgtgaagctaacagtcgctcttgagactgctgaatgtgaattcccccctcacctggatgtatatatagaagacccacacttgcctccctcactaggactacttccaggagcccgggtccacttcagccagttggagaaaagggtttccagatctcacaatgtttattgttgtttccggtcatccacttatgtgcaggtcctgagttttccccctgagaccaccatcagcattcccctgccccacatctacctggctgaacttctgcagggtggtcagtccccattccaggccactgcctcttgccatatcgtctctgtcttcagccttcagctcttctgggtgtgtgcttattgtaccagcatctgccggcagggaaagtgcactcgcctgggctccacttgccctacgcagacagctataagccaggccatcatcaggctcctggtggaggatgggactgccgaagccgtggtgacctgtaggaatcaccatgtggcagcagcactagggctgtgtcctagagagtgggcctccctcctagatttcgtccaagtgccaggcagagtggtcttgcagtttgcagggcctggagcccaacttgagtcttcagccagggttgacgagcccatgaccatgttcctctggacactttgtactagcccctctgtcctccgtcctattgtgctttcttttgagctggaaaggaaaccgtcgaagatcgtcccattagaacctcctcggctacagcgattccagtgtggagagctccctttcctgactcacgtgaaccccaggctccgattgtcctgcctttctatccgagagtcagagtactccagctctctggggatccttgcttcctcctgttaactgaactgcaaggatggcctgagagtccttccttgctgaaaacctgaaggcctaggtcctggctcctctccctacttgttctgtgattgaaccaaggactccagattcacaaactgctactccactataatttcccttctttggtgttctggtgccagcctgccttggcaaaattgtggcaacaggaaatcatggccctattaatatgtcctctgattgggacaaggcacctgcattcacaggcggccctgagcacctgggttctgactttgtcgcaggagctgagggaacaaaagacttgctcgctgtggggtgatggtgacaggcctattgacctgcaatgaagccctctggtgtcatttctcactggtactcatctctggggtcccaggcctcctgactcctagttgtccacctcctaggaactcctagtcgttcatcatcatttcagccctttgccgccagggccaaaggtggaaagtgatttggaagagaagagcttttcgtccagcagaagaaatggtaccaaaatcagtctgtaaaggaagtaaattggggagttggcggcaaggcagagagcatagctatgatggtctcagtcagtaaggctgggccctgcaggaagtcaaatatgaatgcctagaggtgttcacaagcacaaagaaggtgtagggggaggcaagtgccaggcacaagcaggggcaggtgaggactctgggtgactgtgctatagggccccaggctacggttagccctgtaggtttcccaagggcctggcctaaggcagatccttgatcgatataccttgagaccagaaggtgctccgaaatcacaaccgtacaattaggggatctaggaacaattctttggagataccaatgcctttggctggtgttgctgcatttctttactggggactgatagatggagaggtggaaagatgagctgaggcacatctttcagagctactgggaggccatttcttcctggctgttaggatttgttcgtgtttgggagacctttagagcgtggttaaacccatatgttgggatttatgctgcttttatggtagcaataccctatattaagatttgaagtagacccggaaagttagtggccggttagctcagttggttagagcgtggtgctaataacgccaaggtcgcgggttcgaaccccgtacgggccagtgggtggcttttttttgtgtgtgttttgttttctgaccctctgctgttatccggaagtttctacccggagccagttgccttctggtaacagaattatattgcacctactgcttcctattccctgaatcactagcgctcccgaaggcttggagggaggagtctctgggcacccgggtgaaaggaaggttcacgtgcaaccgccgcgtctttttttccctgaagcgctttcatggaggcagatgtttgtcagtcaagggaagtaagaaaggcattggatgaaaacgaagccctaagcctcgtagtcgtggccgagtggttaaggcgatggactagaaatccattggggtctccccgcgcaggttcgaatcctgccgactacgtcatatttttttcttcagcatactgaccatatttctctccaggatgggatgatccagtcggcaccctccaaacctctcatctaggaactctagaatcgagaatttgatttagagtctatgattttggtttgaaatctatgattaacgtctttggacattgaaggaaatccgaggaatggacaagtgatgcaagagccagttgagttaccaaattagttctagaaagatctgaaaaagctcggtccgggttcctagctctatattcttgtagatgaatttcaggaacctttatggcagcttcggcgccgtggcttagttggttaaagcgcctgtctagtaaacaggagatcctgggttcgaatcccagcggtgcctttattcaattgaaacagcgtgattttgcggctaaatccacatcctttcatgtattgtttttatatcagaacgcgtaagagtttctgttctgcactcatagcacccacatttcctgcagagtcaagctgccactcccatgagatcgccactctaaaaggtggttctctaacttaggggcagaaatgttgcataggcctaagggtcctttgcttaactgatgccacaccccactggtgcaggtggactgggtcaggcggccgccccaccctcgatggaaggggctgcccaccttccaggcctctttctccaccctaggacgtcccctagaacctgagccactttgttttgtttggctcttcattatagttctttgggtttggtggctcataatgttatatatatagtatataatataaaaatatatatttaaatatacagtatttaaaatttggcacagcttccagatgcggtcctctaactggtctttcacttgcagttacctccatccctctccaccagcgggatgtcagggtaaggagtaagcagggatccggctggcctggcctggcctggcaccaggtttcgtgcagcagggtgcagaagggctgaggccatgtgaacagagtccaagaaagcatcattcgggagtcgctagggatcctggtgtggaagggcagggcacttttctggagcactgaagctaggctggttaaggaagaaataaatgccagagataaggcaagaaataggatctgtgagctcttggcaggacctaaacctccttggaagataggcagaaagctctcgacaccattccatggcccacgaaccaatgtaagatgagcaaatggcttgaaggaattgctacctccaggtcaagccagggatgcagcactgccgagaccacgtttgtgccaagcactgggctggaccctgtgcagaaccaaatgaacaaggcacgttcccctttcagcactaacggcactgtaagaacagggagaagtggaatctaatctggcctgagggtagagggtgatcagctaagtctgaaacaccatgtagaaacttgccatgtatggccgggcgcggtggctcacgcctgtaatcccagcgctttgggaggccaaggtgggcggatcacgaggtcaggagttccagaccagcagcctggccaacatagtgaaacctggtaacatagtgaaacctcgtctctactaaaaatgcaaaaaattagccaggcgtggtggcaggcgcctgcagtcctagctacttgggaggctgaggcaagagaatcgcttcaaccttggaggggggaggaggtgttgtcagccgagatcgcgccactgcatcccagcctgggcaacaagagtgaaactccgtctcaaaaaaatgaaataaaataaacgaatgatcaaaaaaaaaaaaaaaaaa

SEQ ID NO: 41 - DNA, 5545 bp, Homo sapiens
  mRNA (1)..(5545): XM_035299
atggcatcgtggcgtcgcgacggcgtgtgcgtgtcgcgcttcctagtgccgtttatagggtcccggcacttccgctgtcgggttagaagcggcgcggtcatggcggagcgcggacagcagcctcctcccgcgaaacggctttgctgccggccgggcggcggcggcggcggcgggggcccgcgggcgggtggcgcggcggcggcggcggcgtgcgggggcggcgcggcgctggggttgctgccgccgggcaagacccagagccccgagtcgctgctggacatcgcggcgcgcagggtggcggagaagtggccgttccagcgcgtggaggagcgctttgagcgcatcccggagccggtgcagcgccgcatagtctattggtccttcccccgcagcgagcgggagatctgcatgtactcgtccttcaacaccggcggcggcgccgcgggcggccccggcgacgacagcggtggcggcggcggcgcgggcggcggcggcggcggcggctcctcgtcttccccggccgcaacctcggcggccgcaacctcggccgccgccgccgctgccgccgccgccgccgccgccgccgccgccgcgggggccggggccccgtcggtgggggctgccggggcggcggacggcggcgacgagacgcggctgcctttccgccggggcatcgcgctgttggaaagcggctgcgtagacaacgtcctgcaagtcggtttccacttgagcggcacagtgacagaacctgcaatacaatcggagccagaaactgtttgcaacgtggccatcagctttgatcgttgcaagattacctcagtgacctgcagctgtggaaacaaggacatattttattgtgcccatgttgtggcactgtctttataccgcatccgcaagccagatcaggtcaaactgcatcttcctatttcagagactctctttcaaatgaatagagaccaactgcaaaagtttgtacagtatttgatcacagtgcaccacacagaagttttgccaactgctcaaaaattagcagatgaaattctttcccaaaattcagaaatcaaccaagttcatggtgctcctgatccaacagcaggtgctagtatagatgatgaaaactgctggcacttagatgaagagcaggttcaagaacaggttaaactgttcctttcccagggcgggtaccacggatcagggaagcagcttaatttgctctttgcaaaggtgcgggagatgttaaagatgagggactccaatggggcccgcatgttgaccttgataacagagcaattcatggctgaccctcgcctgtcactttggcggcaacaaggcactgcaatgactgacaaatacaggcagctctgggatgagctgggtgctctgtggatgtgtatagttttaaacccccactgcaagttggagcaaaaggccagttggctaaaacagctgaagaaatggaatagtgttgatgtctgtccatgggaagatggaaatcatggcagtgaattacccaacttaaccaatgctctgcctcagggtgcaaatgccaaccaagattcatcgaacaggccacatcggacagtgttcacccgagccatcgaggcatgcgatctccactggcaggatagccacttgcagcacattatcagcagtgacctatacaccaactactgttaccatgacgacactgaaaactccctcttcgactcccgcgggtggcccctctggcatgaacatgttcctacagcctgtgcaagagtggacgcattacgttctcatgggtaccccagagaagcactgagactagcaatagctattgttaatacattaagacgacagcagcagaaacagttggaaatgttccgaacccaaaaaaaagagctaccccataaaaacataacctcgataaccaatctggagggctgggttggacatcccctggaccctgtgggcactctcttcagtagccttatggaagcctgccgcattgatgatgagaacctctctgggttctcagattttacagagaatatgggacagtgcaagtctctggaataccagcatctacctgcacacaaattcttagaagaaggggaatcctatttaacgctggctgtggaagtagccctgatagggctaggacagcagcgtatcatgcctgatgggctgtacacacaagagaaagtttgccggaatgaggagcagctcatttctaagcttcaggaaattgaattggatgacacactggtgaaaatttttcgcaagcaagcagtcttcctattagaagccggaccatatagtggtttaggtgaaataatccatcgggagagcgttccaatgcacacatttgccaagtatctcttcacctctctcctacctcacgatgctgaattggcatacaaaattgcactgagagcaatgcggttactagtattggaatctactgctccatcaggagacctcacccgcccacaccacattgcatcagttgttcccaaccgctaccctcgctggttcactctaagccacattgagtcccagcagtgtgagctggcatccaccatgctaactgcagccaaaggcgatgttcggaggctggaaacagtattagaatccatccagaaaaacattcactcctcatcacacatcttcaagcttgcccaagatgcatttaaaatagcaactctcatggacagtttgccagacatcactcttttgaaagtgtctctggagctgggcctgcaggttatgcgaatgacactgtcaaccttaaattggcgacggcgggagatggtgaggtggctggtaacgtgtgctactgaagtcggggtttatgccctggacagcatcatgcagacctggtttacactctttactcccaccgaggccacaagtatagttgcaactaccgtgatgtccaacagcaccatcgtccgcctccacctggactgccaccagcaggaaaagctggccagcagcgcccggacacttgcactgcagtgtgccatgaaggatccacagaactgtgccctctctgcgctaaccctttgtgaaaaggatcacatagcttttgagacggcgtaccaaattgttctcgacgctgctacgactggcatgagctatacacagctctttacaatagcacggtacatggagcaccgcgggtaccccatgagggcctacaagctggccaccctggccatgacccatctcaacctgagctacaatcaggacacacaccctgccattaatgatgttttgtgggcctgtgcgcttagccactcccttggtaaaaatgagcttgcagctataatacctctggtggtcaagagtgtcaagtgtgcaacggtactgtcagacattttgcgcagatgcactctgaccactcctggcatggtgggacttcatgggaggaggaactctggtaagctcatgtcactggacaaagcccccttgaggcaactcttggatgccacgatcggggcctacatcaacacaacgcactcacggctcacacacatcagtcctcggcactatagtgagtttatagagttcctcagcaaagcccgagagaccttcttaatggcgcatgatggacacattcagtttacacagtttattgacaacctgaaacaaatctacaaaggcaaaaagaaactgatgatgttggttcgggagaggtttggttgatagatcttgtatgaatggggtggggggtggggatgggagggatggtttgtttttacttgagcctgcctttgtaccctttttaacttaaagaacagagccacaccggtattatatgtgtatagttatattgcgtttgcagactaaattgtcatgttgtgaaagtttgtgtgttttttattttttccctatttctttctttcctttattttattattttttttaattttttttttctggttttgtatgagagagaggttaaaaaggtttggtttacactgagtatatgttgtcaagtggcaaaagtccacatagctctcctgttttctgtatacgttcacagcctcaaaaaaaataattgaaatggctttaaaaaccaaacaaaacacctccatcctgtgataagtacctcgaatggattcagctttactcctttgtaactcatctttacattttcagcatatttaaacaaaccaacaaaatgaaatactaatagtaaaaaggctgacccatgtggctttgcagtgctgttcgtccagaagcatggcacacgatgcttgtgcatgtggaaacttagcgactgtcaacatacattctcagggatttatccaaaaaaattaaaaaaagaatgagagcatttattgtactgtatatatattatagtatatgtctgaatattgaaaatataacattaactaatttataaaaaatattctatgtaatgcaaaatacttgaagctgcagtagcttggttttaaacaaaaacaaaaaaaaaactgagagaaaacctatcagaaggactaaaagtacgccttgcttcagggttggctcaggtggtgaacttcatgctgggcatctatgcagagccaccttttggattgcatggttggactgagatctattgggagaaattatatatgtatatatatttatacaacttatgtatacatatatatatgtatacacacagacacacacacacaccaccaacaaccaccactacaccacacatgcttataacaggcactagaataaagagggacaacaaaatacacagccagagcagcaagccttagcattaagaatatacaatatgccggaattggggttcgtgcctcctagcctaggaaacttaaaagaaatatccttttgacacaaaacacaaaatgttttccaaaacaattgacataatgatacattacgcctttgcagtgagctaataataagctaacctttgtgcacaaataacattatatatattatatatctattctgcataggtattttgactttgtgcaggacagaaagttgtgtaggtatgactgttctacttttcagttttctttttttttaatatattttattttctctagaaattactcaaacaaaagcagccttctatcttgccttgtcttaatgctttaaaataaccaaactggagatctaactaccaaactgttcattatattattaaataccaactttggttacagtatagtgtctttactttagctgatggttctgtaaccttgtgctttttaaagcaatttttatgttttggtgcaaaagttgtccagtgtctcttgttcccttcactagagaacatgcttagaggtatgtttgtaggtatttttgtttagaagaactatttcatgcgctccattttatttattataataggtaaaaagaaaaaaaaaggttgtaattcatcacccatgtaaacatgctcatgaagttgaaagtaattaagatttgatacactgatatcaatttatttatgtaactaaatcactgttttataaacttgttaatgatcaacaatttttgttttgattaaaattagttttttgaaagttga